(19) United States
(12) Patent Application Publication
(10) Pub. No.: US 2026/0366032 A1
(43) Pub. Date: Nov. 19, 2026

(54) INDUCIBLE CONSTRUCTS

(71) Applicant: Outpace Bio, Inc., Seattle, WA (US)

(72) Inventors: Francoise J. HAESELEER, Seattle, WA (US); Robin L. KIRKPATRICK, Seattle, WA (US); Ty CROWL, Seattle, WA (US); Jared Louis HAMMER, Seattle, WA (US); Howell MOFFETT, Seattle, WA (US); Andrew Howen NG, San Francisco, CA (US); Paul Joseph SAMPLE, Seattle, WA (US)

(21) Appl. No.: 19/146,468

(22) PCT Filed: Jan. 9, 2024
(86) PCT No.: PCT/US2024/010926

§ 371 Date: Jul. 8, 2025

Related U.S. Application Data
(60) Provisional application No. 63/479,178, filed on Jan. 9, 2023.
(60) Provisional application No. 63/479,177, filed on Jan. 9, 2023.
(60) Provisional application No. 63/479,176, filed on Jan. 9, 2023.

(51) Int. Cl. C12N 15/63

(52) U.S. Cl. CPC C12N 15/635; C12N 2830/002; C12N 2830/34

(57) ABSTRACT

The disclosure provides inducible constructs comprising transcription initiators and/or transcription regulators. These constructs may be used to drive expression of genes, in response to particular cell states (such as in the context of antigen stimulation). In some embodiments, the constructs are incorporated as a part of regulatory circuits useful to control expression and regulation of cells expressing engineered receptors.

### CROSS-REFERENCE TO RELATED APPLICATIONS

This application claims priority to U.S. provisional patent application No. 63/479,176 filed on Jan. 9, 2023, U.S. provisional patent application No. 63/479,178 filed on Jan. 9, 2023, and U.S. provisional patent application No. 63/479,177 filed on Jan. 9, 2023, the contents of which are incorporated by reference in their entirety herein.

### REFERENCE TO SEQUENCE LISTING

The contents of the electronic sequence listing (OTPC_034_01WO_SeqList_ST26.xml; Size: 8,670,645 bytes; and Date of Creation: Jan. 3, 2024) are herein incorporated by reference in its entirety.

### BACKGROUND

Endogenous or unmodified exhausted immune cells (e.g., T cell) can lose their high proliferative capacity and cytotoxicity, eventually leading to cell death. Numerous factors can lead to immune cell exhaustion, such as persistent antigen exposure (e.g., during infection), and constant alloantigen presence following organ transplantation. In addition, some tumors and cancer-associated cells can actively induce cell exhaustion at the site of the tumor, thus conferring tumor protection from endogenous or unmodified T cell activity. Immune cell exhaustion is also seen in the context of engineered T cell therapies. Engineered immune cells expressing a chimeric antigen receptor (CAR) or T cell receptor (TCR) can undergo exhaustion in the same way that endogenous or unmodified T cells undergo exhaustion, thus diminishing their therapeutic efficacy.

There is an unmet need to increase the efficacy of engineered immune cell therapies by decreasing exhaustion and increasing persistence. The disclosure addresses this need by providing compositions and methods for delaying or reducing immune cell exhaustion.

### SUMMARY

The disclosure provides one or more inducible nucleic acid constructs, wherein each construct comprising a transcription initiator, or a transcription initiator and a transcription regulator. In some embodiments, the construct may be non-naturally occurring and each of the transcription regulator and the transcription initiator comprise a concatemer of transcription factor binding motifs for binding more than one unique transcription factor, or for binding the same transcription factor at more than one site. In some embodiments, the one or more nucleic acid constructs including the transcription initiators or transcription regulators may sense the cell state and may have sequences selected from any one or more sequences of SEQ ID NO: 175-9790. In another aspect provided herein is the use of the one or more nucleic acid constructs for selecting one or more sequences that respond to one or more cell states including antigen receptor activation, cell stimulation, cell exhaustion, cell rest, and epigenetic regulation.

Disclosed herein are nucleic acid constructs comprising a transcription initiator or the transcription initiator and a transcription regulator. In some embodiments, each of the transcription regulator and transcription initiator comprises a concatemer of transcription factor binding motifs for binding more than one unique transcription factor, or for binding the same transcription factor at more than one site, and wherein the construct is non-naturally occurring.

In some embodiments, the nucleic acid constructs comprise the transcription regulator and the transcription initiator. In some embodiments, the transcription regulator is located 5′ of the transcription initiator, or the transcription regulator is located 3′ of the transcription initiator.

In some embodiments, the construct does not comprise the combination of: a transcription regulator comprising an NFAT sequence, an IRF4 sequence, an AICE sequence, an ISRE sequence; and a transcription initiator comprising a human beta globin sequence. In some embodiments, the construct does not comprise the sequence of

(SEQ ID NO: 9786)
GGAGGAAAAACTGTTTCATACAGAAGGCGTGGAGGAAAAACTGTTTCATA
CAGAAGGCGTGGAGGAAAAACTGTTTCATACAGAAGGCGTGGAGGAAAAA
CTGTTTCATACAGAAGGCGTGAATTCAGGGCTGGGCATAAAAGTCAGGGC
AGAGCCATCTATTGCTTACATTTGCTTCTGACACAACTGTGTTCACTAGC
AACCTCAAACAGACACC.

In some embodiments, the construct does not comprise the combination of: a transcription regulator comprising an NFAT sequence; and a transcription initiator comprising a YB-TATA sequence.

In some embodiments, the construct does not comprise the sequence of

(SEQ ID NO: 9787)
GGAGGAAAAACTGTTTCATACAGAAGGCGTGGAGGAAAAACTGTTTCATA
CAGAAGGCGTGGAGGAAAAACTGTTTCATACAGAAGGCGTGGAGGAAAAA
CTGTTTCATACAGAAGGCGTGAATTCCTAGAGGGTATATAATGGGGGCCA
CTAGTCTACTACCAGAAAGCTTGGTACCGAGCTCGGATCCAGCCACC.

In some embodiments, the concatemer of the transcription regulator comprises two or more repeated sequences where the two or more repeated sequences comprise the same sequence, or do not comprise the same sequence.

In some embodiments, the concatemer of the transcription factor regulator binds more than one unique transcription factor, and/or binds the same transcription factor at more than one site.

In some embodiments, the concatemer comprises a linking sequence positioned between the repeated sequences and the linking sequence may comprise one or more of: TACGCT, TGATCT, TGCTTT, and TGCCCGT.

In some embodiments, the transcription regulator comprises a sequence isolated or derived from one or more of the untranslated genomic sequence of BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D.

In some embodiments, the transcription regulator comprises a sequence isolated or derived from one or more of the untranslated genomic sequence of NFAT, NFkB, REL, RELA, IRF2, GATA3 and ATF3.

In some embodiments, the transcription regulator comprises sequences derived from the untranslated genomic sequence of NFAT, optionally comprising a sequence of any one or more of sequences of SEQ ID NOS: 3430-3437, 3802-3803, 3940-3947, 4312-4313, 4808-4815, 4874-4889, 4298-4299, 4364-4379, 8576-8583, 8948-8949, 9444-9445, and 9510-9525.

In some embodiments, the transcription regulator comprises sequences derived from the untranslated genomic sequence of NFkB, optionally comprising a sequence of any one or more of sequences of SEQ ID NO: 550-553, 1060-1063, and 5696-5699.

In some embodiments, the transcription regulator comprises sequences derived from the untranslated genomic sequence of RELA, optionally comprising a sequence of any one or more of sequences of SEQ ID NO: 958-959, 3486-3501, 4426-4427, 4472-4473, 4484-4499, 1468-1469, 3996-4011, 4936-4937, 4982-4983, 4994-5009, 5697-5698, 8632-8647, 9572-9573, 9618-9619, and 9630-9645.

In some embodiments, the transcription regulator comprises sequences derived from the untranslated genomic sequence of IRF2, optionally comprising a sequence of any one or more of sequences of SEQ ID NO: 2384-2447, 4430-4461, 2894-2957, 4940-4971, 7530-7593, and 9576-9607.

In some embodiments, the transcription regulator comprises sequences derived from the untranslated genomic sequence of GATA3, optionally comprising a sequence of any one or more of sequences of SEQ ID NO: 1048-1079, 3566-3709, 1558-1589, 4076-4219, 6194-6225, and 8712-8855.

In some embodiments, the transcription regulator comprises sequences derived from the untranslated genomic sequence of ATF3, optionally comprising a sequence of any one or more of sequences of SEQ ID NO: 124-159, 3140-3147, 634-669, 3650-3657, 5179-5214, and 8286-8293.

In some embodiments, the transcription regulator is inducible or the construct is inducible.

In some embodiments, the transcription initiator includes a classical promoter, including a minimal promoter.

In some embodiments, the minimal promoter includes a sequence isolated or derived from one or more of minimal promoter-1 (“minP1”), YB-TATA and human beta globin.

In some embodiments, the minimal promoter comprises one or more of: MinP1 having a sequence of AGAGGGTATATAAAAGCTCGACTTCCAG (SEQ ID NO: 9788); MinP2=having a sequence of TAGAGGGTATATAATGGGGGCCACTAGTCTACTACCAGAAAGCTTGGTACCGAGCT CGGATCCAGCCACC (SEQ ID NO: 9789); and MinP3=having a sequence of

(SEQ ID NO: 9790)
CTAGAGGGTATATAATGGGGGCCACTAGTCTACTACCAGAAAGCTTGGTA
CCGAGCTCGGATCCAGCCACC.

In some embodiments, the transcription initiator comprises a transcription factor binding motif.

In some embodiments, the transcription initiator comprises a motif isolated or derived from the untranslated genomic sequence of a transcription factor or comprises a mammalian promoter sequence.

In some embodiments, the motif comprises a sequence isolated or derived from one or more of the untranslated genomic sequence of BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D.

In some embodiments, the transcription initiator comprises a sequence isolated or derived from one or more of the untranslated genomic sequence of NFAT, NFkB, REL, RELA, IRF2, GATA3 and ATF3.

In some embodiments, the transcription initiator comprises sequences derived from the untranslated genomic sequence of NFAT, optionally comprising a sequence of any one or more of sequences of SEQ ID NOS: 3430-3437, 3802-3803, 3940-3947, 4312-4313, 4808-4815, 4874-4889, 4298-4299, 4364-4379, 8576-8583, 8948-8949, 9444-9445, and 9510-9525.

In some embodiments, the transcription initiator comprises sequences derived from the untranslated genomic sequence of NFkB, optionally comprising a sequence of any one or more of sequences of SEQ ID NO: 550-553, 1060-1063, and 5696-5699.

In some embodiments, the transcription initiator comprises sequences derived from the untranslated genomic sequence of REL, optionally comprising a sequence of any one or more of sequences of SEQ ID NO: 958-959, 3486-3501, 4426-4427, 4472-4473, 4484-4499, 1468-1469, 3996-4011, 4936-4937, 4982-4983, 4994-5009, 5697-5698, 8632-8647, 9572-9573, 9618-9619, and 9630-9645.

In some embodiments, the transcription initiator comprises sequences derived from the untranslated genomic sequence of RELA, optionally comprising a sequence of any one or more of sequences of SEQ ID NO: 4484-4499, 4994-5009, and 9630-9645.

In some embodiments, the transcription initiator comprises sequences derived from the untranslated genomic sequence of IRF2, optionally comprising a sequence of any one or more of sequences of SEQ ID NO: 2384-2447, 4430-4461, 2894-2957, 4940-4971, 7530-7593, and 9576-9607.

In some embodiments, the transcription initiator comprises sequences derived from the untranslated genomic sequence of GATA3, optionally comprising a sequence of any one or more of sequences of SEQ ID NO: 1048-1079, 3566-3709, 1558-1589, 4076-4219, 6194-6225, and 8712-8855.

In some embodiments, the transcription initiator comprises sequences derived from the untranslated genomic sequence of ATF3, optionally comprising a sequence of any one or more of sequences of SEQ ID NO: 124-159, 3140-3147, 634-669, 3650-3657, 5179-5214, and 8286-8293.

In some embodiments, the transcription initiator comprises the sequence of any one or more of SEQ ID NOS: 175-9781, or concatemers of the same.

In some embodiments, the construct further comprises an integration sequence capable of inducing or increasing insertion of the construct into a target site.

In some embodiments, a vector or a chromosome comprises the target site.

In some embodiments, the integration sequence comprises a first or 5′ integration sequence and a second or 3′ integration sequence.

In some embodiments, the integration sequence comprises a homologous sequence corresponding to an insertion site to facilitate homologous recombination.

In some embodiments, the integration sequence comprises a transposable element to facilitate transposition, and in some embodiments, the transposable element comprises an insertion sequence and/or a transposon sequence.

In some embodiments, the integration sequence comprises a first 5′ terminal repeat sequence or a second 3′ terminal repeat sequence.

In some embodiments, the integration sequence comprises a first 5′ terminal repeat sequence and a second 3′ terminal repeat sequence.

In some embodiments, the first 5′ terminal repeat sequence or the second 3′ terminal repeat sequence comprises a long terminal repeat (LTR) sequence.

In some embodiments, the first 5′ terminal repeat sequence or the second 3′ terminal repeat sequence comprises an inverted terminal repeat (ITR) sequence.

In some embodiments, the construct further comprises a reporter sequence.

In some embodiments, the reporter sequence comprises a fluorescent protein, including a green fluorescent protein (GFP).

Also disclosed herein is a vector that may include the construct as described above. The vector may include a mammalian expression vector capable of expressing the construct in a mammalian cell. The vector may also include an expression vector capable of expressing the construct in a human cell. In some embodiments, the vector may include a delivery vector capable of delivering the construct to a mammalian cell or a human cell

Also disclosed herein is a library that may include a construct as described above or a vector as described above.

In some embodiments, the library may include a plurality of constructs as described above or a plurality of vectors as described above.

Also disclosed herein is a cell including a construct as described above, a vector as described above, or a library as described above. In some embodiments, the cell is an immune cell, a stem cell, a progenitor cell, a precursor cell, or a naive cell, a differentiated cell, a T-cell, a B-cell, a macrophage, or an NK cell, in vitro or ex vivo, a primary cell, or a cultured cell.

Also disclosed herein is a use of a construct as described above, a vector as described above; a library as described above; or a cell as described above, for selecting a sequence that responds to one or more of antigen receptor activation, cell stimulation, cell exhaustion, cell rest, and epigenetic regulation. In some embodiments, cell stimulation may be early stimulation, late stimulation.

In some embodiments, the antigen receptor is a chimeric antigen receptor.

In some embodiments, the cell is a T cell and wherein the antigen receptor is an endogenous or exogenous T cell receptor (TCR).

Also disclosed herein is use of a construct as described above, a vector as described above; a library as described above; or a cell as described above, for regulating transcription of an exogenous protein, in response to one or more of antigen receptor activation, cell stimulation, cell exhaustion, cell rest, and epigenetic regulation.

In some embodiments, the exogenous protein is one or more of an engineered receptor, a cytokine, or a degrader.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawing wherein:

FIG. 1A depicts a schematic of an exemplary CAR degradation feedback loop initiated by the signaling through the CAR.

FIGS. 1B-1D depict graphs of proposed expression over time of a CAR and a degrader in an immune cell in the presence of antigen stimulation, where the expression of the degrader is driven by a cell state of, or nearing, exhaustion.

FIG. 2 is a schematic diagram depicting exemplary patterns of activity induced by constructs of the disclosure in response to either (1) pulsed T-cell stimulation (left-hand panel) or (2) sustained T-cell stimulation sufficient to allow T-cell exhaustion to present (right-hand panel). Each of the pulsed stimulation and the sustained stimulation (with or without exhaustion) are useful transcription dynamics of the inducible constructs of the disclosure. As shown in the exemplary pulsed stimulation condition in the left-hand diagram, activity of the “stim-ON” promoter is lower until the T-cell is stimulated by antigen, at which time activity increases transiently. In the exemplary pulsed stimulation condition, the “stim-ON” activity of the promoter will decrease as the antigen available to the T cell is removed, but will increase again once the antigen stimulation is repeated. As shown in the exemplary pulsed stimulation condition in the left-hand diagram, activity of the “stim-OFF” promoter is higher until the T-cell is stimulated by antigen, at which time activity decreases transiently. In the exemplary pulsed stimulation condition, the “stim-OFF” activity of the promoter will increase as the antigen available to the T cell is removed, but will decrease again once the antigen stimulation is repeated. As shown in the sustained stimulation condition on the right-hand diagram, the activity of the “stim-ON” promoter is lower until the T-cell is stimulated by antigen, at which time activity increases. As shown in the sustained stimulation condition on the right-hand diagram, the activity of the “stim-OFF” promoter is higher until the T-cell is stimulated by antigen, at which time activity decreases. In the sustained stimulation condition on the right-hand diagram, the T-cell may undergo exhaustion after some period of stimulation. In some embodiments of the disclosure, an activity of a stim-ON promoter may be induced at or before the initiation of T-cell exhaustion to prevent or to delay the onset of exhaustion. In some embodiments of the disclosure, an activity of a stim-OFF promoter may be reduced at or before the initiation of T-cell exhaustion to prevent or to delay the onset of exhaustion.

FIG. 3 is a schematic diagram showing a series of exemplary constructs used in a Massively Parallel Reporter Assay (MPRA) (top panel) and the experimental design for collecting and sequencing RNA and DNA at each timepoint for the no-stimulation, stimulation, and rest conditions tested. RNA-seq was used to measure transcription and DNA-seq was used to normalize for Enhancer-Promoter (EP)-pair copy number. Approximately one million combinations of fifty thousand distinct enhancer sequences were use to form unique combinations with nine thousand promoter sequences in the format of the construct depicted in this figure. The library of over one million constructs was tested functionally to identify constructs with inducible patterns of activity under conditions or induced cell states, such as stimulation, no stimulation, pulsed stimulation, sustained stimulation, rest and exhaustion. Briefly, the MPRA included a thaw of T-cells at day 0, transduction of the thawed cells with a “tool” CAR at day 1 and CD19+ enrichment at day 4 (see Example 1 for detailed protocols). Each of the Stimulation or No Stimulation conditions began at day 7, relative to Day 0 thaw. The rest condition began at day 14, relative to the Day 0 thaw. Abbreviations: long terminal repeat (LTR), barcode (BC), 5′ untranslated region (UTR), green fluorescent protein (GFP).

FIG. 4 is a series of graphs depicting four exemplary activity patterns of four EP Pairs from a library comprising hundreds of thousands of EP Pairs. In each plot the activity of an exemplary construct is shown as the transcription rate (left) or log transcription rate (right) as a function of time (expressed in days).

FIG. 5 is a series of graphs depicting exemplary activity patterns of EP Pairs selected for deeper characterization. From the over one million constructs, approximately 174 constructs were selected for further analysis. In each plot the activity of an exemplary construct is shown as the transcription rate (left) or log transcription rate (right) as a function of time (expressed in days).

FIG. 6A-B are a pair of schematic diagrams depicting the functional characterization of EP-Pairs. FIG. 6A depicts the experimental timeline for the procedure described in FIG. 6B. Experimental protocol is provided in Example 1. This initial screen was designed to (1) determine which EP-Pairs can drive protein expression and (2) roughly classify EP-pairings into categories of stim-ON, stim-OFF and oscillatory based on qualitative behavior patterns.

FIG. 7 is a series of graphs depicting the activity of either control constructs or constructs comprising EP-Pairs of the disclosure under both stimulation and no stimulation conditions. For each plot, normalized fluorescence (shown in arbitrary units of fluorescence) and confluence (shown as a percent) are shown as a function of time (measured in days). With respect to the Control constructs, MND activity is known to be constitutive whereas the activity of concatemerized NFkB (10×NFkB) has been shown in this disclosure to be inducible in the presence of antigen and, therefore, to follow a Stim-ON pattern. With respect to the EP-Pair constructs, low confluence in this experiment made it difficult to identify constructs that are active under no stim conditions. However, several EP-pair constructs were observed to have inducible activity under stimulation conditions.

FIG. 8A-C is a series of plots showing the dynamic range of EP-Pairings characterized according to the protocol described in FIG. 6A-B. FIG. 8A shows the maximal expression of each construct in this study, with annotations to the control constructs of MND and 10×NFkB. FIG. 8B shows the minimal expression of each construct in this study, with annotations to the control constructs of MND and 10×NFkB. FIG. 8C shows the range of expression of each construct in this study, with annotations to the control constructs of MND and 10×NFkB. In some embodiments, a construct with a greater dynamic range, including minimal expression under no stimulation conditions, is a desirable construct for stim-ON circuits.

FIG. 9 is a series of diagrams depicting both RNA and protein produced from exemplary constructs comprising an EP-pairing of the disclosure. As shown in the right-hand panel, not all RNA and protein data align qualitatively. However, each construct of the disclosure can be functionally characterized with respect to transcriptional activity as a function of cell activity (e.g., contacting an antigen) or cell state (e.g., stimulation and/or exhaustion). In some embodiments, the presence or absence of a 5′ UTR sequence, or the composition of the 5′ UTR sequence, affects the translational behavior of a transcript produced under the control of a construct of the disclosure.

FIG. 10 is a repeat of the study performed in FIG. 9. Of note, with higher confluency compared the study of FIG. 9, this study provides data for the no-stimulation condition.

FIG. 11 is a pair of schematic diagrams depicting a pooled screen of constructs comprising EP-pairs of the disclosure (see SEQ ID Nos 1-174), each driving expression of a “tool” CAR in a T-cell (top) and an experimental design for the screen (bottom). Control constructs include the constitutive MND promoter and the inducible 10×NFkB promoter.

FIG. 12 is a pair of graphs showing the selection of candidate constructs for enrichment, based, in part, upon the negative selection of exhausted T-cells (identified as being CD39+TIGIT+, in this embodiment).

FIG. 13 is a schematic diagram depicting the experimental design of a confirmational, three donor, assay using the best performing EP-pairs identified in a preliminary screen using a single donor only. In the preliminary screen, the test constructs included 26 constructs comprising EP-Pairs driving a tool CAR construct, and four control constructs (MND and 10×NFkB promoters driving expressing of a tool CAR, each control promoter with and without a barcode). The preliminary screen was designed to identify those constructs that demonstrated most efficacious killing of a target cancer cell in vitro, compared to the activity of each of the control constructs. In this confirmational screen, 9 constructs comprising selected EP-Pairs driving a tool CAR construct (see FIGS. 14 and 15) and 2 control constructs were used (same as in the preliminary screen).

FIG. 14 is a series of graphs depicting target cell killing data from each round of the confirmational study described in FIG. 13. In each round, the ability of each construct to drive expression of a tool CAR and to kill a target cancer cell (H1975 mKate cells) is expressed as the target cell count normalized to time=zero as a function of time (expressed in hours). The data demonstrate that the EP Pairing of “OTP-CSF2” shows comparable killing to the construct comprising the control 10×NFkB promoter constructing driving expression of the same CAR. The data further show that the EP Pairing of “RPS6KA1-RSRP1” may be used a regulated alternative to the constitutive control promoter, MND.

FIG. 15 is a series of graphs depicting cytokine secretion (IL-2 and Interferon-gamma) from T-cells expressing each of the test constructs described in FIG. 13, in each of the three donors. The data demonstrate that the EP Pairings of “OTP-CSF2” and “RPS6KA1-RSRP1” show at least comparable functional activity to the control constructs having the MND and 10×NFkB promoters, respectively. Moreover, when the EP Pairing of “OTP-CSF2” drives expression of the CAR, the T-cell demonstrates higher IL-2 expression than when the MND control drives expression of the same CAR. The EP Pairing of “RPS6KA1-RSRP1” demonstrates higher overall cytokine expression than the other seven constructs tested, which is consistent with the target cell killing data presented in FIG. 14.

FIG. 16A-B is a series of graphs depicting CAR expression after one round of antigen stimulation for each of the control constructs (comprising MND and 10×NFkB, respectively) and two test constructs (comprising the EP Pairing of “OTP-CSF2” and “RPS6KA1-RSRP1” respectively). FIG. 16A shows a series of cell sorting plots for both unstimulated (top) and stimulated (+H1975-mKate target cells, bottom), in which the levels of the CD19 marker are represented on the Y-axis and the levels of CAR expression are represented on the X-axis. See FIG. 11 for a schematic of the constructs/markers used. Shown in this figure is representative data from Donor 2. FIG. 16B is a graph showing the aggregate and quantitative data from all three donors in this study. For each condition and each construct, the data represent the Mean z SEM % tCD19+CAR+ across 3 donors. The data, taken together, demonstrate that (1) the “OTP-CSF2” construct shows Stim ON activity after 1 round of passage stimulation and that (2) “RPS6KA1-RSRP1” has different expression dynamics than the MND control construct, but a similar functional readout as the MND control construct.

FIG. 17 is a table summarizing exemplary activities for exemplary constructs of the disclosure. For example, NFkB (either single or contatemerized to 10×NFkB) as well as “OTP-CSF2” both demonstrate Stim-ON activity, meaning that these promoters drive expression in response to T-cell stimulation or in response to a stimulated cell state of a T-cell. More specifically, both NFkB and “OTP-CSF2” demonstrate early Stim-ON activity, meaning that they respond quickly to T-cell stimulation or respond quickly following the T-cell adoption of a stimulated cell state. Early Stim-ON and late Stim-ON are sub-categories of Stim-ON behavior with the primary difference being a time delay in the onset of activity from late Stim-ON constructs compared to early Stim-ON constructs. Of note, the data provided in this disclosure identify at least one Stim-OFF construct, comprising an IRF2 promoter sequence.

FIG. 18 is a pair of schematic diagrams and a corresponding graph depicting exemplary uses for the constructs of the disclosure within cellular circuits. The data demonstrate that, under chronic stimulation conditions in vitro, the NFkB-regulated MSLN CAR shows improved killing and cytokine production when compared to a the same MSLN CAR under the control of MND constitutive expression.

### DETAILED DESCRIPTION

Immune cells play a role in immune response. For example, T cells, a type of lymphocyte, play a central role in the adaptive immune response. One of the functions of T cells is immune-mediated cell death. CD8+ T cells, also known as killer T cells, are themselves cytotoxic, and also recruit other cell types to kill cancer cells and virus-infected host cells. CD4+ T cells, also known as helper T cells, further activate memory B cells and killer T cells through cytokines to enhance the immune response. Regulatory T cells, also known as suppressor T cells, allow immune cells to recognize invading cells from “self,” thus preventing an autoimmune response.

Over time, T cells undergo a physiological change called exhaustion, characterized by progressive loss of function and changes in transcriptional profiles. T cell exhaustion is mediated by a cascade of signaling molecules culminating in transcription factors that regulate the expression of genes that modulate T cell exhaustion. Exhausted T cells lose their high proliferative capacity and cytotoxicity, eventually leading to cell death. Numerous factors can lead to T cell exhaustion, such as persistent antigen exposure during infection, and constant alloantigen presence following organ transplantation. In addition, some tumors and cancer-associated cells can actively induce T cell exhaustion at the site of the tumor, thus conferring tumor protection from T cell activity. T cell exhaustion is also seen in the context of engineered T cell therapies. Engineered T cells expressing a chimeric antigen receptor (CAR) or T cell receptor (TCR) can undergo exhaustion in the same way that endogenous T cells undergo exhaustion, thus leading to a decrease in persistence.

Likewise, other engineered immune cells, such as engineered NK cells, engineered B cells, and engineered macrophages can undergo exhaustion, and face a similar issue.

### Cell State

Provided herein are constructs that sense (e.g., are responsive to) cell states. The constructs are useful in any context where cell state sensing is desired; in some embodiments the constructs are particularly useful in regulating the expression of exogenous engineered receptors in therapeutic immune cells, so as to decrease exhaustion and increase persistence of the immune cell therapies.

An immune cell, for example a T cell, may demonstrate multiple cell states including an unstimulated state (e.g., before encountering antigen), a stimulated state (e.g., after and during encountering antigen), an exhausted state (e.g., after prolonged stimulation), and a rested state (e.g., after stimulation stops for a period of time). In some embodiments, other cell states may include, but are not limited to, a homeostatic or healthy cell state, a mitotic cell state, a migratory cell state, an apoptotic cell state, a necrotic cell state, a stem or stem-like cell state, a senescent cell state, or the like. In some embodiments, cell states may be organized into different categories including but not limited to functional states, activity states, nutritional states, developmental/maturation states, differentiation states, or the like. In some embodiments, a cell may be defined as having the features of a plurality of cell states.

For example, an engineered T cell expressing a chimeric antigen receptor (CAR T cell), undergoing prolonged antigen stimulation, may be in an exhausted cell state. In such embodiments, one or more constructs of the disclosure, if included in the engineered T cell, along with the CAR, can sense the exhausted cell state of the CAR T cell, wherein the CAR T cell has undergone prolonged antigen stimulation. In some embodiments, the one or more constructs of the disclosure may sense the exhausted cell state of the CAR T cell and lead to transcription of one or more genes through one or more transcription initiators of the one or more constructs by which exhaustion is managed, and persistence increases, as will be described in more detail herein. In some embodiments, the one or more genes whose transcription is regulated may include a degrader, and the transcription of the degrader can lead to a reduction in levels of activated CARs of the cell, leading to a change in the cell state by allowing for rest period, potentially leading to a chance in the cell state from exhausted to “rested”.

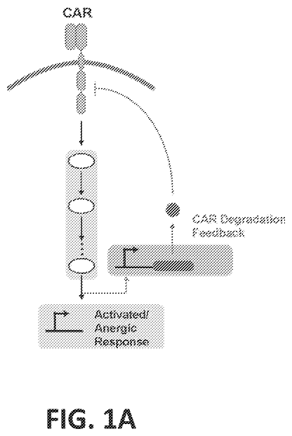

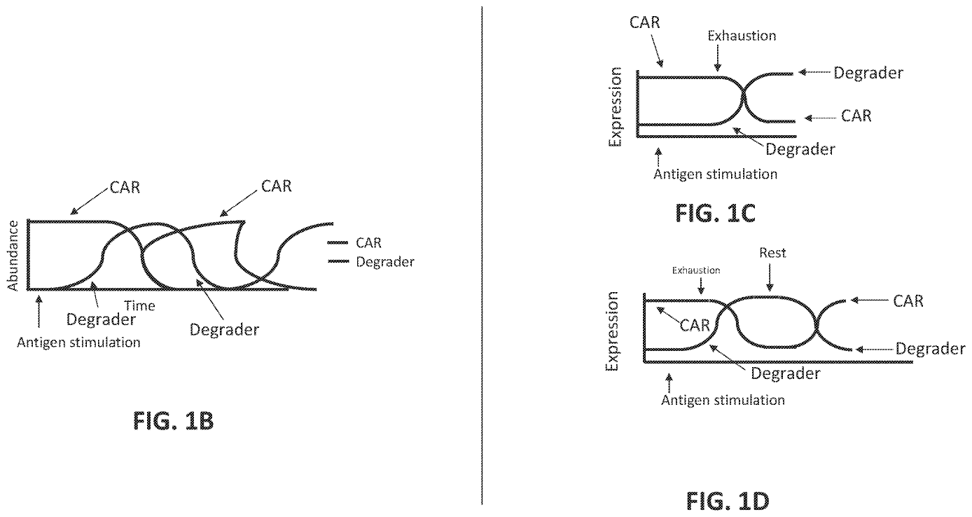

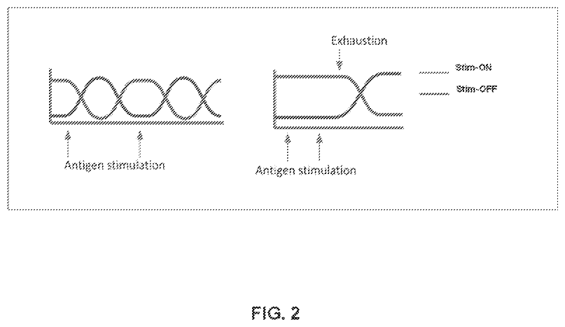

FIG. 1A depicts a schematic of a chimeric antigen receptor (CAR) expressing immune cell. The CAR is stimulated in the presence of an antigen leading to an activation of the immune cell. In some embodiments, the activated response includes upregulation of one or more transcription factors, thus increasing the transcription of a cascade of transcription factor-dependent genes. With prolonged antigen stimulation, the activation can give way to exhaustion of the CAR immune cell. However, the introduction of a feedback loop may be desirable to regulate the response to activation. In some embodiments, the feedback loop may include a CAR degradation feedback loop. The CAR degradation feedback loop may include the upregulation of a polypeptide capable of degrading (e.g., a “degrader” polypeptide) the activated CAR to reduce the activated response. In some embodiments, the CAR degradation feedback loop may include a construct wherein transcription of one or more genes of the construct may be initiated by the signaling through the CAR, as depicted in FIG. 1A. In some embodiments, the construct may include one or more transcription initiators or one or more transcription initiators and one or more transcription regulators that drive transcription of one or more genes of the construct in response to cell state to reduce the activated response of the cell, as will be described in more detail herein.

In some embodiments, the CAR degradation feedback loop may include a degrader, a peptide configured to degrade the activated CAR. FIGS. 1C-1D depict graphs of predicted expression of a CAR and a degrader in response to antigen stimulation using the cell state sensing constructs of the disclosure. Without being held to theory or mechanism, antigen stimulation may lead to high expression of the CAR and an activated response within the immune cell. In the presence of continual antigen stimulation, the immune cell may approach an exhausted cell state, as seen in FIG. 1C. An exhausted cell state may lead to a reduction in efficiency and cell viability of the T cell and a reduction in the expression of the CAR. The activated response within the immune cell may lead to an increase in expression of the degrader. However, if the exhaustion of the CAR immune cell occurs before the increase in expression of the degrader, the CAR immune cell is exhausted, and the presence of the degrader may not have an effect. However, if the exhaustion cell state may be sensed, leading to an increase in transcription of the degrader before the CAR immune cell is exhausted, as seen in FIG. 1D, the CAR may be degraded before the CAR immune cell is exhausted, inducing a cell state of rest (e.g., a length of time wherein the CAR immune cell is not activated), prolonging cell viability. This length of time (e.g., rest) that the CAR immune cell is in a rested cell state may let the CAR immune cell to recover from excessive antigen stimulation before additional antigen stimulation leads to an activated response by the CAR immune cell after rest. Ultimately, a reduction in activation would lead to a reduction in levels of the degrader, and an increase in CAR expression levels.

Taken more broadly, the transcriptional control of the engineered receptor (e.g., CAR or TCR) degradation feedback loop may be engineered to respond to cell state. In some embodiments, one or more constructs may be engineered or generated to provide transcriptional control of one or more genes (e.g., degraders, CARs, or cytokines) in response to cell state. In some embodiments, the one or more constructs may provide transcriptional control of the one or more genes in response to cell state within a CAR T cell.

Dynamic transcriptional control may be controlled through timing of expression, expression dynamics, expression level, or responsiveness to cell state. This dynamic transcriptional control may occur through interactions between transcription initiator promoter sequences and transcription regulator enhancer sequences. FIG. 1D depicts a schematic of construct comprising a transcription initiator promoter sequence operably linked to a gene and one or more transcription regulators. In some embodiments, the transcription regulators may include regulatory elements (“CRE”) including enhancers, insulators, silencers, suppressors, or the like. For example, when a transcription initiator includes a promoter and transcription regulator includes an enhancer, the enhancer may increase transcription through the promoter of a gene operably linked to the promoter.

### Cell State Sensing Constructs

The nucleic acid constructs of the disclosure comprise a transcription initiator or comprise a transcription initiator and a transcription regulator, wherein each of the transcription regulator and transcription initiator comprises a concatemer of transcription factor binding motifs, and wherein the construct is non-naturally occurring. The concatemers comprise sequences that may be derived from known promoter sequences. The concatemer of transcription factor binding motifs (referred to in the Examples as concatemerized sequences) either bind more than one unique transcription factor or bind the same transcription factor at more than one site It can be appreciated that non-naturally occurring sequences may include naturally occurring sequences (including those isolated from or derived therefrom) assembled in a non-naturally occurring way. For example, in some embodiments, one or more naturally occurring sequences that are not adjacent within the genome may be placed adjacent in the construct, thus being assembled in a non-naturally occurring way.

As used herein, a transcription initiator of the disclosure behaves in a promoter-like fashion and supports binding of a transcription factor to initiate transcription of a downstream under its control. As used herein, a transcription regulator of the disclosure further regulates the transcription of the downstream gene. In some embodiments the transcription regulator is located 5′ of the transcription initiator. In some embodiments, the transcription regulator is located 3′ of the transcription initiator.

Transcription binding motifs may be derived from the untranslated genomic regions of the following non-exhaustive list of proteins: BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D.

In some embodiments, when the transcription initiator comprises one or more sequences from the untranslated genomic sequence of NFAT: in some embodiments, the transcription regulator may comprise a sequence of any one or more sequences of SEQ ID NO: 3430-3437, 3802-3803, 3940-3947, 4312-4313, 4808-4815, 4874-4889, 4298-4299, 4364-4379, 8576-8583, 8948-8949, 9444-9445, and 9510-9525.

In some embodiments, the transcription initiator comprises one or more sequences from the untranslated genomic sequence of NFkB; in some embodiments, the transcription regulator may comprise a sequence of any one or more sequences of SEQ ID NO: 550-553, 1060-1063, and 5696-5699.

In some embodiments, when the transcription initiator comprises one or more sequences from the untranslated genomic sequence of REL (including RELA or RELB): in some embodiments, the transcription regulator may comprise a sequence of any one or more sequences of 958-959, 3486-3501, 4426-4427, 4472-4473, 4484-4499, 1468-1469, 3996-4011, 4936-4937, 4982-4983, 4994-5009, 5697-5698, 8632-8647, 9572-9573, 9618-9619, and 9630-9645.

In some embodiments, when the transcription initiator comprises one or more sequences from the untranslated genomic sequence of IRF2; in some embodiments, the transcription regulator may comprise a sequence of any one or more sequences of SEQ ID NO: 2384-2447, 4430-4461, 2894-2957, 4940-4971, 7530-7593, and 9576-9607.

In some embodiments, when the transcription initiator comprises one or more sequences from the untranslated genomic sequence of GATA3: in some embodiments, the transcription regulator may comprise a sequence of any one or more sequences of SEQ ID NO: 1048-1079, 3566-3709, 1558-1589, 4076-4219, 6194-6225, and 8712-8855.

In some embodiments, when the transcription initiator comprises one or more sequences from the untranslated genomic sequence of ATF3; in some embodiments, the transcription regulator may comprise a sequence of any one or more sequences of SEQ ID NO: 124-159, 3140-3147, 634-669, 3650-3657, 5179-5214, and 8286-8293.

In some embodiments, the construct includes a transcription initiator. In other embodiments, the one or more exemplary constructs may include a transcription initiator and a transcription regulator, as will be described in more detail herein. In some embodiments, the one or more constructs may also include an integration sequence capable of inducing or increasing insertion of the construct into a target site, e.g., of a vector or a chromosome. The insertion sequence may comprise multiple insertion sequences. For example, in some embodiments, the insertion sequence may comprise a first integration sequence and a second integration sequence. In some embodiments, the first integration sequence may be a 5′ integration sequence and the second integration sequence may be a 3′ integration sequence. In some embodiments, the insertion sequence may include a homologous sequence that corresponds to an insertion site in order to facilitate homologous recombination. In some embodiments, the integration sequence may include one or more transposable elements that may help facilitate transposition. In some embodiments, the transposable element may include an insertion sequence and/or a transposon sequence. In some embodiments, the integration sequence may comprise a first 5′ terminal repeat sequence or a second 3′ terminal repeat sequence. In some embodiments, the construct may be inducible.

In some embodiments, the construct does not include a transcription initiator comprising a human beta globin sequence and a transcription regulator comprising any of a Nuclear factor of activated T-cells (NFAT) sequence, a Interferon Regulatory Factor 4 (IRF4) sequence, a activating protein 1 (AP-1)-IRF composite elements (AICE) sequence, or a Interferon Stimulation Response Element (ISRE)sequence. In some embodiments, the AICE sequence may include or encompass sequences of or derived from IRF4 or IRF8, each with BATF. In some embodiments, the AICE sequences may be derived from the untranslated region of IRF4, IRF8, and/or BATF. In some embodiments, the IRSE sequence may include a consensus sequence of YAGTTTC(A/T)YTTTYCC” in which “Y” is either C or T. In some embodiments, the construct does not include a sequence comprising SEQ ID NO: 9786.

Also in some embodiments, the construct does not include a transcription initiator comprising a YB-TATA sequence and a transcription regulator comprising a NFAT sequence. In some embodiments, the construct does not include a sequence comprising SEQ ID NO: 9787.

Various constructs may demonstrate dynamic behavior over time in response to antigen stimulation. In some embodiments, the constructs that display similar dynamic behavior may be classified into similar behavior profiles. For example, constructs may demonstrate constitutive behavior wherein antigen stimulation leads to transcription driven from the transcription initiator wherein transcription level stays relatively consistent, regardless of continued antigen stimulation. In some embodiments, constructs may demonstrate “stimulation on” behavior wherein antigen stimulation leads to a marked increase in transcription driven from the transcription initiator. In some embodiments, the “stimulation on” behavior may cause transcription levels to stay high after initial antigen stimulation leads to transcription driven from the transcription initiator. In some embodiments, “stimulation on” behavior may include wherein initial antigen stimulation leads to a marked increase in transcription driven from the transcription initiator (referred to herein as “early stimulation on” behavior) or prolonged initial antigen stimulation leads to a marked increase in transcription driven from the transcription initiator (referred to herein as “late stimulation on” behavior). In some embodiments, the “stimulation on” behavior may also include wherein antigen stimulation leads to a single instance of increased transcription driven from the transcription initiator. In some embodiments, the single instance of increased transcription driven from the transcription initiator may be due to epigenetic regulation (e.g., DNA methylation/de-methylation, histone modifications including acetylation, methylation, phosphorylation, and ubiquitination, or the like), other epigenetic like transcriptional changes or cell regulatory or transcriptional pathways. In other embodiments, the “stimulation on” behavior may also include wherein antigen stimulation leads to multiple instances of increased transcription driven from the transcription initiator. In some embodiments, transcription initiators may demonstrate “stimulation off” behavior wherein initial antigen stimulation leads to a decrease in transcription driven from the transcription initiator. In some embodiments, the “stimulation off” behavior may cause transcription level to stay lower after initial antigen stimulation including transcription levels near or about zero.

In some embodiments, the one or more nucleic acid constructs may be inducible. In some embodiments, the one or more inducible nucleic acid constructs may comprise one or more sequences that may regulate and initiate transcription of a gene, wherein the construct comprises one or more non-naturally occurring sequences. In some embodiments, non-naturally occurring sequences may include sequences that are isolated or derived from naturally occurring sequences or non-naturally occurring sequences. It can be appreciated that non-naturally occurring sequences include naturally occurring sequences (including those isolated from or derived therefrom) assembled in a non-naturally occurring way. For example, in some embodiments, one or more naturally occurring sequences that are not adjacent within the genome may be placed adjacent in the construct, thus being assembled in a non-naturally occurring way.

In some embodiments, the construct may include a transcription initiator and a transcription regulator, and in some embodiments the transcription initiator and the transcription regulator may be engineered to respond to cell state or changes in cell state, as will be described in more detail herein. For example, one construct may include a MND-transcription initiator (e.g., promoter) operably linked to a CAR. The MND-transcription initiator may be constitutively active, wherein antigen stimulation leads to expression of the CAR. A second construct may include a transcription regulator adjacent to a transcription initiator operably linked to a degrader marked by a reporter gene. Antigen stimulation of the CAR leads to an activated response wherein endogenous signaling networks may lead to activation of transcription of the second construct including the degrader. The second construct may “sense” the cell state (e.g., activated through antigen stimulation) or change in cell state leading to transcription of the degrader.

### Exemplary Transcription Initiators

In some embodiments, the transcription initiator of the construct may comprise a nucleotide sequence that initiates transcription of a gene. In some embodiments, it is envisioned that the transcription initiators of the constructs of the disclosure may comprises sequences that are reminiscent of prototypical or classical promoters that have been previously characterized. However, in other embodiments, it is envisioned that the transcription initiator may comprise sequences that are not reminiscent of prototypical or classical promoters but nonetheless support the initiation of transcription and may demonstrate promoter-like function. In some embodiments, the transcription initiator may comprise one or more transcription factor binding motifs. In some embodiments, the transcription factor binding motifs may be isolated or derived from the untranslated regions of a transcription factor or from another mammalian promoter sequence. The transcription initiators may be engineered in that, the sequences have been optimized to drive transcription of a gene. In some embodiments, the transcription initiator may be inducible.

In some embodiments, the transcription initiator may comprise a minimal promoter sequence. The minimal promoter sequence may comprise the minimal number of base pairs within a classical or non-classical promoter to still drive transcription of a gene from that promoter. In some embodiments, the minimal promoter may comprise a sequence isolated or derived from one or more of minimal promoter-1 (“minP1”), YB-TATA, and human beta globin. minP1 may comprise a sequence of: AGAGGGTATATAAAAGCTCGACTTCCAG (SEQ ID NO: 9788). In some embodiments, the minimal promoter may comprise a sequence isolated or derived from minimal promoter 2 (“minP2”) or minimal promoter 3 (“minP3”). In some embodiments, minP2 may comprise a sequence of TAGAGGGTATATAATGGGGGCCACTAGTCTACTACCAGAAAGCTTGGTACCGAGCT CGGATCCAGCCACC (SEQ ID NO: 9789). In some embodiments, minP3 may comprise a sequence of

(SEQ ID NO: 9790)
CTAGAGGGTATATAATGGGGGCCACTAGTCTACTACCAGAAAGCTTGGTA
CCGAGCTCGGATCCAGCCACC.

In some embodiments, the transcription initiator of the construct may be engineered to comprise a concatemer of one or more nucleic acid sequences. In some embodiments, the transcription initiator may comprise a concatemer of any sequence including any sequence of SEQ ID NOS: 175-509, 9793-9800. In some embodiments, the concatemers (e.g., the transcription initiator of the construct) may comprise any sequence including any sequence of SEQ ID NOS: 5147-9790. In some embodiments, the concatemers (e.g., the transcription initiator of the construct) may advantageously drive transcription at a higher rate compared to non-concatemerized transcription initiators. In some embodiments, the concatemer may comprise two or more repeated sequences. The concatemers may be comprised of multiple repeated sequences (e.g., multiple repeats). In some embodiments, the concatemer may comprise at least about 3 repeats, at least about 4 repeats, at least about 5 repeats, at least about 6 repeats, at least about 7 repeats, at least about 8 repeats, at least about 9 repeats, at least about 10 repeats, at least about 11 repeats, at least about 12 repeats, at least about 13 repeats, at least about 14 repeats, or at least about 15 repeats. In some embodiments, the overall length of the concatemer may comprise a length of between about 20 base pairs and about 200 base pairs. In some embodiments, the overall length of the concatemer may comprise a length of no more than 150 base pairs. For example, the overall length of the concatemer may be about 125 base pairs, about 130 base pairs, about 133 base pairs, about 134 base pairs, about 135 base pairs, about 136 base pairs, about 139 base pairs, about 140 base pairs, about 142 base pairs, about 145 base pairs, about 146 base pairs, about 147 base pairs, about 149 base pairs, or about 150 base pairs. The concatemer may comprise a linking sequence positioned between each of the repeated sequences. In some embodiments, the linking sequences may comprise a sequence of one or more of: TACGCT, TGATCT, TGCTTT, or TGCCCGT. In some embodiments, the linking sequence between each repeated sequence may be the same linking sequence or may be a different linking sequence.

In some embodiments, the two or more repeated sequences of the construct may comprise transcription factor binding motifs or motifs derived from or isolated from untranslated genomic sequence of one or more transcription factors or one or more mammalian promoter sequences. In some embodiments, the sequences that comprises transcription factor binding motifs may include transcription factor binding motifs that bind more than one type of transcription factor or may bind the same type or same transcription factor at more than one site along the sequences. For example, in a construct that includes a transcription initiator that comprises four repeated sequences separated by three linking sequences, each of the four repeated sequences may comprise the same transcription factor binding motif so that one type of transcription factor may bind to the multiple repeated sequences or each or some of the repeated sequences may comprise different transcription factor binding motifs so that different types of transcription factors may bind at the repeated sequences.

In some embodiments, the transcription initiators of the disclosure may be made up of concatemers of transcription factor consensus binding motifs. The concatemers may be homogenous (a concatemer of repeated identical motifs); or maybe be heterogenous (a concatemer of a plurality of different motifs). Table 1 provides exemplary transcription factor consensus binding motifs that may be used in one or more concatemers of the disclosure. In some embodiments, the cell sensing constructs of the disclosure may comprise a transcription initiator comprising any one or more of the transcription factor consensus binding motifs of Table 1 and may have a sequence of any one or more of: SEQ ID NOS: 175-509, 9793-9088.

### Transcription Regulators

In some embodiments, the construct may comprise a transcription regulator or transcription initiator and a transcription regulator. In some embodiments, the transcription regulator may directly or indirectly modulate transcription of one or more genes and the transcription regulator may include one or more response elements. For example, in some embodiments, the transcription regulator may include an enhancer, a suppressor, or the like. In some embodiments, the enhancers may be comprise or have sequences reminiscent of prototypical or classical enhancers that have been previously characterized. However, in other embodiments, it is envisioned that the transcription regulators including enhancers may comprise sequences that are not reminiscent of prototypical or classical enhancers but nonetheless support the enhancement of transcription from a promoter and may demonstrate enhancer function. In some embodiments, the transcription regulator may be inducible.

In some embodiments, the suppressors may be reminiscent of prototypical or classical suppressors that have been previously characterized. However, in other embodiments, it is envisioned that the transcription regulators including suppressors may comprise sequences that are not reminiscent of prototypical or classical suppressors but nonetheless support the suppressing of transcription from a promoter and may demonstrate suppressor function.

In some embodiments, the transcription regulator may comprise one or more repeated sequences. In some embodiments, each of the repeated sequences may be the same or each of the repeated sequences or some of the repeated sequences may be different sequences. In some embodiments, the transcription regulator may comprise one or more sequences from the untranslated genomic sequence of one or more of: BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D. In some embodiments, the transcription regulator may comprise one or more of sequences from the untranslated genomic sequence of one or more of: NFAT, NFkB, REL, RELA, IRF2, GATA3 and ATF.

In some embodiments, the transcription regulator comprises one or more sequences from the untranslated genomic sequence of NFAT. In some embodiments, the NFAT transcription regulator may comprise a sequence of any one or more sequences of SEQ ID NO: 3430-3437, 3802-3803, 3940-3947, 4312-4313, 4808-4815, 4874-4889, 4298-4299, 4364-4379, 8576-8583, 8948-8949, 9444-9445, and 9510-9525.

In some embodiments, the transcription regulator comprises one or more sequences from the untranslated genomic sequence of NFkB. In some embodiments, the NFkB transcription regulator may comprise a sequence of any one or more sequences of SEQ ID NO: 550-553, 1060-1063, and 5696-5699.

In some embodiments, when the transcription regulator comprises one or more sequences from the untranslated genomic sequence of REL (including RELA or RELB). In some embodiments, the REL transcription regulator may comprise a sequence of any one or more sequences of 958-959, 3486-3501, 4426-4427, 4472-4473, 4484-4499, 1468-1469, 3996-4011, 4936-4937, 4982-4983, 4994-5009, 5697-5698, 8632-8647, 9572-9573, 9618-9619, and 9630-9645. In some embodiments, the RELA transcription regulator may comprise a sequence of any one or more sequences of 4484-4499, 4994-5009, AND 9630-9645. In some embodiments, the REL, RELA or RELB transcription regulator may be used in combination with a minimal promoter. In some embodiments, the minimal promoter may comprise a sequence isolated or derived from one or more of: minimal promoter-1 (“minP1”), YB-TATA, and human beta globin. In some embodiments, the minimal promoter may comprise a sequence isolated or derived from minimal promoter 2 (“minP2”) or minimal promoter 3 (“minP3”). MinP1 may comprise a sequence of: AGAGGGTATATAAAAGCTCGACTTCCAG (SEQ ID NO: 9788). MinP2 may comprise a sequence of: TAGAGGGTATATAATGGGGGCCACTAGTCTACTACCAGAAAGCTTGGTACCGAGCT CGGATCCAGCCACC (SEQ ID NO: 9789). MinP3 may comprise a sequence of:

(SEQ ID NO: 9790)
CTAGAGGGTATATAATGGGGGCCACTAGTCTACTACCAGAAAGCTTGGTA
CCGAGCTCGGATCCAGCCACC.

In some embodiments, when the transcription regulator comprises one or more sequences from the untranslated genomic sequence of IRF2. In some embodiments, the IRF2 transcription regulator may comprise a sequence of any one or more sequences of SEQ ID NO: 2384-2447, 4430-4461, 2894-2957, 4940-4971, 7530-7593, and 9576-9607. In some embodiments, the IRF2 transcription regulator may be used in combination with a minimal promoter. In some embodiments, the minimal promoter may comprise a sequence isolated or derived from one or more of: minimal promoter-1 (“minP1”), YB-TATA, and human beta globin. In some embodiments, the minimal promoter may comprise a sequence isolated or derived from minimal promoter 2 (“minP2”) or minimal promoter 3 (“minP3”). In some embodiments, MinP1 may comprise a sequence of: AGAGGGTATATAAAAGCTCGACTTCCAG (SEQ ID NO: 9788). In some embodiments, MinP2 may comprise a sequence of: TAGAGGGTATATAATGGGGGCCACTAGTCTACTACCAGAAAGCTTGGTACCGAGCT CGGATCCAGCCACC (SEQ ID NO: 9789). In some embodiments, MinP3 may comprise a sequence of

(SEQ ID NO: 9790)
CTAGAGGGTATATAATGGGGGCCACTAGTCTACTACCAGAAAGCTTGGTA
CCGAGCTCGGATCCAGCCACC.

In some embodiments, the transcription regulator may comprise one or more sequences from the untranslated genomic sequence of GATA3. In some embodiments, the GATA3 transcription regulator may comprise a sequence of any one or more sequences of SEQ ID NO: 1048-1079, 3566-3709, 1558-1589, 4076-4219, 6194-6225, and 8712-8855. In some embodiments, the GATA3 transcription regulator may be used in combination with a minimal promoter. In some embodiments, the minimal promoter may comprise a sequence isolated or derived from one or more of: minimal promoter-1 (“minP1”), YB-TATA, and human beta globin. In some embodiments, the minimal promoter may comprise a sequence isolated or derived from minimal promoter 2 (“minP2”) or minimal promoter 3 (“minP3”). In some embodiments, MinP1 may comprise a sequence of: AGAGGGTATATAAAAGCTCGACTTCCAG (SEQ ID NO: 9788). In some embodiments, MinP2 may comprise a sequence of: TAGAGGGTATATAATGGGGGCCACTAGTCTACTACCAGAAAGCTTGGTACCGAGCT CGGATCCAGCCACC (SEQ ID NO: 9789). In some embodiments, MinP3 may comprise a sequence of

(SEQ ID NO: 9790)
CTAGAGGGTATATAATGGGGGCCACTAGTCTACTACCAGAAAGCTTGGTA
CCGAGCTCGGATCCAGCCACC.

In some embodiments, the transcription regulator comprises one or more sequences from the untranslated genomic sequence of ATF3. In some embodiments, the ATF3 transcription regulator may comprise a sequence of any one or more sequences of SEQ ID NO: 124-159, 3140-3147, 634-669, 3650-3657, 5179-5214, and 8286-8293.

### Exemplary Engineered Enhancer-Promoter Combinations

Also provided herein are engineered (e.g., non-naturally occurring) Enhancer-Promoter (“EP”) combinations or pairings (“EP Pairs”) of the one or more cell sensing constructs disclosed herein. In some embodiments, certain promoters and certain enhancers may be paired together to drive transcription from the promoter, demonstrating predictable and consistent behavior, as will be described in more detail herein. In some embodiments, the engineered enhancer-promoter combinations may include: sequences isolated from or derived from the untranslated region of transcription factors, promoters, and enhancers. In some embodiments, the enhancer-promoter pairs may be comprised of an enhancer sequence and a promoter sequence. The sequences of the enhancer-promoter pairs may be referred to herein as “EP pairing sequences”, or “enhancer-promoter pair sequence”.

In some embodiments, the transcription initiating promoter sequence may be adjacent to the transcription regulating enhancer sequence. For example, the enhancer sequence may be positioned 5′ to the promoter sequence or the enhancer sequence may be positioned 3′ to the promoter sequence. In some embodiments, in the exemplary construct, the enhancer sequence may be adjacent to the promoter sequence. For example, in some embodiments, the enhancer sequence may be located 5′ of the promoter sequence, however in some embodiments, the enhancer sequence may be located 3′ of the promoter sequence. In some embodiments, the promoter sequence may be located 3′ of the enhancer sequence or in some embodiments, the promoter sequence may be located 5′ of the enhancer sequence.

In some embodiments, the enhancer-promoter pair may be combined together in an EP pairing sequence. In some embodiments, the enhancer-promoter pairing sequence may comprise between about 25 base pairs to about 5000 base pairs, including about 100 base pairs to about 1500 base pairs, about 200 base pairs to about 1400 base pairs, about 300 base pairs to about 1300 base pairs, about 400 base pairs to about 1300 base pairs, about 500 base pairs to about 1250 base pairs, about 600 base pairs to about 1250 base pairs, about 700 base pairs to about 1250 base pairs, about 800 base pairs to about 1200 base pairs, about 900 base pairs to about 1100 base pairs, or the like. In some embodiments, the EP pairing sequence may comprise at least about 25 base pairs including about 50 base pairs, about 100 base pairs, about 150 base pairs, about 200 base pairs, about 300 base pairs, about 350 base pairs, about 400 base pairs, about 450 base pairs, about 500 base pairs, about 500 base pairs, about 600 base pairs, about 700 base pairs, about 800 base pairs, about 900 base pairs, about 1000 base pairs, or about any number of base pairs between 25 base pairs to about 1000 base pairs. In some embodiments, the enhancer sequence and the promoter sequence may be linked together by a linker. In some embodiments, the linker may comprise a sequence of one or more of: TACGCT, TGATCT, TGCTTT, or TGCCCGT. In some embodiments, the one or more constructs may further comprise adapter sequences located 5′ and/or 3′ of the enhancer-promoter pairing sequence. In some embodiments, the adapter sequences may comprise a sequence of AAAGCACTCTTAGGCCTCTGCGTCTCATTCTGAAGACTCACGA (SEQ ID NO: 9801), or AACGGAGTCTTCAACGTGAGACGGCTCGATGCCATAGTTCCTT (SEQ ID NO: 9801), although other linkers known in the art are considered.

In some embodiments, the one or more nucleic acid constructs may further comprise a barcode sequence operably linked each EP pairing sequence. In some embodiments, operable linked may include the barcode sequence may be positioned 3′ to the EP pairing sequence, while in other embodiments, the barcode sequence may be positioned 5′ to the EP pairing sequence. In some embodiments, the barcode sequence may be positioned 3′ to the promoter sequence, while in other embodiments, the barcode sequence may be positioned 5′ to the promoter sequence. Likewise, in some embodiments, the barcode sequence may be positioned 3′ to the enhancer sequence, while in other embodiments, the barcode sequence may be positioned 5′ to the enhancer sequence.

In some embodiments, the nucleic acid construct may further comprise a 5′ untranslated region (UTR) positioned 3′ to either the EP pairing sequence or the barcode sequence. In some embodiments, the 5′ UTR maybe positioned 3′ to the enhancer sequence, 3′ to the promoter sequence, or 3′ to the barcode sequence. In some embodiments, the 5′ UTR may be positioned 5′ to the enhancer sequence, 5′ to the promoter sequence, or 5′ to the barcode sequence. In some embodiments, the promoter sequence, the enhancer sequence, or the combination thereof may comprise a sequence that may be isolated or derived from a sequence of one or more human chromosomes including at least chromosome 1, chromosome 2, chromosome 3, chromosome 4, chromosome 5, chromosome 6, chromosome 7, chromosome 8, chromosome 9, chromosome 10, chromosome 11, chromosome 12, chromosome 13, chromosome 14, chromosome 15, chromosome 16, chromosome 17, chromosome 18, chromosome 19, chromosome 20, chromosome 21, chromosome 22, or chromosome 23. In some embodiments, the enhancer sequence may comprise a minimal sequence that demonstrates an enhancer function of activity. In some embodiments, the promoter sequence may include a minimal promoter sequence that wherein the minimal promoter sequence demonstrates promoter function or activity. In some embodiments, the minimal promoter sequence may include any one or more of:

MinP1 having a sequence of AGAGGGTATATAAAAGCTCGACTTCCAG (SEQ ID NO: 9788). MinP2 having a sequence of TAGAGGGTATATAATGGGGGCCACTAGTCTACTACCAGAAAGCTTGGTACCGAGCT CGGATCCAGCCACC (SEQ ID NO: 9789), or MinP3 having a sequence of

(SEQ ID NO: 9790)
CTAGAGGGTATATAATGGGGGCCACTAGTCTACTACCAGAAAGCTTGGTA
CCGAGCTCGGATCCAGCCACC.

In some embodiments, the enhancer sequence and the promoter sequence may each, or in combination, comprise a sequence of at least about 25 base pairs, at least about 50 base pairs, at least about 100 base pairs, at least about 150 base pairs, at least about 200 base pairs, at least about 250 base pairs, at least about 300 base pairs, at least about 350 base pairs, at least about 400 base pairs, at least about 450 base pairs, at least about 500 base pairs, at least about 600 base pairs, at least about 700 base pairs, at least about 800 base pairs, at least about 900 base pairs, at least about 100 base pairs, or any number of base pairs in between.

In some embodiments, the enhancer-promoter pairing sequence may comprise any sequence of Table 1 including any one of SEQ ID NOS: 1-174 or a sequence with at least about 90% sequence identity thereto. For example, the EP pairing sequence may comprise a sequence with at least about 90% sequence identity, at least 91% sequence identity, at least about 92% sequence identity, at least about 93% sequence identity, at least about 94% sequence identity, at least about 95% sequence identity, at least about 96% sequence identity, at least about 97% sequence identity, at least about 98% sequence identity, at least about 99% sequence identity, or at least 99.9% sequence identity to any sequence of SEQ ID NOS: 1-174.

As noted herein, each of SEQ ID NOS: 1-174 are made up of a promoter sequence and an enhancer sequence. Accordingly, in some embodiments, also provided herein are sequences that have at least about 70% sequence identity thereto each of the promoter or enhancer sequences independently. For example, a sequence of an EP pairing of the disclosure may comprise a sequence with at least about 70% identity, at least about 75% sequence identity, at least about 80% sequence identity, at least about 85% sequence identity, at least about 90% sequence identity, at least 95% sequence identity, at least about 96% sequence identity, at least about 97% sequence identity, at least about 98% sequence identity, at least about 99% sequence identity, or at least 99.9% sequence identity to any of the promoter sequence of SEQ ID NOS: 1-174. Likewise, a sequence of an EP pairing of the disclosure may comprise a sequence with at least about 70% identity, at least about 75% sequence identity, at least about 80% sequence identity, at least about 85% sequence identity, at least about 90% sequence identity, at least 95% sequence identity, at least about 96% sequence identity, at least about 97% sequence identity, at least about 98% sequence identity, at least about 99% sequence identity, or at least 99.9% sequence identity to any of the enhancer sequence of SEQ ID NOS: 1-174.

| SEQ ID | | |
|---|---|---|
| NO: | EP Pair | Sequence |
| 1 | GTF2B-MPC2 | TCACACCTTTACCACAACAGCTGTTTACCCAGCACTGA |
| | | AGGCGGCTGCCAACCACAACATGAGGAATACATTTCCT |
| | | CTTCAGAATGGGTGTTGGTGGTGTGTGGGTGCCAATGC |
| | | AGCGTGGGGACTGTGGCCTCACACATGAGTAAGAGCT |
| | | GGTGTCCTATAGCCTTCAATGCCTGTCAAATAACTGTG |
| | | GAACATGTTCAGCCCACACACGTTGAGACTCCTCCTCA |
| | | GACTCCTAGGCGGGCGCTGCGATGGAGGCCGAGGGCC |
| | | TGGACTGGCTCCTGGTGCCACTGCACCAGCTGGTGTCC |
| | | TGGGGCGCGGCCGCGGCCATGGTCTTCGGAGGGGTGG |
| | | TGCCCTACGTCCCGCAGTATCGGGACATTCGCAGGACG |
| | | CAGAACGCCGACGGCTTCTCCACCTACGTGTGCCTGGT |
| | | GCTGCTGGTGGCCAACATTTTGCGGATACTCTTCTGGT |
| | | AAGTTGGGGGCAAAGAAAGTTGACAGACAATCCGTAG |
| | | GCCTACTGCGTCTACGCCACCCGCA |
| 2 | GIGYF2-TNS1 | AGCTCTAACTTATAGATGTACTTCTGTCAATACAGTAA |
| | | TTGGGCTAAGCTTTGTAATTTTTACTGTTCCTTAGCAAC |
| | | AGTTTGCTCGGAACCTCCAACATTGGTTTTTACAGAAT |
| | | AGTCATATGTGAAGTCAATGCTTTATCCCTTACCTTCTG |
| | | GTACAGTCAGAAGGCGAAAGTAACAGACTAAAACAGA |
| | | ATGCTTCACAGGGCTTTGAAATAGATGCTATCATATTT |
| | | TTCCTACTAAATAGATCAGGCTCACTACCAATTAGCAT |
| | | CATCTTAATCTTGCCTAATAGTGAAGCAAAGTCACCAA |
| | | GGATTTGGTGGATTTATCAGCTCCTCCCACAATAGTCA |
| | | TCTGAGGCTAACACCTCTTGGAACCTAGATCGAAAGTC |
| | | CTTTGGTAATGATGTGTCATACATTCTAGTCATCAAAG |
| | | ACACCATTTTCTGGGCCTGAAGTGTTCTGCTGGTTTTTG |
| | | GAGGAAAAAGAAAGTTGACAGACAATCCGTAGGCTCG |
| | | GCGACAACCGCCTCTACTTC |
| 3 | ADO-ENTPD1 | GAGTTGCATTTGTATCTTTGTCAATACAATATCATCTTA |
| | | CCTTGATACACCACTACATGTCTTGGCTATATGACATC |
| | | AGTGTCTTGCACAATGGTATTGTGGAAAGGTCAATACT |
| | | GTGCCAACCCACCTACAGCCTCTTCTGAGGGTACCCAT |
| | | CCTGCCCACACTTTCTGCTGAAGGGACAGGCCCAGGAA |
| | | GCCATACTCAGTACCCAGTGCCCCACTTCCTCTATCCAT |
| | | TCCTCAGCCTCAAACTCCTGGGCCCAGCATACCTGGCT |
| | | AATTTTAAAATTTTTTGTACAGATGGAATCTTGCTTTGT |
| | | TCCTAAGGCTGACCTTAAACTCTTGGCCCCAAGGCATA |
| | | CTCCCTCCTCAGCCTCCCAAAGTGCTGAGGTCACAGGC |
| | | ATGTGACACTGTGCCCAGCCTCCCCTTCAGGTCTTCATT |
| | | CTGCAGTCTCCTGTGTACGTGTAAAATTATGATCAAAT |
| | | AAATAAAGAAAGTTGACAGACAATCCGTAGGCCGTCG |
| | | CCCACGCCTCCATCATCT |
| 4 | CD226-LDLRAD4 | CCTACACTGTGCCCATTTAAAGATTAATAGTATATGCT |
| | | TTAGAATAGTGCTTTACTAATATGATTATGAATCATAA |
| | | GTATAATAGCTTGTGCTTAGTACAACAATAAGAGAATC |
| | | AGGCGGTGAGCTTGCTTGACTGACCACAGAGTGGAAC |
| | | AAACAACCACAGGAAGTCTGCGGAGCCTCTGAGCAAG |
| | | AAAGTTGGCTTATTGCTCAACCTTCCACTTGAGTGCCT |
| | | GAACTCCTGATGCAGTGAAGTCACAGGTTCCGTGATGT |
| | | CACAGGTGGGCAGGCGCACTGGTCTGTAACGGATTGG |
| | | CTTGGCCTTACCCACGAGGCTTTGCGGTGGCTGTTGGC |
| | | TTGGCGGTGGTCGCTTGGCTTGGCGTGGCAGTGATCGC |
| | | TTGGCTTGGCATTTCTGGCTTGGCGGTCCTCCTTTCGCA |
| | | GATTGGAAACCGCGGGCTATCCTGCTGGGAGGTTGTGG |
| | | CCGAGGCAGTAAAGAAAGTTGACAGACAATCCGTAGG |
| | | CCAACCGCGTCGACGCCAGCTTCA |
| 5 | LARS2-SLC12A8 | ATGAGGATTAAATGAGATAATCTGTGAAGAATTATATA |
| | | TGGCATGAACAAGTCATATATAAAGTGCTGAGTAAACT |
| | | TCAGATACTGAAGTTACACAGCAGACTGTGGGCTCCTC |
| | | CAACGCAAAGACTGGGTCTTATTCATCTCTGCAACTTT |
| | | TTTGCCTAGCACGAAGTCTGGCTGAATATTTGGTACAA |
| | | AATACATGTTTACTGGATAACTAAATAATCGCAATAAT |
| | | TATCCTGGCACAGCCGAGAAGTAAGCCCTGCCAGCTCT |
| | | CCCGGGTGCGGGGACGCGCGCGCCCACCTTCGCAAAC |
| | | GAGACTTCCGGGATCCCGGAGGGGGCGGAAGTCGAGG |
| | | CCTCTCTCGCTTCCGGCTCGGCCATTGTTTTTGGTCTAA |
| | | CGGGCAGTAGAGTGTCCGGCTTCGGTGCCGAGTGCCAC |
| | | CGCGAGTGGGCCGAGACGCGGAGGGAGGCGCGGCCGG |
| | | AGCTCGGGTCAAAGAAAGTTGACAGACAATCCGTAGG |
| | | CTCGTCTTCATCTGCCACCCCTTC |
| 6 | TMEM121-ADCK1 | AAGGTAATAAATAAAGTCAAATTAGAGGTACAAAGGC |
| | | AGTTGATAGGTACCCAAAGGGAAGACCAAACAGCAGT |
| | | GTCTGTCAGAGAAAATGCCAGCAGGGGACACCTCCTG |
| | | ACCATACCTCCTGCTGAGGCGGAAGGTGTGCCGTTGGT |
| | | GGTTGACAAAGATGTTTTTTCTGTTGAAACACCAAAGA |
| | | AAGAACATCAACCTCTAAGAAACACTTCCTTTACATGT |
| | | CAGAATCCTAATAAAAGCATCAGTATTGCAAAGACTTT |
| | | CCATGATCCTATACCCACCTCGAAAGCCCCCTCTCACC |
| | | ACAGGAAGTGCACTGACCATTGGAGGCATAAAAGAGA |
| | | TCCTCAAAGAGCCCGATCCTCACTCTCCCTGCACAGCT |
| | | CAGCGGGACCTCAGCCATGAGACTTCTCATCCTGGCCC |
| | | TCCTTGGCATCTGCTCTCTCACTGCATACATTGTGGAAG |
| | | GTAAGTGGAGAAAAGAAAGTTGACAGACAATCCGTAG |
| | | GCCCTCGTCACCAACGACGACTTCT |
| 7 | TTYH2-CTNS | TCCACTTGCCATGAGCTGGTTTTGCAAAGAAGAAAACG |
| | | GTTTCCTTTATATGTGGAGATTAAGAACAAGTTTATCT |
| | | GTAGTTTTGGTGCTCGACTAACTGGGAACCAAGCCACA |
| | | GTTGCTTGTAAAACGGACCCTAAGTTATCTTGATCTTG |
| | | ATGGTTCAGTGCTTGGAAATCTCTGGGGCTTTCGGTGC |
| | | CTGGTAAAAGTTCAATCACTTATCTGATTTGTGGTTGG |
| | | CATCCTCCAGCTGCACTCCCCACTCTCTGTCACCAGGA |
| | | GCTGCCGGGTGCCTGGGATATCCTGGCAGCTCTGCTCA |
| | | AAATGATCTACGACTTCATGAATTTATTTGGCTCCTCCT |
| | | CGGGGCCAGGGTGAGTGTCATGGGTTAATAAGGCCGG |
| | | CCCCGCCTTCAGGAGCGGTCCACTGGGAGATGTGTGCT |
| | | GCGCAGCCCTCTTGCGAAAGCTCTCCCCTGGTGGGACA |
| | | TTCTGGGCAAAGAAAGTTGACAGACAATCCGTAGGCG |
| | | CGACTACCACTGCATCGGCGAC |
| 8 | WDR27-H2BC6 | AAGGCATTTTACTTCTTGAAATATTTAGTTTTGACCTGA |
| | | GAAAGGGGAGAATAAAACGTTTGTACTTTGGGGGACA |
| | | GTATGAGTGCTGGGGTTTCTGTTTCAGTGCAGGCTTGT |
| | | CTATGAGACATGCATATGCATAACTTCATGCAGGGAAA |
| | | GTGTCGTGCACTTTTCTCTCGCTGAAGTTTACTTCCGGG |
| | | GTGACTTAGTTTTTCTGATCAGTTTAGATGTTCATATCT |
| | | TCCTGCTTCTGGAATGGGGGTCTACAGGTGTGAAAGGT |
| | | CAGCCCCCCTGAGGGAGAGGTGGGGCCCGAGCAGGCT |
| | | CCTCCCACTGCGGGAGTGCTGGCCTTTCCCCTGCAGGG |
| | | CTGTGGCTCTGGGACCACTTCCTCTTTCTTCACTCTGAA |
| | | GCCAAGAGCCGACCTTCTGAGCCCTCAAGAAAGATCA |
| | | GAACAGATTCATGGGTGATTTAGCCTATCTGTCCCAGG |
| | | CCAGCGTAAAGAAAGTTGACAGACAATCCGTAGGCTC |
| | | CAGCGTCCGCCACTGCACCTA |
| 9 | GTF2B-MPC2 | CATGTACCTGACGCTTTATTGTTTCTCTAACACACCAGC |
| | | CAGTTCTGACCTCAAAGCCTTCATGCTTGCTGTTTTTTT |
| | | CCACTCAGCATGCTCTACCCTAGCTGCCTGTTTCGTTGG |
| | | CTTCCTCATTTCCTTGAAGTCTTTACTCAAAACACCTTC |
| | | TCAGTCAGACCTCTCCTAGCCAGTATTTAGAACTGCAA |
| | | CTCCCAACACTACCCTTCCCTGTCCCAGCTCTATTATCC |
| | | TGAAGCCAGGACTCGATGCAGATTGGCTACGGATCCA |
| | | AAACGCCCACTAACCTTGGCGCGTCCCAGTCTCCACCC |
| | | CAAACCTAACTCAGGTGAAAATGGCGCCACGCGCGGG |
| | | GGCGGGGCTAGAGGTACAAAGAGAGTGTCTATTGGTT |
| | | GGGAGCGGCACGGGCCGCCTTCTTCGCTTCACCATTGG |
| | | CTCGCTCCGTAAGGCAAGAGAACCCACTAGGGGATGA |
| | | GCCCGAAAAAGAAAGTTGACAGACAATCCGTAGGCCG |
| | | CAGCTTCAGCGTCCTCTTCAA |
| 10 | | CAGAATGATTTATATTCCTTTGGGCATATACCCAGTAA |
| | | TGAAATTGCTGGGTCTGATGGTATTTCTGTCTTTTGGCC |
| | | TTTGAGAAATTGTCTTCCACAATGACTGGACTATTTTAC |
| | | ACTCCCACCAACAGTGTAGAAGTGTTAATTTTTCTTCAT |
| | | AACCTCACTAGCATCTGTTATTTTTGACTCTTTAGTAGT |
| | | AGCCATTCTGACTGATGTGAGATAGTATCTCATTGTTC |
| | | CTGGGCAGCTGAGGAGGAGCTACCTTAAAAAAGGAGG |
| | | TGTGTGCCAGGGAGCTGGGTAGGAGCCTGGCTATATAT |
| | | CTGCCCAGCAGCGGTACTCTCGGGACAGAGATGGCACT |
| | | GATGCAGGAACTGTATAGCACACCAGCCTCCAGGCTG |
| | | GACTCCTTCGTGGCTCAGTGGCTGCAGCCCCACCGGGA |
| | | GTGGAAGGAAGAGGTGCTAGACGCTGTGCGGACCGTG |
| | | GAGGAGTAAAGAAAGTTGACAGACAATCCGTAGGCGC |
| | | CTCTACTGCTTCTACTTCGTC |
| 11 | ITPR2-LAG3 | GACAACCTCGGTGTTCTACAATAGGGACCAAGAGGAA |
| | | CAGGCCCAAAAGGAAAAGCAAGATCAGAGAAAGGCCT |
| | | CAGCCTTAGTCATAGCTCTCAGCCTTAGTCATGGCCCT |
| | | CAGACAAACAAACCTTGGTGGTTCAGAGAGGAAAGAA |
| | | AATGAGGCAGGCCAATCACTTGGTAGGGCTTGTTATCA |
| | | GTGTGGTTTACAAGGACACTTTAAAAAAGATTGTCCAA |
| | | TGAGATCCTTTCTAAGCCATTTAGAAATGACTCACTTT |
| | | GAAACTCTTCCTGTGAGGTCAGATAGTGGTCTAGAGCA |
| | | TAAGACTTAACTTATTGCCGGAAACAGAGAGAGAACA |
| | | GAAGAAGAGAAAGCTCAGCAAATTTTCTTGCCATACTT |
| | | CATGACTTCACTGTGGCTAAGTGTGGGGACCAGACAGG |
| | | ACTCGTGGAGACATCCAGGTGCTGAAGCCTTCAGCTAC |
| | | TGTCTCAGTTTTAAAGAAAGTTGACAGACAATCCGTAG |
| | | GCTCGGCTCCTGCCCCCTCAGCTTC |
| 12 | ETV6-PEX5 | ATTACAGTTGTCTATGGTGTTCAGTACAGTAACATACT |
| | | GCACAGGTTTGTAGTCTAGGAGCAGTAAGTTATGCCAT |
| | | ATAGCCTATGTGTGTAATAGACTATACCATCTAGGTTT |
| | | GTATAAGTGCACTCTGTGATGTTCACACAATGACAAAA |
| | | CCCCCTAAAAATGCATTTCTCAGATTGTGTCACCATTGT |
| | | TGAATGATGCATTATTGTAATTTAAAGATGTTGCTCCA |
| | | CTCCTTCAGAAAGATATGCCCAATAACAAGAGACTATC |
| | | CGAGGGCGGTGCAAATGGAAATATTGAAAGCCGATTG |
| | | GTTCTTCCAAGCTGGAAGTTGTGGAGGAAGGACTAATT |
| | | TGGAACCAATTGCGTGGCGCCTGAGAGCGGCAGCGCG |
| | | GTCAGTGGCGGCGCTGAAGAGACCGGTTGCCGCCATG |
| | | ATAGAACAGCAGAAGCGTAAGGGCCCAGAGTTGCCGC |
| | | TGGTTCCAGTCAAAGAAAGTTGACAGACAATCCGTAG |
| | | GCTGCATCAACTGCATCTGCTACGG |
| 13 | PRRX1-SNRNP40 | GGGTAGAATTTGATACAAAGCCACTGCTATGTGTGGGA |
| | | GGCTGAGATCAGACCAGACATCAGCTTTTTAACATGAG |
| | | GAAGGATGTGGCTGCATCTCAGACATGAGACGTGCAT |
| | | GCATGAATGTTAGGAAGGTTTACAGTGATTCTTAGAAA |
| | | TGTAACCTCTAATCACAGAATTTAGACCTATGCCTCCT |
| | | GGTAAATTTAGAATTGAGCCTTTGTTATGGATGCATTA |
| | | TTTTCCTATATGACCAATGGGAGAGTAGACCTAAGCTC |
| | | CTTCATTTGCATGCAGACTTCACGACAAAATAACGAAT |
| | | CAGAGTTGGAGAAACTAAATCTTCATTTACATAACATT |
| | | GTCTACAAATTCCGAGGATCAGGAGATGTAGATTTCAT |
| | | TTTCTTTCCTAACTGCAGAACAGCAAAGATAGCATGCC |
| | | TGAGCCAGCGAAATCCGCTCCCGCCCCGAAGAAGGGC |
| | | TCCAAGAAGGAAAGAAAGTTGACAGACAATCCGTAGG |
| | | CACAGCTTCGGCTCCTACAGCACC |
| 14 | BACH2-H2BC8 | ATCTCCTCTTGCCATTATCTACTAAACAATCCCTGCCCA |
| | | AACCATGTAGAAACAGAATAAGAAACTTCAGACATAT |
| | | TTTGCAGTGAAGCAGGAAACCTATCAGATGACGTGGCC |
| | | ACTTTCTCTTGTAAACCCACTGAGGGTGTGGGGCTGGA |
| | | ATGCAGCTGCCAACATGTCTGGTTCCTTTCTAGCTCTGA |
| | | GCATCACCAGTACTTAAAGGGACTTTTCATGTAGCATC |
| | | ATCCTCAAAGACTGGATGAATGGACGATGTACCATAG |
| | | AGGAGAGAGAGAGGAGCGTGTGTCTGCACATCTTCCT |
| | | GCAGATCGCAGAGGCAGTGGAGTTTCTTCACAGTAAA |
| | | GGACTGATGCACAGGGACCTCAAGGTCTGTATTTGTGG |
| | | AGCATCACCCTTGGGGTTTCAATCTGACGTTTTGTGATT |
| | | CAGAGCAGTACTTGCAGTACTCTGAAGGATCCTTAAGA |
| | | GTTGGGGAGAAAGAAAGTTGACAGACAATCCGTAGGC |
| | | CCCGCAACAGCGTCTGCGTCCGC |
| 15 | ACVR2A-ARHGEF4 | GCCACAGTGCCCGGCTCCCTGCCCCACTTTCTGAGACC |
| | | CAGATCCACTTCCTGGTTTCCTGTGTGGTCATGTGACCA |
| | | CACTTGGCCAATTGGAGCCTTTTGTTATCACAGGCACA |
| | | GGGATTGGGTCAGGGCTGGGCACGTGATCCTGCCTGAG |
| | | CCAATCATGCTACGAGTTTGGAGACTCCCTGGAAGAAG |
| | | GCCTAGAAGTAAGGAAAACCATCTTGGACGCATGATG |
| | | ATTCCTCCACCGCAACCCGCCCCTTTGCCTACTGCGCG |
| | | GGCAGTTTCTTGCAAACCCGGAAGCAGATCTCTTGGAG |
| | | TGAAGGCTGTTAGTCGCCGCGTGAGTTTCCCTCTATCT |
| | | AGATTAAACCTGCGTTTCGGAGTCCCCTGCTCCTCCGT |
| | | ACCCGATGTCCGGGGGTTCGTATAGCCCTTAAAGTCCC |
| | | CGCACGGCTCCCTCCATTCCATGTAAATTCAGACTTTG |
| | | CCGCGAGAAAAGAAAGTTGACAGACAATCCGTAGGCT |
| | | CTTCTACACCCTCTTCTGCGAC |
| 16 | RYR1-EGLN2 | CAACACCATTACCTATATTGTTTGTGTTTTTCCTTAAGG |
| | | TCTCTTCTTATTACTTGCTAGCCTGACTTGAGTCATCAA |
| | | ATCCAACAAAAATCACGGTTTACACTCTGCTTCAGGCT |
| | | AGCTGCGTTTCATGTACTATATCTGACTACTACATCTGA |
| | | AACATTCTTCTTGGTAGCATATTTCAGTCAGAGTATGA |
| | | AAATTTTCTATGACTAAAGTCACTGTAGTGGCCTTATTC |
| | | CTGAAGCTTCTAAAAAGCCACTGGGAAAGTACTAAATC |
| | | TCTGAGACTAAAGAGGGCGGACTCAGCCTCACTTCCTC |
| | | CTTCTTCTCATCTGGGCTTCCTGTCGTCACAGCATGATC |
| | | ATATTTTTTCACCCTTCACTTCTCCTTTTACACAAATAG |
| | | CCCCGGATATCTGTGTTACCAGCCTTGTCTCGGCCACCT |
| | | CAAGGATAATCACTAAATTCTGCCGAAAGGACTGAGG |
| | | AAAAAGAAAGTTGACAGACAATCCGTAGGCGACATCT |
| | | TCATCTACGTCAACAC |
| 17 | LRRC8C-CD247 | AAAAAGAGGAAGCCCTTGGTTACTTACATTTGTCAGAT |
| | | ACTGTGTCAGGGAGAAGGATGATGACTATCTCACAGGT |
| | | TCCCTTACCTCGGGCATTAAAGTAGAAATGAACAATCA |
| | | GATGTGGTTAAGAGGGGAAAAGGTTACATTTCCTTTAA |
| | | TTCAGATTAAGCTTGCACTGCACCCATGATTGAACAAG |
| | | TGAGTTCTCTCAGTTCCTCTTGTCTTGGGGAGACCTCTA |
| | | ATCCTTGTACCCAGGGCTTTTGACAGCCCCGCAATTTCT |
| | | GCAAATGGTTCCTAATTGATTGGCTTCTTCTCTGTAAGG |
| | | GTGCCTATTACTGTCATAGCTCTTTGTGTGACCCTGTCA |
| | | GCCTCTCCTGTAGGGGGGGATTTTTTTCCCCAGTATTGT |
| | | TAGCAATTAGCATGTGCTATTGTAATAGTTTGGAGTTT |
| | | GAAAGAAATATACAATGTCCCTTTGGGTGAGGAGGTTG |
| | | AAAAAAGAAAGTTGACAGACAATCCGTAGGCTCCAGC |
| | | TACGACACCTTCTTCCG |
| 18 | CD83-RN7SK | TGCCATTCATTCAAGCAAATATTTGCTTATTGTCTACCA |
| | | CTGTGTACTAAATACAAGATCCAGAACTTGCTGTGTCC |
| | | TCACTGTTTTAAGTCAGTATGCATCATCCTTCCAGCATG |
| | | TGTACCATAGCAAGGTGTACAGAGCATTGCTAACCTCA |
| | | AAAGCTCACTTGAGCTTTGGTGTCCAGGGATTTTCTTG |
| | | GGGTTTCATTCTGTAGACATGATTGATGAGTCATAAGC |
| | | TCCTGGCGACTATCAGACATATTAAGCTGTCAGGGAAA |
| | | GAGCCTGTTTAATCCAGTCTTCAATCACGGGTTTCCTCG |
| | | GGCGTTAGCCACTGCCTGCGGAGTGTGGACTGGCAGA |
| | | AAAACGTCACCTTCGGGCCCCCCAGCACCAACGCGCCT |
| | | TAAACCAAGCCCCACAAAGAAGTCACTTCGGCGATGT |
| | | AAACGCGACTTTGCGGCTTCCTGGGCTGCTAAGGTGAA |
| | | GGACGCCAAAGAAAGTTGACAGACAATCCGTAGGCTC |
| | | TGCTGCCACAGCAACCTCATC |
| 19 | PDCD1LG2-C9orf16 | GACAAATGATTTTGGAGTTGTTGCACTAACTTCTGATG |
| | | TGATACAGGTCTAACAGTAGTTATGACTATAAAACCAT |
| | | ACACAGTTTTACAATCACTATCAACCACTTGCTGAGAG |
| | | GAAATAACTTTTTGGAACCAGGTATAAGACTCACAAAA |
| | | AGGTCAAGATTCACAAAAAGATTAAATCTACAATGAA |
| | | ATAAACTATCAGCTTCTCCATAGTTTGTTCTCAGGGGA |
| | | ATCTCCTCTTCACAGGCAACTGGTAACCACCTCATTTG |
| | | GGGATGTTTCTGCCTTGCTAGCAGTGCCAGAGAGAACT |
| | | TCATCATTGTCACCTCATCAAAGACTACTTTTTCAGACA |
| | | TCTCCTGTAGGGCTAGATTCAGAGAGCAGCTTCTGATA |
| | | TTTGGAGGGTAAGTGCCAGAAAAGTTACTGTAATGTTA |
| | | CATGTGGAGAATCAGGACTCCAGCCACAGTGACAGAT |
| | | AGGCATTGAAAAGAAAGTTGACAGACAATCCGTAGGC |
| | | GCTCCGTCGACAACGGCACCTCC |
| 20 | EOMES-FOXP1 | TTTGTTCTCACGTTGGATCTGTCTCTCCCTCTTCTTCTCC |
| | | CCACTCTGCGTGTACGTGCCTGTGCATGTTTGTTTCGTC |
| | | ATCTCTGTGACTACCTACCCACCTACCACTTTATCACTC |
| | | TTTTTTTGGTCCAGTCTTCCCCCGTGTCTGAGACTGTTC |
| | | ATCTGTGGCCCTGCCGCTGTTCCAACCTCGGGGACTTT |
| | | CCCTCACTGCACGTGGTTAAACACTTTGCTGAGAGTCC |
| | | TCTCAAAGCTGCCTCCGCCTCCTGACTGGAGAGCGAAC |
| | | TAGCCCGGGCGGAGCCACAGTCCTAGAGGCTGAGCGC |
| | | AGTCGGAGCTGTCCCATTTACCCGACCCGACGCCGGCG |
| | | TGATGTGGCTTCCGCTGGTGCTGCTCCTGGCTGTGCTGC |
| | | TGCTGGCCGTCCTCTGCAAAGTTTACTTGGGACTATTCT |
| | | CTGGCAGCTCCCCGAATCCTTTCTCCGAAGATGTCAAA |
| | | CGAAAGAAAGTTGACAGACAATCCGTAGGCCCCGGCC |
| | | TCTTCTGCCCCGGCTT |
| 21 | RNF144A-NAGK | TGGTGACCGCCTTCCCTTGGCCTCCGCTCAGCTAAACA |
| | | ATAGACGAGCAAAGTGACTCGATAGCACAGTCTAGTG |
| | | GAGGCAGCTGGGATTGCATCTCTGAGTCCTGGGGCTGA |
| | | TCAGGTGCCCTCTCTGGCTCCGCCTCCCTCTAAACCCTC |
| | | TCTCCACCCTCCCAGCACTCAGAACCCGGCAGTCCTAG |
| | | GTGAGCCACCGGGAAGACTCCTCCAACCTCACCGTAGG |
| | | GATCCTTCCCCTGGTTGGGGGTTGGGGGATTGAGGCTT |
| | | TAGAACTGACTTTGGAATGAAGCAGGAAACCAACCCC |
| | | AGGATCCAGACCATGTGTAGCTAGGTAGGGTTCAAGTT |
| | | GAAGTCTTGGGGGTTGGGCAGGAAGGGGGACTCTGCT |
| | | GCCCCTGGCCTTGCCTGAGAGATCTTGGTTGATGGAGC |
| | | TGTTGTCATGGGAATGGAGAGAGCCCGAACAGGAAGA |
| | | GGGTACAGCTTAAAGAAAGTTGACAGACAATCCGTAG |
| | | GCCAACTCCTTCGACTCCGTCAACC |
| 22 | GGT6-CCL4L2 | GGGTCTCGCTCCTGCTCTCACTCCAGAGCCTCTCCAGC |
| | | CCTCCCACGCTTCCCCTCTCGGGTCCCGCCCCAGGACT |
| | | GCTCTGCCCTGGCCCTTGGCCCTGCCCTTCCCTGACCCG |
| | | CCTCCACCCAGCTCCAGCCTCCCTTTGACCCCACCCCTA |
| | | CCCAGCTCCCAAACTGCCAGTTTCTATCGGGGCCTGGT |
| | | CGCAACTCGTATTGCTCCGCCCCCACGCCCAGGCCCCG |
| | | TCCTATCCAATCAGGGATGGTGGGCTGGAAACCATCCA |
| | | ATCAGGAACGCAGCTGGAGCGAAGAGGACGGTTTCCG |
| | | GGTTTGGTGCGGCCTTTGTCTCTCGCTGCAGTCAGAGC |
| | | TCCAGGTCTGGTTCTTCTCCTAAAGGCCCAGGCTGTGT |
| | | GGCCCCGTGTCCTGCAGGTATTGGGAGATCCACAGCTA |
| | | AGACACCGGGACCTCCTGGAAGCCAAAAATGGTGAGA |
| | | GTGAGGGTAAAGAAAGTTGACAGACAATCCGTAGGCA |
| | | ACCTCTACATCACCGACAACCG |
| 23 | CD79A-ZNF69 | ACTCCTAACCACTAGACTGAATTATAAAGAGGTGACAT |
| | | GAAATAAGCTCTAATAATTGATAATCCTACCTACACCC |
| | | CTGGAAGTGAGCCTTTCAACGTTTCTCTGCTTTGGTTAC |
| | | CAGGAACTCCCACTTCCTGGGTGTTCATGAGATTAGAG |
| | | GAGGCAAATGTATATGTCAACAGGAGCTGGGAGGTGG |
| | | TATAAATGTGTGAAGCTGCTGGCTGTCAAAAACAGTTG |
| | | CTTCCTCCCGCGTTTCTCTCCTGCCACCAGCTGGGGCCT |
| | | GCCTTTTATAGGCGCCGCAGCCAATGAGCACGCTTCTT |
| | | TCCTTCTGCCCGCCCGCCTGGCCAATCGCCGCGCCCCTT |
| | | GTGTAATCTCCGGCTAGTCGGCTGCTTTCGGCTATTTTC |
| | | GCTGTTGCTGGCTTTTTCAGGAGCTGCTCGCTCGCAGC |
| | | CAGAGACGCTGCTTTTTTTTTCCGGGTTCGGAGCCGTTC |
| | | CGGAAAAGAAAGTTGACAGACAATCCGTAGGCCCTCC |
| | | GCGTCTCCCTCGACTCCA |
| 24 | SFXN1-SLC35A4 | GGTGAAGAAAGAGTGGTTAGAATGGTTTCTCCACAGGT |
| | | GACAGCTGAAACAAGTCTTGAAAGAAGAGCAAGACTT |
| | | TGTCATGCAAATGAGGGGAAGTACATTCTAGGCAGCA |
| | | CTAATATATATCATGAGTGATAGCATGAAGGAGTAAA |
| | | AGCAGTGGTGGTTTATAGAAACTGAGTGATTTTTGTGA |
| | | ATCATATAGGAGAGAGGACAGGAGATGAGGTGGAAAA |
| | | GGTAGATCCTTTATTGAAAAGAGGAGGAGTCAAAGAT |
| | | CCTGAAAGGGTTGGGACTGCCTGCAGTCAGTCCCTAGG |
| | | GAACTTCCTGTTGTCACCACACCTCTGAGTCGTCTGAG |
| | | CTCACTGTGAGCAAAATCCCACAGTGGAAACTCTTAAG |
| | | CCTCTGCGAAGTAAATCATTCTTGTGAATGTGACACAC |
| | | GATCTCTCCAGTTTCCATATGTTGAGATTCTACTTATTC |
| | | ATCAGTTTGTTGAAAGAAAGTTGACAGACAATCCGTAG |
| | | GCTCAACCTCTGCGGCAGCATCATC |
| 25 | TXK-SULT1B1 | GGCTTGGTGCTGTCCTCGTGATAGTGAGTTCTGCTGAG |
| | | GTCTGGTTGTTTAAGTGTGCAGCTTCACTGGTGCAATG |
| | | CTTTCTCTGCTTCTGCTTTCCCCATGTGACATGCTGCTC |
| | | TCACTTCCCCTTCCACCACAAGTAAAAGCTCTGACACC |
| | | TCCCCAGAAGCTGAGCAAATGCTGGTGCCCTGCTTCCT |
| | | GTACAGCCTGCAGAACCGTGAGCCACTTAAACCTCTTT |
| | | TTCCTACAGGAATTGGAAAAGGGTAGGAATGGGGGAG |
| | | GAGATGCCAGATCAGCCCTGAGAACCTCTTTACCCATC |
| | | TCAAAGACTTGGGACACTAATGGGAAAGGCTGGGCTA |
| | | ACTTCTGCTGCCCTTCCCCACTGTCTGTCCAGGGGAAC |
| | | CTGGGAAAGACAAATAGGAGCTGAATCCTTCACTGAA |
| | | AGCCCCGTCATCGTATCTATGGGTTGGGCATTAGGAAC |
| | | GGCCTGGCCCAAAGAAAGTTGACAGACAATCCGTAGG |
| | | CCACGTCATCAGCGGCTTCTACAA |
| 26 | CCL5-ALOX12B | TGCATGTTTTTTAAGACAACAGCTTTTGCTTTAGTAGTT |
| | | GGGCGGTCGCAAGTGAACACTGTCCAGTGGTTCTTCTG |
| | | GTGCCCAGACTCGGAGGAGATGGGACTGCTCTCAATGT |
| | | GTTTGTTAGAAACGAAACTGTTCGCACTATAACCCCAT |
| | | AACCCCTTACAATGTTCAGAAAAAAACAAGAAGGGGC |
| | | TTGAAATACCCACTAGTGTGCCGCCTGCTCAGCCCATC |
| | | TGTCCTTCTGCTTTTGTGAAGTTTCCTGCTAGCAGTTTA |
| | | GAGATTGGGCTGTTTCCTCACTGATACATATCCCTTCAC |
| | | ACTTCTATAATTTAACTCTCTCAACTGCATGTGAAAGA |
| | | TCTTAGCTGAAGATGACTGACAGTGTTATTTATTCCAT |
| | | GTTAGAGTTGCCTACGGCAACCCAAGCCCAGAATGACT |
| | | ATGGACCACAGCAAAAATGTGAGTTAACCAAGGTAGC |
| | | ATGGAAGAAAGAAAGTTGACAGACAATCCGTAGGCCT |
| | | GCATCTTCGTCTACTCCAACT |
| 27 | BATF3-RPRD2 | GGTTGCAGTTTACTAAGGCCAGCCAACCCCACAATCCT |
| | | TATAATTCGCATTCCCCCATACTGTTCAAATGCAGAAA |
| | | CTAGTGATTCATCTGTCATCTGTACCTCATTTTCATCTA |
| | | CTAGAGGTGAGCCTTTTGATAGTGATACTGGTCACTGG |
| | | TGAGTCACTAGTTCCAGAGTCCCATTCTGAACATATAC |
| | | TTCCTGTAGCAACTCTCTTAGCTTGTGTTTTCTCAGAAA |
| | | TCCTGTGGGGAGAAAGGAGGCCCTTGGGCACTGTCTGA |
| | | AAAGTGCGATGCCAAATGATCTTTAGGGACAGACAGT |
| | | AAATTATATCCCTTGCTAAAATACCTCTCGGTAGCGAA |
| | | CCACCAAACGTTTCTTTTTTAAGCCCCGCCTCTCAGCCG |
| | | TTACGCCTAGTTCGGCTGTCTCTGATTCAGGAAGCTGG |
| | | CAGTTCCCACTTACTCCTCCCCAGCAAGAGCTAACAGC |
| | | CCTCCTAAAGAAAGTTGACAGACAATCCGTAGGCCGTC |
| | | AACACCTTCAGCGACTACG |
| 28 | MAST2-MMACHC | GGTCATAATTATGCCTAACATAAAACAACAATCCTTCA |
| | | TACAACTGGAGACGCACCAATCCCCAACCCAAATGCTA |
| | | TTACATAAAGTTAACAACACTTAAATGTTGACGTGAAG |
| | | TTAATAAATCTTATGTCACATGATAAAGGAGAAAGGA |
| | | AATAAAATGAAAGTATTTTCTTAGTACAAGCGTATATA |
| | | TGCGCAGACGTGTTTTTAACAAAAGAAAGAGGAAATA |
| | | CTTATCCTTATCAGAGGAAATTTTTGCTAACCTTCCGAG |
| | | TTGTCTCAGAGCCAAACGTAGTAAGCTACCTGTCACCG |
| | | GATGTGCTTGAGCTAAGGGCGCTGAACCACCTCTGTCA |
| | | GAGACGGTGCATCCCCCACCGGATGTGACCCCGGGAT |
| | | GCGGAGCCTGGGGTCAGATCTGCGAGCCAGATAACAG |
| | | CGGCACTCACTTCTCGGGTCTTCGTGAGCATTAAGTCA |
| | | ATGAACCCAGGAAAGAAAGTTGACAGACAATCCGTAG |
| | | GCCTACGGCGACCACAGCCGCTGCG |
| 29 | TENM4-RRAS2 | AACGTCTCCCACCAGGCTCCACCTCTAACATGGGGGAT |
| | | GACATTTCAGCATGAAATTTGGGGGAACAAATATCTAA |
| | | ACTATTCACACCTTACTAATAATACTAAATGTGCACAG |
| | | TTAAATTTCAGATAAAGATTGTTCAATTGGGGCAGACA |
| | | CGTAATTTTTTCCATTGCTCTTTGGGACTCAGATGAATA |
| | | ATCTTAGTGTGGTAGAGATAAGCCTAGCTGGTTTGTCA |
| | | TTCCTTCTGGGTGTTAGAATAGACTGACAGCGCCTGGA |
| | | GATGGGGGTGGGAAGAATATGAATATTATGGGATGCC |
| | | TGGCTCGAAGGGTATAGGGGTGGTAGAGTGGAGATGA |
| | | AGCATTATGGGATGTGACACTGGGTTGTGTTGGGAGTA |
| | | TTATGGTATGTTTGGTTGACCACGGGCTCCTTGCAATTA |
| | | TTATGGGATGTCAGGATGTGGGTAGGAGAGTAAAATT |
| | | ATTATGGGAAAAGAAAGTTGACAGACAATCCGTAGGC |
| | | CACGTCCTCAGCCACAGCTCCAG |
| 30 | OTOGL-SELPLG | CTGAATACTCGTGCTTCCTACTTTGATCTTCTCCTGAAA |
| | | CACGAAATGAATCAGTTCTTTCTCATTTGTGACCTTTCA |
| | | GATGTTTAAATGCAGCTGTCATGTCTGACTACTTGGCA |
| | | CATTACAACATTCTACTGACCCAGGTCTCCTGTCTCTGA |
| | | GTTACCTTTTATTCCAGTTTTCAGCTCTAACAGGACCCT |
| | | TGTAGACTTCTTATTTCCTCCTTTTCTTTAACCTCTTCCT |
| | | TGACCGCCCCCGCCCCTCCCGCCTGCTGTCGCCCAGCT |
| | | CCGCCCCTCCAGGGCCGTGTCCCGCCAAGCTCCTCCCC |
| | | GCTCGACTTTTCCTTCCGGACCCCATCCCTTCAAGCCCC |
| | | ACCCCCTGCAGGGGCTCTCCAGGCTTCCCAACCAGGTC |
| | | CAGCTCCAGGCCCTGTTCCCTGTCCCCAGATCACACCG |
| | | AGTCAGCAGCCTTCCCTAGACCCGCCGCTTCCTCGCTC |
| | | CAAAGAAAGTTGACAGACAATCCGTAGGCTTCATCTAC |
| | | TACCACTACTCCAA |
| 31 | DNAJC5B-LINC00861 | TATTTGCCACCAGGCAGAACAGAATGAGCATTGGTAG |
| | | AGAAGGCTCCCTCTTGGTATGTTACTGATAAACTTGTA |
| | | CTTCCTCATACTTGTTTGTGGTTCTACTGACTTATTTCC |
| | | AAACTTAAGAGTAATGTACTTCGAAAACAACTTGGTTA |
| | | TTCTTTAAATTTCAGGTAAAACCCCTTTTCCTAACACTC |
| | | ATCTTATTTCCACTGTCTTATTTCCTCTCAGATGTTCATT |
| | | CCTTTGGTTTCTCACACAAACCCTGCTGTGGCTGCTTAC |
| | | CATCCTCAGCAAGGGTGCGGAGGAATGGAAACTTTTGC |
| | | CACCAACAGCATTTGCTAGCAACTTCCCTCTCTGTGGTT |
| | | TTTCTGTCATTCCCTTGATAAAAGAACTTGAGCTGCGC |
| | | AAAAGTAACCAATCTTCACCAATCTCATCTTCACATAT |
| | | AAACAGCCGCCTTTCAAGAAGCAAGCTGCCAGAAAAA |
| | | TGATAAAGAAAGTTGACAGACAATCCGTAGGCCGGCG |
| | | TCTGCTGCAACTACAACA |
| 32 | ELOC-LETM2 | TCTTAAAATGCTGAGATTGCATAGTAGCTTTTGGGTAG |
| | | AAGTTAAAAAACCAAACCAAAACAAGAACCTCCAAAA |
| | | ATAACAAAGTGCTGGATTACAGGCATGAGTCACAGTG |
| | | AGCCACGATGCCCAGTCTGTCTCTCATACACACACATA |
| | | CACACAAACAGATACAGGTTGTAACTTACTTTTTTTTTT |
| | | TTTTTTAACCATTTGTAGAAGGCTATTAGTCCATGTCAG |
| | | AATCCTAGAAAAAAACGCACACAGGCGGCGGCCCCAA |
| | | ACCGTTGCCGCCCACTTACCTCAGCTCTGGCTTTACACT |
| | | GGTAGCCGCCTTCCCCTCTGCAGCCGAAACCAACTTCC |
| | | GGTCTCCTCCGGGAAGAAGCCACCGACTCTGTCCTTGA |
| | | TTGGCTGTAAGAGCTGTCCTTTAACGAAATTCTAGCTC |
| | | CGCCTCAGACCGATCCCTCGCTGTTGCGCCTACGTATG |
| | | TTTGACTCAAAGAAAGTTGACAGACAATCCGTAGGCTC |
| | | CCTCTTCTTCACCCACTTCAT |
| 33 | OTP-CSF2 | AACTCTTGGTGTCTGTAGAGTCAGGAGAAAGAAGTATG |
| | | TGTGAAAAGTTAACAAAATTTTGAGAAATTGGAATTGA |
| | | AAGTTAAGTGATAGAGTGTGTGCTTTCAAATTTAGTCA |
| | | TATAAACACTGAAACCCAAACCACCAGAGCCTTCTGTG |
| | | CGGAGCTTTTTGTGGTGTTCTTATTCTTATACTGCCAGT |
| | | CTTGGCTTTGTTAACTCGTTTGAGTTTTGGAGAACTAAC |
| | | TCCTTAACACCCATGTCAATACTTCCCAGTGAACGCAC |
| | | GTGTTCGACCTTTTATTTAAATTTCCATTTCATCACAGC |
| | | AATAAGACACTCGGGGCGGTGCATTTTCTGTGAAATGA |
| | | CTACTGTCCGGGGCCCGAGGTGCTGCCTCGCACAATGC |
| | | ATCAAAGGAGGTTGTGTCCGTGGTCGTAACCTCTTCGC |
| | | GTGAAAGGCACCGCCTGGTCCAGTCTTTGTGAAGGAAC |
| | | AAAATAAAGAAAGTTGACAGACAATCCGTAGGCCGCG |
| | | GCTACGACATCCTCGGCAC |
| 34 | SNAI2-EMC2 | TGTCTTCTAACTCTGCTACCACCACCCTCGTTCAGGTTT |
| | | TGTAGTAAGTATCTGTGGTTTTGTCTGTCAGGAGTCTTC |
| | | CTTCCGGTGGGCACTGCCTCTCCTCCCATTCTATTCATG |
| | | TGGTTCCTACTGGAGATTGCATGCCCATATTTGACCTA |
| | | CCCTATCTGTCTATGGTTGATAGGTCTGCAGGCAGTCA |
| | | TGTGGCCCGAAGTGGGACAGTGAGTTTCTTCTTACTTT |
| | | CCTTGGTCCCGCTACTGGACCCATTTCCTACCTGGGAA |
| | | ATGGAGTCGAAGCTGACTCAAAACGTAACGGCCCAAT |
| | | TGTCCTTGAGACTTCATTCCCCAGCAAGCTCAGCGTGT |
| | | AACGTGCGCTATGGAGCCGAAAGTCGCAGAGCTGAAG |
| | | CAGAAGATCGAGGACACGCTATGTCCTTTTGGCTTCGA |
| | | GGTTTACCCCTTCCAGGTTAGTTTATCCCTCCTGCTGTT |
| | | CTAGGGAAAGAAAGTTGACAGACAATCCGTAGGCAGC |
| | | GGCTGCGACGGCAACCTCAA |
| 35 | RGS6-DCAF11 | GGCCCAACCCTATGGCTTCAGATTCAGTAAGCCTGGGC |
| | | TGGGGCTGGAGAATGTGCAATTCTAACAAGTTTCAGGT |
| | | GATGCTGTTGCTGCCACTCAAGGGACCAGCACTGAGAA |
| | | CCATTGGTCTATGAGATCCCCAAAAGGAGTTGCTGTGG |
| | | TTTGAACATCCTCCAGAAGTTCATGTGTTGAAAGCATG |
| | | CTGCAGTGCAGAGAGGTGGGTCCATGTTTGCCATCCTT |
| | | TATCCTCCCCAGCCACAGCCTCTACAAAAGCAACTAGA |
| | | AGAAGACGGCAGCAGAGGGCGCTTCTTCAAACACCCT |
| | | GTCCAACTCCTGGCACTGACAAGTACTGAGTGAACTCA |
| | | AACCCTCTGTAAAGTAACAGAAGTTAGAAGGGGAAAT |
| | | GTCGCCTCTCTGAAGATTACCCAAAGAAAAAGTGATTT |
| | | GTCATTGCTTTATAGACTGTAAGAAGAGAACATCTCAG |
| | | AAGTGGAGTCAAAGAAAGTTGACAGACAATCCGTAGG |
| | | CATCAACGTCCGCAACGGCAACCA |
| 36 | SLC35F4-DCAF11 | TCAGACTGGCTGGCAGAAAAAAATAAAGTCACATCTG |
| | | GAAACAGCTGCACTAGCAATTAAATAGCATTTAATGTG |
| | | ACTAATATCAAAAGGGTGGGTTATTTTCCCCCTAGCAT |
| | | TAAGGATGCAAAATTTCAGCTTCCAAGCTACTCTATTA |
| | | TTTTTTGAGTCAGTGGGTGTTTAGAAGTCATTCTGATAT |
| | | TTATAAAGAAAAACCCCCAGTGAGTCATAGAAGGGGA |
| | | TTTTCCTGGCTGTCGGTTCTTGGAAAGGTTCATTAATGA |
| | | AAACCCCCAAGCCTGACCACCTAGGGAAAAGGCTCAC |
| | | CGTTCCCATGTGTGGCTGATAAGGGCCAGGAGATTCCA |
| | | CAGTTCAGGTAGTTCCCCCGCCTCCCTGGCATTTTGTGG |
| | | TCACCATTAATCATTTCCTCTGTGTATTTAAGAGCTCTT |
| | | TTGCCAGTGAGCCCAGTACACAGAGAGAAAGGCTAAA |
| | | GTTCTCTGAAAGAAAGTTGACAGACAATCCGTAGGCTG |
| | | CAACGGCACCGACTTCTACTA |
| 37 | UTRN-FGD2 | GCCTGCGCATCTTCCCTGAGCTCATTCCTGTGTGCATAA |
| | | AGTGGGGCTGAAGTAATGAGGGTTGGGGGTGTTTTTTC |
| | | TAGAAAACAGACTAGAAGTTATATCTAAATGCGTGAGT |
| | | CAGCTCCATGAGTCAGTTCCTCAAGAGATACTGGTTGA |
| | | CACCAGGACCGGTTCCAGCCCTGGGAGCCCCACCCTTG |
| | | GCGTGTGACGATACATGACATGGAGGTGTGATGGCTTT |
| | | ATCCTGCAACCCAACGTCTCATCTTCCCATAGTAAAGA |
| | | TGACGGCGCCTTGAGGTAAGCTACAGGCAACACCACTT |
| | | CCGCGTTTCTCTTGCGCCCTGGTCCAAGATGGCGGATG |
| | | AAGCCACGCGACGTGTTGTGTCTGAGATCCCGGTGCTG |
| | | AAGACTAACGCCGGACCCCGAGATCGTGAGTTGTGGG |
| | | TGCAGCGACTGAAGGAGGAATATCAGTCCCTTATCCGG |
| | | GTTAGTTGAAAGAAAGTTGACAGACAATCCGTAGGCCT |
| | | ACATCGGCAACAGCTGCATCA |
| 38 | SYF2-THEM5 | TTATTAAAATCAAGAACAGAAAGGCCCTGGTTGTGGG |
| | | GGCAGCTGCCGTGCACCAGTAGCAGCAAGGAGGCAGT |
| | | CACATGGTGGTTTGGAGCTCAGGCTTCCCAGCCGCACT |
| | | GCTGTGGTTTCAACCTGACTCTGCCATTTGGCAGCTGT |
| | | GTGACCTGGAACATGTTACTTAACCTCTCTTTGTTTCTG |
| | | TTTCCTTATGTCCAAAATGGGAATGATAACAATGGTTC |
| | | TACTCCTTACACACCACCCTCACAAACCAACTAAACAC |
| | | ACTGTTACAACTCGTCTAACAAACAGCATTTCAGAAAT |
| | | ATATGTTTTTCTCTACAATGTTTCAGCAAAATACAGCTT |
| | | CATATAGTCTCTCACGCTCAACCTGTCAAAACCGAATC |
| | | CACACTGTCACACCCAGTCCCAAGCTCCCTGACGTCTG |
| | | CATCCTCAGACTCCGTAACACAGAAGTAACCAGACTGC |
| | | CATGGTAGAAAGAAAGTTGACAGACAATCCGTAGGCC |
| | | GTCAGCGTCCACGACTGCTTCG |
| 39 | EIF2D-TMEM79 | TGAGAGAGAGGTTAGCACCCATAGTTCTTCCGCGAGAA |
| | | GGGGGCGGGGATTGAAACTTGATTGATGGCATTTCCGA |
| | | CCAGTGAGAACGTCTGAGTCAGGGGCCCTTGAGTAGTA |
| | | TGTGTCTTTTGGCAGTAGAGGGTTGGTCCTTACTTTGAC |
| | | TGACCCTCTTATCATCCAATAAACATGCCTACTTGCGTT |
| | | GGGGGCGGGGCGACTGAGTGCAGAAAAGACGGGATCT |
| | | TTCCTGGGCGGGCCCTCTTCTGCCGCACTGCTGGTGGG |
| | | AGGGGAAATTGGGGACCCGAGTTGGTGGATGACGTAG |
| | | CCCTCACGTGACCAAGAGCTGACGTGTGCAGAAGTCCT |
| | | TCTTGTCCTGGTCGTTGTTCCCGTCTGAGTACCAGCTCC |
| | | CCACTGCCCTGAGGGCGGGCCGGCCTGCGGCGGAGGG |
| | | AAAAAGGAAGAGGAGAAGGAAATTGTCCCGAATCCCT |
| | | GCAGGTCAGAAAGAAAGTTGACAGACAATCCGTAGGC |
| | | TCGCCGGCTTCTGCTACCGCGAC |
| 40 | | TCTGTGAATGACAAATGATAAGTGCAGTTGTGATTAGG |
| | | GCTATCCATGCCTTTGAGGAATCGTTTTTATGCCTGTCT |
| | | CTCTGAAGAGAATAATCATTTGATGAAGAAAAGACTG |
| | | AGTCATACCCTAGAGTCAGAACTATATTCTTTTTCCAA |
| | | CTTTTTTGCATGTGTGTGCATGCATGGGCACACACACA |
| | | CCCCTAAAGAATTTCTGACTCACTGTAAATTGGTATTG |
| | | AATCCTGCTCGGCGCCCCTTACGCCACTGGCTCAGCCT |
| | | CAGAGACTTCGCGAATATGGCTTTGCGCACTGCGGCGG |
| | | GGCTGCGCCGCAACCGAGCTCAGCCCTGTGGCTCCTCC |
| | | TCGCTGCCCTGCGCGGATTGGTGGCAGACGGCAACCGG |
| | | GCCGCTGATTGGGCGGCGAAGGAGCCATTCGGGGAGA |
| | | CTCTGGTGGGTTCGGCTGCCCCAAGAGTGATAAGTTCG |
| | | GCTTCAGACAAAGAAAGTTGACAGACAATCCGTAGGC |
| | | CGGCTGCGTCATCTACTTCATCC |
| 41 | XRRA1-EIF4G2 | AAACGTTTGCCAACCCCTGGCTTGCAGAATTACTAAGA |
| | | GCTGAAGAAACTGAACCTAGGCTTAGCTTCCAGGAATA |
| | | ATTCTTAAAGTCTTACAGCAGAATTGAGCTTCTAAGAA |
| | | AGTGGCTGCCCCTACTATAATCAGGAAGATGTAGAAAC |
| | | AGGAAGTTGCCTAAGAGAGGAGGAAGCCACCACTGCC |
| | | TGTGCTGGCTCCAGAATGTACTCTACCTTTGCTATGATT |
| | | CATCCTAGCAGACAGTGTCAGTGTTGATGACTAACGGT |
| | | TGAGTAGCATATACAGTGTGGATACTCTGGACAAAGG |
| | | GATGATTCATTGTCCTGGGTGAGATGGTGTGAGATTTC |
| | | ATCAGGCTACTCAGAATGGTAAACAATTTGAAACTTTA |
| | | TTTCTGAAATTTTCCATTTAATATTTTTGAACTGTGGTT |
| | | GACTGTGGGTAACTGAAACCAAGGAAAGTGAAACTGG |
| | | GGATAAGCAAAAGAAAGTTGACAGACAATCCGTAGGC |
| | | CTCTTCTACTGCACCCACAACAT |
| 42 | RAB30-IFITM1 | TTATTTACATTCATGACAACAGAACCGAAGCCACAACA |
| | | TAGGGTCTATTGTTCACCTCATAGGGGTGACCGAATCT |
| | | ATTTATCCATGTGGTTTGGGAGCTCACCACTGTGCATT |
| | | GAAGGGCCTCCTGTTGGCAACATCACCAAGAGCATTTC |
| | | AGTCACTGTTACTACAATCTGAACAATTTGGGTTCCAC |
| | | GCTGTGGTTTTGTAATTTCTATTTTTAAAACTTGAACCA |
| | | TTCCTAAATTATTTTAAAGATCAAAGAAAACTTTCGTC |
| | | ATACTCCGTATTTGATAAGGAACAAATAGGAAGTGTGA |
| | | TGACTCAGGTTTGCCCTGAGGGGATGGGCCATCAGTTG |
| | | CAAATCGTGGAATTTCCTCTGACATAATGAAAAGATGA |
| | | GGGTGCATAAGTTCTCTAGTAGGGTGATGATATAAAAA |
| | | GCCACCGGAGCACTCCATAAGGCACAAACTTTCAGAG |
| | | ACAGCAGAAAAGAAAGTTGACAGACAATCCGTAGGCC |
| | | AACATCACCTACTACCCCGACT |
| 43 | RASL11B-CD38 | GTATGATGATTCATACAGATACTGCTTTATGTGAGAGG |
| | | ATAGGCCAGAATGGAACCAAATTTCTAACTTTTCTATG |
| | | AATTGGAGTTACTAATACCTGTCTCTTGGATGTGACCA |
| | | TTAAATGAGGGATCAAAGTAAACTCACTTTTTGTAAAG |
| | | TAAGTTCAGTGCAGATACTGTCTTCTGTAGAGCAAATA |
| | | TAAAAAGAAGTTGCTTCTCATTTCAGGAGGAGAACAAT |
| | | CATCCTCTCTACCAATCAGCAGGATGTGGGTGGGGCCA |
| | | GATAAGAGAATAAAAGCAGGCTGCCCGAGCCAGCATT |
| | | GGCAACCCGCTCGGGTCCCCTTCCACACTGTGGAAGCT |
| | | TTGTTCTTTCGCTCTTGCTACTGCTAGCTCTTTGGGTCC |
| | | ACGCTGCTTTTATGAGCTGTAACACTCACCGCGAAGAT |
| | | CTGCAGCTTCACTCCTGAGCCCAGCGAGACCACGAGCC |
| | | CACCGGGAAAAGAAAGTTGACAGACAATCCGTAGGCG |
| | | TCCACGTCGTCGCCGCCTGCGG |
| 44 | NDUFB11- | TGCAGCACTGTGTATTCATGATCTTTTCAGATTTGCTTA |
| | (chr101623051101623281) | TTGGTGCAGAAAGGGGAAAGAGTCTAAAGTTTCTTGA |
| | | ATCTTTGCCAGACACTGTACTATGCCTCTGAATGCATC |
| | | ATGCCTTAATCACGCCTCACAGCAACTATTGCTACTTTC |
| | | ATCTTTCAGACAAGGAAACAGGTTCAGAAAGGCTAAC |
| | | CAAGTTGTGACAGGCACCTAGCCTGAATCAGTGGATTT |
| | | CTTCCTAGTCTAGGGCCCCCGGGGCGTATGACGCCGGA |
| | | GCCCTCTGACCGCACCTCTGACCACAACAAACCCCTAC |
| | | TCCACCCGTCTTGTTTGTCCCACCCTTGGTGACGCAGA |
| | | GCCCCAGCCCAGACCCCGCCCAAAGCACTCATTTAACT |
| | | GGTATTGCGGAGCCACGAGGCTTCTGCTTACTGCAACT |
| | | CGCTCCGGCCGCTGGGCGTAGCTGCGACTCGGCGGAGT |
| | | CCCGGCGGAAAGAAAGTTGACAGACAATCCGTAGGCG |
| | | CACCGACAACGCCGACATCTTC |
| 45 | ITPR2-OASL | GGAATAAAAGCTGGCCACCTGAGCCAGCCCCAGCAGC |
| | | CGCTCGGCTCCACTTCCATGCCATGGAATCTTTGTTTTT |
| | | TCACTCTTTGCAATGAATCTTGCTGCTGCTCACTCTTTA |
| | | GTGAGCACTACCTTTATGAGCTGTAACACTCACCACGA |
| | | AGGTCTGCGGCTTCACTCCTGAAGTCAGCAAGACCACG |
| | | AACCCACCAGGAAGAAGAAACAACCCTGTACGTGCCA |
| | | TCTCCTTGGGTCCTGTCTTCTCCACCTTCTACGTTTCAA |
| | | CCGGAAATAGTCGTACCCATTTAAAGCCACGTCGGTAC |
| | | TGGCCTCGGTCCCGCCCCCCACACTGCGGAACCGTTTC |
| | | CGGGGTCAGGGCATTCCGTCCCGCCCCGCCGCCGGTGC |
| | | AGTGTTGGAAGCTCCGGTTCTCCCGGAAGTGGCCCAGG |
| | | TCTCTCTGTCGGGGTCCCCTCCATCTCGCTGCTGCTGAA |
| | | GGCCGCAAAGAAAGTTGACAGACAATCCGTAGGCTCA |
| | | TCTTCTACTACGACATCATC |
| 46 | SUGT1-FLT1 | GCAAAACAAGGTCAGGTTGATGTTCTCAGGGTTGAGTC |
| | | AAATCCACCCATTTTCCACTAGGTGGCAGACCTTGTTA |
| | | AAAAGTTACGGAAAAGGAAAGAAAGACAAAGAAAAT |
| | | AATGAGTCTTCTGAAGCTGACCAATAGATGTCTCTTTG |
| | | CAACCACATATCCCTGAGCTGTCGCTTAAAATACACAC |
| | | AAATTCAACACTCCCTAGTCTTTCTTTTTCATTTTACAG |
| | | ATATCCTGATTGCGGGAGCGCCACGCACCGCCCACCTA |
| | | GCTCCGTCCACCGTCCAGCTCGCTCCGCCCAGCTCGCT |
| | | CCGCCCCCTTCCCCGAAAGCCCCTCCGCAGAGCCGCGT |
| | | GCACGTGCGTGGCGGATACCGCCCCCTCTGCTCTCTTG |
| | | GCCAATGAGAAAAAGCTACGTAACTCTCCACCGCCCG |
| | | GTGGCGGGTCACGTGACTGCGTCTCCCCGCCCTCTCAC |
| | | CCCGCTGCCTAAAGAAAGTTGACAGACAATCCGTAGG |
| | | CAACTTCATCCACGCCCACTTCTT |
| 47 | NEK6-SEC16A | GCAAATTCCTGGAACTGCTTTTACTGTTTTCTGTGACTC |
| | | TTATTAGGAACCACACTCATGAGTCATACAACCAGTAA |
| | | GGCTTACCAAATATTTACCATGAGCAGAATCCTAAGCC |
| | | AGGTCCCGAGAGGGCGCACATAAAGTCAAAACTGAAA |
| | | GGAAGTCCCCAAAGTTATCTGATCCAGCTTCCTGCTTT |
| | | CAGGTAGGTGAATGTCAATAAAGTGAGCTAGTTGTTAG |
| | | AATCCTCGCTTGTGGTTCTCCACTCCTGATTGGTCAGGT |
| | | TGCCCCGCCATCACGCCTCCCTCTGCTTTCAAGGTTGA |
| | | GAAGGAAGCAGGGGTAGGGGGGTGGGGCCAAGTGGC |
| | | GCGTGCGCGATACTGTTGCTGCCCCTTTGCTGCTATTGC |
| | | GTTGCAAAAAAAATCCTGACTAGGTAGCCTTGGACCTT |
| | | TTCTGTGCTAGCGAGTCTAAAGGAAAGAAAGATTTCTG |
| | | CAACTGAAAAAGAAAGTTGACAGACAATCCGTAGGCC |
| | | CTCATCTCCTTCTTCAACATCT |
| 48 | RPS6KC1-LAMC2 | GAACTCAGTTGCTGCCACACCTCATGGTCTGAGGAAGC |
| | | CGGCCCTTCTACTCAGAGACAGTGCCTGAGAGGAGACT |
| | | GCTGAAGCCTCAGCTCAGCAGTATGTGAGCTTGTCTGC |
| | | CTCTGCAAACTGTCACTTTATCCAGCATCTGTAGGTTTG |
| | | CATTGCCAGATACCCACATCAAAGCCTAACCTCTACAG |
| | | GTGGCTAATGGAACACAGCAAAGAAAAAAACCACTAA |
| | | GGTCCTGGGCCTCTGCCTCACCCCCACGCTGCCTCAGC |
| | | TGGGGCAGCAGGCCCAGGCCTCTTGCATAAGCGCCCTG |
| | | TGGGGGACGAGGCTGCAGCTGTGCCTGGCCACCCCATC |
| | | CCCCAGCATCACGAGTGTTCAGTGGGCTGTCCCCGGGC |
| | | CCTGGGTGGAGGCCAAGCCTGGCCCATAAATAGGGGT |
| | | CTCCTCAGTGCCCTCCGCTCCTCCTGCACACCTCCCTCG |
| | | CTCTCCCAAAAGAAAGTTGACAGACAATCCGTAGGCCC |
| | | TCATCGCCAGCCCCAACGACT |
| 49 | TSTD2-SEMA4D | AGAAAATATATGGATGATATGTTCTTTGCTGTCAACAC |
| | | TTAATTAAAAGAATAAATTTTGCGTTTGTTGGCTCTGGT |
| | | TGCTCATAATTGACTTCTCTCTGGTACAGACTGACATC |
| | | ATTGACCACAGCTTTGCAGTGGAATGGATGCAGGATTT |
| | | TGAGGCTTTCCAAGATGCCTTGAATCAAGAGACTACAT |
| | | ACGTCAGTAACCTGACTCGTTCAATGAGCCTGGTGTTA |
| | | GTCCTGCTTCAGGCCTGCTGGCCTCCCAGCACTGCCTG |
| | | CCCCTCCCTGTCGGGGGACATCGCCTCCACACCGGCTG |
| | | GGGAAGGAGCCCAGGGGTGGGGCTGGTGGGTGGGGCT |
| | | GGTGGTTGGGGCAGCCAGAGAAGTAAGAGGGAAGTGA |
| | | GAAGCCGGGTGGGGCAGGCTGGAAGGAAGACGAACCT |
| | | ACGAAGCAGAGGTAGGAGGAAGAAAGGAAAGTAGGA |
| | | GTGTTGAAAGGTAAAGAAAGTTGACAGACAATCCGTA |
| | | GGCCGCCTGCTGCCACACCGGCGGCA |
| 50 | LTBP1-RETSAT | CATGTTATAAATCATTGTGGAAGGCTGAACAAAACAA |
| | | GTGTGGATCCTTGGGCAGGAAATCCACTAAGAATAAC |
| | | AAACCTGAAAGTTGACTATCACTAGTGGAGATGACATC |
| | | AACATTGACTTTGGTTGCTATTAATAAACACACTAAAA |
| | | ATGCAATAGGTTTAATGACTGTAAATGCTGTCTCCCAT |
| | | CTCAGCACAAACTGCTGTGTCTGGGAATAAATGAGTAT |
| | | TTATTCCTCGGGAGTTGTAGTCTCTAGCGACTGGCCCC |
| | | GCCCCGTGTGCAGCTTTCGCGGCCTGCGTCTGGAGAAA |
| | | ACCCTAGCGGGTGGCTCCGTGCGGCCAGAGCTCTAGAG |
| | | AGTGGTGCCGCCTTCCAACCTTCTTCCCCAAGCCCTGG |
| | | TGGCCGGCTCCGCCTCTTCTCGAATCTTTTCCACAGCCC |
| | | AAAATGGCCGCAGAGGTGTATTTTGGCGATCTAGAGCT |
| | | CTTCGAGCCAAAGAAAGTTGACAGACAATCCGTAGGC |
| | | AACGGCCACTGCTTCCTCCCCTC |
| 51 | KLRG1-TCP11L2 | TCATGCAAGTATTTAAGTCTACTGGAAAGGTGTGAGCA |
| | | TCATTTACCTTACCATTTTTTTCCATTAGTACAGAGCAT |
| | | AAAAAACTAGTTATATAAAGTTGTTTCTGTGAATCAGT |
| | | AGTTGCTTTGCTGTTTTCTTCCTCCAGGTGGGGCTCTTG |
| | | TTGTTTATCAGACTAGTTTTTGTTTTGACAATAGCTTTA |
| | | AAAAAATTAGAAGAGTTACCTAAGTTTCTTTTTTTTTTC |
| | | CTACCCAAGTCCCTGCGTGGTAGGAATTTAGACCCTGA |
| | | GGAAACTCCATTGTGTGTTTCTAAGCTGCTTAGCTGTC |
| | | AGTGATGCAGCTTTGCTTTCAGAGTAACAGAGGAACTC |
| | | CCAGCTGTGTGGGTGATGGGCTTTGTGATGTAACAGAG |
| | | AGCGCGTTCCTGCAAGCAGCCTTGAGGCTGGGAGGGG |
| | | TCCACCTAAGCCTTATGCTCCTTTCCCCTGAGGTTCTAC |
| | | AGATAAAGAAAGTTGACAGACAATCCGTAGGCAACAT |
| | | CAACATCGCCATCGACTT |
| 52 | STK40-ST7L | GACAGGGTTTCACCGTGTTAGCCAGGATAGTCTCGATC |
| | | TCCTGGCCTCATGATCCACCCACCTCGGCCTCCTAAAG |
| | | TGCTGGGGTTACAGGCGTGAGCCACAATGACTGGCCTA |
| | | GAATTTTCTACATACATAATGAAGTAATCTCTAAATAA |
| | | TGACTCATTTCTTCCTATTCAATCTTTTTGTCTCATTGCA |
| | | TTGACTAAAACCTCAAGTTCAATATTAGCTAACTCATT |
| | | TCCTCACTTCCGCTCCCGGGGGGCGAGGTTGCTGCAGT |
| | | GCATTCGGTGGTAGTTTGGTGTCAGTCCTGTGTCTTGTC |
| | | GGTCCTTCTAAACGTGTCCCCTTACGATGCTGGCTCGC |
| | | AACCACCTGGAAGTTGACTAAGGGGAGAAGGAGTCTC |
| | | GTTGGAAAACGGAGGTTTGGAGGGACAACGGCCTCTC |
| | | TTTTCGTTCTTGTCAGCGGAGCCTTCTTGGTCTTGGGGC |
| | | TGTCCTAAAGAAAGTTGACAGACAATCCGTAGGCGCC |
| | | GCCTCGGCCGCAGCATCGGC |
| 53 | ZNF562-RAET1L | TTGTGCCCCATGTATGTATGTGTGCATTTAGTTCTATAC |
| | | AACTTTATCACGTGTTAAGATGTGTCAAAATACAGAAC |
| | | CATTCTGTCACCACAAAGATCCTTCCTGTTGCCTTTTAT |
| | | AACCACACCTCCCTTGCTCCCTCTACTTCCTCATCTCTA |
| | | ACCCCAGGAACCACTAATCTGTTCTTTGTCTCTATAATT |
| | | TGTCATTTCAAGAATGTTATGTAAATGAAATAATATTC |
| | | CTGTGGGGAGAAAGGAGGCCCTTGGGCACTGTCTGAA |
| | | AAGTGCGATGCCAAATGATCTTTAGGGACAGACAGTA |
| | | AATTATATCCCTTGCTAAAATACCTCTCGGTAGCGAAC |
| | | CACCAAACGTTTCTTTTTTAAGCCCCGCCTCTCAGCCGT |
| | | TACGCCTAGTTCGGCTGTCTCTGATTCAGGAAGCTGGC |
| | | AGTTCCCACTTACTCCTCCCCAGCAAGAGCTAACAGCC |
| | | CTCCTAAAGAAAGTTGACAGACAATCCGTAGGCCCCCC |
| | | AACAGCAGCCACATCCGC |
| 54 | GPR65-RN7SL1 | CAAATCATGGTCCAAATGGCCAGGAACCTGGGGACAC |
| | | ATAGTGTGTTCTGGCAGAGGAGAAATTCTAGGTAGCCA |
| | | GGATACAGAGGCCACCTTGTGACTCACCAGTGTGTCAG |
| | | AGTAAGTAAGTCACTTCATTACCTAAACCCTCAGTTTC |
| | | CTCACTTGCAGAATGAATGATTAGAGCTTTCTCTTGAG |
| | | GTTGTTGGGAGGTCCAGATTAGATAATGTCTGTGAAAG |
| | | AATTCCTTCATTGGTCCCTGGCTAATTCACCAATTTACA |
| | | AACAGCAGGAAATAGAAACTTAAGAGAAATACACACT |
| | | TCTGAGAAACTGAAACGACAGGGGAAAGGAGGTCTCA |
| | | CTGAGCACCGTCCCAGCATCCGGACACCACAGCGGCCC |
| | | TTCGCTCCACGCAGAAAACCACACTTCTCAAACCTTCA |
| | | CTCAACACTTCCTTCCCCAAAGCCAGAAGATGCACAAG |
| | | GAGGAACATGAAAGAAAGTTGACAGACAATCCGTAGG |
| | | CATCGGCGTCAACGGCAGCTTCTT |
| 55 | EED-GAS2 | CTCGGGGTGTCTGCCCAATGGATTTGTCACAGAATCAG |
| | | AGGCCCAGTGAGTGACCAAGGCTTGCCTGAGCAGGAT |
| | | ATGAAAGCCGCGCTGCTAGAATCTAAGCTGGACCGCA |
| | | GCATAGGCCACGCGCTTTCCATCCTACCTGCCCTGTAA |
| | | CGTGACTGACGTTAGCTGCGGTGGGGCGAGGAGCGCC |
| | | GTCTTGTGTCCTGAGTTTGATCACCTCGTCAGACCACA |
| | | AGTTGTCCTTCTGTCATTCCCTTGATAAAAGAACTTGA |
| | | GCTGCGCAAAAGTAACCAATCTTCACCAATCTCATCTT |
| | | CACATATAAACAGCCGCCTTTCAAGAAGCAAGCTGCCA |
| | | GAAAAATGATGCACGATGCTCTCTAAACTGGGTCATTC |
| | | TCCACTTGGAGGGCTCAGGGCACGGTTGACTTTCCCCG |
| | | TCTGTCTCCTATACCACAGGCTCTGGGCATCACCAGCG |
| | | GCCCCAGGGAAAAAGAAAGTTGACAGACAATCCGTAG |
| | | GCGTCGTCAACCGCGCCCGCTGCGG |
| 56 | PAG1-NCALD | GGCCAAGGTCTCTTGGGCAAACTGCCAGCAGGTCCCTG |
| | | TGGGAGGCAATTATGATGTCATGTCTTTCCTGCTGTAA |
| | | CCACATACTCTCAGACTCCAGATAGTCCTAAGGCAGCT |
| | | GTGACAAGCATCTGTATTAGCTTACTAGTGAGGCAGGG |
| | | TTCATGGGTCCAAAAAACTTGGCACATCAGTGCTTCTG |
| | | GGCAGCAAAATGGATTGGGGGAGGGGTGAGGCAAAGG |
| | | TGTTCCTCCCTCCGTCTTCACCCGCCCTGGGCTGCAGAG |
| | | GAACAGGGAGTGGTTTTGGTGGTTGGCACAGGGGTAG |
| | | ATGTTGATGGCACGAGGCCCTATCATCTGACAGCTGCC |
| | | TGACCCACGGTGGGGAGGGCCCTGGAGGGAGTGCTGC |
| | | GGGAGCAGGAGGGGCAGTTCCTATAGGCAGCCTGACT |
| | | CCCATGGTGAGCCCACTGCCACCACCACATTGCCCTGG |
| | | GGCAGCCCTGCAAAGAAAGTTGACAGACAATCCGTAG |
| | | GCCGCTTCTACCGCAGCTACCACCT |
| 57 | DPP4-LTBP1 | AAGTCAAATTATCAAAAAAAGGAACTGAGCTGGTGAT |
| | | GTGCACTAACACAGCAAATCACAGGAAAGGGGAACCC |
| | | CAGGTAAATTACAGCCTTCTGACCTAGGAAGACGTGTG |
| | | GTTTGCGTCTCTGAGTTACAGAAACACAGGAAATGCTT |
| | | ACTGGACCAGTCAATTTCAGAATTTTGGGTCCCAAGCT |
| | | AGGCTGACTCACCTTCAGAATGGAAACCACGTGACAG |
| | | CCCTTTCCTTGGTCCCGCTACTGGACCCATTTCCTACCT |
| | | GGGAAATGGAGTCGAAGCTGACTCAAAACGTAACGGC |
| | | CCAATTGTCCTTGAGACTTCATTCCCCAGCAAGCTCAG |
| | | CGTGTAACGTGCGCTATGGAGCCGAAAGTCGCAGAGC |
| | | TGAAGCAGAAGATCGAGGACACGCTATGTCCTTTTGGC |
| | | TTCGAGGTTTACCCCTTCCAGGTTAGTTTATCCCTCCTG |
| | | CTGTTCTAGGGAAAGAAAGTTGACAGACAATCCGTAG |
| | | GCGGCCGCGCCAGCGCCAACTGCGG |
| 58 | BTBD11-C3AR1 | TTTACCAAGGGTAGTGGAAGTGGGGCCCACATTGTGAG |
| | | AAATACAACCATAAGTAAAAAGTGCTAAATGGCCTCTC |
| | | TTTAACCAGAGACCACAGTCTGTAACTTCCCCTGCTTTC |
| | | AGTGGTTACCTCTGGGTTTCCCCTGTTGTGATTTATAGA |
| | | AAATGACTATCACTTTGGAATTGAGCTGGTGGATTTCT |
| | | ACAAAGATTCTTGGTATTTCATGGTTATTTTCTGATGTT |
| | | CCTAGCAGCCCAGCTCGGGACGCGGGGGTGAGCACGA |
| | | CTGGCTGGCCCAGTTCTGCGCCAACGCCGCCGCAGCGT |
| | | GCGCGCACCGCAGCCGGGGCTCACGGGAGCCCCTGCC |
| | | TGTCTACTGCACCCGGGGCGGCGGGGAGGGGAGAGG |
| | | TCATTAAAAAATAACCTCAGGAACGAGGCGGGGAAGA |
| | | GTGAAGAGCCGCGGACAGTTGGACAGGCTCTTTCTCTA |
| | | AGAGTGGGTAAAGAAAGTTGACAGACAATCCGTAGGC |
| | | GACCCCCGCAACGTCTTCTGCCA |
| 59 | B3GALT2-TMEM79 | GACAAAGTCTCTTTCCCCTGGAACTTAACATTTTCAGT |
| | | GACACAGCTCAGAAGCACCTAAATGAATGTATAGTATC |
| | | GTTTTAAGTACTTACCAGTGGTGTGAAGAAAAACAAAT |
| | | TAGGGGAAAGGGGTTGAAAGCCTTGGGTACACGAGTG |
| | | CTATTTTAGGTAGGCTGGTCTGGAAAGAACTTTTTGAG |
| | | GTGACATTTTAGTAGAAACCTGAATTATGTTCTTATTTA |
| | | ATTCCTGAAGCTTCTAAAAAGCCACTGGGAAAGTACTA |
| | | AATCTCTGAGACTAAAGAGGGCGGACTCAGCCTCACTT |
| | | CCTCCTTCTTCTCATCTGGGCTTCCTGTCGTCACAGCAT |
| | | GATCATATTTTTTCACCCTTCACTTCTCCTTTTACACAA |
| | | ATAGCCCCGGATATCTGTGTTACCAGCCTTGTCTCGGC |
| | | CACCTCAAGGATAATCACTAAATTCTGCCGAAAGGACT |
| | | GAGGAAAAAGAAAGTTGACAGACAATCCGTAGGCCAA |
| | | CATCCACCCCACCAACGGCT |
| 60 | MACF1-XCL2 | ATTACACCTAGGGAAGATTTACTTCTTATCCTCTGCATC |
| | | CCACGTCACCCCCACCCCCCCCAGCTGAGCTGCACCCC |
| | | ACGCTCCCAGCAGCCCTCACGCTGAGGAATAAGCAGCT |
| | | CAGCTGCCTCTCCCGCATCCTGCCGAGCTGCAGCCAAG |
| | | ACCAGAGGGACTTTCCCCACCCCCACCACACATGTGTG |
| | | TCTATCCACAGAGGAACATCTATCAGGGGCTCCTGGAG |
| | | ATCCTAGACCCTGCTATACCCTGGTCTGTCAGTCAACC |
| | | AGGCATCCATCCCTGACCCTGCAGGAGCGTCCCTCCCC |
| | | CCACCTTGGTCACATGGCTGGCCGCCACTGAGCCTCAG |
| | | AAGTCAATCTCCAATGCCCCAAATACTGTTCAGCCTGG |
| | | AAACCACAGAGAGCCCTTCTTGCCACCCAACAGGAAC |
| | | AGAGCAAGTGGTTCTGGCCTCCCTGGGATTTGGATCCT |
| | | GGTTTCCAAAAGAAAGTTGACAGACAATCCGTAGGCG |
| | | CGACGGCCACTGCGTCTACTAC |
| 61 | TRIB2-AFF3 | TCAGTTATGTCCAATCTGAACAGCTGAGTGTTTACAGG |
| | | ACATCACGTGCCATCTTATGGTTTAATATTTACAAATG |
| | | CCTAGTTTTAAGGTAACAAAGCAGCTGATTGTATCTAC |
| | | AACATGCTATTCTATGACACCACATCAAAAGCTTTAAA |
| | | AAGAAAGCTCATCTCTATTTTCTTAGTAGCATCTAACA |
| | | GATATCCAAATACCAAGAATCTTAACTTGTGAATGGTG |
| | | GTTCCTTAGTGTCAAAACAGCCGGAAATCAAGTCCGTT |
| | | TATCTCAAACTTTAGCATTTTGGGAATAAATGATATTT |
| | | GCTATGCTGGTTAAATTAGATTTTAGTTAAATTTCCTGC |
| | | TGAAGCTCTAGTACGATAAGCAACTTGACCTAAGTGTA |
| | | AAGTTGAGACTTCCTTCAGGTTTATATAGCTTGTGCGC |
| | | CGCTTGGGTACCTCGGATGTGAGGGCGATCTGGCTGCG |
| | | ACATCTGAAAGAAAGTTGACAGACAATCCGTAGGCAC |
| | | TGCGGCAGCGTCAGCGGCGAC |
| 62 | CLIC5-SOX4 | AGGTCAGGGTTTCTGTTTCCTGCTGCTGGGATTCCAAG |
| | | TACTGTATGTGCTTTAAAACTTTGCTTCCGCTCATTTAA |
| | | AGTGAGACTCTTTTATTTACCCTAAAAGGAACTTGATT |
| | | GACTGTTCCAGGTTTCAGAAATATGAACTCTCTAGATG |
| | | AATGTTGCCTGTTAAAGTTTTCAGCTTCACTCTGATACC |
| | | TCTGGGCACTCGTATAAGCTCTCACAGAAAAGCTTAAA |
| | | TCCTGGTACCTGCTGGAGAGGTGGTGGGTGGCTGGAAG |
| | | CGGCTGGCTAGGAGGCGGGGCGTGGGGCGGTGGAACT |
| | | CCTGGGCTGCGGCATTCACGTGATCTGCACGGGCGCAG |
| | | ATGTAGGCACCGGTCCGAGTGCCTGCCCTCTGTCCCCG |
| | | CGGCTGGGTCTCGTCTGCTCCGGTTCCTGGGCTCCTAAT |
| | | TCTTGGTCCAGCTTCTTCCAGGTCAGTGTGCGGGCCTTC |
| | | CACGCAAAGAAAGTTGACAGACAATCCGTAGGCGCCT |
| | | ACGACGGCCTCGGCTACTT |
| 63 | FAM104B- | CCATGTATCTATCATCCCTGTCCCAGTGATGATTAATAT |
| | (chr101623051101623281) | ATAGCCAATTCTGCTCCATCTCTACTCCCACCTCTTCCC |
| | | CTTCCTGTTATTATTATTTGTTATTCAAGACAGTGTCTC |
| | | ACTCTGTCTCCAAGGCTAGAGTGCAGTGACGCAATCAC |
| | | AGCTCACCGCAGCCTCAATCTCCCTGGGCTTAGGTGAT |
| | | CCTCTTAACTCAGCTGGAACCACAGGCGCACACCATCT |
| | | CCTAACTGCCGTTTCAATATTCATATTGATCACAGGAG |
| | | CTCCTGCCTGCCTGGATGAAGAGGAGGTCAAGACTTTG |
| | | TCCCCCACTCCGCAAGATACCCTCTCTGTTCCGGAGCG |
| | | GTGGGTCCCTCCCCTGTTAGGACCTTGTCTCCCTCAGG |
| | | ACTGGACCTGGATCCTGGGCCTGCAGTCAGATTGCCAG |
| | | TTTCACTTAGAGGTGGAAATGTCAACCCACTGGTTGGA |
| | | ATGGGAAAGAAAGTTGACAGACAATCCGTAGGCCCGG |
| | | CAACTTCAGCGCCTTCAAC |
| 64 | CNEP1R1-NDRG4 | TTTCTTCTTGGTTTTTGTTTTGTTTTGTTTTGAGACAAGT |
| | | TCTCACTCTGTCACTCAGGCTGGAGTATAGTGGTGCAA |
| | | TCGCGGCTCACTGCAGCCTCAACCTCCCAGGCACAGGT |
| | | AATCCTCCCACCTCAGCTTCCTGAATAGCTGAAACCAC |
| | | AGGTGTGTGCCACCACACCCTGCTAATGTTTAAATTAT |
| | | CTGTAGAGACGGAGCCTCACTATGTTGCCCAGGCTGCT |
| | | TCCTCCAGCCCAACGGAAGAATGCGAGGAGCGCGCCC |
| | | GCGTCAGGGCCCCGCGCTTCTCAGCTGGGCGGAGTAGC |
| | | CGTTCTCGGTGCGTGAGCCTGCTGGCATCGGTCCTGCA |
| | | GACTGATTTCCCGCTTGTGGCCTTGATTGAAGAATCCC |
| | | ATCTCTTCTCACATCTTAAAACGAAAAAAGACTGGGAG |
| | | AAAGAGCCAGCGTAAACAGGGCGACGTACTGGGACGG |
| | | CCCCGGCGAAAGAAAGTTGACAGACAATCCGTAGGCT |
| | | ACGACAGCTTCCACTGCAGCTT |
| 65 | RORB-SMIM27 | GGAAGTCCTGGCCAGAGCACTCAAAGAGAAAGAAATA |
| | | AAGGGCATCCAAATTGGTAAAGAGGAAGTCAAACTGT |
| | | TGCTGTTTGCTGATGATATGATCATATACCTAGAAAAC |
| | | CCTAAGGATTCATCTTGAAAGCTCCTAGAACTGGTAAA |
| | | CAAATTCAGCAAAGTTTCAGGATACAAAATTAATGTAC |
| | | ACAAATCAGTAGCTCTGCTATACACCAACATCAACCAA |
| | | TAGCTCCTTCTGGAGGTAGGGCGGGGCCAGAACAGCG |
| | | GCGGGGGCGGAGTTCCGGCTGGGGGCGGGGCGGGGCG |
| | | GAGCGGGAGCTGCGGGATTCCGGCTTCAGGGGCGGGG |
| | | CACCGGCCTCTGGGCGGAGATCTGCTGCCGCGTTCTAC |
| | | CCTTCCGGCCCGTGTTCTATCCGCCGCCTCCACCTTCCA |
| | | TCCGGCGCCGGCTTTCGGCGCGACGGTCGCCGCGTTCC |
| | | ATCGTCGCGCGGAAAGAAAGTTGACAGACAATCCGTA |
| | | GGCCTGCTTCCACACCGTCAACTACT |
| 66 | ERCC6L2-SEMA4D | GTGCACAGGCTAAATATCTTCCCACAGTGACTCAAAAA |
| | | ATCTCACAGTCAGAAGGGCTCTAAAGGCCTTGACTCAA |
| | | CCTCCACCCACTATAGGAATCTCCCACAGATGGTTTCC |
| | | AATTTCTGTTCGAATACCACCAGTGATGGAGAATCCAC |
| | | CACATCACAAGGCAATCCCCCACACAGTTGGAAAGTTC |
| | | CTCCACTTTGAACTGAGGGCTTCTGAGTAGCTTCCACTT |
| | | CTCCTGGTTGGGCAAGAGAAGAGCCGCGCCCACCACG |
| | | GAACATCTCTGCGCATGCGCCGGGGGCCAGCTTCTGGT |
| | | ACTTTTGACGCGTCTGCTAACGTAGTCCCTCAGTGCGC |
| | | ATCCGGACGTAGGAGGTGGAGGTTGTGGAATTCGCCGT |
| | | TCGAAAGCAGGGACTAAAAGCCCCACTTCGTCTTACGT |
| | | TCCGAAAGGAAGGCGTCTGTTGAGCCTTTCTCTCAGTC |
| | | GTGAGGGAAAAGAAAGTTGACAGACAATCCGTAGGCC |
| | | AGCACCGGCGGCGACGTCCGCG |
| 67 | RPS6KA1-FCMR | CCCAACTTTGTGCTATAAACAACACTATTCAAAAACTC |
| | | AACACATTCAACAAAATAGACCATATCCTGAAAGGTC |
| | | AACACGTTGCTTCCTGGAAAAATAAGATTGCTTTAGAT |
| | | CTCTGCTCAGTGACTCCAGGTTATCTTATTATGACCAA |
| | | ATCCAAAACAGCCCATGGGCAGACCGTTACCAAATAT |
| | | AGAATTAGCAGTTAGAAACCAGAAACAGGAACTGGAA |
| | | GATGCTCCTCATACACACACCCCAAACCCTCAAACCTC |
| | | CAGGGCTTCCTGCCTGTGAACCGAAAGGGGGAGTGCG |
| | | AATTTCTTGGCCCTGTCGGCAGGTGCTTTCTCAAAGGC |
| | | CCCACAGTCCTCCACTTCCTGGGGAGGTAGCTGCAGAA |
| | | TAAAACCAGCAGAGACTCCTTTTCTCCTAACCGTCCCG |
| | | GCCACCGCTGCCTCAGCCTCTGCCTCCCAGCCTCTTTCT |
| | | GAGGGAAAGGAAAAGAAAGTTGACAGACAATCCGTAG |
| | | GCGCCCCAACGTCCACTACCACAAC |
| 68 | AP1M1-GADD45B | TTAAGTAGAAGGAATCATATCCAGAACCTGGACAGGT |
| | | CAGCTCATTTAAGCATCCTTGCCCCATGAAAAGAGGGA |
| | | GCCCTTTCATTATGACCTTTACTCTCTTTCCAGAACCTC |
| | | TCTCTGAGTTATAACTCATGTTTATGACTCAGTCTGAGA |
| | | CTTCTTTTTATAATTTCCTCTGTTTCTCTTCCCTTCTTCT |
| | | GCTCCCCACCACCCCCGCCCGCTGTGGGCTGAGTTGAT |
| | | CCTTAGCCTGAGAATAGTTAGCAAACAAGGGAGGTTGT |
| | | CATTTCCTCATCGTCAAGCTTTGTTCCTCGTGGGGGCTA |
| | | GAAATCTCTTTCCAGTTCCAGATTGTGAAGGGTTCCTG |
| | | AGTAAGCAGCGTGTCTCCATCCCCCTCTCTAGGGGCTC |
| | | TTGGATGGACCTTGCACTCTAGAAGGGACAATGGACTT |
| | | CTGGCTTTGGCCACTTTACTTCCTGCCAGGTAAGTACC |
| | | GTGGAAAGAAAGTTGACAGACAATCCGTAGGCTTCTG |
| | | CGGCTGCGGCCGCCACAG |
| 69 | CPM-CHD4 | GAGCCACTGCACTCAACCAGTATATGGAGATCCCCCCC |
| | | AGCACTTTGAATATGTCATTCTCCTGCTTTCCGTCCTCT |
| | | GTGGTTTGAGATGAGAAGCCAGCCATTGATTGTATTGA |
| | | TACTCCCATGCGTGTGATGAGTCACTTTTCTCTTGCTGC |
| | | TTTCAAGATGTTCTCTGTTTTTTGGCTTTCAGCAGTTTA |
| | | AATATGATGTGTCTGGGTGTGGACCTTTTTGTATTTGTC |
| | | CTGCCCCAGAGGCTGAGACCGACACGCCCTCCCCGAA |
| | | GGGCAGACCCGCCTTGAGGAAGGCGGATCCGGGTAGG |
| | | GACCGCCGCCTGGCCCTCACCCGACCCCCGAGAAAGC |
| | | AACCCAGCGCGCCGCCCGCTCCTCACGTGTCCCTCCCG |
| | | GCCCCGGGGCCACCTCACGTTCTGCTTCCGTCTGACCC |
| | | CTCCGACTTCCGGTAAAGAGTCCCTATCCGCACCTCCG |
| | | CTCCCACAAAGAAAGTTGACAGACAATCCGTAGGCCT |
| | | GCGACCGCGTCGCCAGCCGCA |
| 70 | IL22RA1-UFC1 | GCTGCAATCTGATTACTTATTGACAAGGAACTTGAAAC |
| | | TGAAGATGTAATCTTACCAATCATTGAAAACAATTTTG |
| | | AGAAAAGAATGTTTCAAGGAAGTAATCAGCCACAATC |
| | | TCATGGTGACAATCTAGTGGAGTAACTATGTCATAATA |
| | | TTAGCTGGTATAGCAACAGTAAGTGCCTAACCACATTC |
| | | AGCATACAAGAAATATCTCTTCAGTGTCGCTACACCAC |
| | | TGGTCCTGGGCGGGCCCTCTTCTGCCGCACTGCTGGTG |
| | | GGAGGGGAAATTGGGGACCCGAGTTGGTGGATGACGT |
| | | AGCCCTCACGTGACCAAGAGCTGACGTGTGCAGAAGT |
| | | CCTTCTTGTCCTGGTCGTTGTTCCCGTCTGAGTACCAGC |
| | | TCCCCACTGCCCTGAGGGCGGGCCGGCCTGCGGCGGA |
| | | GGGAAAAAGGAAGAGGAGAAGGAAATTGTCCCGAATC |
| | | CCTGCAGGTCAGAAAGAAAGTTGACAGACAATCCGTA |
| | | GGCCTGCTGCTTCAACCTCCGCGCCC |
| 71 | | TGACCCAGTTCACAATAAACTGGTTGCCATCTTGCCTC |
| | | ATGTAGCATTTCCTCTGCTTTCTTCATCACCATCTTGGG |
| | | TTTCACTTTGCCTCTTTTCTGTGTTGGATCCTCTGTTTTC |
| | | TGAAACCTATGACATGCATTTTTTTCTGTTTACTTTTTT |
| | | GTTTTGCTAACCCACATCCTCCAGTGGCTTTCTGAGAA |
| | | AAAAGTACATGGGGCATATGTTTCTCGAGAGCATACTC |
| | | CTGGTTGCCTACAAAGGGCAATGTTCCAGTGTGTTTTC |
| | | TCTTCGAGTGTGCTTCAGCCGCACAGTACTTCCTGTTTA |
| | | TTCAAACATCTTTTTTACCACTCGGCAACCCCCGCTTCG |
| | | CAGAAACAGCCAGAACCCATCTACAGCAAGAAGACGG |
| | | AAATCCAAAGGCAGACAGTACGGGCTCCCTTCGCCAA |
| | | ACTCTTCATTTTCTCTGTAAGTGTCCAGAGAAAAAGAA |
| | | ACATAAAGAAAGTTGACAGACAATCCGTAGGCCGGCA |
| | | ACGTCGACGGCCTCGTCG |
| 72 | SLMAP-LIPH | TCGCTTCACAGTGGAGAACTCTCATAAACACTGACTTG |
| | | CCTAGGTGACCAAGGTTAACATCACCAGTGATGAGTCA |
| | | TGTTGATAGCAAGTAACCTCTGATAAAATGAGATGAGA |
| | | AGCACACTTCAACTCTGTGGCCTTCTTCTCCAAAACAC |
| | | CTAGCCACATTTGAATGAGAAAACCACCAGACAAAAC |
| | | CAAATTGAGCAACATTCTACAAAATACATGAAAGCTAC |
| | | TCCTCCTCTGATTTAAAGGTGAGCGCAGACCATCCCCC |
| | | CGGCAAGCCCAGCCCGCGACTTAGCTCTCGCCACCCCA |
| | | CGCAGAAGTGCTTTAACAGGATAAAAGTGAGGGGGGA |
| | | AGCCAGGCAAAGGTCAGAGAGGATGAGCGGATGCTTT |
| | | GCTGGCACCCCAGGGGGGACGTCAGGTACCATTTATGC |
| | | CTCTGTCTTTCAGCTCCGTGTCAGGTCTCTGTGTTTGTT |
| | | CCCCATTATTAAAGAAAGTTGACAGACAATCCGTAGGC |
| | | CAACGTCTCCTTCAGCAGCGTCA |
| 73 | HS3ST3B1-SNX11 | CTTTAAACCACAGAATATGCAAAATGGTTCTAAGATTA |
| | | TATGTCCCTACTACTACCAACCACAGATCTAAGTAAAG |
| | | TTCAGAGTAGCTTTGTAGTTCCTGTTATTCTTAGAATAC |
| | | ACTCTACTAAGGGTGTACAGTAAGAGTACTATGTTTAA |
| | | AAGATACCTCAATTAGTTACTTTTCCCTCTGATCATATT |
| | | GTCAACATGATGTACATTTAGAGGAGATAGCATGTTTT |
| | | TCCTTTTGCTCATGACCTTTTCTCTCGCCACACCCTGGG |
| | | GCTCTTGGGAGGAGCTGAGATCTGCGCTGCAGTGCCAG |
| | | GATGCTGACTCTGTTGGTGCTGGCTTCTGCTGCCTTCCT |
| | | GGGTCCCGTCACATCCTTCTTGCTCAACCACTGGGTGC |
| | | ACAGGATGGAAACTTCTATTCCCTCTCTGGAAGACAGC |
| | | GCGTGGCTTGGCTTCACAGAGTTGTGGCTGGAGACCGA |
| | | AGCAAAAGAAAGTTGACAGACAATCCGTAGGCAGCTC |
| | | CAGCCTCCCCCTCTACCC |
| 74 | MTOR-DENND2D | TAGTCTGTTCTCACATTGCTACTAAAGACTTACCTGAG |
| | | ACTGCGTAATTTCTAAATGGAAGAGGTTAAATTGACTC |
| | | ACAGTTTCACATGGCTGGGCAGGCCTCACAATCATGGC |
| | | TGAAGGCAAAGGGGAAGCAAAGGCATGTCTTACGTGG |
| | | TGGCAGACAAGAGAGAGTAGGAGCCAAGTGAAAGGG |
| | | GTTTCCCCTTGATAAAACCATCAGAGCTCATGGACTTA |
| | | TTCGCTCCTCTAAACAGACCACGTTTTTCTCCGCTCTTC |
| | | CCCTTCCCACTCCCTGAAATCTAGACTAAAACTCTTTTA |
| | | AGATAACTGCGAGGTATAAAGAACGTCTAGAGCCTAA |
| | | GCGGCCCGTTGCCAGATTCTACTCCTCAGGCCTCAAAT |
| | | GTGAGCGCCCCAAACATTCTAGAGCTGAACCTGGTACG |
| | | CTCGCCCACCAAACTGCCGTGCTCGGGGCTTAGTATCT |
| | | CACGCGCGGCAAAGAAAGTTGACAGACAATCCGTAGG |
| | | CCTGCAGCTACGCCAGCAGCGGCG |
| 75 | ASB9- | AGCCAGAAGGGAGAAGGATCACATCCCCCTCCGTGGC |
| | (chr103585417103585647) | CAGGCTCTGAGAGACAGAAATCCAGTGAAGGACAGAG |
| | | CATGGGGCTGAGTGTCTGTAGCCCACTGCATTGAGGGC |
| | | AGGAGAGCTCAGGGCAGGTCTGAGTCATCACCATTTTC |
| | | TGTGTGTTTCTTCCTAATAAGTGAAATAATGTGTAGTGT |
| | | CTAGCACGGTGCCTGACAGCTCACAGAAAATCCCCAAC |
| | | TTCTCCTTTCTTCTGTCCCATGCCTCTCCGTCCCCGAGG |
| | | GGGGCCGGGCCTCCTTGTGACGCCCTCGCGCCGTCCTC |
| | | GCAGAGTACGTGGCCGAGCACTGGGCAGAGGACACCT |
| | | TCTTTGGGTACCAGTACCTCAACGGCGTCAACCCCGGC |
| | | CTGATCCGCCGCTGCACGCGGATCCCAGACAAGTTCCC |
| | | CGTCACAGACGACATGGTGGCTCCGTTCCTGGGCGAGG |
| | | GAACGTGCTAAAGAAAGTTGACAGACAATCCGTAGGC |
| | | ACATCTACTTCATCATCAGCTAC |
| 76 | KRT14-PCGF2 | GCCACATTCAAAACATTCCATTCCTTTTTAAGCTGAGT |
| | | AATATTCTACTGTATATAAAGACTACATTTTGCTTGCCC |
| | | ATTCATCTGCTGAAGGACACTGGGTTGCTTCTGCCACG |
| | | TTTTGGCTACTGTGAGTAACACTGCTATGGATATTTCCA |
| | | TACGAGTACAGTCATGCACCACATAATGACGTTTTGGT |
| | | CAACCAGGGACCACATTTACAATGGTAGTCCCATAAGA |
| | | TCCTCGGCCCACGCCTGCTTCCGCCCATTGGCTGCCCG |
| | | CGTTTCAAGACCCGCCCCCATATTGGAGAGCTTCTTCC |
| | | GGTAGCCACTGGAAAGCCCGCAGCGCGAGCCGGCTAC |
| | | GCTCCCTGGCGGAGCTCTGGACGCCCCACCTACTCCCA |
| | | GCGTTCAGTGCGGTGCCCTGGCCGCCATTGTTTGAATT |
| | | TGAAAACGGTAACATCGCAGTGCTGCTCGCGGGTCTGG |
| | | CTAGTCAAAAGAAAGTTGACAGACAATCCGTAGGCTC |
| | | AGCAACAGCATCATCGACGAC |
| 77 | SFXN4-KIF20B | CTTCCAGACTCCTCACCAGTTATTCATGTCAAGTGGAT |
| | | GACAAATTTACTGCTCACCACTTTCCCGCTGGCTAATTT |
| | | ACTTTAACCGCTGCTCTTTAGAGATACCACTGAGTGGA |
| | | TCTATAATATGTCAGCAGTGTATTTTTGACTAGCATCTG |
| | | TTGACACACTTGTGAGCCAGGCATGAAGTTTTTTTTCA |
| | | ATCTTCTATCAGTTGGTCATAGGGAATCCCCTATAAGC |
| | | TCCTGAAGCTGGCCCACGCATGCGCTCTTCAAATTTTT |
| | | GAGACAGTTTACCCAGAATGCAGTGCTCAAAGGAAAC |
| | | GCGTGCGCAGTGTGGTCAGGTTGTTTCGCTGGGTGAGT |
| | | AAAATGAAATCTTAGAGGCGTTGTGGGCTGGCCCAGTT |
| | | GATGACGTCACCATACCACAGCTTCTAGTGCTATTCTG |
| | | CGCCGGTATCCGACCGCCGGGCGCGGTGGCGCGTGCCT |
| | | GTAGTCCAAAGAAAGTTGACAGACAATCCGTAGGCAT |
| | | CCACCTCAACTTCTACATCGG |
| 78 | GOLM2-MIR4513 | CACCTATTCTGGTGACTCGGAATAGGTGCCATTTATTT |
| | | ATCTGCAATCAGGAAATAAAGTGACACTTCTGAGTCAC |
| | | AAAGTTCAACTCTTCTGAGTTGAACTATGTGAAAGTTC |
| | | TTTACTAATACCACACAGATCTTTTCCATTCTGATATAG |
| | | GAACTGTGTGCATGTCCTCATCTGTATATTTCAGTGCTA |
| | | GAAAGGTGTCAGCATAGCAGTCTCTCAATAAACATTCT |
| | | TCCTGGTTGGGCAAGAGAAGAGCCGCGCCCACCACGG |
| | | AACATCTCTGCGCATGCGCCGGGGGCCAGCTTCTGGTA |
| | | CTTTTGACGCGTCTGCTAACGTAGTCCCTCAGTGCGCA |
| | | TCCGGACGTAGGAGGTGGAGGTTGTGGAATTCGCCGTT |
| | | CGAAAGCAGGGACTAAAAGCCCCACTTCGTCTTACGTT |
| | | CCGAAAGGAAGGCGTCTGTTGAGCCTTTCTCTCAGTCG |
| | | TGAGGGAAAAGAAAGTTGACAGACAATCCGTAGGCAC |
| | | CTCAACTACTCCTGCAGCGCC |
| 79 | VCAM1-RSRP1 | GTTTGTACCCGTCCTTTTTGGGAAGGCTTTTCAGATATT |
| | | CAAAAGGACTTGTGTGTTGTGATCTAAGCTATATTTAC |
| | | TTTAGGGGGAACCTTAAGCCCAGTGACATTGTGGTTCT |
| | | TGCAGACTCATAGAGATACTGCTTTGATGGTCTTAGAA |
| | | ACAGAGTGGCATCTGCAATTCAAGTCTGTCTTTCTTAC |
| | | CTCTTCAGTGTCCCTTTCAGTGATATATAGTTAAAACCA |
| | | TCCTTAGGTCTTGTCACCCCCACCTTTTCCTGCTCATCA |
| | | CACTTGAACCACACTGGCTTGCTTTCTGTTCTTGAAGTT |
| | | CTCTCAGCCTTGACCATTTCCCCCTCAGATAGCTACGTG |
| | | GCTGTCTGGCACTGATCACATGTCACCGCCTCTGAGAG |
| | | GCAGGGCTTCCTTGACCAGCCTATCTAAAAATAGTAAC |
| | | TCATCACATTCTCACCCTTTCTCCTATTATATGTTATTTT |
| | | GAAAGAAAGTTGACAGACAATCCGTAGGCCCAGCAAC |
| | | TGCAGCGCCTACTTC |
| 80 | TANC1-TNS1 | GTTTTGCTTGATTGGCCCCTTTTTAGGCCTGCCCCCACA |
| | | GTGCCTCATTCTATTTTTATGTTAGACACTGTTTCTCTTT |
| | | CGTATTGCCAGATGCCCTTTCTACTGTGGCTCACCAAA |
| | | TCCTCCAGCAACAGCTGTTCTACCAGCCCACTCTCGGC |
| | | TACATGCAGATCGAATGTGATTCATAATTAGTAAGAAT |
| | | ACCTAAGTGTGTCAGGAGCCTGAAGCATGTAGGGAGA |
| | | TCCTGTGAGTCTACCTTACCGTAAGGTGAAATCTTAAC |
| | | CACCTTAACTGCTGAAACCCAACCACAGCTGCTGTGAG |
| | | AGGGCAAGGGGTGGAGGTGCTCAGGCCCAGAAATTTG |
| | | CATTCCTGCTTCCCCGTGATGAGGACCCAAAGAAAAAA |
| | | AAACATTGAGCCACAGGTAGGGTTGCCAGATTTAGCCA |
| | | TAACAATACAGCACGCCCATTTAAACTTGAATTTCATA |
| | | TAAACACAAAGAAAGTTGACAGACAATCCGTAGGCCC |
| | | CCTCGACTGCGTCTCCGACTG |
| 81 | PTPRD-VLDLR-AS1 | AATGTCACACCATGTCACACCACATCACACTATGTCAT |
| | | GCCAGGGATGTCACGCCACATCACACCATGAAACTCAT |
| | | CATCACACTAGGGATGTCGTACCCTGCCACACCCCATC |
| | | CTGCCACATCACACCATGTCACACCGCATCACATCACA |
| | | CCAAGGATGTGGCACCCTGTCACAGCACGTCACACGTC |
| | | ACATCATGTCACACCACACCTCATCACACCACAGATGT |
| | | CATCCTGCGCCTCGGCTTCACTAAGCGCAGTCAGGTGG |
| | | CTGTCCCCGCACGGTTCCCAGCTAATTCCCCGCTACCG |
| | | GGTTGCGGCCGGAAGCCGGGCGCCGCGGCTCTGCTTCC |
| | | CTCGGGGTGAGTAGGGGCAGCTCGCCGGAGGCCGGCG |
| | | CAGAGCTCGGGCCGACCAGCCTGGCAGGGCCGCACCG |
| | | CGCCTGGAGCGCGAGGGAACCGGCATGGACACGCCGG |
| | | GAGTCGGCCTCAAAGAAAGTTGACAGACAATCCGTAG |
| | | GCTCTGCAGCGACTTCAGCGCCTTC |
| 82 | RTRAF-CD80 | GAAATTCCCCTCTTACCTCTGAGTCATGCTCACTTGAG |
| | | AGAGTCTCCTGATTGGATGTTGACTACCTGTAGGAGAA |
| | | AAGATGCAGGAATGTAGAGTAGAGTTGGTTTTCTCACT |
| | | CATTTAAAATGTTAAGTCATGTCATTGCTCTGCTCAAA |
| | | ATGCCTCCCCATTTTACTCAGCATGAAAACCAAAGTCC |
| | | TTGAAATGGCCTGTGTAGCCTCTGTATGCTCTAGCATCT |
| | | CTCCTCTCCCTGCGCCGCTCCAGAGCGCAACAACCATT |
| | | TTCCCAGCCAGGAGCACACCGTGTCCACGCGCCACAGC |
| | | GATCTCACTGATTGGTCGGGCTCCTGGTAAACAAGGAC |
| | | CGGGCAGCCAATGGGAGGGATGTGCACGAGGGCAGCA |
| | | CGAGCCTCCGGGCCAGCGCTCGCGTGGCTCTTCTGGCC |
| | | CGGGCTACTATATAGAGACGTTTCCGCCTCCTGCTTGA |
| | | AACTAACCAAAGAAAGTTGACAGACAATCCGTAGGCC |
| | | TGCTACTCCTCCTGCTGCCACG |
| 83 | PDE4B-TMCC2 | CTGGATAGAATTCAGGGAGCTATGAACTGAAATGGAA |
| | | AAAACATTACATCTTGATTTTCACAAACCTCTAACTGA |
| | | AATTTATCATTTCCTTCAGTATGAGGGTAAGCAACAAG |
| | | CCACAGCAATGTTAACAGTGCCTGTGGATTTATCACAC |
| | | AGAAACCACAGATACTTTAAACAATTGTTTATGTTATT |
| | | ATTTATTAGAGATGAGGTTTTGCTCTGTCACTCAGGCT |
| | | GGATCCTGTAACACACCTTCTTTCCCCTCCGGCTTTCCT |
| | | TCCCCTTCTCTCCCGCCTTCTCCTTATTCATACCAGAAG |
| | | CGCCTCAGCTCTGATTGGCTGGAGCTCTGTGCTATCTC |
| | | AGCCAATCACAAGCCGGGCTGTGCTCCTACACCATCCG |
| | | AAGAGCGAATCGTGCAGAGACCGTGTCTACGATTGGC |
| | | CTCTCCCTGACAAGGATTTAATTTTGAATTTTTCTTTAT |
| | | GGCGTGGAAAGAAAGTTGACAGACAATCCGTAGGCCC |
| | | TGCGGCATCTCCTGCCACTTC |
| 84 | GNA13-SNX11 | ACCTGCTGATGGTCAGGTTCACAGGAGCGTGTTAAATT |
| | | CATCACTGACATTACTCGTTCATGAGCACAATAGATTC |
| | | ATGTGTTTCCCACAAGGTACATGCTCATGAGTAATGCA |
| | | GTGAGTAACCCTTACACAGGTTTTGCATGTTTGACCCC |
| | | CTTTCTGTTCCACGCATTTTTACCACCATCAGTTGTAAC |
| | | ACAGCCTAGCATATTCCAGTTTAGGCTGTACTCATTTA |
| | | GTCCTTGCACCAATCAGCGCTCTGTAAAATGCAGGAAT |
| | | CTAAAAGTAGCCAATCACAGGGAGGATTGAAAAAAGG |
| | | GCACTCTGATAGGACAGAAATAGCAAATGGGCAGGGA |
| | | CAAACAAGGGTGTAAAAGCTGGCCCCCTGCCCCCCACC |
| | | CATTCCTGTTAGCCCGCAGCAGCAACCTGCTCAGGTCG |
| | | GTTTCCACGCTATGGAAGCCTTGTTCTTTCGCGTTCTTT |
| | | CGCTCTTCAAAGAAAGTTGACAGACAATCCGTAGGCG |
| | | GCGACGTCGGCTACGGCAGCTA |
| 85 | ALOX15B- | CAGGTTCAGTGTGATCTGGAATGCCTGCGAGGCCCCCT |
| | CCDC144A | TGCTCATCGACCTGCCTTTAATGGATGGGTGGTCTGAG |
| | | TTTCTGGAGATGGGCAAGATGGAGCAGTCTCCACACTT |
| | | GGCTCTTCAGCCCCGGCGCCCCCCACAGCCGCCTCTGC |
| | | TTTCCTGGAGATACCGCAGGCTCCTAACCTCAGGCCGG |
| | | CTTGTGCTGCTCCACCCTCTCCCTCCCCCACAGACACA |
| | | GGTCCTCTCTACCAATCAGCAGGATGTGGGTGGGGCCA |
| | | GATAAGAGAATAAAAGCAGGCTGCCCGAGCCAGCATT |
| | | GGCAACCCGCTCGGGTCCCCTTCCACACTGTGGAAGCT |
| | | TTGTTCTTTCGCTCTTGCTACTGCTAGCTCTTTGGGTCC |
| | | ACGCTGCTTTTATGAGCTGTAACACTCACCGCGAAGAT |
| | | CTGCAGCTTCACTCCTGAGCCCAGCGAGACCACGAGCC |
| | | CACCGGGAAAAGAAAGTTGACAGACAATCCGTAGGCA |
| | | CCCCTGCCACATCATCCGCGTC |
| 86 | FTHL17- | ACCATCCTCCTCCAAAGTTAGCATCAGACGCCACAAGT |
| | not_found_ | TTAAGGCTCAGCCCTCCATGAGATTTCCCTTGCTTCAG |
| | chr4852159648521826 | ACACTGGCAGCATCTTGGGGATCCCCCAGCCACCCACA |
| | | CTTCTGACTAACTGGCTCTAAATTGGGGGATTCCCACA |
| | | AACTGTTTGGGTTTCATAATTTACTAGAAGGAACTCAC |
| | | AGAATGAAAATAAACCTGGCCAGGTGTGGTGGCTCAT |
| | | GCCTCCTCGCGGCCGGCCGGCGACGCTGGCGACGCTTT |
| | | CGCCCCTGAGGTAGTTTGGCGACCGCGAAGAAGGAAA |
| | | AAGGGCGGGCGGGCGGCTGTCCTCTCACCGTCCTCACC |
| | | CCGCGAGGCCCGGCCCGCTCCTCCGTCGTGGATTTCGC |
| | | GGCGATCCCCCCGGCAGCTCTTTGCAAAGCTGCTTGAA |
| | | ACTTCTCCCAAACTCGGCATGGATACGACTGCGGCGGC |
| | | GGCGCTGCCTAAAGAAAGTTGACAGACAATCCGTAGG |
| | | CCGGCCGCAGCACCCGCCTCCTCG |
| 87 | AGL-THEM5 | GTTAAACTGCTTCTTAAGAAGACTTGCCTCCCTGCAAT |
| | | GTTTACTGAAGGGAATGGATTTACCCAGACAGGCCTGG |
| | | CCTTTGGTAGAAACGCTGCACAGCTCTGCGTAGAAGCA |
| | | GTTAGTCATCTGTACTTGCAGAGGCTGTAAAACAAATG |
| | | TGGTGAACTGTAAAAGATTAAAGAGCCTGTTTTCCTGC |
| | | CAGCTACCAGAAAGTTTCACATATACTTATTAAAGTTC |
| | | TTTCCTGACACTGAACATGTGGGTCACCGGCGAGACAG |
| | | TGTGGCAATATTTTCCCTGTAATGTACCAAGTCTTGCCA |
| | | AAGCAGTGAACATTATGACACAACTTTTTGTCACAGCT |
| | | GGCTCCTAATAGGACAGTGCCAGCCAATTCAAGCCCAG |
| | | TCCTTTCTGTGTTTATTCCCATCTCTCCCAAATATTTGG |
| | | AAACTGATGTCTTGACTCATGGGTGTATTCACAAATTC |
| | | TGTTACAAAGAAAGTTGACAGACAATCCGTAGGCCGG |
| | | CTCCGTCCTCGACCCCGGCT |
| 88 | RBMS3-LARS2 | TCCATGTGCCGCTAATAAAATATATTTTGAAAGAATAA |
| | | GGGTGTGGTTGGGAGTCACTGTTGACAAAGACCAGAA |
| | | TGTACACGAGCTCCCCCCTTGGTCTCAGGTGTACTACA |
| | | GAGACCTCGAGATGAGGATGGTGTGGAGGGTGATATG |
| | | TGCCACCTTGTGGAGTACTCGTGAGTTTTTCAGAAGAA |
| | | CTGAACTTTTTCTGAGTGGGTCAAGGAAGAAGCCAAAC |
| | | CATGTCCTCTGCGCCCACTAAGATGCATGAAATAAAAA |
| | | TTTCCGTGACTCGCCCTTTGCAGTGGAGAACTGAAACA |
| | | GGCACACCAGGGAATTGGAGCGGAGGAGGGTAACTCA |
| | | AACTCAGAGTGAGAGGGTTTGCAGGGGGCCGATTTGG |
| | | GGCCAACAGGCTTCCCAGCAGGCCCCCGGCGCGGGAC |
| | | AGCGGAAGGCGAAACGCTTTCAAGAGACCCCGCTGCC |
| | | AACATCCCCACGCCAAAGAAAGTTGACAGACAATCCG |
| | | TAGGCTCCTACGGCAGCGTCGACCACTT |
| 89 | SCGB1A1-CAVIN3 | GGGAATCCTAGGAAAGTCACCTCAATGTAGGAAGAAC |
| | | ATATTCAGGCAGAAAAGAGAGCAGTACCAGCAGCTGC |
| | | TGGAAGATCTAAGAGAAGCTTGACACTCCTGGCCCCAA |
| | | ACACTGCCTGGCTACAACACGATATCCAGGGACAGAT |
| | | ACCTTCCATGTACAGCAAGCTGTGGAGTGGCAAGAAA |
| | | GCAAAGCACGTACCAAAAGCAGGCAGGAAGCAGAGCT |
| | | GGCTGGTTCCTAAGCCAACTCCTTACTGGTTCAGGTAT |
| | | GTGCTCGTCTCTAGAGTAAACCAATCAGAGCCCCACTT |
| | | CTTACTCCTGGGATTTGGTAAACGGAAGAGACCTCTCC |
| | | TGTTCCGGGAGTAGGCCGGGACCGTGGATGAGTGACA |
| | | GTTCGACTTTCCAATAGGCAGAGCCCTAGGACCTGCCC |
| | | ACGTCTGGTTCAGGGGCTAGAAAAGAGCGTCGATGCC |
| | | GGCGGCAGTGATGAGAAAGAAAGTTGACAGACAATCC |
| | | GTAGGCTCCCTCGTCCGCGCCGGCTTCGT |
| 90 | OXR1-ZBTB10 | ATTAACTGTAGGATGAATCTTTGTCTAGCAGGGAACTG |
| | | TGGATTCTGTGGTTTTTGTTCTTAATTCTTTTATGTGTCT |
| | | TTGTAGATCAAAGGTTGTCTTATATAGGTGCTATTGGT |
| | | AGCAATTTACTCAAATACAAAGACTCTGTGGTTTGAAA |
| | | GAGATTAGTGTTTTAAATGAAAGACGAAATCTTTTTCT |
| | | TAGAATCCTTGTCTTAGAGGCAGTATGATATTTCCTTCT |
| | | CCTAATAAAAGCATCAGTATTGCAAAGACTTTCCATGA |
| | | TCCTATACCCACCTCGAAAGCCCCCTCTCACCACAGGA |
| | | AGTGCACTGACCATTGGAGGCATAAAAGAGATCCTCA |
| | | AAGAGCCCGATCCTCACTCTCCCTGCACAGCTCAGCGG |
| | | GACCTCAGCCATGAGACTTCTCATCCTGGCCCTCCTTG |
| | | GCATCTGCTCTCTCACTGCATACATTGTGGAAGGTAAG |
| | | TGGAGAAAAGAAAGTTGACAGACAATCCGTAGGCTGC |
| | | AACGGCTTCATCTCCGCCTG |
| 91 | RNF115-XCL2 | AAGCCATAACAGACTAAATGAATATTTTACCAATTTAG |
| | | TGGGTAGGGTGGGGAGCTATTCTCTCATGAATGGATAA |
| | | TGACCTTAAGTAACTGTGACTGCTCTGTATGGCAGCAT |
| | | TGCAAAGGTCAATGTGGGAACATCTTTCTCAGAAAGGA |
| | | AGTGTGGTGGGGGTTGTATTTTAATTCAACCATTTGTGT |
| | | TCTTGTGTATTTCACAACAAAACCTAATGAGACCAAAT |
| | | GTCCTCTGCTCCATGATTGGGACTGAAGTAAGGAGGTA |
| | | GCGGCCCAATGGGCTGTGGGAACGGTCCTCGGCGGGTT |
| | | GAGGGGCGGGGATATGCAAATATGGTTTGAAAAGCCG |
| | | GCGGGAAATCCGAGTTTCGCGGGAGGACCTTGGCGCG |
| | | TAAACCGTATCCCTTCATTCATTGTCAGCAGCAGCTTCC |
| | | TGGAGCCATTTTTCAGCTGCCGGCCGCAGCACCCGGGC |
| | | TGCCGCCGAAAGAAAGTTGACAGACAATCCGTAGGCT |
| | | CATCCGCAACTACTACCCCACC |
| 92 | LRP11-RN7SK | GTCTGTTCCCTTATGTACAAAATGTATAAAAAGGACAG |
| | | TTCATGCCCCTAGTAGGTTGATACCCTAGTGGATGAAC |
| | | CGTGTATTTAGTATGAAAGATAAAGCTCAGATTTCTAG |
| | | TCCAATGACTCATTAAAAAATATTGTGTGGTTTGATGC |
| | | TTGTCTAAACATCAGGAAAACAAATATACTTTTTGTAT |
| | | TACTTTATGAATCAAACACATCATTGTTTCTTGTTGAGA |
| | | TTCCTCTGTGGCAGGAGCCGGGAGGAAGGGAGCGAGC |
| | | CAAGAGGGGTGCTGCCCACCGGAAACGATGGCGCGAG |
| | | GCCGCAGAGCTAAATGGGGGCCTCTCCAGGGAGTGCT |
| | | CTGTTCACGGCTCCATCGCTGTTAGTAAGTATCTTGTGA |
| | | TTTCGGAATTTAAATGAGGTTGTGTTTAACCTGCATAA |
| | | CATCTGGCTTTTAAAATCTGACTTTATTTTCCTTTTATTT |
| | | CTGTGCAAAAGAAAGTTGACAGACAATCCGTAGGCCA |
| | | TCCGCAGCCGCCACTGCTGCG |
| 93 | (TBD)-CD53 | CAGTTCTTAGTCCCTCACTTCCTAGCTGTGTGATGGTGA |
| | | GGGGAAGTTGTTTAGCATCTGTAAACCTCAGTTTTCTT |
| | | ATTAGTAAATGAGATGAATCAAGTTTTTACCTCATCAA |
| | | GTTGATATGGAGCTTTCAGTATGATGCTTGACACATAG |
| | | TTAGCATTCATTAAATGTTAGCAAAAATGATGATGATG |
| | | ACTGCAATGACAACAAGCTGCCAATAATTGTCTTGGCC |
| | | CTCCTAGGACCCAGCTACGGAGGCAATAAAAACTTGGT |
| | | GGAGTCTAAGTCCTGGGAAGGGAAAAAAAGGACGGAG |
| | | GGGGCGGGGCCTACAGACAACCCCGCCCATCACAAAG |
| | | ACCCCGCCTTCCAGAACGCTGACGTCACAATTTGGCCC |
| | | CGCCCCTCATTGTCAGTCCCGCGGAGCCAGAGGAGGCG |
| | | CCGGTCGCTGGCAGCTGAGCGGCGGAACCCTTCGCTGG |
| | | CGGGAGCGCAAAGAAAGTTGACAGACAATCCGTAGGC |
| | | CCGGCTACGACGGCGGCGGCGTC |
| 94 | ZNF281-CDKN2C | GTGTACAATACACTGGTTGGTATCTGTTCTCAGCACAA |
| | | AAAACTATGTGAAGCCAGTGTTCAGGGAAGAAACCAC |
| | | AACCATGGTAATCAGCAGTATCTTTACCTTCTCTCATAT |
| | | GGTTATCTGACTCATCAATTACTCTTATCTTCATCTGAA |
| | | ATGGGACAGTCATACTCTTCTTCATTATAGCTATCAGA |
| | | CAACTGCCCTTGTGCTGAACCACCCACATTAATGTATT |
| | | TTCCTCCGCCACCGGCCCTGCTCGGCCGCCATCGCTGT |
| | | GAGGCGGCTGCCCGCGACAGCTCCTCCTCCGCCTGCCA |
| | | AATCTCGCGATGGCAGCCGCACAGTTGCGGACCAGTG |
| | | GCCTGCTGCTACCAAATCTCGCGAAACTCGTTTTCCCTC |
| | | CCGGGAAGTTGCCAGAACTAACTCTGGGCAGCATGGC |
| | | GTCTTTCCGGCTTCTCCAAACCCTTGCGAAAAACTTTAT |
| | | TGGCAAAAAAGAAAGTTGACAGACAATCCGTAGGCGG |
| | | CTGCAACCACGTCACCCTCCC |
| 95 | BRCA2-COG3 | TCCCATCATCCTGTTATAACCATTCCTTTGGCTCTGGCT |
| | | TTTTCTCAGGCCTCCTCACCATATTGCATCTCATGCTCA |
| | | GAAATAACTGGCTCTGTAGTCATTTCCTGCCCCACCTG |
| | | TGTGTGCCTTCCATTGAAATACAAGGTTTGTTAGCTAT |
| | | AGTGGTGGTGAAAGGCAGGAACTTCAGCTTCAGACGA |
| | | CCTGGGCTCAAATCCTGTTTCTGTACCATACAAAGCCA |
| | | ATCCTCCAACTTGCCTTTTTCCCTGACAAACAATGACTT |
| | | GTACCGGGTAAGTAAATCTTATCGAGTGACTGCTATTT |
| | | GAGATACAGATAACCTTAAGCAAATAGTTCTCCCTGGC |
| | | AAATTCTTTTTTCCTTTTTTTTTTTTTCTTTTTTTGAGAC |
| | | AGTCTAATTCTGTCACTGAGGCACGAGGCTGTAGTGCA |
| | | GTGGCACTATCATGGCTCACTGCAGCCTCCCCGCCCCC |
| | | TCAGAAAGAAAGTTGACAGACAATCCGTAGGCCACCA |
| | | GCACCGGCCACTCCAGCT |
| 96 | PUM1-TMEM79 | ATGCACATCTCCGCTCCCTCCCTTACCAACCCCCTGCTG |
| | | TTATGTCCTGTGTTCAAAGTGTCCAGGAGGCAGCTGGG |
| | | ACCACTGGTTCCCAGGGTTGTGTTAAAAGACTGAGCCA |
| | | CATGTTAATTGGATGTGGTGTGAGAATATCACCCTGAG |
| | | GGTAACGCTTCCCTTTTGCACTTCTCAAGATTGTGCAG |
| | | GGTGGAGCAGCTATTGTGGACACGCACAGGGTGACAT |
| | | CATCCTGCCGGGGCGTGGCCCAACCGGGAACTGGGGC |
| | | GTCGAACTCCCCTTCCCAGTCTGGAGCCACCCATTCCA |
| | | GGCGGGGCGGGAGGCTGATCACGTGACCAGCGAGCGG |
| | | TCTCACCCTCCTCATATGCCAGTACCACCTCCTCCAGTC |
| | | CCAGAGGGGGGATGTCTTACTCCTTTCCGTGAAGCAAA |
| | | CCCTTTGAGTACGGTTTGCGTCCACAAAGCGTAGGGTG |
| | | AGAACCTTGAAAGAAAGTTGACAGACAATCCGTAGGC |
| | | GCGTCTTCATCTACTGCCGCCAC |
| 97 | SNTB1-MROH6 | GACAGATTTCAAAAATTATTTGAAGAGTATATCCAAGT |
| | | GGGTCTTTGAAAATTGTAAGATCTACTCAGTTCTTAAA |
| | | ATGAATCAGACATGTGAACTTGTGTGGTCTAGCGGTTA |
| | | GAAGTGTAGACTAGTGGGAGTACTTCTGGGGTTATTTG |
| | | TTCATCTTTTACTGACTTACATGACCTTAAGAAAGTCAC |
| | | TTGAGCCTCAACATTTCCTGCAACTTCACAGAAAGGTT |
| | | GTCCTTGCCCACACACCCCTGACACACCGGCGTCGCCA |
| | | GCCAATGGCCGGGGTCCTATAAACGCTACGGTCCGCGC |
| | | GCTCTCTGGCAAGAGGCAAGAGGTAGCAACAGCGAGC |
| | | GTGCCGGTCGCTAGTCGCGGGTCCCCGAGTGAGCACGC |
| | | CAGGGAGCAGGAGACCAAACGACGGGGGTCGGAGTCA |
| | | GAGTCGCAGTGGGAGTCCCCGGACCGGAGCACGAGCC |
| | | TGAGCGGGAGAAAGAAAGTTGACAGACAATCCGTAGG |
| | | CCTCCCCTACACCTTCGTCCTCGA |
| 98 | ABCA4-ZNF683 | TAAAGAAATTAAAAACAGAACTACCATATATGACCCA |
| | | GCCATTCCTTTTCTGGGCATATACCCAAAGGAAATGAA |
| | | ATCACCACCTCATAAAGATGTCTGCACTCCCATGTTCA |
| | | CTGCAGCATGATTCACAATAACTAAGATATGGAAACA |
| | | ACCTAAGTGTCTGATGATGGACAAATAGAGAAAGAAG |
| | | TGGCTGTAAGTATACCACATTTGGTTTATACAAAATGA |
| | | ATATTTCCTGTCACTAACACATTGCCCAATCAGAGCTG |
| | | GATATTTTGAATTCTTTATTTGCATGAAAGGCCTATAA |
| | | AAGGAGAGACTCTAGACACGAGCTTTTATTTAAGTGCG |
| | | TTCATTCTCACTGCTGTTATTGTTTTCTGACAGCATGCC |
| | | TGAACCAGCTAAGTCAGCTCCTGCTCCGAAGAAGGGTT |
| | | CCAAGAAGGCTGTGACCAAGGCGCAGAAGAAGGATGG |
| | | CAAGAAGCGCAAAAGAAAGTTGACAGACAATCCGTAG |
| | | GCACCGCGACCTCGACTGCCCCGGC |
| 99 | CDK6-VPS50 | TGGGCATGGATGAATGTTCCAGGGGCAAAAATCAAAG |
| | | AGACCAGATATACTAAATCACTGGGGGAAACTTTCAGT |
| | | GACTCAACTTGGTACAGAGTAAGAAACATAAGAAGGG |
| | | TGTAGCCCCTTGTGCATTTATGAAATAATGGAGACTGA |
| | | AAAATGCTTACTAAGCAAAACAAAACCAAAACCAAAA |
| | | CCACTAAAAATGTGTTTTCTGCTTTCACAGTGACTTTCA |
| | | TGTTTCCTGAACATGCCATGCTAGAAAGCTACTTACGA |
| | | CAGTTGACCTCATCACTGCCATCCTTGCAGTCAGGGTC |
| | | CCCATCACATCGCCACTTCTTATGGATGCACTCGCCAG |
| | | AGCCGCACTGGATTTCGCTGGCTGGACACTTGGTGTGT |
| | | ATGACTGGCTGACGGCCACACTGCTCCAGGGACTCATC |
| | | AGATTGGTCGGAGCAGTCTGCATCATCGTCGCATACCC |
| | | AGCTGATGGGAAAGAAAGTTGACAGACAATCCGTAGG |
| | | CCTCGGCACCCCCTACAACCCCAG |
| 100 | KLHL15- | TCATTTGATGTTACCTCAATTTCCGTTTTCATTCTCATA |
| | (chr107716924107717154) | CAAGAAGCAGGGCTCAACCACCTTTGATGTGGTTTCCA |
| | | GTTCTCCACCTCCTCCCAGTTCCTCAAGGTGCTTGATCC |
| | | AAATATCTGCCCCATACAACCACCTCCTGGTGACCACC |
| | | TCTCTATGGGACAGCTAGATACAACTTGCTTGAAGCAC |
| | | TCCACTGACCCCCACATTCTGCATGGACAACACAGATA |
| | | TCCTGGCCTGCGGTCGGGGTTCGGCCGGGGGAGGGGG |
| | | TCCCCGTTTCCCTCTCCTCATCCTCGTCTCCTCTGGCTCT |
| | | GGTGACGTTGCGGTTTCCTCCCCCCTCCAGCGGAGCCT |
| | | CAGCGCCCGCCGCGTCCCTCCCTCCGCCCCTTTCGCCC |
| | | ACACCTACCCGCCCCCGCCGGCCCGGCTCTCAGTAGCG |
| | | TCGCCCGAGGCTGCAGCAGCGCATCCCGGGGCATGGC |
| | | GCGGCGAAAGAAAGTTGACAGACAATCCGTAGGCGCT |
| | | TCCGCGACGCCTACGACCGC |
| 101 | PRSS1-BPGM | TTAGGCATTAACCTGAAACTAGCCTGTACAGGAAGCTA |
| | | TACACTTGCCTCACCTCTTAGCTAACATAGATCAAAGA |
| | | TTTCCTGAAGCCTTTGTAATAACAGGAAATAAAACAAG |
| | | GGCTGTGAGTGGTACAGGTGAGAGAAACACACTCGAG |
| | | AGGCTGCTACCGGCAGGCGGACACAGCAGGGCAGGGC |
| | | CAGGCGTGCAGAGCAAGCAGCCAAGCTGACCTAATGG |
| | | TTGGGTCCTGATGAGAAGGAGCCAGCCATGGGAACTT |
| | | GGGGGAACAGCACTCCAGGTAGAGAGACCAGCAAGTG |
| | | CAAAGACCCTGCAGAAGGAAGGAGCTTAGAGTCTGTG |
| | | AGAACAAAGAGAGAGGAAGTGGGTTGGGCGGGGGGG |
| | | GTGGGCAGGACCAGCCAGCACAGGATCTGTGGAGGAG |
| | | GAAGGTGATCTAATCTCTGTTTTGAAAGTTCCTTCTGTC |
| | | TGCTGTGTAGAGAATTAAAGAAAGTTGACAGACAATC |
| | | CGTAGGCCCCTGCTGCATCTACGGCCTCTT |
| 102 | ARRDC2-ZNF665 | CATAATACAAATGCCATAATAGAGACCAGTTAAAACC |
| | | CAGTACTATTCCGTCTGTGGAGCAGTGCTTGGTTTCAC |
| | | ACCATTTTCAGTCTGTCTAGAATTGTTTCCTGTGTGTTA |
| | | GTCACTGTTACCACCCTGTCCCCAATTACAAACTAAGC |
| | | TCCTTTAAGAGATAGAGCGTAGGTTATTCAATTTTTGT |
| | | GCCGTGCCTCACCTTGTCTAGGACGCAGCAAATATTAG |
| | | ATTCCTTGAGGTCAGATTTGGAGCTTCTCATTGCACGC |
| | | GGAGATTATTATTGCATCGGGTTCCAAGCCAATGGGAA |
| | | GCCCGGGGGAGGGGTTTGGCATGAGGAAGCGTTGGTT |
| | | ACAGCAGCTGATTGGCTGCAGCCAAGACTGTGAAAGG |
| | | ATAAAGAGGCGCGAGGCGGAATTGGGGTCTGCTCTAA |
| | | GCTGCAGCAAGAGAAACTGTGTGTGAGGGGAAGAGGC |
| | | CTGTTTCGCTGTAAAGAAAGTTGACAGACAATCCGTAG |
| | | GCTCTCCTTCAGCGCCTGCGACTTC |
| 103 | GSAP-GNB2 | AATGAGCTGAGCTGTTTGTTTCCTCTGAGCCATATACA |
| | | GATGCTTTTTGAACTATCTTTGTGTTTGAGGGAATTTTT |
| | | TTTCATGGTATCAGCACACCACTTCCTCCAAGTCATTAC |
| | | TTTGAAACAGTTTTGTAGAGGGAGATATTCCTGCTGGC |
| | | CACCAGCCTAGCCAAGCAAAGTGATGACTGAGGGATG |
| | | GCAGTAAAGCTTTATTAGTGCACTTCATGTTTTTGGATG |
| | | TCCTGGCTTCGAGGCGGGCGCCGGAGGGGGAACTCTG |
| | | ACCATGATTGGTTCCTCCTAACCAGGTGACCGCAGGTC |
| | | GCGTTCCGGTTGGCAGGCACCGGCCAGGGCCAGCGGC |
| | | CTTCAGTCGGTAGGCGGCGGCGGAAGGAGGAGGAGCT |
| | | CCAGCCGCGCTCTCTCCCGGCAGTGGCTGTGCTTCACA |
| | | GCGCTTGTTGGGTTCCCCACCCCTTTTAAATAAGCCGG |
| | | GCTGGTCACAAAGAAAGTTGACAGACAATCCGTAGGC |
| | | CGACTGCGGCATCGCCGACGCCT |
| 104 | SMC6-SCRN3 | TAAGCCACTGAAATGTTAAGGCTGTTATTTCAAAATAA |
| | | TCTGTCTTATCCTGACTGGTAGACTAATTTAGAAACCC |
| | | CACTAGAATGTTCTCCTAACAGTCTCCTCTGTGACAGTT |
| | | TTCACAACTGCAATCACATAGGTATATGTGAGTCATTT |
| | | GTTTCATGACCTCCTCCTACTAGAATGTAGCCTCCACC |
| | | AGGGCTTGTTCACTTCTGGAACCCTAGCACCCAGCACA |
| | | CTCCTACATCTGCAGAGGATTAAGGTGGCTGTTTCTCC |
| | | CTGGAGGTGGAGTGGGTGGGTCACTGCACAGGAGCCT |
| | | ATAGTTGTTGGTCTTTTAAACTCTTATTGGTGTAACCAG |
| | | CCACGGAACTCTGAGGCAAGGGGTTGGGGGTGGGAAG |
| | | GGAAACAGAGAAAAGGCAAGTGAAACAGAAGGGGAG |
| | | GTGCAGTTTCAGAACCCAGCCAGCCTCTCTCTTGCTGC |
| | | CTAGCCTCCTAAAGAAAGTTGACAGACAATCCGTAGGC |
| | | TCGGCTACCGCGACGTCGACAAC |
| 105 | NR3C1-RAI14 | TCATGTTTAGTAGCATTAAGTACATTCTTACTGGTGCA |
| | | ACCACCATTACCACCTGTCTCCAGAACTTTTTCATCATT |
| | | TCATACTGAAGCTCTGTATCCATCAAACATTCTCTTTGC |
| | | CCCAGAAAGAAGCCTCGTATCTGTTGTCAGTCACTTCC |
| | | TGTTCTCCTCTCCCTGAACCACCCCGACCCCCCACCCCA |
| | | CCACAGCAACCACCATTCTACCCGGTCTCTAGGAAATT |
| | | CCTAGAGAGAGAGAGAGAAAGGGAAGTTCTAGTTTGG |
| | | GGTGAGGGGGCAGAGAAAGAATCTGGAGGGATGAAG |
| | | GTCAGGCAGCAGTAAGGGCTTCCTAACACCTAAACAC |
| | | AGAGTGACACAGATACCCCCCACCGCTATACCACCGCT |
| | | ATCTACAGCTGCGTGGCTTCCGGGGGTGGGACAATCGT |
| | | CTTAAGGCTTTCCATGACGTGAGTCCTCGCCAGAACTA |
| | | AGGCTGTGGAAAGAAAGTTGACAGACAATCCGTAGGC |
| | | CCACCACTTCGGCAGCGACATCG |
| 106 | ITPKB-CD55 | TCTACAAAGTATTTTATATGCTATACGCCCAGTAAACT |
| | | CACTGAGTATCTAATCTACAAATTTCTTTGGCTGAGAT |
| | | GGGGTATCCAAAGGGAGCCAACGTAGCTGGAAAAGTA |
| | | ACTACAGCCAGAGACCTTTCACATTATAAACTGAGCAT |
| | | CTGGTACATGGTAGCTATTTTAAGTTTCTCTGACCTTGC |
| | | CTGAGCGTCAGCAGGAAGTCATACATGAAAAACCCTTC |
| | | ACTCCTAGCCGCTGATTGGCTGACTGGGGCGCAGCTTG |
| | | ACGGCGTCGGGCTGGAGAGCCGCAGTCCCGGCTGCAG |
| | | CACCTGGGAGAAGGCAGACCGTGTGAGGGGGCCTGTG |
| | | GCCCCAGCGTGCTGTGGCCTCCGGGAGTGGGAAGTGG |
| | | AGGCAGGAGCCTTCCTTACACTTCGCCATGAGTTTCCT |
| | | GATCGACTCCAGCATCATGATTACCTCCCAGGTGAGTC |
| | | ACCGCCTCCCGAAAGAAAGTTGACAGACAATCCGTAG |
| | | GCGCTACTACGGCAACTCCGACGGC |
| 107 | GJA5-GPR89B | GAACTCTGGGGGAAAAGACAGAAAATATCAACAAGAA |
| | | ATCTTGCAATTATTAGAAGCAGTATGGAAACCCCACAA |
| | | GGTGGCAGTTATGCATTGCAGAGGACACCAGCGAGCTT |
| | | CCACCTTGCTGGGTTTGGGGAATTCCCGCACTGACTCA |
| | | GAGGCTCAAAAAGCAGCATCTGCCCCTTTCCAGGCATC |
| | | AGTCACAGCTCCTCTGCTCCCTCAAGCACCTGATCTTG |
| | | GACTCCTAACTGTTAGTTGGCAAAGGATGTACCATCTC |
| | | CTATTAAACGGGCAAGGACAAGTTCCCTTTTCTGAGGT |
| | | CCCTTAAGATCTCTTCCTGGGGTGATCTAGGCTTGCTGT |
| | | TTTATGAGGGAGGGACATATTTACACAGGGGCAAGAA |
| | | CTAGTCTTTTAATCCATTGTTGCATAGGGGAGGCGTGG |
| | | GCACTTGGAGTTGTCTTAGGACTGATGTCTGAATTAAC |
| | | TAAATGAACAAAGAAAGTTGACAGACAATCCGTAGGC |
| | | CTGCAGCTGCTTCTCCCACCGCT |
| 108 | RBMS1-SCRN3 | GTGGTGGTTTAACAGTATTTTAAAGACACAGGGATTCT |
| | | TTTTCCCCCTTTTATTGAATTTAGGTCAAAGAAAGATA |
| | | AAGTTAAACTATAGTGCTACTGGGAGAAGAAGGACTC |
| | | GTGGCTGTAAGCGGTTTGTGGTTTTGTCACTATGCTAA |
| | | CCTTAACAATCTATGTTACAATAATAGCTTCCTCAAAA |
| | | TGAACATCTGTTGCTTGGTTAAGCATTATTATAATGAT |
| | | GAGTCCTCCGCCACCGGCCCTGCTCGGCCGCCATCGCT |
| | | GTGAGGCGGCTGCCCGCGACAGCTCCTCCTCCGCCTGC |
| | | CAAATCTCGCGATGGCAGCCGCACAGTTGCGGACCAGT |
| | | GGCCTGCTGCTACCAAATCTCGCGAAACTCGTTTTCCC |
| | | TCCCGGGAAGTTGCCAGAACTAACTCTGGGCAGCATGG |
| | | CGTCTTTCCGGCTTCTCCAAACCCTTGCGAAAAACTTTA |
| | | TTGGCAAAAAAGAAAGTTGACAGACAATCCGTAGGCG |
| | | CTCCGTCATCGTCACCTACTCC |
| 109 | CWC15-KCNJ1 | TTTCCGAGAAATTGTCAAACTGTTTTCCAAGATGGTTG |
| | | AAACATTTTCTTCCCCATCAGCAGTGTATGAAGGTTCC |
| | | AGGTGCCCCACATCCTTGCCAATACCTGATATGTTTCA |
| | | GTCTTACTCATTTTAGACTTTCTAATAAATACTTGGTCA |
| | | TGTCTTATTGTGGTTTCATTTTGCATATCCCCAATGACT |
| | | AATAATGTTGAGCATCTTTTCATGTCCTTAGTCTGTTGT |
| | | CCTGTTGGCCACTTTAGTTCATCGTTGCTACCGCCGGCG |
| | | TCCAGAAGACTACAGTTCCCAGCAGGCCCAGGGAAAG |
| | | ACAGGATACCCTGGGTCGGCTCCTCCACGTGACCACCC |
| | | ACTATGGCTTCCTAGTGTCAGGGCCAGCTGTGTAGTGG |
| | | CTCGGTGTGATTTGTTAGCTCTTTGAGGCAGGGTACCC |
| | | TCCTCAGGATTTCGATATGCAAAAAATCAAATCTCTCA |
| | | TGACCAAAGAAAGTTGACAGACAATCCGTAGGCTCCC |
| | | CGGCTACCTCGGCTGCGGC |
| 110 | CLN8-SLA | ACCCTGGCGTCCCCCAGAGGTAACCCCTCCCAGGAACT |
| | | GGGCGTTTGCCACTCCCCTGCACGGGTCTACACCTCTG |
| | | CTACAAACGTGTGTCCCTAAGCAGCGGACGCCTTTTGA |
| | | AAACAAGTCAACTGTATCATGCTACGATATTTTTTCTG |
| | | ATGTTTGTGCTGTTTCTGAGACAGCCTTATGACACCTTC |
| | | CCCTATTCTGTAGCATTCTGTTGGCTCACTTTGCCACAG |
| | | TCCTGAATCCGTCTGTCTGGCTCTGCAGGGCCCGATAC |
| | | CCAGGGCTTCCGCTGTCACTCAGACATGGGGGGCGGG |
| | | GCCTAGCATTCCATCCAATCAGAGGCATTGTGGCGGGT |
| | | CCCTGCCCACTGTTCCTCCAGACACTGAGGGGGTCGCA |
| | | TTCCTTACCTCACCTTTGTCCCTGCGCGGGCTGCGGCTG |
| | | GGATCCGGTCTTTCCAGCCCCGAGAGGGACCTGGTTCC |
| | | TCTGCCAAAGAAAGTTGACAGACAATCCGTAGGCAAC |
| | | CGCATCGGCGCCTTCAGCAG |
| 111 | INSR-PTPRK | TTTGTGTTTTAACTGTCAGATTGAGAGTAGCTCTGTTGT |
| | | AGCCCCTTTGCAGGGTTCCCATGAGACTTTTCCTAGGT |
| | | AGTTACGTTGACTCGCTTAGCAACATAGCAAATTACAG |
| | | AAACGGAGTAAGAACGTAAGAAATCGCAAAGCACGTG |
| | | ACGTATGGGATCTCACTGTTCTTGGCCCTTCTCCCGACC |
| | | CGTAGCCCTGCTCTGCTACGACTGCGTGGGTGAGTCGT |
| | | CTCCTCAGGGCGCAGCGCAGACTTGGTGAGGTGATTAT |
| | | TTTGGCACCTGTTGCCATGGCTCCTCCCTCTCTCCTGCC |
| | | GGGGGCCGGAGGCGGACCGGGGGCTGGCAGTCGGCAG |
| | | CCTCCGCCCCCTCAACCTTCGCGGGGCGCGGGCCGCAG |
| | | CTTTTCGGTTCACAGCGGGCAGGGAAAGCCGCGGGAA |
| | | GGGTACTCCAGGCGAGAGGCGGACGCGAGTCGTCGTG |
| | | GCAGGAAAAAAAGAAAGTTGACAGACAATCCGTAGGC |
| | | CTACCCCAACAACGGCATCGACT |
| 112 | EIF3G-OLFM2 | GAGTTACTGGGACTTGGAGCAAGGGCCTATTTCAGCGT |
| | | AGTTTCCGCTTCTTGTCACATGACGGGAGTCAGTGGGC |
| | | CGCGCCTTCCCACTGCGACTCCCCGTGGGTCCGCAAAC |
| | | CGACACGTCACTCCTCGGCCCTCAACCAAACTGCTTGG |
| | | GGCACGTTCTTTCCGCGCTCTTGACTCCTACGCGCCGG |
| | | CCTATGACAGCTCGGCGGGAGCATACAGACCTGCCTCT |
| | | AGTCCTAGAGACACCCTGCTGGCCTCCGAGAACTCTGC |
| | | CAGCATCCATGCGGCTGCTCAGGGTCGCATCTGGTCAG |
| | | GGCCAGTGCTGTCTGTGGCCGGCAGTTACTGGTTAACT |
| | | CATCCTGTTCCTGTTTAACTTGTTCCCTGGGGATGGCGG |
| | | CAGCCTCTGACCTGTCCAGGTGCCCTGTCCAGCTGACT |
| | | GCAAGGACAGAGAGGAGTCCTGCCCAGCTCTTGGATC |
| | | AGTCTGCTAAAGAAAGTTGACAGACAATCCGTAGGCC |
| | | CTCAACGGCATCAGCAACGACG |
| 113 | FEZ1-SIGIRR | GATGTATCACAAATAAGAGGGCAAATAGGGAGATGGA |
| | | ATAGAATGGGGGAAAGCAAAAGTTTTATTTACAGTAA |
| | | CAGCAGCTCATAACAGCCACACTCTTTTATCTGTGAAT |
| | | TCATCACTTGAGGCTCTGTTTTTGTTAGCACAGCTCTAT |
| | | TTCCTGGAGCAGGACTTAATATGACAGGAAGCCATTCT |
| | | GCTACTCATGAGGAGAATCTAAAACCAGGTTACTTCTT |
| | | TGCTCCTCCTTCTGCATTTCACACACATTTAGCTATCTC |
| | | TGTCGCTCTGTCACACACACACACACGCACATACACAC |
| | | CATACACACACACACGCACACCCGTCCGGGTCCCTCTC |
| | | GGTGTGTCTTCCTTCAAGATGACAGGGCGCTGGAGAAA |
| | | AGGCTGCTGGAATCAGCTGGTAGGTACAGTAGTAACCC |
| | | GAGCCCCACCCACCGGCTGCCTTTGCTTGCTCAGGTTT |
| | | CTCCGGTCAAAGAAAGTTGACAGACAATCCGTAGGCG |
| | | TCGTCCGCGGCGGCGGCCGCTG |
| 114 | | GATGCCAAGAAATTTTAAGTGTTACGTGTAACTGAGGG |
| | | CATGGAAGAGCAAGTTTATCTCCCTATATGGCCACCCA |
| | | CGTTTTCCTTTCTGTCTGACTGCTGGAAGGAAAGGGGG |
| | | AGGTAACTAGAATCTTTCCACACACTCATCCCTGAAGC |
| | | ACTACCCATAAAGTATTACCACTGAAGAGTGAGGACAT |
| | | AGACAGTTCACTGTATTCCTCCCCCAATTCCTAGAAGA |
| | | GTTCCTTCTGTGGGTGTTGCAGCACACCAGCCCAGCCT |
| | | CGCCTAGCTCTCTGAAAGAAAGCCTGCAACAGAAGGG |
| | | AGAGAAGCGGCCCCAAGTGCAGCTGAAAGCACTCCTT |
| | | GGGAGTCAGCCAAGACCCTCCATCCAAGCAGGGCTGG |
| | | ATCCTTGCACTTGGCCATGATAAGGAGATGCTTCCAGG |
| | | TGGCAGCAAGACTTGGCCACCACAGAGGCCTTCTTGAG |
| | | GCCCCCCGTATAAAGAAAGTTGACAGACAATCCGTAG |
| | | GCTGCATCGGCTACGACCCCTTCGG |
| 115 | LPIN1-EIF2AK3 | GAAATATAGTTTGAATTCCACCTCAAGACCTTGAGCTT |
| | | ATGACTTCTGTCTAGCTCTGTGTATCATTCATACTTCCC |
| | | ACTAATTTTTAACAAAATGACTGGGAGAGGGAGAAAG |
| | | TGAAACCCAGTGGACTGAGGCTTGAGTGAGCATTACCT |
| | | CCTGGGCCTGTTGATTTGCGTCTCCCTGGTGACCTTCTT |
| | | CTCTGACCAGCATCCGTGGGCTTAGGGAAAGCACGGTG |
| | | GTCCTGAAAAGGCAAGGAAAAGGAGAAATGTTGCACA |
| | | GTCTTATGGTCTGTCCCCTGCCATTCTGTTCCCCTGAGG |
| | | TGTCAGAGCAGCTTCTCTGTCTTCCCAGGGATGAGTTT |
| | | ACACAGACATTCCCTGTGGCCCCCTGAGGCCTCCACCT |
| | | CTGAAATCTGCAGAACAGCCAGGAAACAAGCCCGGCC |
| | | CATGGCGCCACCTACTGCCTGCCTGCCTCCGCTCATGC |
| | | ACCCTGGGAAAGAAAGTTGACAGACAATCCGTAGGCC |
| | | TTCAGCCTCGACTTCAGCAGCT |
| 116 | DIABLO-KLRG1 | GCACCTTCGGCTATATAAAATTTTCAGTGGAGACAAAA |
| | | CCTTAAAATGTAACACTACATTTGTGTCACTTAGATCA |
| | | AAACTGAAACAGTGTAAAATATACTTTTGTTATGACTT |
| | | TTATCTTACTCACTGACTAGTAAGAAATAATATTTTGTA |
| | | TTTTTTCCCAATATACTTTTTTTTTGAGACAGAGTCTGG |
| | | CTCAGTCACCCAGGCTGGAGTGCAGTGGCGTGATCTCA |
| | | TCCTCCCTCCGTCTTCACCCGCCCTGGGCTGCAGAGGA |
| | | ACAGGGAGTGGTTTTGGTGGTTGGCACAGGGGTAGAT |
| | | GTTGATGGCACGAGGCCCTATCATCTGACAGCTGCCTG |
| | | ACCCACGGTGGGGAGGGCCCTGGAGGGAGTGCTGCGG |
| | | GAGCAGGAGGGGCAGTTCCTATAGGCAGCCTGACTCC |
| | | CATGGTGAGCCCACTGCCACCACCACATTGCCCTGGGG |
| | | CAGCCCTGCAAAGAAAGTTGACAGACAATCCGTAGGC |
| | | CAGCGGCATCACCAACTACCGCG |
| 117 | PLB1-IL1A | GAGGTAATATATCTAAGAGGTTGGCACAGTGCCTGGCA |
| | | CATAGAAAGTGTTCCATGAAGTTTCTATTGCCTCTGTTA |
| | | TGACTGAGTCATCCAGGCCAAGGACTGTACCTCAAATC |
| | | ACTCTCAGGCTGACAGATCTCTCTACAGTTTTTGACACT |
| | | CCACAAAGGAGTCAGAAGAGTTTCCTCAGTTCTTCTTT |
| | | TACAAACCAGTGTTGAAGACATTTCCTGAGGAACCCCT |
| | | TCCTGCTGATTCTGGGAACCTCACTTCCTCCGCCCTTGG |
| | | GCTTGGTGGGGTTGGGTAGGGTTGGGTGGGGCTGTGGT |
| | | TTTCTTTTAGGAGGCAGCAGGCCAGGCCTGGAGACCAG |
| | | AGCTTAAGTGGGCCTGGGCAGGCTGGGGTTGAAACTCT |
| | | TCACCCCTTGCGGTCTGTACTGCCTCCCAACTGAGCAG |
| | | CCAGGGAGAAGGCCTAGAGCCTGTGCCTTTCAGCTAGA |
| | | TAGCTAAAGAAAGTTGACAGACAATCCGTAGGCCCTA |
| | | CTTCTACAACTTCAGCATC |
| 118 | TBATA-TSPAN14 | CTGGATAGAATTCAGGGAGCTATGAACTGAAATGGAA |
| | | AAAACATTACATCTTGATTTTCACAAACCTCTAACTGA |
| | | AATTTATCATTTCCTTCAGTATGAGGGTAAGCAACAAG |
| | | CCACAGCAATGTTAACAGTGCCTGTGGATTTATCACAC |
| | | AGAAACCACAGATACTTTAAACAATTGTTTATGTTATT |
| | | ATTTATTAGAGATGAGGTTTTGCTCTGTCACTCAGGCT |
| | | GGATCCTGTAACACACCTTCTTTCCCCTCCGGCTTTCCT |
| | | TCCCCTTCTCTCCCGCCTTCTCCTTATTCATACCAGAAG |
| | | CGCCTCAGCTCTGATTGGCTGGAGCTCTGTGCTATCTC |
| | | AGCCAATCACAAGCCGGGCTGTGCTCCTACACCATCCG |
| | | AAGAGCGAATCGTGCAGAGACCGTGTCTACGATTGGC |
| | | CTCTCCCTGACAAGGATTTAATTTTGAATTTTTCTTTAT |
| | | GGCGTGGAAAGAAAGTTGACAGACAATCCGTAGGCCC |
| | | TGCGGCATCTCCTGCCACTTC |
| 119 | ERN1-RAB37 | ATTTCTTTTTAAAAAATCAATATGTCAGTATGTTTAATT |
| | | CTTTGCCTTCTACTTTTAAACTTCTCCGTAAAGCAACCT |
| | | TTTTCGATGACCTGCTCCACCCTGACTCATTCTGATTAC |
| | | CTGCTCCACCCAGACTTATTTCGATTACTTACTCCACCC |
| | | TGACTCATTTTCCACCCTGACTCATTCCAATTTCCTGCT |
| | | CTGTTATAACCATTTTCCCCAACAAACCACTCACCTCCT |
| | | TTCGTCCAGACAATGAAACGAAGAGGTGGAACGCGCC |
| | | CAGTGACTACGCCCAGTAGCATTAAAATCTGGCTTTTC |
| | | AGAATCGAGGTGACTCATTCCCCGGTACATTCCCCAGT |
| | | AACTAACAGTATATAAGCCAGCCTTTCAGCACAGAGGT |
| | | GATTTCATCTTCCAGGATCCACCTTGATTAAATCTCTTG |
| | | TCCCCAGCCCTCCTGGTCCCCAATGGCAGCAGCCGCCA |
| | | TCAAAGAAAGTTGACAGACAATCCGTAGGCTCCCCCGC |
| | | GTCAACGACATCCTC |
| 120 | BCL2L11-SLC4A5 | CATCGTCATCATCATCACCTAATCCTTACATGACTACTG |
| | | TGAAGTAGCAAATGTAATCGATTGTAGAGGGAAGGAG |
| | | ATCCCGGTTCAAACAGCATAAGTGACTCACCCAGCATC |
| | | ACAGAGCTAGCGTGTGGGTGGAACTGAGAGTCATGCA |
| | | TCAGCTCCTGCACCACAGCTGCCTCCCTGGCAGGGAGC |
| | | AGGAGAACAGGGTCGTGGCCAACATCCCCTTGCTCACT |
| | | CCTTCCTTAGTGTCAAAACAGCCGGAAATCAAGTCCGT |
| | | TTATCTCAAACTTTAGCATTTTGGGAATAAATGATATTT |
| | | GCTATGCTGGTTAAATTAGATTTTAGTTAAATTTCCTGC |
| | | TGAAGCTCTAGTACGATAAGCAACTTGACCTAAGTGTA |
| | | AAGTTGAGACTTCCTTCAGGTTTATATAGCTTGTGCGC |
| | | CGCTTGGGTACCTCGGATGTGAGGGCGATCTGGCTGCG |
| | | ACATCTGAAAGAAAGTTGACAGACAATCCGTAGGCCA |
| | | GCGGCTACTTCTGCGGCCACA |
| 121 | FAM50B-DSE | AAGGAGCTGTAAGCTCTTCTCAGGCCAGAGACCCAGG |
| | | GTGCTCTCTGGGCAGGAGGTGGCTCTGCTCTGTCTGCC |
| | | GAACTCTGGGCGTCAGCACTGGGGCGTGCTGGGGCCTC |
| | | AACCCAGCCCCACCTCCAAGGCAGGCGGAGAGCACTG |
| | | CTGATGGATTCCCCAGCACTGCACTGTGCGAGGCCCAG |
| | | GGCGGGGACCCCAGCAGGCAGCGTGGTAGGGTTTGCT |
| | | GGGGATCCTGTAGACCTCTATGAACCAAATATGCTTCA |
| | | GGCTTGGGTTGAGCACAGGAGAGGGAGGAGGGAAAGG |
| | | TCACTGGGGCTGGGAGTGCCTACTTCCCTCTGTGAGTG |
| | | TCACCCCAGTCCACCCAGTACCATACCTTTCTCTCTCTG |
| | | GACCCACTTCCTTTTGCTGCCGGCTCCTCCCCATTGAAT |
| | | AACAGCCAAGTTGCTTTGGTTTCTATTTCTTTGTTAAGT |
| | | CGTTCCCTCAAAGAAAGTTGACAGACAATCCGTAGGCA |
| | | CGCCAGCACCGTCTCCATCTTC |
| 122 | TOM1L2-CCL4 | TTTATGTTACTTTTTGCACTCTGAGCTCATGATTCAGCC |
| | | GCAAGGGAATCACCTGGAGGGCTTGTTAAAAGTGGAT |
| | | CCTACCCTCAGAGTTTCTGTTTCAGTAGGTTTGGGGTG |
| | | GGAAGTAGCATTCTACATTTCTGTCAAGTTCTCTAGTG |
| | | ATGCTGATGCTACTGGTTCACTTCGAGAACTGTAGTTC |
| | | TTGCTTTGAAGTTCTGTTTCTTTAATCTTTTCTTTATCTT |
| | | TCCTTTCTTCTCGCCTGCACCTCCCTCATCTTTCCTCTAT |
| | | GATGACATCGCCCTGGGGAAGAGAAGCTGAGAGGAAC |
| | | TCCTCACTCAGCTAGCTTCAGGAGCATGACGTCATCTC |
| | | TACCATGGAAATTCCACTCACTCTCCTGTGCCCCCACA |
| | | TTTGTCCTAGGCCTCAGAGTCCCTATAAAGAGAGATTC |
| | | CCAAGTCAGTATCAGCACAGGACACAGCTGGGTTCTGA |
| | | AGCTTAAAGAAAGTTGACAGACAATCCGTAGGCATCA |
| | | TCAGCTCCCACGACAGCAT |
| 123 | RNF112-LINC00324 | CTTCCTTCCTGAATCTTCCCCATCTTCTTGTGGCCCCAT |
| | | CACTGCCCCACTCTGTAGGATACCACAAGAGAACATTG |
| | | CTTGATCATTACTAGACTGTGAATCATCCCTCTTCCCCT |
| | | TCCACCCATCATATTATGAAATCCTCTTGAAGAGACCC |
| | | TCAACCCTCCTTTCATAACTTTGAAACTCCAGCACTCA |
| | | GCATGGTGCTTGTTGCCCAGTTGGGTTGGTGTCCCATA |
| | | TCCTAAAATCTAAACAGCCTGTGCTTGTGCTGACTTAT |
| | | GGCCACCACCCTGAACCACTTCCTGGCTTCCTGGCTTT |
| | | GCTAGCTGTTGAAACAGTGGAACTTGCCTCATGTGTGT |
| | | GTATGTGTGTGTGTGTGATTGTGGGTGTGGGTAGTTTA |
| | | ACCTTTTTATATCTTAGCTGCCTGTAAACTCAGTGATTG |
| | | CTACGTTCAGATCAAACCTTGAACTTTTTTTTTTCTGGT |
| | | GCCAAAAGAAAGTTGACAGACAATCCGTAGGCTCGAC |
| | | ACCCTCGGCTACGTCCGC |
| 124 | CAMK4-ARL15 | ACACATGGTCTGCTGGTCATTCAGAGTCACCTTTGGGT |
| | | CTATTGACTAGGTTGCACACAGTTCAATTGTGTGTTTTC |
| | | TTTTTCACAGTGCCCCTCCACTCACTGAAAAGGAAGTT |
| | | GAGGTAAGTGACTCAAGTGTGTTATGAAATTTAGTGCC |
| | | CGTGCTTGACTGGGCTTATTCAAAACATTTCTTCCTCCA |
| | | GACTCTAGCTCTGACATTTTTAGACAAAAATGAGAAAG |
| | | TCCTGGCATTTTAACCCCTGAGTGGATGCTGACAGAAG |
| | | TGCTCAACTTCAGATATTTCTGCCCCCTCCTCTTCCTTC |
| | | CTGGCACCACATTCCTCACGCACCCTCTCCCTGATATTC |
| | | TGTCCCATCCTGGGACTAAACATGTGCTGTGTCTTTAA |
| | | ATGGGCGGAAGTGGCTGGCAGTGCTTTGCTTGGGTACT |
| | | GCATTGATTAGGAGATTATATGGAGCTGCGGGAGCTGC |
| | | CGCCAAAGAAAGTTGACAGACAATCCGTAGGCCTTCG |
| | | ACCTCAACCGCTCCAACT |
| 125 | LYVE1-GRAMD1B | AACATCCACGTGGCCACCCCACCCAATACCTCGATCTC |
| | | CCCGTTCCCTGCCCATCTTGCCTCTAACTTTTCTTCTAC |
| | | CCCAACTCAGCCACTTCCCTGCCCCCATGGTCGCCCCA |
| | | CTCCTATTCAACACCAGTGACTCCAGCACCTCTGAAAC |
| | | TCAACCACAAACATCCCATCTTCTCACCACAACCTTCT |
| | | AGCTTTCTAGGCAAAAGTGCATCGACTTTTGCATCCCA |
| | | TTCCTCAAAATATCCGCCGGAGGAGCTTTGCTAGAGCG |
| | | TTGGTCTTGGTGTCAGAGAGAATTCGCTTTCCTTTTCTG |
| | | TTTCCCGCGGTGTCCTTAACCAAAGGCCTCCTCTCTTCA |
| | | CCCGCCCCGACCAAAAGGTGGCGTCTCCCTGAGGAAA |
| | | CTCCCTCCCCGCCAGGCAGATTACGTTTACAAAGTCCT |
| | | GAGAAGAGAATCGAAACAGAAACCAAAGTCAGGCAA |
| | | ACTCTGTAAAAGAAAGTTGACAGACAATCCGTAGGCCT |
| | | GCGGCGTCGGCATCCGCGCCT |
| 126 | HHLA2-SATB1 | GGTGGGTCCCTTCCCCAAATGTGCCTCCTTGCAGAGAG |
| | | CCATGGTTAGTAGCTCCTCCTCCTCCTATGCCCACTGCA |
| | | TACAAGCACTACCTGGAGCTTACAGAGGTGAAGTCACT |
| | | TCCTGTCTGTAAGTAGGACATAGGCGAGGAGGGGAGA |
| | | TGAACTATGCAGGAAGTAGGACCCAGAGATTAAGATG |
| | | TCATTATGTAAAATAGGATTTTTTTTTTTTTTTTTTTTTG |
| | | ATCCTAGAGGAGCCGACCAAACAGGGAACAAGACGAC |
| | | TTGCAGCCGCCCTACAAGGGTTTGGTTGCGGCCAAGCT |
| | | GCCTCTTTAAACTGGCTTTCCTCCCCCGAGGAGGGAGC |
| | | AGGGAGAGAGGGTGGAGTGTGGTTTGCGGCCAAACCT |
| | | AAAGGCTGCAATTAACCAGCCGCCCGGCCTTCGCTACT |
| | | CCGGACAGACGAACGTCTTAAAGGACGTCTTCGCTCCT |
| | | CGGGAGTCGAAAGAAAGTTGACAGACAATCCGTAGGC |
| | | TTCACCGGCCTCCCCTCCCACGG |
| 127 | CUBN-GPR158 | GGGCCCCACCATCCTGCACCTGGTGCAGAAAAACCCTG |
| | | TGAAGCTGCACTACAGAAAGACACCACCAGGTGGCAG |
| | | GCCTGGAGATTGCATGGAGGCCCCGCCCCCCCAACCAA |
| | | TTCTTTGATAATAGCACAGTGTTGAAGAGAGGGGGCCA |
| | | TAAAAGACTGAATTCCTGTTCATGCCAGGCTGGCTCTG |
| | | CCCAACATATATGAGACTGCAAGTTCTGCCACTGTGGG |
| | | CTGTCCTCTTGTTTTAAAATAGTTCTGTTAAGAAATCGT |
| | | TTTCGTGATAGTTTTGTCTGTTCTTTTAGGCTTATCTTG |
| | | AGTAATAAAATAGTACAGGAAGTGAGAGCGCCACAAA |
| | | CAACAGTGGGTTTGCTAGTTCTTTCGACGTCTGTAGAA |
| | | CACTTGCTACGTATTCTGTGCTGACAATACGAACACAT |
| | | GTAAGAAGTGATGATCCTGATGGATTCTCATATACTGT |
| | | ACTATTCTAAAGAAAGTTGACAGACAATCCGTAGGCTT |
| | | CAACTACAGCATCTGCGCCAG |
| 128 | RRAGC-CD53 | TTATCATAAAACTAAAACATCCTATGAGTAGTAAGTCA |
| | | CCAAGGCCAGCATCTCCAGCATGTGCTCCACTTGTTAA |
| | | AATGACGCAAGTGAGTCACTGAATAGCTCAGCATCATT |
| | | TTGTCACCTCTGTGGGTGTTCTGAAAACAGATCAAGTC |
| | | GTATCATTTCTGTATCCAAAGAGAGAATTCAATTAAAT |
| | | TACATTGTTTTTTGAAACTATCACTGTTTGTTAAAACTA |
| | | ATCCTTACTCACTTTTTTGATATTGGATTTGGAGGCAGG |
| | | ATGAAAGTCTTTCTTGCACATGAAGTTGGCGAAGGATT |
| | | TCCCCATCTTGGAACTGGAACTAGGGAAAGCAGGGGC |
| | | TAGATCTGTTAGCAACGTAAACAAACTCAGCCGCCTAA |
| | | CCGGAACTGCGTTTCCAGCAGCAAGGGGGAACTTCCG |
| | | AGATCAAAGGTGACAGCTTCCGGCAACTGATGCCTCCA |
| | | CTGGCCACAAAGAAAGTTGACAGACAATCCGTAGGCG |
| | | GCTTCTACGGCGGCAACAACAA |
| 129 | WIPF1-CCNYL1 | TTGATTGCTCACTGCCTGTCTTGGAAGGCTGTAACTCTT |
| | | TGACTCACACACCCAGGGTCTGTGTTGACTTTTGTTTAT |
| | | CAGTATATTTCTAGAAGTTAGTCCAGTGTTTGAATTCA |
| | | ATAGGTGACTAATTGTTGAATGAATACTGCACTTTCTC |
| | | TTTGAAACTCAGCTACAGTTTCACTTTGCTGAAGAGTT |
| | | CTGCACTACTTAACTTTTTGAAACTAATGTTCTATGTAT |
| | | CCTCTGCTTTATGTGAATTTCTCACTTTAACTTGTTTGC |
| | | TCCATTTTGGGTGCAGACCCAGCCCCATGTGCCTGACC |
| | | ACAAACCAACATTTCCTGTCTCAATTCTCCTATTCATAT |
| | | AACGTTTGAGGACGTTTGTTAAGTTTTAAGAATTCCCT |
| | | CCCACCCCCAGATTTTCGCAGGGTTTTGTTGATTTCCCC |
| | | TCAACTTTCCACAGGTATCTTAAAAGCTTTGCTCACTCA |
| | | TAAAGAAAGTTGACAGACAATCCGTAGGCAACGTCAT |
| | | CTCCTACAGCGACAA |
| 130 | TMEM8B-MIR1302-8 | ATCACATCAGTTCTAAGAATTTGACGAGTAAAAAGCAC |
| | | TGGGCAGGTTTTAAGTCAATAGTGGCCTTCCAATTTCT |
| | | AAAGTTCTTTCAGAGTACAAACACATTGCTTATACTCC |
| | | TCTGGCATGGTTTTGCTCATTTGCAGGAAAACCATATTT |
| | | TGTACCACATCCTCACATATTGATATGTTGAGGAAATA |
| | | CACAGCAAACACCACTTCCCATCCATCTGGTAAGCTTG |
| | | TTCCTTGCTAACCTCCAGCTGTGCCCCTTAGCAAGCCG |
| | | ACTTCCAGATCTACTCGGAGTACTGCAATAACCACCCC |
| | | AACGCCTGCGTGGAGCTCTCCCGGCTCACCAAGCTCAG |
| | | CAAGTACGTGTACTTCTTCGAGGCCTGCCGGCTGCTGC |
| | | AGAAGATGATTGACATCTCCCTGGATGGCTTCCTGCTG |
| | | ACTCCGGTGCAGAAGATCTGCAAGTACCCTCTGCAGCT |
| | | GGCCGAGAAAGAAAGTTGACAGACAATCCGTAGGCGG |
| | | CGTCAGCAACATCGTCTGCCC |
| 131 | DAPL1-CFLAR | ATGTGTATATACCATGTTTTGCTTTTCATTCATCTGTTG |
| | | ATGAACACTTGAGTTCTTCCCCTCTTTCAGTTATTGTGA |
| | | GTAATGCCTCTATAAACGGTGGTGTACAAGTATCTGTT |
| | | TGAGTCCCTGGTTTCACTTCTTTTATGTCTAGCAGTGGA |
| | | AATGCTGGGCCATATTCTATATTTAACTTTTTTTTTTAG |
| | | AGACAAGGTCTCTTTCTGTCACCTAGGCTGGGTACATC |
| | | CTTTACCTATGTGCGCTGGGCAATGGGGACTCTGGGCC |
| | | TCCCCAGGGCCCTAACCCAGGCCTCTGCTCCCCCACCC |
| | | CCTTAGCACAGGATGTGGTCTTTGCATCTTCATTTGCCT |
| | | GCATTTGCATATAGCCTCTCAGCACTAGCCTTCAATGG |
| | | ACCCTTCAAAGTCATGTTCTTGTCCCTCACCCGGAAGT |
| | | CTCCTGGCCACATTCTAGGTGGGGAGACTGACGGCTGG |
| | | AGGAAAGAAAGTTGACAGACAATCCGTAGGCTGCATC |
| | | ACCTCCTACACCTTCGG |
| 132 | RPLP1-WWTR1 | AACTGCACCACGGAGAACTTGGTTTTGTGACAGGCCAT |
| | | CACACACCACTGTTCCCTTGCCTCATGTTGGGTAATTG |
| | | GTCCAGAAATGACTCAGAGGAAAGATCACCACCTACT |
| | | GTATCACCCATATCACTTCCTGTAACACCTGGGTTTTTC |
| | | TTGGAATGTTTCCATCCAAATGTATATCATATCTGACCT |
| | | GATCTGTTTGCAAGGGCTGATAAGATTATAGCCTGGGG |
| | | TTCCTAATTTCAAGCTCTAGCTCTTTCTCAGTCGCCAGC |
| | | AATAAAGGACTCCTGAATTAGTCTCAGAGTGTGGCATT |
| | | CTCTCTATGACTTGCTCGGTTACGACATTTGGAAGCCC |
| | | CAGAGAGATTCGCCACCGGGTGAAGACCTGACTCATTC |
| | | CGGGCTTCCCCAGACAGACAGCTGGCTTACAGGGCCAC |
| | | CTGAAGACGTTCCAGGGCTCCACAGGCCACTGTCTTCT |
| | | GGAGGGAAAGAAAGTTGACAGACAATCCGTAGGCCTA |
| | | CTTCGACAGCGCCGTCAGCG |
| 133 | TG-SLA | CAGGTGGCTATTTATGTATCTGCATTGCCCTGCTATGCT |
| | | GTGAACTCCTCAGGGCAGAGACTGGGTCATATTCATTC |
| | | TGTGTCCCCGGTGTCTGACAGTGCCTGGCACAAAGAAG |
| | | GTGCTCACGTTGGGGACTGAATAAAGTTCCCCTGGCAG |
| | | AAACCCAAAAGGTTCAAAATGACTGTTCTTTCCTGTAG |
| | | TCACAGAAGCTCACAGGGCATTTGACTTGCCCTGTATG |
| | | CTCCTGCCGACACCAGAGCAGTTAAAGGGCGTGCCAG |
| | | AGGCCGAGAGGCGGAGCCCCGGGCCGCCGCGCAGACG |
| | | CCCCGCCCCGCCCGGCGGGCTGCTGGGAGTCGAGGACT |
| | | GGGTTGCTCTCCTCCCTTCCCCGGTCCCGCCCCCGTCTG |
| | | TCTGTGTCGACTGCGGACGCTATCGATAAAGTTGCTGT |
| | | TTTGACAACATGGCGGCGCCCATGGTCCGTGGCCCGGC |
| | | AGTGCTCGAAAGAAAGTTGACAGACAATCCGTAGGCT |
| | | CCACCTCATCGGCGCCTTCTTC |
| 134 | SPRY1-CXCL8 | GTTCTTTTGATTTGGAATAAAATGTGCTACTCTTATGCA |
| | | AGGTTACACTCACACTCATAGCAGGAGTTAGAAATAG |
| | | GTCTTCTTGTTCTGCACCAGGTGCTCTAGAAAGAGCAG |
| | | TATAGTTTCTTGTTCCCCTAATGTTTACTAAAAACTTGT |
| | | TGCTACTTGTCTTGTGGGTTTTTCAATTCTGGCTTCATG |
| | | TTTTAACCCTACTAGACACTCAGCAAATATGAATTAGC |
| | | TCCTGGGCCGGGAGGAGGGGAAGTGGGGAAGGTGGAG |
| | | GGAAGGCCGGGCACAGGGGTGAAGGCCCAGAGACCAG |
| | | CAGAACGGCATCCCAGCCACGACGGCCACTTTGCTCTG |
| | | TCTGCTCTCCGCCACGGCCCTGCTCTGTTCCCTGGGACA |
| | | CCCCCGCCCCCACCTCCTCAGGCTGCCTGATCTGCCCA |
| | | GCTTTCCAGCTTTCCTCTGGATTCCGGCCTCTGGTCATC |
| | | CCTCCCAAAGAAAGTTGACAGACAATCCGTAGGCCCGT |
| | | CGACTGCAGCGTCCACGGC |
| 135 | FGD4-ATXN2 | GTTGCTCTTTGACATGTTCCAGTTCCCGTCATACACAGT |
| | | CAATACACGTCTCTCTCTTTTTATCTTTGTTTCTCTTTCC |
| | | CTCGCCTTTGCTAAGTTTCTGTTCATCTTGATTCATATT |
| | | TCCCACTCCTGTATGACCACGAGTGACTGTGGCCAGGA |
| | | TCACAGAGTGGGAGGCTGCCTGCTAGGAATCCCTCTCA |
| | | GCAGGCTGGGTGCCCAGAGGAGAGTGCTGTGTACTCTC |
| | | CTAAGCCCGGTACAGGACTCATTTGCATGGCCCCTGAC |
| | | GCCATGTGACGCAGCCGGCTCCTCCTATATAAAGAAGC |
| | | AGGAGCCAAAATATCTCCGAGTCTGGGTTGGACTGGCG |
| | | GCCGTGGAGTTTGTGACATACGAGGTGACACCCCTCGA |
| | | GTCACTTCCCTTCAACTCCAGCTGGAGCGCCTGCTTGG |
| | | CTTTGGGTTCGTTCTGCAGCCTTCGCCCCGCTCCTAGCC |
| | | TCAAAAGAAAGTTGACAGACAATCCGTAGGCGACGCC |
| | | GGCTGCTTCAACCACTA |
| 136 | P2RY10- | ACTCCAGCCTGGGCAACAAGAACGAAACTCCATCTCA |
| | (chr107825635107825865) | AAACAAAACAAAACATCATCATGACTGAGACTCACTTT |
| | | CAGAGAAGCTGATTCAATTGGCGTGGGAGTATGAGAA |
| | | AGGGAGGGACCAGGATATTGGTGTTTGGGGTTTTTCCA |
| | | GTTGCTACAGGTGATTTCAATTGCAGACATCACTAGCT |
| | | AAAGCAGTGGTTCTCAACCTTGCCTGCCCATTGAAAAC |
| | | ACCTTCCTTCTTCTCGCCTGCACCTCCCTCATCTTTCCTC |
| | | TATGATGACATCGCCCTGGGGAAGAGAAGCTGAGAGG |
| | | AACTCCTCACTCAGCTAGCTTCAGGAGCCATGACATCA |
| | | TCTCTACCATGGAAATTCCACTCACTCTCCTGTGCCCCC |
| | | ACATTTGTCCTAGGCCTCAGAGTCCCTATAAAGAGAGA |
| | | TTCCCAACTCAGTATCAGCACAGGACACAGCTAGGTTC |
| | | TGAAGCTTAAAGAAAGTTGACAGACAATCCGTAGGCG |
| | | CGGCTTCCACACCGACGTCTCC |
| 137 | TMEM92-PRR11 | CCAATCTTATAGAAATCAAAGCTCAAGGAGCAGCATA |
| | | CCTCCAGTTGCATAGCTCACTGATATTGAAACTGGAAC |
| | | TTGAATACAAACACTGTTACTTTAGCCCCATCCCCTTCC |
| | | TGCCAGACCACCAAGGTTCCCATCAAAAATATAAGCTG |
| | | ATATGAGCTACAGAGATATCTGTGAAATATAAACCTTG |
| | | GCTTTCATAAAGTTGAATTTGATTCACCAAGATGCTGC |
| | | AGTCCTGACAGCGGGTACCGCATGACTCAGAGAGGCC |
| | | GTTGTGAGGGTGAGTCAGCTGAGAGTGTTCAGCACACA |
| | | TGACGGTGATGTGCAGGGTTGGTAGGCACCTTGCCTGG |
| | | TTCTTGTTTCCTTCCCACACTCAGTGCGTGACACCAGTT |
| | | TATCCAAGCTCTTTCTGCTTCCTAGATTCACAGGCTCTT |
| | | TGCCTCTTCCTTTCACTGGCTCTTTTGGGATTATTTTCC |
| | | ATCTTCAAAGAAAGTTGACAGACAATCCGTAGGCCAG |
| | | CTACTGCCACCTCTTCGACC |
| 138 | AGPS-ICOS | TGACTGAGCTGGGGCTGATGGTCGGCACGGTTCTGCCC |
| | | AACACTGAACACAATTCTGCTGCCACAACGCTGTGCTC |
| | | ATCACAAAAACCACATAATGTGTGGTTAGCATCTCAAA |
| | | GCTCTTCTCTCCTTTGTAGTCACAAAGACAGCATGTGA |
| | | GAGTCCAGGATGTTTATTAACAGACATTCAAAGCACAT |
| | | ATGTCACCCAGCACAGTGGTTCTCTCCTGTAATCCCAG |
| | | CATCCTAGTCCGCGCGGCCGAGCCCGTGTATGCAAATC |
| | | AGCCATGTGACGGCAAAAGCCGCCTGGCCCAGCCCTGT |
| | | TCCTCAGTCCATATATGGGCAGCGACGTCACGGGTATT |
| | | GAAGACCTGCCCATAAATACTTAGAGCAACACTTTCCG |
| | | TCTAACTGAGCGAGGAGCAATTGATTAATAGCTCGGCG |
| | | AGGGGACTCACTGACTGTTATAATAACACTACACCAGC |
| | | AACTCCTGAAAGAAAGTTGACAGACAATCCGTAGGCA |
| | | TCTGCTTCTACGTCAGCACCCT |
| 139 | KLF6-EGR2 | ACTCAACTCTTGGAACCTTGTATTCTCAGGGTGCAATA |
| | | TGAGAAACAAATCCCACAGATTGCATTGATTGGTTATT |
| | | TGTTTTGGCCACTCTGTAAAACACATCTTCCTGTTTATG |
| | | CCACTTATCTTGCCTGTAACATAAGAACTTTAGTTCTGC |
| | | ATATGTGGTCAGTGGTTTGCACTTTTTCCTCCCAAGATC |
| | | TACATGCAAAAGTTGCTGTTGCTGCTGCTGCTACAAAT |
| | | CCTCCGCGCTTGGGCCCGCCCTCCTCACAGCCCCGGAG |
| | | CGCGGCCTGCCGGGGAGGTGGATCTCGCGCTCCCCACA |
| | | CAGTCACACTCCGCGCACTCACACACTTGGAAGCGCCT |
| | | CCCCACGTCCCTCCCCTGCCCTCCTCTTGCTCTGCTCTT |
| | | CTCTCCCTCACCACAAATAGTCGCCGACCTACTGCCAA |
| | | TCCGCCTCTCCTCTCTCTCTCTCTCTGTCTCAACATGAC |
| | | TGAAAAGAAAGTTGACAGACAATCCGTAGGCTCCTCG |
| | | GCAACTACGTCGACGCC |
| 140 | HAVCR1-ITK | ACTGGATTTGCTGGAACCACTCCCACACAGGGCACATA |
| | | CTTAGGCCGACCTTTTGTAGTATTGATGTCATCAACCAT |
| | | CATCTTATGACAGCCTCTTGGTCCTCTAAGTTTCTTGTA |
| | | CCAGCACCTGTGCCAATGAAGTAGTGGTTTTCACTGTG |
| | | GTGGGTATTAATAGTACAGTATCCAGTCTTTGCTTCTCT |
| | | TACTTTCATTTAACTTGCTCAAGGCAAACTAAAACCTT |
| | | CCTCCCCGATTATTTACAGCCTCAGCAGAGCACGGGGC |
| | | GGGGGCAGAGGGGCCCGCCCGGGAGGGCTGCTACTTC |
| | | TTAAAACCTCTGCGGGCTGCTTAGTCACAGCCCCCCTT |
| | | GCTTGGGTGTGTCCTTCGCTCGCTCCCTCCCTCCGTCTT |
| | | AGGTCACTGTTTTCAACCTCGAATAAAAACTGCAGCCA |
| | | ACTTCCGAGGCAGCCTCATTGCCCAGCGGACCCCAGCC |
| | | TCTGCAAAGAAAGTTGACAGACAATCCGTAGGCCAAC |
| | | GGCCACTCCTTCATCATCT |
| 141 | P2RY6-CD44 | CCTGTACTTATACCCCTAAACCTAAAACAAAAGTGAAA |
| | | ACAAGTCACTAGAGAATTTTAGTGGGTGCCTTCTTCCC |
| | | AGTTCTAACCACATAACCCCTGTCTCTGATCAGACAAG |
| | | CACTGCTGGAGGCCAACACAGAAACATAGCTGGCAAT |
| | | GGCTCACAGGGCATTTCCCATCCAGTAATGACCTGTCT |
| | | GCCTCTACTTAACTCCCAACACCACAGTGGAAAGGGCA |
| | | CCTTCCTATTTGCAGGGCAGAGTGAGAAGTCTAGAGCC |
| | | TTCCTACTCAAAGTATGGTTTGCAACCAGTAGCATCTG |
| | | CATCTCCTTGGAACTTGTTAGAAATGTAGAATCTCTGA |
| | | CCTCACCCCAGACCTACAGAGTCAGAATCTGCATGCTA |
| | | ATAAGCCCCCAGGTCCTGTGTGCTCCACAATGCATTTG |
| | | AGGAGCACTCATGTGGAATGCAGACTTGCCCCTTTCCT |
| | | TTCTGGCATAAAGAAAGTTGACAGACAATCCGTAGGCC |
| | | TACATCCGCAGCCTCGTCGTCC |
| 142 | ARHGAP20-SORL1 | TGGTGCACATCCCTGCTCTGCTGCGTGATATCTTGCAA |
| | | GAACTTCCCCACTCAGAGCCTCAGTCTGCCCTCTGTGA |
| | | AATGGGAGAATGATGGCACGTTACTTACCGTGGTGGG |
| | | GAGAAAGCGCAGGTACCTCCTAGATACTCTCTCTCCCA |
| | | CCCCACCTCCGGCGGCCAACGGCCAAGCAAACCTCCA |
| | | GCCAAGATTTGGGGTATGTGGGCAGGGCTCCGGCGAA |
| | | GGGGATCCTAATGAAGTCACGCGCACCGCGCTGCCGA |
| | | ACTCGGTGCCCCGCGCTGGGCGGGGGGCGGGTAGGCC |
| | | TGTCCATGTAAATCACATCATTTGCGTGCCCCGCCCCG |
| | | CCGACGCCACTTGATTCCAACATGCAAGAAACGCCCCC |
| | | TCCCAGACCCGGCCCCTCACACTGCGTTCCCCCTCCCG |
| | | CGCAAAGGGGGCGGGGCGGAGGATGCAAATGAACCTC |
| | | GTGTAGGCCACGGAAAAGAAAGTTGACAGACAATCCG |
| | | TAGGCGCGACTACACCGACGGCGTCGTC |
| 143 | LTBP4-ZNF626 | GATTTGTTCCACTTTTGCTGGCACACCCCTGGTCTGCCT |
| | | AGAAGTTCCACAAGGACACCCCCGCCCCATCTGATTTG |
| | | TCACTGCTGTGTCTCTAGGGTCCAGAGCAGTACTGCTC |
| | | TGGGACTGGGATCTGGGGATGCAAGGCTCTGCCAGCC |
| | | GTGACCACCAGGGGGCATCCACGGGTCAGAAACCACG |
| | | GCTTGGAGACCCACCCGGCCAGGCCACAGAGAGAGGT |
| | | GTGCTCCTCTTTCCACCAATAGGAGGGGCGAATGACTC |
| | | CACTGAGGCCACGCCCAATGTTCAAGTCTATAAAAGTC |
| | | GGTGCCGGAGGCTCCCAGCTCAGATCGCCGAAGCGTC |
| | | GGACTACCGTTGGTTTCCGCAACTTCCTGGATTATCCTC |
| | | GCCAAGGACTTTGCAATATATTTTTCCGCCTTTTCTGGA |
| | | AGGATTTCGCTGCTTCCCGAAGGTCTTGGACGAGCGCT |
| | | CTAGCTCTGAAAGAAAGTTGACAGACAATCCGTAGGCT |
| | | CTACTGCCACATCCGCTCCTGC |
| 144 | RPAP2-SLC2A1 | GTGGGGGCTGTGGCTGCGGTTAACAGGCCCCCTCTGAC |
| | | TACCACAAACTTTCCATGGGTCACTTTCTCACCAAACC |
| | | AAAAGTAGTCATTGCAGAAGCTGCACTGTAAGACTCAT |
| | | GGACCTACAGACCGAGCTGTACAGTAACCTTACACAAC |
| | | CACCCCCACCCCTCTGTGAGTCATCCCCACCCCACGCC |
| | | TCCACCTCCAGCAGTAGAACTGAAGCATAGGGCATGTG |
| | | GGTCCTTTCCCCCTCTCAGCGCCGTCCTCAGTGGCCAC |
| | | ACCAAGAACGAGGCCATGTCTTCCTGGGAAGGGCCTC |
| | | AGATGTCAGCAAATGCCCTGGTGTCTTGGGCTGGGCTG |
| | | GGGGCACCAGGGTGAGGTGGTGGGGGGAGCCAACCTC |
| | | ACTGCCCCTCCCCTTCCTGCCTGCCCTTCTTCCGGGGCA |
| | | CCCAGCAGCTCGGTCCTAGGGCGATGTTGACAGACAG |
| | | ACAGAGGGGCAAAGAAAGTTGACAGACAATCCGTAGG |
| | | CCGGCGACTACCACATCGTCAACC |
| 145 | CRTAM-TSPAN32 | CTGCAGGTTGATTATACAAGACACAATTATATCCTCAG |
| | | AGAACCAGGGGCTTTCCAATTATGGGCTTTTGTTCTCA |
| | | GGCTTGTTCTCTTGTGGTCACAAGATGGCTGCCACAGC |
| | | AACAGTCCTCACACACCCTCTGTGAGCAGGAGGGATGT |
| | | CTTCTTCTCACCTCACACATCCTCTATGAGCAGGAAGG |
| | | GTGTCTCCTCCTCACCTATCTTTTCTTTTTTTTAAACCAG |
| | | TCCTCTTGTTTACTCCTTTTGCAACTCAAGCCAGTCCTG |
| | | CTGCCTTTTCATGCCCAAGCAGCATCTGTCCCTCCCCAA |
| | | GCACTGGGTGGGGCTCCTAGACCAGCTAAAGGCGCCG |
| | | AGCATCTGTGGGTGTTGCAGCACACCAGCCCAGCCTCG |
| | | CCTAGCTCTCTGAAAGAAAGCCTGCAACAGAAGGGAG |
| | | AGAAGCGGCCCCAAGTGCAGCTGAAAGCACTCCTTGG |
| | | GAGTCAGAAAGAAAGTTGACAGACAATCCGTAGGCTC |
| | | ATCACCAACCGCCACAGCCAC |
| 146 | RPS6KA1-RSRP1 | AACAGTTCTCTCAAGTTGTTTTGATAACCAAGAATTTCT |
| | | AAGAAGAAAGTGTGTTAGATGTGACATTGACGTATTCT |
| | | AACTGGGACAGAAGGAAGGTCTATAAATGATCTTGCC |
| | | ATAAAGGTCAGGCCTCATCCTGCCACAACCGCTGTTAG |
| | | CTGTTACGTGCCGCAGCAGAGCTCCCCCATAGTCTCAG |
| | | CTGATACTGAAATTCATCCCACTCCCCTCTCACCCCCA |
| | | GCTCCTGTGTGTCCTTCGCTCGCTCCCTCCCTCCGTCTT |
| | | AGGTCACTGTTTTCAACCTCGAATAAAAACTGCAGCCA |
| | | ACTTCCGAGGCAGCCTCATTGCCCAGCGGACCCCAGCC |
| | | TCTGCCAGGTTCGGTCCGCCATCCTCGTCCCGTCCTCCG |
| | | CCGGCCCCTGCCCCGCGCCCAGGGATCCTCCAGCTCCT |
| | | TTCGCCCGCGCCCTCCGTTCGCTCCGGACACCATGGAC |
| | | AAGTTTAAAGAAAGTTGACAGACAATCCGTAGGCTCG |
| | | ACTTCTCCCACTTCAACGCC |
| 147 | LAYN-CD44 | CTTTTTCTTTTCAGAGATGGCCTGCCCAGAAAAGAGGA |
| | | ATCTAGAGAGGCAGTCTAGCCACAGCAGCCTTGCTGAG |
| | | CTGCGGTTGGCTCCACCCAGTTTGATCTTCTGAGTGGCT |
| | | TTGTTTACACTGTGAAGGTAAAACCACATGCTCAAGCC |
| | | TCAGCAATGGTGGATGCTTCTCCCTCCACCAAATTCTA |
| | | GTATCCCAGGTCGAGCTCAGACTGCTGTGCTGGCAGTA |
| | | ATCCTCCTTGCAGGGAGTGGGGCCGGAGAGGGTAGGG |
| | | GAGAAACCAAGATAAGTGATTGGAGTCAGGGCAAATG |
| | | CTGAGAAACATCTCATGTGGTTTCCTGTGTCATACTCTT |
| | | ATTTTCCAGCACCCTCTGTACAGGAAATCACAGGGTTG |
| | | TTGAGCATCTTGATAAGCTTCACTCAGTCATCAGGTTTC |
| | | ACTCCAGATCCTGCAGGCATCTCCCCATCCTCAGCTGT |
| | | TTGCCAGAAAGAAAGTTGACAGACAATCCGTAGGCCT |
| | | ACTCCGGCATCTACCACGGCT |
| 148 | OR5B21-MRGPRX2 | TTATCATTCTTCACTGGTGTGGTGGTGACAGGTGAAAT |
| | | TTCAGCAGGCTGATCATACAAGGTAAGGAAATGGAAA |
| | | CAATGAGTTTAGATGGTTTGAAGTCTGGTTAAGACAGG |
| | | AAGGAAAGAGATAATATCTAAGTAGAGTCATATGTCG |
| | | GGTCAATAGATCTTAAAATACAGATAGAAGACAGTAA |
| | | CACATTTTGTAACTTGAGGTAACAAAATTATGTTGAAA |
| | | GGGAGTCCTCACCTGGCAGAAGGCATAGTTTTGATGTG |
| | | AACAGTTGGGGGAAGCTTGTTTCTCTACACCCTCAAGC |
| | | ACGCCCACAGCAGGAGCTGAGCAGCAGGCCAGACGGC |
| | | ACAGAGCCCTCCTGGGACTGGTTATTTACATTAACCAA |
| | | GGTGGAAACTTCTCCCGGAGATAGGTGCTGCCAAGAA |
| | | CCCTAGAGGAAGTAAGACGCTTTATCTTTTGCACGCAC |
| | | GCCAAGTCTTCAGAAAGAAAGTTGACAGACAATCCGT |
| | | AGGCTCTTCGTCCGCTGCATCCACAAC |
| 149 | BTG1-MIR7851 | TAATCTTTCTGGCTACTTCCTGCTGAGAGGGGGTCATC |
| | | GTTATGGGGCACCGAATGCAGCACTGGAGTGGAAAAA |
| | | GTCGATTTGTTCCCAGTAGCACTCTCTGTTTCGGGGGTG |
| | | TAGAGGCAGTACCTGCTAAAACATAATAGTAGGCAAC |
| | | AGCAACACATATAAATAGGTTGCTGCTACTTCCTTCAG |
| | | AAGTTTCGGTTGTCTAGTCTTCAGTTCACAGGGCTTTAA |
| | | GATCCTCAAATGTCCCCTGGGAGGGGGTCAGGTCACAG |
| | | CGCAGGGAGGAAGCTAGTCCTCTCAAGGCCTGTCTGGA |
| | | ATCTGTTTATCAGGTTCCACTCCGCTGCCCGAGATCGCT |
| | | TCTTCCGTTACTCAGTGCCCGGGGGAGGGACCTGGCGC |
| | | ACTATCCAATCAGGGCCGCGGGGCGGGGTCGTGAGAA |
| | | CTGTCAATCAGGCGCACTGACCGGAGGAGGGTGCAAG |
| | | GTTCAAAGAAAAGAAAGTTGACAGACAATCCGTAGGC |
| | | TCTCCGACGCCGGCATCTCCACC |
| 150 | CD70-ZNF284 | GACAGAAACCGCTAATGATGGGCCTGAAACTCTACTTT |
| | | GGAGTGGCTGTGGTTTCCTCTTCCAGCCTGTGGGTTTGC |
| | | AAGAGAAGCTGGGTTTGCAACTGAGTTTGAATGGGAA |
| | | AGAGATTTTGCTCAGGGCTGCAGACTGCGTGCATGGAT |
| | | TCTGCTTTCTGTTTTCAAAGCTCAAAGACTGTCCTTTCT |
| | | AATCATTGCCATGGTGCAGTGTTGGAGTCTTTTTATGCT |
| | | TCCTCCTGGTGTTGTTTTGTTGCAAGGGCAGCATTGTGG |
| | | GGCTGTGGTTTGAATTGCTTTTTTGTGTCTCTTGAATAC |
| | | ACGAAGTTCCTCCTATAGTCATTGGCTCTGCTGCCTCTG |
| | | ATGAGGCACTTCCGTGTTCCCCACATTTGAATTTGCTCC |
| | | CGATTCGAGAGAGGCAGGCATCCGCGGCAGCAGCTTC |
| | | CAACCCAGACAGGCTTAAGCCGCGCGAGGGTGCCCGG |
| | | GTGGAAAGAAAGTTGACAGACAATCCGTAGGCACTTC |
| | | AACAGCGGCGGCGCCGGC |
| 151 | CD226-SLC66A2 | AAAATATTTGAGCAGCTAGTTCACAAAAGAGGATATG |
| | | AAACTGGCCAATATAAGAGCTCAACATCTTACTCATCA |
| | | GAGAAATGCAAATGAAAAGTACAATGAGATAATGCAA |
| | | TGCACCCACCACACCAGAACTGCTAAATTTTTTAAAAG |
| | | GAACTTGTAATCACAAGTGTTGATGAGAATGTATAGTG |
| | | ACTGGAACTCTCATATACTGCTGTGATGTAAATTGGTA |
| | | CAATTCCTCTTTGGCTTTAGGAAACGTGTTCAACTCTCT |
| | | GTTAAGGGTTTTGTGGTAAAATGGTATCTACTGCTGCC |
| | | CGTACCTTTCCTCCCCCCGAATTCCAGGGACTCGTACTC |
| | | ATTTGTTATCTAACCAATCCATAAAAGACTCTGGATTG |
| | | GAGGAAGCAGTTATGATGTCAGAGTTATGAAATAAAG |
| | | TCTCCCCCAGGAGAAGAGGGAGAAAAACATAGCAGAG |
| | | GGGTTGTACAAAAGAAAGTTGACAGACAATCCGTAGG |
| | | CGACTGCTTCCACACCACCGTCTG |
| 152 | IL7R-ATP6V0E1 | AGGAGTAAGACAAGGAAGTGATAAAAACTGATGTGTG |
| | | TTATTGATTTCAGCCAACTATTGCTGACAGTTCAAAGT |
| | | CTGCAGTGTAGTTACCGGAATCCCACCTCAAGCTCACG |
| | | TGATGATGTTTCTGTTTCTCTGCCACAAAGCTTTCTTTA |
| | | AAGCTCAGTTAACAACACAGCCTGAAATTTCAGAGGTG |
| | | TTAAGAACTCTAGGCAACCCTCAACCAGTGGGAGAAG |
| | | GCATCCTGTGGCTGAGTCCACACAGTCAAAGGCTTGTG |
| | | AGAATCTGGTGGATGCTGGACCTTGCTGCTGCTGCTAC |
| | | TGCTGTTTCCAGGGGCTGCAGAGCATGGACTGTTAAAT |
| | | CTTGCACTTCTTCTGAGTGAGGTAATTAGTTTGCTTTAT |
| | | ATACAATAGTTAGATGTTTTTATAGCTCATTGTGGTTTT |
| | | CCCCTCTCTCTTACCTCCTACAAGTTTGTATTCATGAGC |
| | | AATTATAAAGAAAGTTGACAGACAATCCGTAGGCACT |
| | | GCGGCTTCAGCGCCCGCGAC |
| 153 | RIC1-PTGDS | GTGAGGCTTGGCTGGAAGAGAGGAAGTTGACAAACAC |
| | | TTTCAATATGCCTAATCTCCACCAATATTTGGAAATCA |
| | | GCTCTAAGATAAGACCATTGCTTGGTGATTCTGTCTGC |
| | | TGGGAACTAACAAAGCAACTGTTCAATAAACCATGTG |
| | | ATCTGGGCATGAAATACAAAATGCCCAAGTCATGACA |
| | | GTTTTCAGTGTTCCTACAGTAGACTAGGCAGTGTGAAT |
| | | GAGGATCCTTGCTAACCTCCAGCTGTGCCCCTTAGCAA |
| | | GCCGACTTCCAGATCTACTCGGAGTACTGCAATAACCA |
| | | CCCCAACGCCTGCGTGGAGCTCTCCCGGCTCACCAAGC |
| | | TCAGCAAGTACGTGTACTTCTTCGAGGCCTGCCGGCTG |
| | | CTGCAGAAGATGATTGACATCTCCCTGGATGGCTTCCT |
| | | GCTGACTCCGGTGCAGAAGATCTGCAAGTACCCTCTGC |
| | | AGCTGGCCGAGAAAGAAAGTTGACAGACAATCCGTAG |
| | | GCCTCCTTCTCCGTCAACATCGCCG |
| 154 | GPN1-CAPG | CAGCAGACACGGACCTCGCAGGGGGCAGCGAACCCAC |
| | | GTGACACAGTCTTCAAGTCCTTTGGAGAGCCCCAGGAA |
| | | GGAACAACAGCGTGTACACCCTGTGATGGAATGTTCTC |
| | | TAGGGCGGTTCAGTGTGAATGGAATGTGGGGCCGGTG |
| | | CCATTCTAATTGGTTCTGTTTCCCTCTAGTGGTTGATCG |
| | | CGGAGATTTCGGCTTCTCCATCAGGACAAGTTCAGATA |
| | | GCCTCCTGGAGGTGGGCTAAGACAGACTCCCTCAGGTT |
| | | CCCACATGGAGCAGCCTCTGCCTGCCAAGCCCTTCCTT |
| | | GCTCTCGTCTCCCAGATCATCTGTCTTGTCAGCCTCCAT |
| | | GCCCTGTGATGTCACAGGCCTAAATGTGATGTCACTTT |
| | | GTCCCACATGCCCTGGGGAGGTAGCACAAAGGGAGGA |
| | | CCATGGTCTTCTAACTGAGGAACCTGAGCAGATGCCTT |
| | | GACCCAGACAAAGAAAGTTGACAGACAATCCGTAGGC |
| | | TGCCTCAACGACAGCTTCTGCTT |
| 155 | CEACAM21-ZNF441 | GAAACTCAGTGGGTGACAGGATGCCCACACCTCTTGGA |
| | | GGAGGACAGAAAGGTTTGTGGGCTGGTGTCAGATACA |
| | | AGCTTGGCACCATCTTTGATTAGGAGCTAATGTTAGCA |
| | | GAAAGATCAAGTGTCAGTCATGACCAGAGTTGTTTTCT |
| | | ACTATCTTGAGAAGGTCACCGGATTGCTGCCCCTTCGT |
| | | TTTTGAAACCTGATCTGGGGACCACAAACAGCCACAAT |
| | | GAATCCTGAACAGTGTCAGACCTAGGAGGTGCCCCACT |
| | | ACCACCCTGTCTTTATCAATGTTGTCACCAAAGCTGTC |
| | | ACAAACAATGGGGTGTGGGCTGTCACATGGCCCTGCCT |
| | | AAGTAACCACATTCTCGCTTCCTCCTTCCACACACAGC |
| | | CATTGGGGGTTGCTCGGATCCGGGACTGCCGCAGGGG |
| | | GTGCCACAGCAGTGCCTGGCAGCGTGGGCTGGGACCTT |
| | | GTCACTAAAGAAAGAAAGTTGACAGACAATCCGTAGG |
| | | CCGCGACGGCATCGACGACTGCTC |
| 156 | ATP2B1-ZCCHC8 | GCCTGCGTGGAAGGATCTAAGATGGCCTTATTTCCTTC |
| | | CTTAGCCACTCAGAAGGTTGGGTGGCTTTGTGGTCTGG |
| | | GGTTTAAGGGAGGAGTTGTACAACTCTAAGAGGTGGTC |
| | | AAAGTAAGGGAAACTCATTATGAGCTCTGTAGATTCTT |
| | | TCTTCCACTAACTTGACCTTACTCCATAATACTAAGAG |
| | | ACTTCCCTCGCCTGGTCTTTCTGTTTTCAGATGAGCTAC |
| | | CTCCTTGCTCCACATCAGTCACAAATGAGGCCCTGAAT |
| | | AGACTTGGGACCTCTACCTGCCTGCGCAGATGGGAGGA |
| | | GTCTGTGGTGTTTTGTATAAACAACCCTCCCTCTGAGA |
| | | CACACACACATACACATACACATGCACACACACACTCA |
| | | TATACACATGCAGAAGCTGTGACACGTGCGGAAGCTGT |
| | | GGTAAGTGCATCCTCCTTCAGTCTCAGTTCTGAAAATA |
| | | GATCATCAAAGAAAGTTGACAGACAATCCGTAGGCCG |
| | | GCTACTTCAACCGCCCCCACT |
| 157 | G0S2-MMACHC | TTACTTACCCTGTGACAAGCCCTTGGGACGGGCAGTCG |
| | | CCCCTCTCTGGCCTATGACTGCTGGCTGCCCCCACTGA |
| | | CACCTTATACCCTTGATCTGATCATAAATAAGAAGCAG |
| | | CCCGGGGACCTCGAGACTGAAACTGATTTAGAGACTTC |
| | | TGGGAGCTTTTTTCACTTCCCTCCTCCCATCCGGCCAGG |
| | | ACAGATCCCCGCCCAGAAAGGGGCTGTTGACTGCAGC |
| | | GGTCCTGAGCTGATTCAACATTCCCCTGAACTTCTCTCT |
| | | TGCTGTTTTTCTTCCTAGTTCTGAGAAATCGAGAAACAT |
| | | GATAAGGAATTGGCTGACTATTTTTATCCTTTTTCCCCT |
| | | GAAGCTCGTAGAGAAATGTGGTAAGTTTAGAAATGAC |
| | | ACGTCAACTTTGTAAAGAGGGAAATGGTGGCTAGAGG |
| | | AAGGAGTAATCTGATCTGTTTGTTGCCAAGGGTTTAGA |
| | | ATCATTCAAAGAAAGTTGACAGACAATCCGTAGGCTCC |
| | | GCTCCTACTCCTACCCCGTC |
| 158 | RAB27B-NEDD4L | ACCCCCAACCCAGTCCTCCTAGGATTGTTTGCTTTGTGA |
| | | AACATTAAAAGTGATTTCAGCCACTCATTCTGTGAAAA |
| | | AGAAAACTTGCAGTAGGAGTATCTGAAGAACATGAAT |
| | | TAGTCTTGGCAGAGCTGTGTGGGCGGTGTCTTGTGGGC |
| | | AGCACTGCTGAGGTGACAGCAAGGGGAGGAGGATGGA |
| | | TTGGAGCCTTATATACAGCAAGCGAAAGGGATTCAGA |
| | | GTCTTCCTCACAGGTGGGCACTGAAGGACCAGCGGGAT |
| | | CTACCTGGGTACCAAAAAGGTAACCCGGCCAAGTTTCC |
| | | AGATGAGGTAGACAAGGTTTCTTCCTCAGCCAGGGCCT |
| | | CGCCTGGTAGTTTGAACAATTCTAGTAGAGAGAAAGG |
| | | ATGGACAACAAGGGGAGAGGAGAGGAGAAGCCAGCC |
| | | TGACCAAGGATGCTGTTACCTTCCAGAGCAGTCTCCCT |
| | | GTCCAGATCACCAAAAGAAAGTTGACAGACAATCCGT |
| | | AGGCCCCCCTCCGTCAACCTCGTCAGC |
| 159 | RBPJ-HADH | CCATTCTACCCCAGCCCACACATACGGCCTTGTGGAAA |
| | | CTCCCTGTGGCTTGCTGATTAAAGAAGAAAATGTTCAA |
| | | GACTAGTTTCCAGATGACTCTTCCCTGGTCTAAGGACA |
| | | CTAGCACAATTAGGACTATTGTTGCAACTTTCACCCGC |
| | | TTAGGGGCGGCCTTGAAAGACAATTGAGAAGGAAAAT |
| | | CTTCCCAGGGGCAGAACTCTAAGCAGTACATCTCATGA |
| | | TCCTCCTAGCCATCTTGCTATTCCTTTCTTCACGTGCAA |
| | | AACTTGTTGGAGGCCAGGGAGAGATTCTCTGTCCATTT |
| | | CCTCTTGCTGGGTGAAGTGTCCCACAAGGCAGAGTGCA |
| | | CAAGCATGCGTGACCTCTTGGAGGAGACTAGAAAGGA |
| | | GGCATAAGCTACGATGATGTCACTGACATTAATTGTTG |
| | | GGCTCTTGTTTTTAAAGGAACATGGCAACCCGTGTGTG |
| | | TCTCATCCCAAAGAAAGTTGACAGACAATCCGTAGGCT |
| | | CCCTCCGCAACGTCAACTACCT |
| 160 | RDX-MICAL2 | GGCAATACTTGGTTGTGGAATTGGTGAATCATAGGGTT |
| | | GGTGTATGTTTAACTTTCTAAGAATCTGCCAAACTGTTT |
| | | TCCAAAATGGTTGTACCATTTTGTGCCCTCACCAGTGT |
| | | GTGTTACAGTTGCTTCACGTCCTCACCAGCATTTGCTAT |
| | | TGTTGGTCATTTTCAGCTTAGCCATTCGAGTGGGTGTG |
| | | AAATGTTACTGCTGTGGTTTTAATTTGCATCTCCTGATT |
| | | CCTCAACAATAATATCAGCTATGCCATCTTTCACTATTT |
| | | TAGCCAGTATCGAGTTGAATGAACATAGAAAAATACA |
| | | AAACTGAATTCTTCCCTGTAAATTCCCCGTTTTGACGAC |
| | | GCACTTGTAGCCACGTAGCCACGCCTACTTAAGACAAT |
| | | TACAAAAGGCGAAGAAGACTGACTCAGGCTTAAGCTG |
| | | CCAGCCAGAGAGGGAGTCATTTCATTGGCGTTTGAGTC |
| | | AGCAAAAAGAAAGTTGACAGACAATCCGTAGGCCCGA |
| | | CCTCATCCCCTCCACCTTC |
| 161 | EPAS1-IFIH1 | TTTAACTTGAGATTTGTGGCTATCACTTTTGTTTAATTG |
| | | AGTAAACCCCAATAAGAGTCACAGGTGACCCCCCCAC |
| | | AAAAAAGTTATAAGTAATTATATATGCAGAAACATTGT |
| | | TAGCTTGGACTGACAAGCAGAAAAGCAGTACAAAGTG |
| | | AAAAGAGACTTTTGTCTCCCCCAAATTTTACTGGAAGG |
| | | AAGGCTAAAAACAACCACGAAGCCACACACCTTGCTG |
| | | TTTTTCCTGTGAGTCACACCCTGAAACACAGGCTCTCTT |
| | | CCTGTCAGGACTGAGTCAGGTAGAAGAGTCGATAAAA |
| | | CCACCTGATCAAGGAAAAGGAAGGCACAGCGGAGCGC |
| | | AGAGTGAGAACCACCAACCGAGGCGCCGGGCAGCGAC |
| | | CCCTGCAGCGGAGACAGAGACTGAGCGGCCCGGCCCC |
| | | GCCATGCCTGCGCTCTGGCTGGGCTGCTGCCTCTGCTTC |
| | | TCGCTCCTCCTGAAAGAAAGTTGACAGACAATCCGTAG |
| | | GCTCGACCTCCCCAACTCCGTCATC |
| 162 | EPHA8-SH2D2A | GCAGGGATACAGCACTTCTGAGAAATGTGTGGCTAGAT |
| | | GATTTCACCGTTGTGGTGAAATCATGAACATCAGAGAG |
| | | TGCACTTACACAAATCTAGATGGTATGGCCTACTACGC |
| | | ACCTAGGCTATATGGTGTAGCCTATTGCTCCTAGACTA |
| | | AAAATCTGTACAGCGTGTTACTGTACTGAATACCATAG |
| | | ACAACTGTAGCACAATGGCATTCATGTATCTAAACATA |
| | | GATCCTATGCAGGGGCTGGGGACCATCAAGGATGATG |
| | | TGTGGCTGGGGTGGGAGGAGCAGTGACAGGGCTGGGG |
| | | CAAGGAGGACTTCAGAAGCCAATTGGAGCCAGGCTTG |
| | | TCTCCCAGCAGCCAATATGGGAGCTGGGGCTCCTGGTA |
| | | AGCGAGTGGCTGCTCAGAAACCCTGCGGGGAGGAGGG |
| | | GCTAGCTAGGCTGGGCCAGGATCCCTGTATAAATGGCC |
| | | CAGGAGCTGTGCAAAGAAAGTTGACAGACAATCCGTA |
| | | GGCGACAACAACATCGACCGCATCTC |
| 163 | LEPROT-TXNIP | AGGGAACTTCTGTGTCTGAGTGGGAGGCAGGCGTCATT |
| | | GGCCTCTACAGGAAAGGTCTGATGCTGCCTGGCATGTG |
| | | ACCTCAGCTCAGCCAATCAGATGCTCCTGCCCAGAGAG |
| | | GGGAGACTCCATTCTGGTAGTGGCTGCAGCAACCAGTT |
| | | TCAGGAGAGGAGTGACTTTCGTTCAGTGTTCAGGGGTG |
| | | GAGACCACAGAGTCCTCACCACAATGGTTCCGTGGCAT |
| | | AATCCTTACTTGGCCACTAGGAAAGCACCTCCCCCTTT |
| | | CCAAAAATGCTCCGGAAGTGCCTTCGCCCTCCGTAAAG |
| | | ATGGCCGGGGCAGTCGGCACGAGGGAGGCGGGGATGC |
| | | GCCTGCGCAACAAGTTCGGCGGGGAAGATGGCGGATG |
| | | ACAAGGTGAGTGGCCGTGGGGGTGATCTGCAGCCGGG |
| | | CGTGGTCTTCACTCTCCTTGACCTTTTTAGTTCCGGGGA |
| | | GAAGCGACTCAAAGAAAGTTGACAGACAATCCGTAGG |
| | | CTCATCACCGCCCGCTACCTCCGC |
| 164 | FOXO3-TIAM2 | TGACCATTTTCTGCTGCTGCAATCCGGATGCCTCCAAA |
| | | TCTCAAGTGACACTCAGTTTCTTAGGTTTTTTCCTTCCT |
| | | CAAAAATGAAACCTCTCTGGGCATCCCCTTACAACCTT |
| | | CTCTTTAGCTTTGAGTTGAGTTTCTTTCCTGTATTCTAA |
| | | TTAAAGTGACCATAGTGTTAGGTTTTTCCATTTCTCTAG |
| | | AAGATTGGCTTTTTGGTAGTAGTGTTTTCTGGATCTCTC |
| | | CTCCGCCACCGGCCCTGCTCGGCCGCCATCGCTGTGAG |
| | | GCGGCTGCCCGCGACAGCTCCTCCTCCGCCTGCCAAAT |
| | | CTCGCGATGGCAGCCGCACAGTTGCGGACCAGTGGCCT |
| | | GCTGCTACCAAATCTCGCGAAACTCGTTTTCCCTCCCG |
| | | GGAAGTTGCCAGAACTAACTCTGGGCAGCATGGCGTCT |
| | | TTCCGGCTTCTCCAAACCCTTGCGAAAAACTTTATTGG |
| | | CAAAAAAGAAAGTTGACAGACAATCCGTAGGCTGCCA |
| | | CACCATCGACTGCTTCGA |
| 165 | PFKFB2-LY9 | GTACTAAGTGTGACCTCAGGCATGTTGCTTTCCTTCCCT |
| | | GGACTGCTTTCAAGAGAATAAAAAATGTACTTAACATG |
| | | ATCAGCACGGGGCCTGGCTCATTGCAGATACTGTGCAA |
| | | AGGTGATTCAACTAAGACATGAACACTAAAAAGGAGA |
| | | CACTCTTAACTCATGCACCACTAAGAACTTGCAGAGGA |
| | | CTCATCCCTGTGGTTAGAGCTTTGGAGAGTAAGGAAAT |
| | | GATCCTCCGCGCCACAGTTGGGTCTTCCGGGTTTCTGTC |
| | | TCAACTATGCACGCCCCTCCCCCGTGCGCGCCCCTCAC |
| | | CTGGCCTCCCAGCTACCCGAAGTGACAGGTGGACGGA |
| | | GGGAGGAGTGGCCAGTGGAGGCATCAGTTGCCGGAAG |
| | | CTGTCACCTTTGATCTCGGAAGTTCCCCCTTGCTGCTGG |
| | | AAACGCAGTTCCGGTTAGGCGGCTGAGTTTGTTTACGT |
| | | TGCTAACAAAAGAAAGTTGACAGACAATCCGTAGGCC |
| | | GCGACTACAGCACCGTCCGCAC |
| 166 | MFF-ARHGEF4 | TCTGTGGGGTAAGTTCCACTTGCTTAGGATCAAGATGT |
| | | ATTGACTTTGAGGGAGGAAACCGATGAATGGTAAGAA |
| | | GCCATAAACTGCAAACACTAGCTAGTGGTTTAAGGTGT |
| | | GGTGGTATAGCTACTGAGCTCTGTGTAACCAGAAGCTA |
| | | AAAGACAAGAGTCTAACCGATGAGGGATGTTTGTAAG |
| | | AGATCTTGACTTTCGATGTTTTTGGAGAAATCTTTTTTT |
| | | TTTTCCTGAACCAGAATAAGGGTAATGTTTTCCTCTGTC |
| | | TTCAAAATCATCAATAATCCATGCATTGTTTAACTCAT |
| | | GTCATAAGCAATAATGCCTTTCATATAGCCATTGGCAT |
| | | CAAAGAAGAAACACCCCCTTGATTTGATGGTAAGCGTG |
| | | ACACTACATAAACTCCCAGAAAACCCACTTCCTTTCCA |
| | | GCAAATAGAAAACAACCGAGAGCCTGAATTCACTGTC |
| | | AGCTTTGAAAAAGAAAGTTGACAGACAATCCGTAGGC |
| | | CGTCTTCAGCGCCGTCTACAGCG |
| 167 | NBEAL1-MYO3B | AGGAGGATTGCTTGAGTGTGGGAGGTTGAGACTATGGT |
| | | GAGTCAAACTGCACCACTCCACTCCAACCTGAGCAACA |
| | | GAGTGAAACCTTGTCTCAGGAAAAACACACAAAAACC |
| | | CTTCCTTCTGACCCCTCCCTACTAAATGAATTTTGGATT |
| | | TCGTAGAATGTTATTCAGAACCTGACTCTAATCTATCTT |
| | | TCAGTGCTTACTCATCTTTTTCTTACACTGAGCACCTAA |
| | | TCCTATAACTACACTCTTTGTCTAAAAGGGCCAAGTTC |
| | | TCATCAGAACACTTTGCTGAGCCCCGGGTGACCGAATC |
| | | CCTAGCTCTGGGGTGAGCGACATGCCTCCAAGGAGCTG |
| | | CAGCCAGGAGACAGCCAGGCGAAGGCGGGGCTCTGCC |
| | | TCTGGGCTCCCCGAGGGGCGGAGGCCGGCGCGCGGGT |
| | | CACGTGGGGGCGCGGGAGGCGGGGCTTGCACACGCTG |
| | | GTCACGCGGAAAGAAAGTTGACAGACAATCCGTAGGC |
| | | TGCAGCTACGGCTACTGCACCAT |
| 168 | PHACTR2-E2F3 | TCATTTTAGTATTGACTCTACCTCATCTGGTCTCCTCCT |
| | | CAAATTGTATTAGCCGCCTGTCATGCTGACATCCTGTA |
| | | TACTTGTGTCATTCAACTGTCCTATATAATCATTCACTA |
| | | AGTGCTGGGGCACAGCAGTCTTCAATTTAACTTAACCA |
| | | GAGCCCTGAAAACAGCAATGGTTAAGAAATTCCCTCCT |
| | | ACCTTCCCCACACTTAGCTAAGACTTGCTGTCCACTCTT |
| | | CCTAGCCAGGGTTCCATAAACAAATCCTTCTTAGTTAG |
| | | CCTTGGTAGCTCAACACCTTTGGTATCTCACTCACTTAA |
| | | CTGCCACGCCTACTCTTGTTGGATCCGGTTATAAAGTA |
| | | AACTATGTTCTTTGAGAACTGTTGCATACAGATGAGTT |
| | | GGCAGCCGGTCTGAGCTGGCCCACAGACTCATAAAATC |
| | | AACAGGGCCTCGGGTACCCTCACCTAGCATATCCAAAC |
| | | TCTTAAAGAAAGTTGACAGACAATCCGTAGGCGACCG |
| | | CTACATCAGCTACGTCTC |
| 169 | NEK7-LUZP1 | GAAACAAACAATGTCCTCCATCTGGATTGTGCCTGCTG |
| | | TAGGAACAGGAAGTAGGCTCTGCTCATGTCTGGGTATC |
| | | CACTGCTTTCAGATCACAGACAATCAGTCACTGCTCCT |
| | | CACACAGCTGCTAAGTGGTGTGATCAGGACTGAAACCC |
| | | CAGTGCCTCTGACTTCAAAGCCTATGCTGCTCCACTGC |
| | | ACCACAGCATCCATTTCATCAAAGTCAGGAATTCCCCT |
| | | GTTCCTTAGAGTCCAGGTTCCTGCTTCCAGCCTTCTGGT |
| | | AGACCAGGCCCTGCCACAAGTGAGAGTTGGCCGGTTG |
| | | GGTATCTGGTTTAGTCACTCGCCTCCACACTTTCACATC |
| | | CTCTATCAAAACCTGCACACTTGGGTTGTGACCACACA |
| | | TGCTCTTCCTGTTTGTACAGAAAGTGCCATTCAGAAAG |
| | | CTCGCCAGCGAGAAGAGAGCTCCACGCTCAGCCTCAG |
| | | AAGAAAGAAAAGAAAGTTGACAGACAATCCGTAGGCC |
| | | AACTTCGGCCACGGCTGCGACG |
| 170 | CYTIP-BUB1 | TGCTGCCACACAAGCCCCTGTGCCCTCCCATCTTTAGT |
| | | GAGAGCCTCCTTTGTACCAGGCACCAGGCTTTAGCAAT |
| | | GACTAACATGTGGTCTTCACCTTCTTAGAGCTGTGTTCA |
| | | TTTTTGTGTGGTGTCATTTGCCTTGTGGTCAGCTTTGCT |
| | | AAAACTGATCAAACTCCAAGCTGTCTTCCAGGCCCACT |
| | | CTTATGTTTTCTGTTGATTCTCTGACTTGAGCTTAGAGT |
| | | CCTAATATTTCTGTTTGTTGGCAAAACCCTTTTGGGTTG |
| | | TTCATCACCTTTTATGTCATTATAGATGTAAACAACATG |
| | | TGTGCTCCAGATGGACTCTGAAATCCAGGCTCCCACTT |
| | | TATGACACACTCCTCTACAACACCAGAGACTCCCAAAC |
| | | ACAAGGCCTTATATTGACTCATTTCAGCTCACATCCTG |
| | | GCGACTCTCAAGAGAGAAACCTCAGAGTGACTAAAAT |
| | | CTCCAAAGAAAGTTGACAGACAATCCGTAGGCTCTCCA |
| | | GCAACCACTGCGACTGC |
| 171 | NMB-LOXL1-AS1 | GGCAGAGTCTTCAGTTGCTTATCACAGGGACTTCCTTG |
| | | CAGAGCTGTTTGAGTGCCCCTCTGGCATGGCAGTTGGC |
| | | TTCTTCTGCAATGAGTGATCTAAGAGCAAGAGCAAGGA |
| | | GGAAGCCACAATGCATGTAATGACTTATTCTCACGGGT |
| | | TGCACATTGTCACTGCTGCCACATTGAATTCGCTAAAA |
| | | CTCAGTTACTGACTACAGACCACACCCTAGGAGAGAA |
| | | GAATCCTCCTAGATATGCACATGTCATCAAAGGATAGC |
| | | ATTTTACAGAGTCAACACCACTCACTTCATAGAACAAG |
| | | GGGCCAGTGGCTTTTCAGAAGAAGGGTCTGTTCTCGGC |
| | | CCCACACCAGAACGCCAGCCGCCAGGTCCCGCTGCTCA |
| | | GCAGGTCTCAAGCCCGCGCTCACTCGGGCTCCGGCTGC |
| | | TGACAGCTGCGAGCCCGCGCGTGTATACCCGCTCAACG |
| | | CTGGGACGTAAAGAAAGTTGACAGACAATCCGTAGGC |
| | | CCGCGGCATCGTCTCCGGCTTCA |
| 172 | ODAPH-CCSER1 | TGGCTTCTAGGGAACGAGTGCGGAAATGCGTGCGGGT |
| | | GTGGGAGCGCACCACTCAGTAGGGTAGGACCTCTCCCA |
| | | GCGCTAGGTCACTGCAGCATTCTTCCCACCATCTGCTC |
| | | CTGTCACCAGCCCCTAACTCCGCCCCTTTGGGCCCGCC |
| | | CCTTGTTCCGAAGCCAATAGCAACCCCTGACCGCCCTA |
| | | CACCGCCCAGAAGCCTTTCCACACCCCCTCGTCCTGGG |
| | | AGCTCCTTGCACCAATCAGCGCTCTGTAAAATGCAGGA |
| | | ATCTAAAAGTAGCCAATCACAGGGAGGATTGAAAAAA |
| | | GGGCACTCTGATAGGACAGAAATAGCAAATGGGCAGG |
| | | GACAAACAAGGGTGTAAAAGCTGGCCCCCTGCCCCCC |
| | | ACCCATTCCTGTTAGCCCGCAGCAGCAACCTGCTCAGG |
| | | TCGGTTTCCACGCTATGGAAGCCTTGTTCTTTCGCGTTC |
| | | TTTCGCTCTTCAAAGAAAGTTGACAGACAATCCGTAGG |
| | | CGACTCCGGCGACCTCTGCAGCGT |
| 173 | RAI1-LGALS9 | ACACATGGTCTGCTGGTCATTCAGAGTCACCTTTGGGT |
| | | CTATTGACTAGGTTGCACACAGTTCAATTGTGTGTTTTC |
| | | TTTTTCACAGTGCCCCTCCACTCACTGAAAAGGAAGTT |
| | | GAGGTAAGTGACTCAAGTGTGTTATGAAATTTAGTGCC |
| | | CGTGCTTGACTGGGCTTATTCAAAACATTTCTTCCTCCA |
| | | GACTCTAGCTCTGACATTTTTAGACAAAAATGAGAAAG |
| | | TCCTTTCAGGTACCACTTCCAGAGAAATACCAGCTTGA |
| | | AGCCCAGCTACTGCCACCTCAGGCCCATAGGCACACTG |
| | | GGGCCCATTTGCTCCCAGGCTTCAGTGGGAGGCGACGA |
| | | CTCAGCACCTTCGACTCCAGCCTCGCAGCGGCCCCGCC |
| | | CCACAGAGGCCTGGCCCCGCCCCTCCGCGCTCAGGCCC |
| | | CGCCCCCAGCTCCGAGGGCGGCTGGCCCGGTCGCGGTC |
| | | GCGGCTAAAGAAAGTTGACAGACAATCCGTAGGCACT |
| | | ACTGCATCATCTACGCCCGC |
| 174 | LYVE1-USH1C | ACCCCCGGTGTGAAACTCTTTCTGAGAAAGACTGGCCC |
| | | TTTTCTTTAATGTAAAAAAATATATATAACAAAAATAG |
| | | ATTTCTTCTCAAGACTCTTGTTTATTCAGATGATATCAA |
| | | TTCTGTTTCCAGCACAACATGTTGTAGCTGCTACTCAG |
| | | AGATTTTTAGAAAGTCAGATCAACGCGACTGCAATGGG |
| | | TTTTTGCCGTGAGTGAGCTGTGCTTCTCACTTGCTTACT |
| | | TCCTGCGGGGAGGATCCTGATCGAGGAGCTGAGGGAC |
| | | CGATTTCCCTACCTGAGTGAAAGCTACTTAATCACCAC |
| | | CGATGCCGCCGGCTCCATCGCCACAGCTACACCGGATG |
| | | GTGAGGAAGTGGAGGGAGGGGTGAGAGTGAGAACTGG |
| | | TTTTTTTGGGAAAGCCCCTTAGATATAGCTGACAAAAC |
| | | TTGTTCTGCAAATATCCAGAAGGCAGCCACTCTTATAT |
| | | TCCCCTCAAAAGAAAGTTGACAGACAATCCGTAGGCG |
| | | GCTTCGCCGGCAACTCCAACTT |
| 175 | RFTN1-PPARG | ACCCCCGGTGTGAAACTCTTTCTGAGAAAGACTGGCCC |
| | | TTTTCTTTAATGTAAAAAAATATATATAACAAAAATAG |
| | | ATTTCTTCTCAAGACTCTTGTTTATTCAGATGATATCAA |
| | | TTCTGTTTCCAGCACAACATGTTGTAGCTGCTACTCAG |
| | | AGATTTTTAGAAAGTCAGATCAACGCGACTGCAATGGG |
| | | TTTTTGCCGTGAGTGAGCTGTGCTTCTCACTTGCTTACT |
| | | TCCTGCGGGGAGGATCCTGATCGAGGAGCTGAGGGAC |
| | | CGATTTCCCTACCTGAGTGAAAGCTACTTAATCACCAC |
| | | CGATGCCGCCGGCTCCATCGCCACAGCTACACCGGATG |
| | | GTGAGGAAGTGGAGGGAGGGGTGAGAGTGAGAACTGG |
| | | TTTTTTTGGGAAAGCCCCTTAGATATAGCTGACAAAAC |
| | | TTGTTCTGCAAATATCCAGAAGGCAGCCACTCTTATAT |
| | | TCCCCTCAAAAGAAAGTTGACAGACAATCCGTAGGCG |
| | | GCTTCGCCGGCAACTCCAACTT |

In some embodiments, the nucleic acid construct may include a reporter gene, having a reporter sequence, operably linked to the EP pairing sequence. For example, when the nucleic acid construct is used in a screening assay, the reporter gene may be used as a readout of transcription through the EP pairing sequence, wherein the reporter gene may be tracked and measured. In some embodiments, the reporter sequence may include a fluorescent protein including green fluorescent protein (GFP), red fluorescent protein (RFP), or may include blue, orange, far-red, cyan, or yellow fluorescent proteins.

### Observable Behaviors of the Nucleic Acid Constructs

Various nucleic acid constructs of the disclosure may demonstrate dynamic behavior over time in response to antigen stimulation. In some embodiments, the nucleic acid constructs that display similar dynamic behavior may be classified into similar dynamic behavior profiles. For example, some nucleic acid constructs may demonstrate constitutive behavior, wherein antigen stimulation leads to transcription driven from the EP pairing, and wherein transcription level stays relatively consistent, regardless of continued antigen stimulation. In some embodiments, some nucleic acid constructs may demonstrate “stimulation on” behavior wherein antigen stimulation leads to a marked increase in transcription driven from the EP pairing. In some embodiments, the “stimulation on” behavior may cause transcription levels to stay high even after initial antigen stimulation leads to transcription driven from the transcription initiator. In some embodiments, “stimulation on” behavior may include where initial antigen stimulation leads to a marked increase in transcription driven from the EP pairing (referred to herein as “early stimulation on” behavior) or prolonged initial antigen stimulation leads to a marked increase in transcription driven from the EP pairing (referred to herein as “late stimulation on” behavior). In some embodiments, the “stimulation on” behavior may also include where antigen stimulation leads to a single instance of increased transcription over time driven from the EP pairing. In some embodiments, the single instance of increased transcription driven from the EP pairing may be due to epigenetic regulation (e.g., DNA methylation/de-methylation, histone modifications including acetylation, methylation, phosphorylation, and ubiquitination, or the like), other epigenetic like transcriptional changes or cell regulatory or transcriptional pathways. In other embodiments, the “stimulation on” behavior may also include where antigen stimulation leads to multiple instances of increased transcription over time driven from the EP pairing. In some embodiments, transcription initiators may demonstrate “stimulation off” behavior, where initial antigen stimulation leads to a decrease in transcription driven from the EP pairing. In some embodiments, the “stimulation off” behavior may cause transcription level, after initial antigen stimulation, to be near or about zero.

In some embodiments, the one or more nucleic acid constructs may contribute to a rested cell state, wherein antigen stimulation is removed for a period of time. Transcription through the EP pairing of the constructs may still occur. In some embodiments, in a rested cell state, transcription through the EP pairing may still occur at reduced rate of transcription or an increased rate of transcription compared to the rate of transcription occurring during a non-rested cell state.

In some embodiments, cells that contain the one or more nucleic acid constructs may demonstrate an exhausted cell state, with chronic or consistent antigen stimulation. In some embodiments, the one or more constructs may sense the exhausted cell state and increase or decrease transcription through the EP pairing. It can be appreciated that each of the unique EP pairs of the constructs may demonstrate many types of behaviors (e.g., behavior profiles) depending on the EP pair, and each of the types of behavior that result from the EP pairs disclosed herein are considered within this application. Advantageously, each of these behavior profiles of the distinct EP pairs may provide dynamic transcriptional regulation of a reporter gene or gene operably linked to the EP pair of the construct in response to cell state.

### Library of Constructs

In some embodiments, a library may be generated that comprises a plurality of nucleic acid constructs, wherein each construct includes a different enhancer-promoter (EP) pair and a unique barcode sequence specific to the EP pair. Each enhancer-promoter pair may be operably linked to the unique barcode sequence and a reporter gene. In some embodiments, the library may be used to screen or test for EP pairs that demonstrate a specific behavior or behavior profile as will be described in more detail herein. For example, the library may be used to screen for EP pairs that lead to large increases in transcription of the reporter gene over time through the promoter of the EP pair. In some embodiments, the library may be used to screen for EP pairs that lead to sharp decreases in transcription of the reporter gene over time through the promoter of the EP pair. In some embodiments, the library may be used to screen for EP pairs that lead to gradual increase in transcription over time through the promoter of the EP pair. In some embodiments, the library may be used to screen for EP pairs that lead to gradual decreases in transcription over time through the promoter of the EP pair. In some embodiments, the library may be used to screen for EP pairs that lead to increases in transcription over time through the promoter of the EP pair. In some embodiments, the library may be used to screen for EP pairs that lead to multiple increase in transcription over time through the promoter of the EP pair, or the like. In some embodiments, the library includes nucleic acid constructs comprising EP pairing sequences of any of the sequences of SEQ ID NO: 1-174. In some embodiments, the library comprises a plurality of vectors wherein each vector includes a nucleic acid construct having a different enhancer-promoter pair and a unique barcode.

### Cell State Sensing Constructs and Expression of Engineered Receptors

As described above, the one or more nucleic acid constructs of the disclosure may induce transcription through one or more promoters of the disclosure in response to the cell state and/or changes in cell state. In some embodiments, the transcription regulating enhancer sequence and/or the transcription initiator promoter sequence of the one or more nucleic acid constructs may be operably linked to a sequence encoding one or more of: a CAR, an exogenous T-cell receptor (TCR), a cytokine, a degrader, or the like. For example, the transcription regulating enhancer sequence (“enhancer”) and the transcription initiator promoter sequence (“promoter”) are operably linked to a sequence encoding a degrader marked by a reporter gene.

In some embodiments, the cell state sensing constructs may drive expression of an engineered antigen receptor including T cell Receptors (“TCRs”), Chimeric antigen receptors (“CARs” including all generations), B cell Receptors (“BCRs”), co-receptors of a TCR or BCR and/or any combination or hybrid thereof. In some embodiments, the engineered receptors may signal, or generate other outcomes (e.g., a dominant negative antigen sink). In some embodiments, antigen receptors may include one or more of: a DHD and/or binding site for a degrader, a binding site for an effector protein of a LOCKR switch, or a binding site for a direct or indirect signaling transducer of a safety switch. In some embodiments, antigen receptors may comprise a fully human sequence, a humanized sequence, a sequence isolated or derived from a mammalian sequence or a non-human sequence (including a mouse sequence), a syngeneic sequence, a chimeric sequence, a recombinant sequence or a sequence derived from any species.

One example of an antigen receptor may include a chimeric antigen receptor (CAR), as seen in FIG. 1A. In some embodiments, a CAR may comprise: an extracellular domain that contains a binder that specifically and selectively binds a target antigen, a transmembrane domain and an intracellular domain that contains at least one signaling domain. In some embodiments, one or more sequences of the CAR may be naturally occurring or non-naturally occurring. In some embodiments, one or more sequences of the CAR may be endogenous or exogenous to the cell expressing it. In some embodiments, one or more sequences of the CAR may be a fully human sequence, a humanized sequence, a sequence isolated or derived from a mammalian sequence or a non-human sequence (including a mouse sequence), and/or a syngeneic sequence. In some embodiments, the CARs may be specific for one or more target antigens. For example, some CARs may have one or more binders, wherein each binder is specific for a distinct epitope on the same target antigen. In some embodiments, CARs contain a spacer to attain an optimal distance of the immune synapse. The extracellular domain of the CAR may comprise the spacer, which is positioned between the T-cell surface and a binder. If there is more than one binder, the spacer may be positioned between the T-cell surface and a first binder. In some embodiments, binders may have any structure, including but not limited to: (1) an antibody or any fragment thereof: (2) an antibody mimetic or any portion thereof; and/or (3) a de novo designed protein. Exemplary binders may include, but are not limited to: scFv, VH, VHH, monoclonal antibody and any combination thereof. In some embodiments, transmembrane domains may be isolated or derived from any protein, including any transmembrane protein. Exemplary transmembrane domains may comprise a sequence isolated or derived from a CD4 or a CD8 protein. In some embodiments, CARs may be of any CAR “generation” including, but not limited to: Gen I, Gen II, Gen III, Gen IV, and Gen V. In some embodiments, intracellular domains of the CAR may contain one or more of immunoreceptor tyrosine-based activation motif (ITAM) repeats (e.g., a single CD3ζ intracellular domain) (Gen I), ITAM repeats and one co-stimulatory molecule (e.g., CD27 or CD137 sequence) (Gen II), ITAM repeats, a first co-stimulatory molecule and a second co-stimulatory molecule (e.g., CD134 or CD137) (Gen III). ITAM repeats, a co-stimulatory molecule and a constitutively or inducibly expressed chemokine (e.g. WT IL-12) (Gen IV: also referred to as a T cell redirected for universal cytokine-mediated killing (TRUCK)), and/or ITAM repeats, a co-stimulatory molecule and a intracellular domain of cytokine receptors (e.g. WT IL-2Rβ chain fragment) (Gen V).

In some embodiments, CARs can signal or generate other outcomes (e.g., a dominant negative antigen sink). CARs may include a DHD and/or binding site for a degrader, a binding site for an effector protein of a LOCKR switch, a logic gate or switch, or a binding site for a direct or indirect signaling transducer of a safety switch. In some embodiments, CARs may be split CARs in which a final activity depends upon the binding and interaction of two domains or two receptors.

To avoid CAR T cell exhaustion, it would be beneficial to have a system including one or more exemplary nucleic acid constructs that may sense exhaustion and shut down stimulation of the CAR. Disclosed herein are one or more exemplary engineered (non-naturally occurring) constructs and methods of use that may be used to shut down stimulation antigen receptors including chimeric antigen receptors in response to sensing the cell state including exhaustion.

In some embodiments, the cell state sensing constructs may drive expression of a fusion protein having an engineered receptor (e.g., CAR or TCR) and degradation domain. For example, in some embodiments, the polypeptide may be fused to a degradation domain (DHD) (e.g., degrader) for targeted degradation of a molecule of interest. In some embodiments, the degradation domain comprises a degron. As provided herein, a degron is a minimal element of a protein that is sufficient for cell-mediated degradation. In some embodiments, the cell-mediated degradation may be dependent on ubiquitin. In some embodiments, the cell-mediated degradation may be not dependent on ubiquitin.

In some embodiments, the one or more nucleic acid constructs may include a polynucleotide set that includes a polynucleotide encoding a degradation initiator fused to a binding element that is specific for a native motif on an endogenous molecule of interest. The binding domain may, for example, recognize and bind a specific region on the molecule of interest or a specific modification on the molecule of interest. Interaction of the binding element with the native motif on the molecule recruits the degradation initiator to the molecule to initiate degradation.

In some embodiments, the degradation domain comprises a ligase or ligase-binding domain. In some embodiments, the polypeptides of the one or more constructs may be used as binding elements, wherein the polypeptides are fused to a ligase domain (or functional variant thereof). Binding of the polypeptides of the one or more nucleic acid constructs to the protein of interest brings the modified ligase into proximity with the targeted protein, thereby facilitating ubiquitination and subsequent degradation of the protein target.

In some embodiments, the ligase of the one or more nucleic acid constructs may include an E3 ligase or a variant thereof E3 ligases may control substrate specificity and the topology of ubiquitination. For example, in a cellular ubiquitin proteasome system, an E3 ligase protein recruits an E2 ubiquitin-conjugating enzyme that has been loaded with ubiquitin, recognizes a protein substrate, and assists or directly catalyzes the transfer of ubiquitin from the E2 to the protein substrate, thereby targeting the protein for degradation. An E3 ligase domain and/or a variant thereof may be selected based on the intended protein targeted for degradation. For example, an E3 ligase and/or a set of E3 ligases may be selected for modulating the stability and/or half-life of a targeted transmembrane protein. In another example, an E3 ligase and/or a set of E3 ligases may be selected for modulating the stability and/or half-life of a cytoplasmic protein (e.g., a transcription factor). In some embodiments, the one or more nucleic acid constructs may include one or more synthetic degraders that may be useful for regulating the activity of a range of target molecules (e.g., proteins) in various synthetic biology applications, such as therapeutic applications. In one embodiment, the one or more synthetic degraders may be used in a cell therapy application to program a population of cells for performing and/or modulating a therapeutic function.

In some embodiments, the one or more synthetic degraders may be used to regulate and/or degrade T cell receptors (TCRs) and/or chimeric antigen receptors (CARs) cells used in cancer therapy. More specifically, the synthetic degrader system of the disclosure may be used to degrade CARs and/or TCRs and inhibit excessive CAR/TCR signaling that may result in undesirable exhaustion phenotypes observed in many existing immune cell therapies.

In some embodiments, the cell state sensing nucleic acid constructs may drive expression of one or more cytokines. For example, in some embodiments, the transcription initiator and/or transcription regulator may be operably linked to one or more cytokines. The one or more cytokines may include IL-2, IL-12, IL-15, IL-18, IL-21, IL-23, Interferon alpha (α), Interferon beta (β), Interferon gamma (γ), and Interferon omega (ω).

### Inducible Receptor Systems

Provided herein are inducible receptor systems engineered to drive expression and degradation of cell surface receptors, including antigen receptors. In some embodiments, the inducible receptor systems disclosed herein may be used to drive expression or degradation of antigen receptors such as CARs and TCRs. In some embodiments, the inducible receptor systems disclosed herein may be used to drive expression or degradation of engineered receptors in response to sensing of cell state. In some embodiments, the inducible receptor systems may comprise one or more of the exemplary constructs disclosed herein. In some embodiments, the inducible receptor systems may comprise a circuit, wherein the one or more exemplary constructs may comprise different circuit components including a response component and an effector component, wherein the response component may be capable of modifying the activity of the effector component in response to a signal from a receptor including an antigen receptor, as will be described in more detail herein.

Disclosed herein are the one or more nucleic acid constructs that make up the inducible receptor systems of the disclosure. The nucleic acid constructs of the disclosure may be non-naturally occurring and comprise a response component comprising a transcription initiator or comprising a transcription initiator and a transcription regulator (e.g., a transcription initiator-transcription regulator pair). In the inducible receptor systems of the disclosure, the response elements are operably linked to an effector element (e.g., a gene of interest).

In some embodiments, each of the transcription regulators and transcription initiators may comprise a concatemer of transcription factor binding motifs. The concatemers comprise sequences that may be derived from known promoter sequences. The concatemer of transcription factor binding motifs (referred to in the Examples as concatemerized sequences) either bind more than one unique transcription factor or bind the same transcription factor at more than one site. It can be appreciated that non-naturally occurring sequences may include naturally occurring sequences (including those isolated from or derived therefrom) assembled in a non-naturally occurring way. For example, in some embodiments, one or more naturally occurring sequences that are not adjacent within the genome may be placed adjacent in the construct, thus being assembled in a non-naturally occurring way.

As used herein, a transcription initiator of the disclosure behaves in a promoter-like fashion and supports binding of a transcription factor to initiate transcription of a downstream under its control. As used herein, a transcription regulator of the disclosure may further regulates the transcription of the downstream gene. In some embodiments the transcription regulator is located 5′ of the transcription initiator. In some embodiments, the transcription regulator is located 3′ of the transcription initiator.

Transcription binding motifs may be derived from the untranslated genomic regions of the following non-exhaustive list of proteins: BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D.

In some embodiments, when the transcription initiator comprises one or more sequences from the untranslated genomic sequence of NFAT: in some embodiments, the transcription regulator may comprise a sequence of any one or more sequences of SEQ ID NO: 3430-3437, 3802-3803, 3940-3947, 4312-4313, 4808-4815, 4874-4889, 4298-4299, 4364-4379, 8576-8583, 8948-8949, 9444-9445, and 9510-9525.

In some embodiments, the transcription initiator comprises one or more sequences from the untranslated genomic sequence of NFkB; in some embodiments, the transcription regulator may comprise a sequence of any one or more sequences of SEQ ID NO: 550-553, 1060-1063, and 5696-5699.

In some embodiments, when the transcription initiator comprises one or more sequences from the untranslated genomic sequence of REL (including RELA or RELB); in some embodiments, the transcription regulator may comprise a sequence of any one or more sequences of 958-959, 3486-3501, 4426-4427, 4472-4473, 4484-4499, 1468-1469, 3996-4011, 4936-4937, 4982-4983, 4994-5009, 5697-5698, 8632-8647, 9572-9573, 9618-9619, and 9630-9645.

In some embodiments, the transcription initiator comprises sequences derived from the untranslated genomic sequence of RELA, optionally comprising a sequence of any one or more of sequences of SEQ ID NO: 4484-4499, 4994-5009, and 9630-9645.

In some embodiments, when the transcription initiator comprises one or more sequences from the untranslated genomic sequence of IRF2; in some embodiments, the transcription regulator may comprise a sequence of any one or more sequences of SEQ ID NO: 2384-2447, 4430-4461, 2894-2957, 4940-4971, 7530-7593, and 9576-9607.

In some embodiments, when the transcription initiator comprises one or more sequences from the untranslated genomic sequence of GATA3; in some embodiments, the transcription regulator may comprise a sequence of any one or more sequences of SEQ ID NO: 1048-1079, 3566-3709, 1558-1589, 4076-4219, 6194-6225, and 8712-8855.

In some embodiments, when the transcription initiator comprises one or more sequences from the untranslated genomic sequence of ATF3; in some embodiments, the transcription regulator may comprise a sequence of any one or more sequences of SEQ ID NO: 124-159, 3140-3147, 634-669, 3650-3657, 5179-5214, and 8286-8293.

In some embodiments, the one or more constructs may also include an integration sequence capable of inducing or increasing insertion of the construct into a target site, e.g., of a vector or a chromosome. The insertion sequence may comprise multiple insertion sequences. For example, in some embodiments, the insertion sequence may comprise a first integration sequence and a second integration sequence. In some embodiments, the first integration sequence may be a 5′ integration sequence and the second integration sequence may be a 3′ integration sequence. In some embodiments, the insertion sequence may include a homologous sequence that corresponds to an insertion site in order to facilitate homologous recombination. In some embodiments, the integration sequence may include one or more transposable elements that may help facilitate transposition. In some embodiments, the transposable element may include an insertion sequence and/or a transposon sequence. In some embodiments, the integration sequence may comprise a first 5′ terminal repeat sequence or a second 3′ terminal repeat sequence. In some embodiments, the construct may be inducible.

In some embodiments, the nucleic acid construct does not include a transcription initiator comprising a human beta globin sequence and a transcription regulator comprising any of a NFAT sequence, a IRF4 sequence, a AICE sequence, or a ISRE sequence. In some embodiments, the AICE sequence may include or encompass sequences of or derived from IRF4 or IRF8, each with BATF. In some embodiments, the AICE sequences may be derived from the untranslated region of IRF4, IRF8, and/or BATF. In some embodiments, the IRSE sequence may include a consensus sequence of YAGTTTC(A/T)YTTTYCC” in which “Y” is either C or T. In some embodiments, the construct does not include a sequence comprising SEQ ID NO: 9786.

Also in some embodiments, the construct does not include a transcription initiator comprising a YB-TATA sequence and a transcription regulator comprising a NFAT sequence. In some embodiments, the construct does not include a sequence comprising SEQ ID NO: 9787.

### Method of Identifying Nucleic Acid Constructs to Drive Gene Expression

Provided herein is a method of identifying transcription initiators of nucleic acid constructs that drive expression of genes in response to cell state. In some embodiments, the method may be used to identify promoters that may demonstrate certain behaviors such as delayed activity (e.g.,) after antigen stimulation, oscillation after antigen stimulation, a reduction in transcription driven by the promoter after antigen stimulation, an increase in transcription driven by the promoter after antigen stimulation, or the like. In some embodiments, oscillation may include changes in expression of a reporter gene driven by the promoter. A promoter may be identified from a library of constructs wherein each construct comprises one or more unique promoters. In some embodiments, the promoter library may include a plurality of concatemers. In some embodiments, the method of identifying transcription initiators that drive expression of genes in response to cell state may be seen in the exemplary schematic. In some embodiments, the library of constructs may be generated, wherein each construct comprises one or more concatemerized sequence driving expression of a reporter gene (e.g., fluorescent protein). In some embodiments, the library of constructs may be transduced into different types of cells including primary T cells and stimulated with antigens. The primary T cells may be sorted by expression of the reporter gene and sequenced for verification. It can be appreciated that screening of the library of constructs may use high throughput protocols known in the art. It can also be appreciated that analysis of the screening results along with the designing of the library of constructs may use high throughput analysis or machine learning techniques.

In designing the promoter library to generate the one or more constructs, a consensus motif may be identified. In some embodiments, each consensus motif may comprise a sequence including one or more transcription factor binding sites or sequences isolated or derived from the untranslated genomic sequence of one or more transcription factors. Once the consensus motif is identified, the 5′ nucleotides and the 3′ nucleotides may be truncated, leaving the consensus motif. At nucleotide positions within the consensus motif that have variability, a “N” may be placed in the sequence so that any nucleotide may be used. At nucleotide positions within the consensus motif that have some variability, another letter (e.g., S, R, or W) may be used to indicate that only specific nucleotides may be placed there. For example, the letter “S” may indicate that A or T may be substituted at that position, the letter “W” may indicate that G or C may be substituted at that position, the letter “R” may indicate that A or G may be substituted there or the like. In some embodiments, the reverse complement of the consensus motif may be added to the construct. In some embodiments, multiple copies of the consensus motif may be generated and linked together by a linking sequence or a spacer to create a concatemer. In some embodiments, the spacer may include at least 2 base pairs, at least 3 base pairs, at least 4 base pairs, at least 5 base pairs, at least 6 base pairs, at least 7 base pairs, at least 8 base pairs, at least 9 base pairs, at least 10 base pairs or the like. In some embodiments, the spacer may include a 6 base pair spacer. Exemplary spacers may comprise sequences of TACGCT, TGATCT, TGCTTT, or TGCCCG. In some embodiments, the same spacer may separate each copy of the consensus motif in the concatemer, or different spacers may separate each copy of the consensus motif in the concatemer.

In some embodiments, the construct may comprise adapter sequences located 5′ and/or 3′ of the transcription initiator and/or transcription regulator. In some embodiments, the adapter sequences may comprise a sequence of AAAGCACTCTTAGGCCTCTGCGTCTCATTCTGAAGACTCACGA (SEQ ID NO: 9801), or AACGGAGTCTTCAACGTGAGACGGCTCGATGCCATAGTTCCTT (SEQ ID NO: 9801).

In some embodiments, the consensus motif may be identified after being isolated or derived from the untranslated genomic sequence of one or more transcription factors. In some embodiments, the consensus motif may be identified after being isolated or derived from the untranslated genomic sequence of one or more sequences to which transcription factors bind. For example, the consensus motif may include any one or more sequences of Table 1 including any one or more sequences of SEQ ID NO: 175-509, 9793-9800. The transcription initiator may comprise any one or more sequences of Table 1 including any one or more sequences of SEQ ID NO: 175-509, 9793-9800.

TABLE 1
Exemplary consensus motifs

| Transcription | | |
|---|---|---|
| Factor | Consensus Motif | SEQ ID NO: |
| JUNB | RATGAWTCAT | 175 |
| JUND | NNTGAWTCATSNNNRRANT | 176 |
| BATF | TGAWTCATSNYNRRA | 177 |
| FOS | GATGAWTCAYNNN | 178 |
| BATF | DATGAWTCAT | 179 |
| ATF3 | DATGAWTCATHN | 180 |
| NFE2L1 | ATGACTCAY | 181 |
| JUNB | NNRTGAWTCATNNNN | 182 |
| FRA2 | GGATGACTCATC | 183 |
| IRF4 | NNWNNNGAANTGANSWSN | 184 |
| FOSL2 | NATGAWTCABNN | 185 |
| FOS | NDATGAWTCAYN | 186 |
| ZSCAN5C | GNTTTRTTNACTCAC | 187 |
| ZBTB37 | RTCGGCYAANSCGGCAN | 188 |
| ZNF442 | ACTCTTTCTCTGCTGCAAYACCTGCTGTY | 189 |
| ZBTB20 | NMYATACRTTNN | 190 |
| SMAD3 | YGTCTAGACA | 191 |
| FRA1 | NNATGAWTCATH | 192 |
| AICE | GAAATGAGTCA | 193 |
| JUN-AP1 | GATGACTCATCN | 194 |
| JDP2 | ATGASTCAT | 195 |
| HOXD11 | RTCGTAAAAN | 196 |
| FOSL2 | NRTGASTCATCCNN | 197 |
| ZBTB7C | NCGACCACCGNN | 198 |
| POU1F1 | NNNATGMATASTTAATN | 199 |
| GRHL2 | NNNAACTGGTTTNNN | 200 |
| ZBTB7B | NCGACCACCGAA | 201 |
| NR2E3 | AAAGTCAAAAGTCA | 202 |
| HOXC9 | GTCGTAAAAN | 203 |
| TBX3 | NAGGTGTSAN | 204 |
| ZNF266 | NNNRCTCACAGGYCC | 205 |
| ZNF669 | GTCATCR | 206 |
| HOXC11 | NTTTWACGACN | 207 |
| HOXA10 | RGTCGTAAAAN | 208 |
| FOSL1 | NGGTGAWTCAT | 209 |
| ZNF587 | CANNTGGCGCCCAACATKGGNCNN | 210 |
| ATF1 | TKRCGTCAYNN | 211 |
| ZNF652 | TTAACCCTTTVNKKN | 212 |
| ATF2 | ATGACGTCAY | 213 |
| ZNF440 | ARTKGTTCTGCTNNN | 214 |
| PKNOX2 | TGACAGGTGTCA | 215 |
| SMAD5 | YGTCTAGACA | 9791 |
| RFX1 | GGTTGCCATGGCAA | 216 |
| HOXB9 | GTCGTAAAAN | 9792 |
| ZNF433 | ACYAWTGTRRTMATT | 217 |
| PKNOX1 | TGACAGWTGTCA | 218 |
| PBX2 | NTGATTGACAGN | 219 |
| ATF1 | GATGACGTCA | 220 |
| MYCN | NNCCACGTGGNN | 221 |
| SOX7 | AACAATRTNCAKTGTT | 222 |
| ZBTB22 | NKCACTAYNRTAGTGMN | 223 |
| BACH2 | NNTGACTCAGCANN | 224 |
| NFKB2 | NGGGGASTCCCCN | 225 |
| OSR2 | NCNGCTNCTGN | 226 |
| BACH2 | TGCTGAGTCA | 227 |
| TGIF2LY | TGACAGCTGTCA | 228 |
| DUX4 | TAAYYYAATCA | 229 |
| MAFK | TGCTGAWTCAGCA | 230 |
| VDR | GRGTTCATYGRGTTCA | 231 |
| IRF:BATF | CTTTCANTATGACTV | 232 |
| GLI3 | GACCACCCANG | 233 |
| JUN | CATNCCSNNNNGRTGAWKYM | 234 |
| CREB1 | NNTGACGTCANN | 235 |
| PBX3 | CCTGTCAATCAN | 236 |
| GLII | GACCACCCAMG | 237 |
| MAFG | NSSNTGCTGASTCAGCANSSN | 238 |
| MAFA | TGCTGACTCA | 239 |
| ATF7 | NGRTGACGTCAY | 240 |
| HOXA11 | GGTCGTAAAAN | 241 |
| PRDM14 | AGGTCTCTAACC | 242 |
| RXRA | NRRGGTCA | 243 |
| ZNF350 | CNGCYYTTTTSTNACCNNNCN | 244 |
| NRF | GTGCGCATGCGC | 245 |
| CREB3L4 | NRTGACGTCAYN | 246 |
| NF-E2 | GATGACTCAGCA | 247 |
| MYB | GGCVGTTR | 248 |
| ZBTB43 | NGTGCCNNNNNNNYAGCACN | 249 |
| NFE2L2 | ATGACTCAGCANSS | 250 |
| GATA:SCL | CGGCTGCNGNNNNCAGATAA | 251 |
| PPARD | NRRGGTCAAAGGTCAN | 252 |
| IRF4 | ACTGAAACCA | 253 |
| SPDEF | AMCCGGATGTS | 254 |
| BZIP:IRF | NAGTTTCABTHTGACTNS | 255 |
| ISL2 | YTAAKTGC | 256 |
| TGIF2 | TGTCANYT | 257 |
| KLF5 | GCCCCRCCCH | 258 |
| RELB | NNATTCCCCNN | 259 |
| AMYB | TGGCAGTTGG | 260 |
| KLF10 | GGGGGTGTGTCC | 261 |
| MAFF | TGCTGACTCAGCANS | 262 |
| ELF1 | NNNASCCGGAAGYGW | 263 |
| NR2F1 | RRGGTCAAAGGTCAN | 264 |
| ESR1 | NNNCCAAGGTCANN | 265 |
| ZNF736 | AAAARCCCCAGA | 266 |
| COUP-TFII | AGRGGTCA | 267 |
| ZNF26 | TGATGAC | 268 |
| TCF7 | AWATCAAAG | 269 |
| BACH1 | ASSNTGCTGAGTCAT | 270 |
| POU3F3 | SSTATGCSAATTW | 271 |
| GATA3 | NSGATARNNATCSWN | 272 |
| TFAP4 | ASCAGCTGAT | 273 |
| NFE2L2 | ASSSTGCTGAGTCAT | 274 |
| BCL11A | NTTTCAWTTCCYCNT | 275 |
| ANHX | TTGACASG | 276 |
| ZNF467 | TGGGGAAGGGCM | 277 |
| NKX2-3 | NNNAAGTGWN | 278 |
| GATA | NAGATSNBNATCTNN | 279 |
| ZNF282 | CTCCCW | 280 |
| ZNF561 | TTTCTGC | 281 |
| MAFB | SNTGCTGAWTCAGCASWTTY | 282 |
| GLI2 | GACCACCCANG | 9793 |
| IRF6 | RGTSTCGNNNNNNYGASACT | 283 |
| MAFB | NNTGCTGAWTCAGCANSS | 284 |
| ATOH1 | AACAGCTGTC | 285 |
| NRF2 | HTGCTGAGTCAT | 286 |
| ASCL1 | NNAGCAGCTGWNN | 287 |
| ZIM3 | NNTRGNTTTCTGTTNNYN | 288 |
| SALL4 | CCCSCCCNNC | 289 |
| NFE2 | NATGACTCAGCANSTNN | 290 |
| STAT1 | NYCNTTTCCNGGAANNCNNNN | 291 |
| ZKSCAN5 | TCACCTCCN | 292 |
| LYL1 | NNGRNARCAGMTGN | 293 |
| TIISRE | ACTTTCGTTTCT | 294 |
| ZKSCAN7 | NYAAACTGTNRAGYGCN | 295 |
| KLF14 | GGCCMCGCCCMCYN | 296 |
| GATA5 | WGATAANN | 297 |
| ZNF439 | CAGTCACCYTCSGG | 298 |
| CREB3 | NNRTGACGTCAYCM | 299 |
| EAR2 | NRBCARRGGTCA | 300 |
| FOXP1 | NNGTAAACAGNN | 301 |
| STAT3 | CNTTTMCYGGAARTWANNNN | 302 |
| NR1H4 | NNGGTCATTNN | 303 |
| PATZI | CCCCKCCC | 304 |
| FOXK2 | WCHTGTTTACAT | 305 |
| OVOL2 | NNSCCGTTANNNN | 306 |
| RXR | TAGGGCAAAGGTCA | 307 |
| AP4 | NAHCAGCTGD | 308 |
| MOUSE_ | ACTYKNATTCGTGNTACTTC | 309 |
| RECOMBINATION_ | | |
| HOTSPOT | | |
| GATA1 | CAGATAAGGN | 310 |
| ZNF141 | NNNNNNNNGGKGGGRGCGTCCCCC | 311 |
| HIC1 | TGCCAGCB | 312 |
| ZNF821 | NTGTCYGTCTGTCCN | 313 |
| ZNF174 | NGNCRRTCACTYGCCN | 314 |
| MAF | NGTCAGCAN | 315 |
| MYOD | AGCAGCTGCTGC | 316 |
| CHR | CGGTTTCAAA | 317 |
| C-JUN-CRE | ATGACGTCATCC | 318 |
| GATA2 | NNSGATAAGNN | 319 |
| STAT6 | NTTCCWWAGAA | 320 |
| ELK1 | ACCGGAAGTN | 321 |
| MSX1 | TAATTGWNNTTTAATTGN | 322 |
| HAND2 | TGACANARRCCAGRC | 323 |
| MSX2 | TAATTGWSSTTTAATTGC | 324 |
| PBX3 | NNNNGCCAATWRNNN | 325 |
| ZNF12 | YNYTYWTYNCNGCNGCNW | 326 |
| KLF1 | VDGGGYGGGGCY | 327 |
| GATA2 | NCCTTATCTC | 328 |
| SP5 | RGKGGGCGGAGC | 329 |
| RUNX2 | NRACCGCAAACCGCAN | 330 |
| MYB | NNCAACTGNN | 331 |
| ETS:RUNX | ACAGGATGTGGT | 332 |
| BMYB | NHAACBGYYV | 333 |
| ESRRB | TCAAGGTCASN | 334 |
| ZNF681 | YCNNCGTCCTTN | 335 |
| HNF4A | NRGNNCAAAGKYCAN | 336 |
| NRL | GTCAGCANNNN | 337 |
| ZNF77 | ACTYCAC | 338 |
| AP-2ALPHA | ATGCCCTGAGGC | 339 |
| NKX2-5 | NTYAAGTGGN | 340 |
| ZNF223 | YGCCMYCTDWTGGC | 341 |
| RFX6 | TGTTKCCTAGCAACM | 342 |
| ZSCAN22 | WMCAGTCSGAKGGAGGAGGC | 343 |
| ZNF740 | NCCCCCCCAC | 344 |
| NF1:FOXA1 | NNTGTTTATTTTGGCA | 345 |
| GATA1 | NWAGATAAGNN | 346 |
| BARX2 | NTAATKRTTSSN | 347 |
| TCF3 | ACATCAAAGG | 348 |
| ZKSCAN3 | GGCTAGCCC | 349 |
| STAT5B | NNTTCYMRGAAN | 350 |
| ZNF75D | GTGGGAAAGCCT | 351 |
| WT1 | CYCCNCCYCCTCCCCCNCCY | 352 |
| MAZ | GGGGGGGG | 353 |
| MEIS1 | VGCTGWCAVB | 354 |
| ZNF431 | NNGNCTTCYAGCCYN | 355 |
| ZNF708 | NNGCTGTACCTNCYNNNNNNN | 356 |
| STAT2 | NTNAGTTTCRNTTTC | 357 |
| NFIX | YTGGCNNNNTGCCAA | 358 |
| FOXL1 | RTAAACA | 359 |
| IRF2 | WGAAAGYGAAASCNSSNC | 360 |
| ZNF398 | CTSYYTCCCTCCCT | 361 |
| CREB5 | VVATGACGTCAT | 362 |
| BARX1 | TAATNGNTSTTTAATNG | 363 |
| PRDM9 | ADGGYAGYAGCATCT | 364 |
| TAL1 | NCTTATCTWNNNNNNWCAG | 365 |
| IRF1 | AAANNGAAAGTGAAAWTRNNN | 366 |
| USF1 | GGTCACGTGA | 367 |
| FXR | AGGTCANTGACCTN | 368 |
| ZBTB2 | NTTTCCGGTAAN | 369 |
| ZNF304 | CCCCCKCCCCRSTCWRGCCYNNNN | 370 |
| OLIG3 | AMCATATGNY | 371 |
| KLF9 | NNCCACACCCACN | 372 |
| ZNF232 | CTTAATCTACRTTTAACAY | 373 |
| TFEB | RTCACGTGAY | 374 |
| ZNF468 | CRCCCCCTCCCN | 375 |
| ZNF684 | ACAGTCCACCCCTT | 376 |
| FEV | ACCGGAAGTN | 377 |
| ASCL1 | NNVVCAGCTGBN | 9794 |
| MLX | ATCACGTGAY | 378 |
| ALX3 | NNTAATTRNN | 379 |
| UNKNOWN-ESC- | CACAGCAGGGGG | 380 |
| ELEMENT | | |
| ZNF302 | AGTTGAGTGACTGYRGTT | 381 |
| ZNF263 | CYCCYCCYCNNCCTCCTCCYY | 382 |
| ZNF649 | TTATAT | 383 |
| ETV4 | ACCGGAAGTN | 9795 |
| KLF12 | CCNNGCCCCGCCCCN | 384 |
| MYF6 | AACARCTGTT | 385 |
| OVOL1 | NNSACCGTTATNYN | 386 |
| FOXC2 | NGTMAATATTKACN | 387 |
| NEUROG2 | ACCATCTGTT | 388 |
| INSM1 | YGCCCCCTGRCA | 389 |
| ZNF695 | TTCCTCY | 390 |
| ZNF41 | CCTCATGGTGYCYTSYTCCCTTGTG | 391 |
| ZNF563 | NTCCNNNCNGGCARCTGY | 392 |
| GATA | NNNNNBAGATASYATCTVHN | 393 |
| ZNF200 | AYTTCC | 394 |
| BACHI | GTCACGTG | 395 |
| ZNF250 | YNNTGCANTTTTTNN | 396 |
| IRF5 | CCGAAACCGAAACY | 397 |
| ATF3 | GGTCACGTGRW | 398 |
| RBPJ1 | HTTTCCCAWG | 399 |
| ZNF285 | GCCAATTYCYRTGGTGTAAAT | 400 |
| IRF3 | AGTTTCAKTTTC | 401 |
| SCL | ANCAGCTG | 402 |
| KLF14 | RGKGGGCGKGGC | 403 |
| TGIF1 | YTGWCADY | 404 |
| GATA4 | WGATAANN | 405 |
| ELK4 | CCGGAAGYGN | 9796 |
| KLF6 | MKGGGYGTGGCC | 406 |
| MITF | RTCATGTGAC | 407 |
| ZNF140 | CAATTCCGCTCMNNNNNNNNNNNN | 408 |
| ELF2 | NANSCGGAAGTN | 409 |
| PPARA | VNAGGKCAAAGGTCA | 410 |
| TFEC | RNCACRTGAY | 411 |
| RORC | AAYTAGGTCA | 412 |
| GATA4 | NBWGATAAGR | 413 |
| COUP-TFII | GKBCARAGGTCA | 414 |
| ZNF662 | RKCTAGCCCNNKCCTACC | 415 |
| RORA | AASCTAGGTCARDNN | 416 |
| PGR | NGNACANNNYGTNCY | 417 |
| TBX20 | GGTGYTGACAGW | 418 |
| ZSCAN31 | NNNTTAATTGNTTKTN | 419 |
| ZNF707 | ACCCCACTCCTGGTM | 420 |
| IRF8 | NCGAAACCGAAACY | 421 |
| NFATC3 | WNTTTCCRTN | 422 |
| ZBTB45 | NMTATAGGNGN | 423 |
| ATF2 | NRRTGACGTCAT | 424 |
| ZNF766 | GGTTTMT | 425 |
| FOXP2 | NWGTAAACARN | 426 |
| GABPA | NRCCGGAAGTG | 427 |
| ASCL2 | NRCAGCTGYN | 428 |
| ELF3 | ANCAGGAAGT | 429 |
| SPDEF | ACATCCTGGT | 430 |
| EHF | AVCAGGAAGT | 431 |
| PRDM10 | TGGTACATTCCA | 432 |
| REL | SGGRNWTTCC | 433 |
| ZNF605 | NNNYNKGGYNTCTNNNNCCCY | 434 |
| ZNF419 | ASTMCAGCCCTAKACTCTCCY | 435 |
| GATA3 | AGATWTNDNNDWAGATAAWN | 436 |
| PPARE | TGACCTTTGCCCCA | 437 |
| MEF2D | GCTATTTTTAGC | 438 |
| ETS | AACCGGAAGT | 439 |
| MAZ | WCCCCKCCC | 440 |
| GCM2 | NATGCGGGTN | 441 |
| GATA6 | YCTTATCTBN | 44 |
| KLF17 | MMCCACGCACCCMTY | 443 |
| ZNF341 | GCTWTTCCNNCNNCCNCCCNN | 444 |
| ERF | ACCGGAAGTR | 445 |
| ETV2 | AACCGGAAATR | 446 |
| SP9 | NCCACGCCCMCN | 447 |
| ELF5 | ANSMGGAAGTN | 448 |
| ELF1 | AACCGGAAGT | 9797 |
| ELF5 | ACVAGGAAGT | 449 |
| NFAT:AP1 | GAATGGAAAAAATGAGTCAT | 450 |
| MEIS3 | NTGACAGN | 451 |
| RBPJ:EBOX | GGGRAARRGRMCAGMTG | 452 |
| SP8 | RCCACGCCCMCY | 453 |
| NEUROD1 | GCCATCTGTT | 454 |
| ZNF891 | NNNGNNCNWNGGCTTCYAGCC | 455 |
| RORA | AWNNAGGTCA | 456 |
| TBOX:SMAD | AGGTGHCAGACA | 457 |
| ERRG | GTGACCTTGRVN | 458 |
| MAFK | GCTGASTCAGCA | 459 |
| ZNF157 | TRTAYTGCSNNCCYTNCCANGSCM | 460 |
| AP-2GAMMA | WCCTWAGGWCAS | 46 |
| IRX3 | SACATGNSSSSNCATGTS | 462 |
| ZNF71 | NAGTTTCRTTTTCY | 463 |
| IRF8 | GRAAWTGAAAWT | 464 |
| NFATC1 | ATTTTCCATT | 465 |
| PU.1:IRF8 | GGAAGTGAAAWT | 466 |
| ETSI-DISTAL | AACAGGAAGT | 467 |
| RXRG | GGTCAAAGGTCA | 468 |
| SP3 | NCCACGCCCMC | 469 |
| ETV2 | NNAYTTCCTGHN | 470 |
| ZFP82 | AGAATTAGTGAACTGGAAGAT | 471 |
| RORC | SAABTAGGTCAV | 472 |
| ISRE | AGTTTCAGTTTC | 473 |
| NFATC4 | WNTTTCCRYN | 474 |
| TLX? | CTGGCAGGCTGCCA | 475 |
| ELK4 | NRYTTCCGGY | 476 |
| IRF7 | NCGAAARYGAAANT | 477 |
| ELF4 | ACTTCCKGKT | 478 |
| ELK1 | HACTTCCGGY | 479 |
| REL | GGAAATTCCC | 480 |
| IRF9 | AACGAAACCGAAACT | 481 |
| IRF2 | GAAAWYGAAAWY | 482 |
| ESRRB | GTGACCTTGA | 483 |
| EWS:FLI1-FUSION | AACAGGAAAT | 484 |
| RUNX1 | AAACCACAAA | 485 |
| EWS:ERG-FUSION | ATTTCCTGTN | 486 |
| ETV4 | ACCGGAAGTG | 487 |
| REL | AGGGGATTTCCC | 488 |
| IRF1 | GAAAGTGAAAGT | 489 |
| KLF16 | GMCACGCCCCC | 490 |
| EGR4 | NNMCGCCCACGCANNN | 491 |
| RELA | NGGGRMTTTCCMNN | 492 |
| RUNX1 | CAAACCACAG | 493 |
| RUNX3 | AACCRCAA | 494 |
| RUNX2 | NWAACCACADNN | 495 |
| GABPA | AACCGGAAGT | 496 |
| NR2F2 | NNAAGGTCANN | 497 |
| NR4A1 | NAAAGGTCAN | 498 |
| RUNX1 | NWAACCACARN | 9798 |
| ERG | ACAGGAAGTG | 499 |
| FLI1 | NRYTTCCGGH | 500 |
| PU.1 | AGAGGAAGTG | 501 |
| EGR1 | NNNNNNNNCCGCCCMCGCNN | 502 |
| RUNX1 | GCTGTGGTTT | 503 |
| ETV1 | AACCGGAAGT | 9799 |
| ETS1 | ACAGGAAGTG | 9800 |
| WT1 | MCTCCCMCRCAB | 504 |
| EGR3 | CKCCCCCNC | 505 |
| EGR2 | NGCGTGGGCGGR | 506 |
| EGR2 | CGCCCMCNC | 507 |
| EGR1 | TGCGTGGGYG | 508 |
| NUR77 | TGACCTTTNCNT | 509 |

The identified consensus motifs of Table 1 comprise some variability within the identified sequence. In some embodiments, the identified consensus motifs of Table 1 may allow for one or more nucleotide substitutions within the identified consensus motifs. For example, within the consensus motifs of Table 1, “N” may allow for any nucleotide to be substituted at that position including A, G, C, T: “S” may allow for either G or C to be substituted at that position: “R” may allow for either A or G to be substituted at that position: and “W” may allow for A or T to be substituted at that position.

Within each identified consensus motifs, specific motifs Were identified that reduced the variability of the consensus motifs. Table 2 lists exemplary specific motifs that exist within the identified consensus motifs of Table 1. The transcription initiator and the regulators of the disclosure may comprise any one or more of the specific exemplary motifs of Table 2.

TABLE 2
Exemplary Motifs of the Consensus Motifs

| Motif | Motif | Motif |
|---|---|---|
| AATGAGTCAT (SEQ ID | AAACTGTTA (SEQ ID | ACTTCCGGT (SEQ ID NO: |
| NO: 510) | NO: 2360 | 3343 |
| ATGACTCATT (SEQ ID | TAACAGTTT (SEQ ID | AACAACTGTT (SEQ ID NO: |
| NO: 511) | NO: 2361 | 3346 |
| AATGACTCAT (SEQ ID | AAACTGTTC (SEQ ID | AACAGTTGTT (SEQ ID NO: |
| NO: 512) | NO: 2362 | 3347 |
| ATGAGTCATT (SEQ ID | GAACAGTTT (SEQ ID | AACAGCTGTT (SEQ ID NO: |
| NO: 513) | NO: 2363 | 3348 |
| GATGAGTCAT (SEQ ID | AAACTGTTG (SEQ ID | AACAGCTGTT (SEQ ID NO: |
| NO: 514) | NO: 2364 | 3349 |
| ATGACTCATC (SEQ ID | CAACAGTTT (SEQ ID | ACCATCTGTT (SEQ ID NO: |
| NO: 515) | NO: 2365 | 3350 |
| GATGACTCAT (SEQ ID | CAACCGCCA (SEQ ID | AACAGATGGT (SEQ ID NO: |
| NO: 516) | NO: 2366 | 3351 |
| ATGAGTCATC (SEQ ID | TGGCGGTTG (SEQ ID | TTCCTCC (SEQ ID NO: 3376 |
| NO: 517) | NO: 2367 | |
| GATGAGTCAC (SEQ ID | CAACCGCCC (SEQ ID | GGAGGAA (SEQ ID NO: |
| NO: 736) | NO: 2368 | 3377 |
| GTGACTCATC (SEQ ID | GGGCGGTTG (SEQ ID | TTCCTCT (SEQ ID NO: 3378 |
| NO: 737) | NO: 2369 | |
| GATGAGTCAT (SEQ ID | CAACCGCCG (SEQ ID | AGAGGAA (SEQ ID NO: |
| NO: 616) | NO: 2370 | 3379 |
| ATGACTCATC (SEQ ID | CGGCGGTTG (SEQ ID | ACTTCC (SEQ ID NO: 3636 |
| NO: 627) | NO: 2371 | |
| GATGACTCAC (SEQ ID | CAACCGCTA (SEQ ID | GGAAGT (SEQ ID NO: 3637 |
| NO: 618) | NO: 2372 | |
| GTGAGTCATC (SEQ ID | TAGCGGTTG (SEQ ID | ATTTCC (SEQ ID NO: 3638 |
| NO: 619) | NO: 2373 | |
| GATGACTCAT (SEQ ID | CAACCGCTC (SEQ ID | GGAAAT (SEQ ID NO: 3639 |
| NO: 516) | NO: 2374 | |
| ATGAGTCATC (SEQ ID | GAGCGGTTG (SEQ ID | GTCACGTG (SEQ ID NO: |
| NO: 517) | NO: 2375 | 3640 |
| AATGAGTCAT (SEQ ID | CAACCGCTG (SEQ ID | CACGTGAC (SEQ ID NO: |
| NO: 622) | NO: 2376 | 3641 |
| ATGACTCATT (SEQ ID | CAGCGGTTG (SEQ ID | ATTTCCCAGG (SEQ ID NO: |
| NO: 623) | NO: 2377 | 3658 |
| AATGACTCAT (SEQ ID | CAACCGTCA (SEQ ID | CCTGGGAAAT (SEQ ID NO: |
| NO: 624) | NO: 2378 | 3659 |
| ATGAGTCATT (SEQ ID | TGACGGTTG (SEQ ID | ATTTCCCACG (SEQ ID NO: |
| NO: 625) | NO: 2379 | 3660 |
| GATGAGTCAT (SEQ ID | CAACCGTCC (SEQ ID | CGTGGGAAAT (SEQ ID NO: |
| NO: 626) | NO: 2380 | 3661 |
| ATGACTCATC (SEQ ID | GGACGGTTG (SEQ ID | CTTTCCCAGG (SEQ ID NO: |
| NO: 617) | NO: 2381 | 3662 |
| GATGACTCAT (SEQ ID | CAACCGTCG (SEQ ID | CCTGGGAAAG (SEQ ID NO: |
| NO: 620) | NO: 2382 | 3663 |
| ATGAGTCATC (SEQ ID | CGACGGTTG (SEQ ID | CTTTCCCACG (SEQ ID NO: |
| NO: 629) | NO: 2383 | 3664 |
| TATGAGTCAT (SEQ ID | CAACCGTTA (SEQ ID | CGTGGGAAAG (SEQ ID NO: |
| NO: 630) | NO: 2384 | 3665 |
| ATGACTCATA (SEQ ID | TAACGGTTG (SEQ ID | TTTTCCCAGG (SEQ ID NO: |
| NO: 631) | NO: 2385 | 3666 |
| TATGACTCAT (SEQ ID | CAACCGTTC (SEQ ID | CCTGGGAAAA (SEQ ID NO: |
| NO: 632) | NO: 2386 | 3667 |
| ATGAGTCATA (SEQ ID | GAACGGTTG (SEQ ID | TTTTCCCACG (SEQ ID NO: |
| NO: 633) | NO: 2387 | 3668 |
| ATGACTCAC (SEQ ID | CAACCGTTG (SEQ ID | CGTGGGAAAA (SEQ ID NO: |
| NO: 670) | NO: 2388 | 3669 |
| GTGAGTCAT (SEQ ID | CAACGGTTG (SEQ ID | AACAGCTG (SEQ ID NO: |
| NO: 671) | NO: 2389 | 3690 |
| ATGACTCAT (SEQ ID | CAACGGCCA (SEQ ID | CAGCTGTT (SEQ ID NO: |
| NO: 676) | NO: 2390 | 3691 |
| ATGAGTCAT (SEQ ID | TGGCCGTTG (SEQ ID | CTGACAAC (SEQ ID NO: |
| NO: 677) | NO: 2391 | 3708 |
| ATGAGTCAT (SEQ ID | CAACGGCCC (SEQ ID | GTTGTCAG (SEQ ID NO: |
| NO: 683) | NO: 2392 | 3709 |
| ATGACTCAT (SEQ ID | GGGCCGTTG (SEQ ID | CTGACAAT (SEQ ID NO: |
| NO: 672) | NO: 2393 | 3710 |
| ATGACTCAT (SEQ ID | CAACGGCCG (SEQ ID | ATTGTCAG (SEQ ID NO: |
| NO: 675) | NO: 2394 | 3711 |
| ATGAGTCAT (SEQ ID | CGGCCGTTG (SEQ ID | CTGACAGC (SEQ ID NO: |
| NO: 677) | NO: 2395 | 3712 |
| GTGAGTCAT (SEQ ID | CAACGGCTA (SEQ ID | GCTGTCAG (SEQ ID NO: |
| NO: 678) | NO: 2396 | 3713 |
| ATGACTCAC (SEQ ID | TAGCCGTTG (SEQ ID | CTGACAGT (SEQ ID NO: |
| NO: 679) | NO: 2397 | 3714 |
| GTGACTCAT (SEQ ID | CAACGGCTC (SEQ ID | ACTGTCAG (SEQ ID NO: |
| NO: 680) | NO: 2398 | 3715 |
| ATGAGTCAC (SEQ ID | GAGCCGTTG (SEQ ID | CTGACATC (SEQ ID NO: |
| NO: 681) | NO: 2399 | 3716 |
| ATGAGTCAC (SEQ ID | CAACGGCTG (SEQ ID | GATGTCAG (SEQ ID NO: |
| NO: 716) | NO: 2400 | 3717 |
| GTGACTCAT (SEQ ID | CAGCCGTTG (SEQ ID | CTGACATT (SEQ ID NO: |
| NO: 717) | NO: 2401 | 3718 |
| ATGAGTCAG (SEQ ID | CAACGGTCA (SEQ ID | AATGTCAG (SEQ ID NO: |
| NO: 718) | NO: 2402 | 3719 |
| CTGACTCAT (SEQ ID | TGACCGTTG (SEQ ID | CTGTCAAC (SEQ ID NO: |
| NO: 719) | NO: 2403 | 3720 |
| ATGAGTCAT (SEQ ID | CAACGGTCC (SEQ ID | GTTGACAG (SEQ ID NO: |
| NO: 720) | NO: 2404 | 3721 |
| ATGACTCAT (SEQ ID | GGACCGTTG (SEQ ID | CTGTCAAT (SEQ ID NO: |
| NO: 721) | NO: 2405 | 3722 |
| ATGACTCAC (SEQ ID | CAACGGTCG (SEQ ID | ATTGACAG (SEQ ID NO: |
| NO: 722) | NO: 2406 | 3723 |
| GTGAGTCAT (SEQ ID | CGACCGTTG (SEQ ID | CTGTCAGC (SEQ ID NO: |
| NO: 723) | NO: 2407 | 3724 |
| ATGACTCAG (SEQ ID | CAACGGTTA (SEQ ID | GCTGACAG (SEQ ID NO: |
| NO: 724) | NO: 2408 | 3725 |
| CTGAGTCAT (SEQ ID | TAACCGTTG (SEQ ID | CTGTCAGT (SEQ ID NO: |
| NO: 725) | NO: 2409 | 3726 |
| ATGACTCAT (SEQ ID | CAACGGTTC (SEQ ID | ACTGACAG (SEQ ID NO: |
| NO: 726) | NO: 2410 | 3727 |
| ATGAGTCAT (SEQ ID | GAACCGTTG (SEQ ID | CTGTCATC (SEQ ID NO: |
| NO: 727) | NO: 2411 | 3728 |
| AATGAGTCAC (SEQ ID | CAACGGTTG (SEQ ID | GATGACAG (SEQ ID NO: |
| NO: 728) | NO: 2412 | 3729 |
| GTGACTCATT (SEQ ID | CAACCGTTG (SEQ ID | CTGTCATT (SEQ ID NO: |
| NO: 729) | NO: 2413 | 3740 |
| AATGAGTCAT (SEQ ID | CAACTGCCA (SEQ ID | AATGACAG (SEQ ID NO: |
| NO: 730) | NO: 2414 | 3741 |
| ATGACTCATT (SEQ ID | TGGCAGTTG (SEQ ID | TTGACAAC (SEQ ID NO: |
| NO: 810) | NO: 2415 | 3742 |
| AATGACTCAC (SEQ ID | CAACTGCCC (SEQ ID | GTTGTCAA (SEQ ID NO: |
| NO: 732) | NO: 2416 | 3743 |
| GTGAGTCATT (SEQ ID | GGGCAGTTG (SEQ ID | TTGACAAT (SEQ ID NO: |
| NO: 733) | NO: 2417 | 3744 |
| AATGACTCAT (SEQ ID | CAACTGCCG (SEQ ID | ATTGTCAA (SEQ ID NO: |
| NO: 805) | NO: 2418 | 3735 |
| ATGAGTCATT (SEQ ID | CGGCAGTTG (SEQ ID | TTGACAGC (SEQ ID NO: |
| NO: 735) | NO: 2419 | 3736 |
| GATGAGTCAC (SEQ ID | CAACTGCTA (SEQ ID | GCTGTCAA (SEQ ID NO: |
| NO: 736) | NO: 2420 | 3737 |
| GTGACTCATC (SEQ ID | TAGCAGTTG (SEQ ID | TTGACAGT (SEQ ID NO: |
| NO: 737) | NO: 2421 | 3738 |
| GATGAGTCAT (SEQ ID | CAACTGCTC (SEQ ID | ACTGTCAA (SEQ ID NO: |
| NO: 643) | NO: 2422 | 3739 |
| ATGACTCATC (SEQ ID | GAGCAGTTG (SEQ ID | TTGACATC (SEQ ID NO: |
| NO: 627) | NO: 2423 | 3740 |
| GATGACTCAC (SEQ ID | CAACTGCTG (SEQ ID | GATGTCAA (SEQ ID NO: |
| NO: 618) | NO: 2424 | 3741 |
| GTGAGTCATC (SEQ ID | CAGCAGTTG (SEQ ID | TTGACATT (SEQ ID NO: |
| NO: 741) | NO: 2425 | 3742 |
| GATGACTCAT (SEQ ID | CAACTGTCA (SEQ ID | AATGTCAA (SEQ ID NO: |
| NO: 803) | NO: 2426 | 3743 |
| ATGAGTCATC (SEQ ID | TGACAGTTG (SEQ ID | TTGTCAAC (SEQ ID NO: |
| NO: 802) | NO: 2427 | 3744 |
| TATGAGTCAC (SEQ ID | CAACTGTCC (SEQ ID | GTTGACAA (SEQ ID NO: |
| NO: 744) | NO: 2428 | 3745 |
| GTGACTCATA (SEQ ID | GGACAGTTG (SEQ ID | TTGTCAAT (SEQ ID NO: |
| NO: 745) | NO: 2429 | 3746 |
| TATGAGTCAT (SEQ ID | CAACTGTCG (SEQ ID | ATTGACAA (SEQ ID NO: |
| NO: 807) | NO: 2430 | 3747 |
| ATGACTCATA (SEQ ID | CGACAGTTG (SEQ ID | TTGTCAGC (SEQ ID NO: |
| NO: 747) | NO: 2431 | 3748 |
| TATGACTCAC (SEQ ID | CAACTGTTA (SEQ ID | GCTGACAA (SEQ ID NO: |
| NO: 748) | NO: 2432 | 3749 |
| GTGAGTCATA (SEQ ID | TAACAGTTG (SEQ ID | TTGTCAGT (SEQ ID NO: |
| NO: 749) | NO: 2433 | 3750 |
| TATGACTCAT (SEQ ID | CAACTGTTC (SEQ ID | ACTGACAA (SEQ ID NO: |
| NO: 801) | NO: 2434 | 3751 |
| ATGAGTCATA (SEQ ID | GAACAGTTG (SEQ ID | TTGTCATC (SEQ ID NO: |
| NO: 800) | NO: 2435 | 3752 |
| ACATACATT (SEQ ID | CAACTGTTG (SEQ ID | GATGACAA (SEQ ID NO: |
| NO: 780) | NO: 2436 | 3753 |
| AATGTATGT (SEQ ID | CAACAGTTG (SEQ ID | TTGTCATT (SEQ ID NO: |
| NO: 781) | NO: 2437 | 3754 |
| ACATACGTT (SEQ ID | TAACCGCCA (SEQ ID | AATGACAA (SEQ ID NO: |
| NO: 782) | NO: 2438 | 3755 |
| AACGTATGT (SEQ ID | TGGCGGTTA (SEQ ID | AGATAA (SEQ ID NO: 3756 |
| NO: 783) | NO: 2439 | |
| ATATACATT (SEQ ID | TAACCGCCC (SEQ ID | TTATCT (SEQ ID NO: 3757 |
| NO: 784) | NO: 2440 | |
| AATGTATAT (SEQ ID | GGGCGGTTA (SEQ ID | TGATAA (SEQ ID NO: 3758 |
| NO: 785) | NO: 2441 | |
| ATATACGTT (SEQ ID | TAACCGCCG (SEQ ID | TTATCA (SEQ ID NO: 3759 |
| NO: 786) | NO: 2442 | |
| AACGTATAT (SEQ ID | CGGCGGTTA (SEQ ID | CCGGAAGCG (SEQ ID NO: |
| NO: 787) | NO: 2443 | 3760 |
| CCATACATT (SEQ ID | TAACCGCTA (SEQ ID | CGCTTCCGG (SEQ ID NO: |
| NO: 788) | NO: 2444 | 3761 |
| AATGTATGG (SEQ ID | TAGCGGTTA (SEQ ID | CCGGAAGTG (SEQ ID NO: |
| NO: 789) | NO: 2445 | 3762 |
| CCATACGTT (SEQ ID | TAACCGCTC (SEQ ID | CACTTCCGG (SEQ ID NO: |
| NO: 790) | NO: 2446 | 3763 |
| AACGTATGG (SEQ ID | GAGCGGTTA (SEQ ID | ATCATGTGAC (SEQ ID NO: |
| NO: 791) | NO: 2447 | 3780 |
| CTATACATT (SEQ ID | TAACCGCTG (SEQ ID | GTCACATGAT (SEQ ID NO: |
| NO: 792) | NO: 2448 | 3781 |
| AATGTATAG (SEQ ID | CAGCGGTTA (SEQ ID | GTCATGTGAC (SEQ ID NO: |
| NO: 793) | NO: 2449 | 3782 |
| CTATACGTT (SEQ ID | TAACCGTCA (SEQ ID | GTCACATGAC (SEQ ID NO: |
| NO: 794) | NO: 2450 | 3783 |
| AACGTATAG (SEQ ID | TGACGGTTA (SEQ ID | AAGCGGAAGT (SEQ ID NO: |
| NO: 795) | NO: 2451 | 3784 |
| CGTCTAGACA (SEQ ID | TAACCGTCC (SEQ ID | ACTTCCGCTT (SEQ ID NO: |
| NO: 936) | NO: 2452 | 3789 |
| TGTCTAGACG (SEQ ID | GGACGGTTA (SEQ ID | AACCGGAAGT (SEQ ID NO: |
| NO: 797) | NO: 2453 | 3790 |
| TGTCTAGACA (SEQ ID | TAACCGTCG (SEQ ID | ACTTCCGGTT (SEQ ID NO: |
| NO: 798) | NO: 2454 | 4225 |
| TGTCTAGACA (SEQ ID | CGACGGTTA (SEQ ID | ACCACATGAC (SEQ ID NO: |
| NO: 799) | NO: 2455 | 3804 |
| ATGAGTCATA (SEQ ID | TAACCGTTA (SEQ ID | GTCATGTGGT (SEQ ID NO: |
| NO: 800) | NO: 2456 | 3805 |
| TATGACTCAT (SEQ ID | TAACGGTTA (SEQ ID | ACCACATGAT (SEQ ID NO: |
| NO: 801) | NO: 2457 | 3806 |
| ATGAGTCATC (SEQ ID | TAACCGTTC (SEQ ID | ATCATGTGGT (SEQ ID NO: |
| NO: 802) | NO: 2458 | 3807 |
| GATGACTCAT (SEQ ID | GAACGGTTA (SEQ ID | ACCACGTGAC (SEQ ID NO: |
| NO: 803) | NO: 2459 | 3808 |
| ATGAGTCATT (SEQ ID | TAACCGTTG (SEQ ID | GTCACGTGGT (SEQ ID NO: |
| NO: 804) | NO: 2460 | 3809 |
| AATGACTCAT (SEQ ID | CAACGGTTA (SEQ ID | ACCACGTGAT (SEQ ID NO: |
| NO: 805) | NO: 2461 | 3810 |
| ATGACTCATA (SEQ ID | TAACGGCCA (SEQ ID | ATCACGTGGT (SEQ ID NO: |
| NO: 806) | NO: 2462 | 3811 |
| TATGAGTCAT (SEQ ID | TGGCCGTTA (SEQ ID | GCCACATGAC (SEQ ID NO: |
| NO: 807) | NO: 2463 | 3812 |
| ATGACTCATC (SEQ ID | TAACGGCCC (SEQ ID | GTCATGTGGC (SEQ ID NO: |
| NO: 808) | NO: 2464 | 3813 |
| GATGAGTCAT (SEQ ID | GGGCCGTTA (SEQ ID | GCCACATGAT (SEQ ID NO: |
| NO: 809) | NO: 2465 | 3814 |
| ATGACTCATT (SEQ ID | TAACGGCCG (SEQ ID | ATCATGTGGC (SEQ ID NO: |
| NO: 810) | NO: 2466 | 3815 |
| AATGAGTCAT (SEQ ID | CGGCCGTTA (SEQ ID | GCCACGTGAC (SEQ ID NO: |
| NO: 811) | NO: 2467 | 3816 |
| ATGAGTCAT 9SEQ ID | TAACGGCTA (SEQ ID | GTCACGTGGC (SEQ ID NO: |
| NO: 816) | NO: 2468 | 3817 |
| ATGACTCAT 9SEQ ID | TAGCCGTTA (SEQ ID | GCCACGTGAT (SEQ ID NO: |
| NO: 817) | NO: 2469 | 3818 |
| ATGACTCAT (SEQ ID | TAACGGCTC (SEQ ID | ATCACGTGGC (SEQ ID NO: |
| NO: 818) | NO: 2470 | 3819 |
| ATGAGTCAT (SEQ ID | GAGCCGTTA (SEQ ID | AACTAGGTCA (SEQ ID NO: |
| NO: 819) | NO: 2471 | 3820 |
| ATCGTAAAA (SEQ ID | TAACGGCTG (SEQ ID | TGACCTAGTT (SEQ ID NO: |
| NO: 820) | NO: 2472 | 3821 |
| TTTTACGAT (SEQ ID NO: | CAGCCGTTA (SEQ ID | AATTAGGTCA (SEQ ID NO: |
| 821) | NO: 2473 | 3822 |
| GTCGTAAAA (SEQ ID | TAACGGTCA (SEQ ID | TGACCTAATT (SEQ ID NO: |
| NO: 822) | NO: 2474 | 3823 |
| TTTTACGAC (SEQ ID | TGACCGTTA (SEQ ID | CAGATAAGA (SEQ ID NO: |
| NO: 823) | NO: 2475 | 3824 |
| CGACCACCG (SEQ ID | TAACGGTCC (SEQ ID | TCTTATCTG (SEQ ID NO: |
| NO: 832) | NO: 2476 | 3825 |
| CGGTGGTCG (SEQ ID | GGACCGTTA (SEQ ID | CAGATAAGG (SEQ ID NO: |
| NO: 833) | NO: 2477 | 3826 |
| AACTGGTTT (SEQ ID | TAACGGTCG (SEQ ID | CCTTATCTG (SEQ ID NO: |
| NO: 842) | NO: 2478) | 3827) |
| AAACCAGTT (SEQ ID | CGACCGTTA (SEQ ID | CTGATAAGA (SEQ ID NO: |
| NO: 843) | NO: 2479) | 3828) |
| CGACCACCGAA (SEQ ID | TAACGGTTA (SEQ ID | TCTTATCAG (SEQ ID NO: |
| NO: 844) | NO: 2480) | 3829) |
| TTCGGTGGTCG (SEQ ID | TAACCGTTA (SEQ ID | CTGATAAGG (SEQ ID NO: |
| NO: 845) | NO: 2481) | 3830) |
| GTCGTAAAA (SEQ ID | TAACGGTTC (SEQ ID | CCTTATCAG (SEQ ID NO: |
| NO: 848) | NO: 2482) | 3831) |
| TTTTACGAC (SEQ ID | GAACCGTTA (SEQ ID | GAGATAAGA (SEQ ID NO: |
| NO: 849) | NO: 2483) | 3832) |
| AGGTGTGA (SEQ ID NO: | TAACGGTTG (SEQ ID | TCTTATCTC (SEQ ID NO: |
| 850) | NO: 2484) | 3833 |
| TCACACCT (SEQ ID NO: | CAACCGTTA (SEQ ID | GAGATAAGG (SEQ ID NO: |
| 851) | NO: 2485) | 3834 |
| AGGTGTCA (SEQ ID NO: | TAACTGCCA (SEQ ID | CCTTATCTC (SEQ ID NO: |
| 852) | NO: 2486) | 3835 |
| TGACACCT (SEQ ID NO: | TGGCAGTTA (SEQ ID | GTGATAAGA (SEQ ID NO: |
| 853) | NO: 2487) | 3836 |
| GTCATCA (SEQ ID NO: | TAACTGCCC (SEQ ID | TCTTATCAC (SEQ ID NO: |
| 862) | NO: 2488) | 3837 |
| TGATGAC (SEQ ID NO: | GGGCAGTTA (SEQ ID | GTGATAAGG (SEQ ID NO: |
| 863) | NO: 2489) | 3838 |
| GTCATCG (SEQ ID NO | TAACTGCCG (SEQ ID | CCTTATCAC (SEQ ID NO: |
| 864) | NO: 2490) | 3839 |
| CGATGAC (SEQ ID NO | CGGCAGTTA (SEQ ID | TAGATAAGA (SEQ ID NO: |
| 865) | NO: 2491) | 3940 |
| TTTAACGAC (SEQ ID NO | TAACTGCTA (SEQ ID | TCTTATCTA (SEQ ID NO: |
| 866) | NO: 2492) | 3841 |
| GTCGTTAAA (SEQ ID NO | TAGCAGTTA (SEQ ID | TAGATAAGG (SEQ ID NO: |
| 867) | NO: 2493) | 3842 |
| TTTTACGAC (SEQ ID NO | TAACTGCTC (SEQ ID | CCTTATCTA (SEQ ID NO: |
| 868) | NO: 2494) | 3843 |
| GTCGTAAAA (SEQ ID | GAGCAGTTA (SEQ ID | TTGATAAGA (SEQ ID NO: |
| NO: 869) | NO: 2495) | 3844 |
| AGTCGTAAAA (SEQ ID | TAACTGCTG (SEQ ID | TCTTATCAA (SEQ ID NO: |
| NO: 870) | NO: 2496) | 3845 |
| TTTTACGACT (SEQ ID | CAGCAGTTA (SEQ ID | TTGATAAGG (SEQ ID NO: |
| NO: 871) | NO: 2497) | 3846 |
| GGTCGTAAAA (SEQ ID | TAACTGTCA (SEQ ID | CCTTATCAA (SEQ ID NO: |
| NO: 872) | NO: 2498) | 3847 |
| TTTTACGACC (SEQ ID | TGACAGTTA (SEQ ID | ACTTTCCAT (SEQ ID NO: |
| NO: 873) | NO: 2499) | 3940 |
| GGTGAGTCAT (SEQ ID | TAACTGTCC (SEQ ID | ATGGAAAGT (SEQ ID NO: |
| NO: 874) | NO: 2500) | 3941 |
| ATGACTCACC (SEQ ID | GGACAGTTA (SEQ ID | ACTTTCCGT (SEQ ID NO: |
| NO: 875) | NO: 2501) | 3942 |
| GGTGACTCAT (SEQ ID | TAACTGTCG (SEQ ID | ACGGAAAGT (SEQ ID NO: |
| NO: 876) | NO: 2502) | 3943 |
| ATGAGTCACC (SEQ ID | CGACAGTTA (SEQ ID | TCTTTCCAT (SEQ ID NO: |
| NO: 877) | NO: 2503) | 3944 |
| TGACGTCAC (SEQ ID | TAACTGTTA (SEQ ID | ATGGAAAGA (SEQ ID NO: |
| NO: 882) | NO: 2504) | 3945 |
| GTGACGTCA (SEQ ID | TAACAGTTA (SEQ ID | TCTTTCCGT (SEQ ID NO: |
| NO: 883 | NO: 2505 | 3946 |
| TGACGTCAT (SEQ ID | TAACTGTTC (SEQ ID | ACGGAAAGA (SEQ ID NO: |
| NO: 884 | NO: 2506 | 3947 |
| ATGACGTCA (SEQ ID | GAACAGTTA (SEQ ID | ATATAGGGG (SEQ ID NO: |
| NO: 885 | NO: 2507 | 3948 |
| TGGCGTCAC (SEQ ID | TAACTGTTG (SEQ ID | CCCCTATAT (SEQ ID NO: |
| NO: 886 | NO: 2508 | 3949 |
| GTGACGCCA (SEQ ID | CAACAGTTA (SEQ ID | CTATAGGGG (SEQ ID NO: |
| NO: 887 | NO: 2509 | 3950 |
| TGGCGTCAT (SEQ ID | TCAAGGTCAA (SEQ | CCCCTATAG (SEQ ID NO: |
| NO: 888 | ID NO: 2510 | 3951 |
| ATGACGCCA (SEQ ID | TTGACCTTGA (SEQ | GGTTTAT (SEQ ID NO: 3960 |
| NO: 889 | ID NO: 2511 | |
| TTACGTCAC (SEQ ID | TCAAGGTCAT (SEQ | ATAAACC (SEQ ID NO: 3961 |
| NO: 890 | ID NO: 2512 | |
| GTGACGTAA (SEQ ID | ATGACCTTGA (SEQ | GGTTTCT (SEQ ID NO: 3962 |
| NO: 891 | ID NO: 2513 | |
| TTACGTCAT (SEQ ID | GTCAGCA (SEQ ID | AGAAACC (SEQ ID NO: |
| NO: 892 | NO: 2534 | 3963 |
| ATGACGTAA (SEQ ID | TGCTGAC (SEQ ID | AGTAAACAA (SEQ ID NO: |
| NO: 893 | NO: 2535 | 3964 |
| TTGCGTCAC (SEQ ID | ACTCCAC (SEQ ID | TTGTTTACT (SEQ ID NO: |
| NO: 894 | NO: 2536 | 3965 |
| GTGACGCAA (SEQ ID | GTGGAGT (SEQ ID | AGTAAACAG (SEQ ID NO: |
| NO: 895 | NO: 2537 | 3966 |
| TTGCGTCAT (SEQ ID | ACTTCAC (SEQ ID | CTGTTTACT (SEQ ID NO: |
| NO: 896 | NO: 2538 | 3967 |
| ATGACGCAA (SEQ ID | GTGAAGT (SEQ ID | TGTAAACAA (SEQ ID NO: |
| NO: 897 | NO: 2539 | 3968 |
| ATGACGTCAC (SEQ ID | TCAAGTGG (SEQ ID | TTGTTTACA (SEQ ID NO: |
| NO: 922 | NO: 2542 | 3969 |
| GTGACGTCAT (SEQ ID | CCACTTGA (SEQ ID | TGTAAACAG (SEQ ID NO: |
| NO: 923 | NO: 2543 | 3970 |
| ATGACGTCAT (SEQ ID | TTAAGTGG (SEQ ID | CTGTTTACA (SEQ ID NO: |
| NO: 924 | NO: 2544 | 3971 |
| ATGACGTCAT (SEQ ID | CCACTTAA (SEQ ID | ACCGGAAGTG (SEQ ID NO: |
| NO: 925 | NO: 2545 | 3972 |
| CGTCTAGACA (SEQ ID | CCCCCCCAC (SEQ ID | CACTTCCGGT (SEQ ID NO: |
| NO: 936 | NO: 2682 | 3973 |
| TGTCTAGACG (SEQ ID | GTGGGGGGG (SEQ | GCCGGAAGTG (SEQ ID NO: |
| NO: 937 | ID NO: 2683 | 3974 |
| TGTCTAGACA (SEQ ID | GAGATAAG (SEQ ID | CACTTCCGGC (SEQ ID NO: |
| NO: 938 | NO: 2686 | 3975 |
| TGTCTAGACA (SEQ ID | CTTATCTC (SEQ ID | ACAGCTGC (SEQ ID NO: |
| NO: 939 | NO: 2687 | 3976 |
| GTCGTAAAA (SEQ ID | CAGATAAG (SEQ ID | GCAGCTGT (SEQ ID NO: |
| NO: 942 | NO: 2688 | 3982 |
| TTTTACGAC (SEQ ID | CTTATCTG (SEQ ID | ACAGCTGT (SEQ ID NO: |
| NO: 943 | NO: 2689 | 3978 |
| TGATTGACAG (SEQ ID | TAATGATTAA (SEQ | ACAGCTGT (SEQ ID NO: |
| NO: 1012 | ID NO: 2690 | 3978 |
| CTGTCAATCA (SEQ ID | TTAATCATTA (SEQ | GCAGCTGC (SEQ ID NO: |
| NO: 1013 | ID NO: 2691 | 3980 |
| GATGACGTCA (SEQ ID | TAATGATTAT (SEQ | GCAGCTGC (SEQ ID NO: |
| NO: 1014 | ID NO: 2692 | 3980 |
| TGACGTCATC (SEQ ID | ATAATCATTA (SEQ | GCAGCTGT (SEQ ID NO: |
| NO: 1015 | ID NO: 2693 | 3982 |
| CCACGTGG (SEQ ID NO: | TAATGATTTA (SEQ | ACAGCTGC (SEQ ID NO: |
| 1016 | ID NO: 2694 | 3983 |
| CCACGTGG (SEQ ID NO: | TAAATCATTA (SEQ | AGCAGGAAGT (SEQ ID NO: |
| 1017 | ID NO: 2695 | 3984 |
| TGACTCAGCA (SEQ ID | TAATGATTTT (SEQ | ACTTCCTGCT (SEQ ID NO: |
| NO: 1058 | ID NO: 2696 | 3985 |
| TGCTGAGTCA (SEQ ID | AAAATCATTA (SEQ | ACATCCTGGT (SEQ ID NO: |
| NO: 1059 | ID NO: 2697 | 3986 |
| CAGCTGCTG (SEQ ID | TAATGGTTAA (SEQ | ACCAGGATGT (SEQ ID NO: |
| NO: 1064 | ID NO: 2698 | 3987 |
| CAGCAGCTG (SEQ ID | TTAACCATTA (SEQ | AACAGGAAGT (SEQ ID NO: |
| NO: 1065 | ID NO: 2699 | 3988 |
| TGCTGAGTCA (SEQ ID | TAATGGTTAT (SEQ | ACTTCCTGTT (SEQ ID NO: |
| NO: 1066 | ID NO: 2700 | 3989 |
| TGACTCAGCA (SEQ ID | ATAACCATTA (SEQ | ACCAGGAAGT (SEQ ID NO: |
| NO: 1067 | ID NO: 2701 | 3990 |
| TGACGTCA (SEQ ID NO: | TAATGGTTTA (SEQ | ACTTCCTGGT (SEQ ID NO: |
| 1242 | ID NO: 2702 | 3991 |
| TGACGTCA (SEQ ID NO: | TAAACCATTA (SEQ | AGCAGGAAGT (SEQ ID NO: |
| 1243 | ID NO: 2703 | 3992 |
| TGCTGACTCA (SEQ ID | TAATGGTTTT (SEQ | ACTTCCTGCT (SEQ ID NO: |
| NO: 1314 | ID NO: 2704 | 3993 |
| TGAGTCAGCA (SEQ ID | AAAACCATTA (SEQ | GGGATATTCC (SEQ ID NO: |
| NO: 1315 | ID NO: 2705 | 3996 |
| GGTCGTAAAA (SEQ ID | TAATTATTAA (SEQ | GGAATATCCC (SEQ ID NO: |
| NO: 1324 | ID NO: 2706 | 3997 |
| TTTTACGACC (SEQ ID | TTAATAATTA (SEQ | GGGATTTTCC (SEQ ID NO: |
| NO: 1325 | ID NO: 2707 | 3998 |
| AAGGTCA (SEQ ID NO: | TAATTATTAT (SEQ | GGAAAATCCC (SEQ ID NO: |
| 1328 | ID NO: 2708 | 3999 |
| TGACCTT (SEQ ID NO: | ATAATAATTA (SEQ | GGGGTATTCC (SEQ ID NO: |
| 1329 | ID NO: 2709 | 4000 |
| AGGGTCA (SEQ ID NO: | TAATTATTTA (SEQ | GGAATACCCC (SEQ ID NO: |
| 1330 | ID NO: 2710 | 4001 |
| TGACCCT (SEQ ID NO: | TAAATAATTA (SEQ | GGGGTTTTCC (SEQ ID NO: |
| 1331 | ID NO: 2711 | 4002 |
| GAGGTCA (SEQ ID NO: | TAATTATTTT (SEQ | GGAAAACCCC(SEQ ID NO: |
| 1332 | ID NO: 2712 | 4003 |
| TGACCTC (SEQ ID NO: | AAAATAATTA (SEQ | CGGATATTCC (SEQ ID NO: |
| 1333 | ID NO: 2713 | 4004 |
| GGGGTCA (SEQ ID NO: | TAATTGTTAA (SEQ | GGAATATCCG (SEQ ID NO: |
| 1334 | ID NO: 2714 | 4005 |
| TGACCCC (SEQ ID NO: | TTAACAATTA (SEQ | CGGATTTTCC (SEQ ID NO: |
| 1335 | ID NO: 2715 | 4006 |
| ATGACGTCAC (SEQ ID | TAATTGTTAT (SEQ | GGAAAATCCG (SEQ ID NO: |
| NO: 1354 | ID NO: 2716 | 4007 |
| GTGACGTCAT (SEQ ID | ATAACAATTA (SEQ | CGGGTATTCC (SEQ ID NO: |
| NO: 1355 | ID NO: 2717 | 4008 |
| ATGACGTCAT (SEQ ID | TAATTGTTTA (SEQ | GGAATACCCG (SEQ ID NO: |
| NO: 1356 | ID NO: 2718 | 4009 |
| ATGACGTCAT (SEQ ID | TAAACAATTA (SEQ | CGGGTTTTCC (SEQ ID NO: |
| NO: 1357 | ID NO: 2719 | 4010 |
| GTGACGTCAC (SEQ ID | TAATTGTTTT (SEQ | GGAAAACCCG (SEQ ID NO: |
| NO: 1358 | ID NO: 2720 | 4011 |
| GTGACGTCAC (SEQ ID | AAAACAATTA (SEQ | AACCGGAAGT (SEQ ID NO: |
| NO: 1359 | ID NO: 2721 | 4224 |
| GTGACGTCAT (SEQ ID | ACATCAAAGG (SEQ | ACTTCCGGTT (SEQ ID NO: |
| NO: 1360 | ID NO: 2722 | 4225 |
| ATGACGTCAC (SEQ ID | CCTTTGATGT (SEQ | GCCCCGCCC (SEQ ID NO: |
| NO: 1361 | ID NO: 2723 | 4226 |
| GGCAGTTA (SEQ ID NO: | GGCTAGCCC (SEQ ID | GGGCGGGGC (SEQ ID NO: |
| 1364 | NO: 2724 | 4227 |
| TAACTGCC (SEQ ID NO: | GGGCTAGCC (SEQ ID | GCCCCTCCC (SEQ ID NO: |
| 1365 | NO: 2725 | 4228 |
| GGCAGTTG (SEQ ID NO: | TTCCAAGAA (SEQ ID | GGGAGGGGC (SEQ ID NO: |
| 1366 | NO: 2726 | 4229 |
| CAACTGCC (SEQ ID NO: | TTCTTGGAA (SEQ ID | CCCCCGCCC (SEQ ID NO: |
| 1367 | NO: 2727 | 4230 |
| GGCCGTTA (SEQ ID NO: | TTCCAGGAA (SEQ ID | GGGCGGGGG (SEQ ID NO: |
| 1368 | NO: 2728 | 4231 |
| TAACGGCC (SEQ ID NO: | TTCCTGGAA (SEQ ID | CCCCCTCCC (SEQ ID NO: |
| 1369 | NO: 2729 | 4232 |
| GGCCGTTG (SEQ ID NO: | TTCCCAGAA (SEQ ID | GGGAGGGGG (SEQ ID NO: |
| 1370 | NO: 2730 | 4233 |
| CAACGGCC (SEQ ID NO: | TTCTGGGAA (SEQ ID | ATGCGGGT (SEQ ID NO: |
| 1371 | NO: 2731 | 4234 |
| GGCGGTTA (SEQ ID NO: | TTCCCGGAA (SEQ ID | ACCCGCAT (SEQ ID NO: |
| 1372 | NO: 2732 | 4235 |
| TAACCGCC (SEQ ID NO: | TTCCGGGAA (SEQ ID | CCTTATCTC (SEQ ID NO: |
| 1373 | NO: 2733 | 4236 |
| GGCGGTTG (SEQ ID NO: | TTCTAAGAA (SEQ ID | GAGATAAGG (SEQ ID NO: |
| 1374 | NO: 2734 | 4237 |
| CAACCGCC (SEQ ID NO: | TTCTTAGAA (SEQ ID | CCTTATCTG (SEQ ID NO: |
| 1375 | NO: 2735 | 4238 |
| ACTGAAACCA (SEQ ID | TTCTAGGAA (SEQ ID | CAGATAAGG (SEQ ID NO: |
| NO: 1398 | NO: 2736 | 4239 |
| TGGTTTCAGT (SEQ ID | TTCCTAGAA (SEQ ID | CCTTATCTT (SEQ ID NO: |
| NO: 1399 | NO: 2737 | 4240 |
| CTAAGTGC (SEQ ID NO: | TTCTCAGAA (SEQ ID | AAGATAAGG (SEQ ID NO: |
| 1444 | NO: 2738 | 4241 |
| GCACTTAG (SEQ ID NO: | TTCTGAGAA (SEQ ID | TCTTATCTC (SEQ ID NO: |
| 1445 | NO: 2739 | 4242 |
| CTAATTGC (SEQ ID NO: | TTCTCGGAA (SEQ ID | GAGATAAGA (SEQ ID NO: |
| 1446 | NO: 2740 | 4243 |
| GCAATTAG (SEQ ID NO: | TTCCGAGAA (SEQ ID | TCTTATCTG (SEQ ID NO: |
| 1447 | NO: 2741 | 4244 |
| TTAAGTGC (SEQ ID NO: | GGGGGGGG (SEQ ID | CAGATAAGA (SEQ ID NO: |
| 1448 | NO: 2760 | 4245 |
| GCACTTAA (SEQ ID NO: | CCCCCCCC (SEQ ID | TCTTATCTT (SEQ ID NO: |
| 1449 | NO: 2761 | 4246 |
| TTAATTGC (SEQ ID NO: | AGCTGACAAC (SEQ | AAGATAAGA (SEQ ID NO: |
| 1450 | ID NO: 2762 | 4247 |
| GCAATTAA (SEQ ID NO: | GTTGTCAGCT (SEQ | ACCGGAAGTA (SEQ ID NO: |
| 1451 | ID NO: 2763 | 4284 |
| TGTCAGCT (SEQ ID NO: | AGCTGACAAG (SEQ | TACTTCCGGT (SEQ ID NO: |
| 1452 | ID NO: 2764 | 4285 |
| AGCTGACA (SEQ ID NO: | CTTGTCAGCT (SEQ | ACCGGAAGTG (SEQ ID NO: |
| 1453 | ID NO: 2765 | 4286 |
| TGTCAGTT (SEQ ID NO: | AGCTGACAAT (SEQ | CACTTCCGGT (SEQ ID NO: |
| 1454 | ID NO: 2766 | 4287 |
| AACTGACA (SEQ ID NO: | ATTGTCAGCT (SEQ | CCACGCCCAC (SEQ ID NO: |
| 1455 | ID NO: 2767 | 4292 |
| GCCCCACCCA (SEQ ID | AGCTGACACC (SEQ | GTGGGCGTGG (SEQ ID NO: |
| NO: 1456 | ID NO: 2768 | 4293 |
| TGGGTGGGGC (SEQ ID | GGTGTCAGCT (SEQ | CCACGCCCCC (SEQ ID NO: |
| NO: 1457 | ID NO: 2769 | 4294 |
| GCCCCACCCC (SEQ ID | AGCTGACACG (SEQ | GGGGGCGTGG (SEQ ID NO: |
| NO: 1458 | ID NO: 2770 | 4295 |
| GGGGTGGGGC (SEQ ID | CGTGTCAGCT (SEQ | ACGAGGAAGT (SEQ ID NO: |
| NO: 1459 | ID NO: 2771 | 4296 |
| GCCCCACCCT (SEQ ID | AGCTGACACT (SEQ | ACTTCCTCGT (SEQ ID NO: |
| NO: 1460 | ID NO: 2772 | 4297 |
| AGGGTGGGGC (SEQ ID | AGTGTCAGCT (SEQ | ACGCGGAAGT (SEQ ID NO: |
| NO: 1461 | ID NO: 2773 | 4298 |
| GCCCCGCCCA (SEQ ID | AGCTGACAGC (SEQ | ACTTCCGCGT (SEQ ID NO: |
| NO: 1462 | ID NO: 2774 | 4299 |
| TGGGCGGGGC (SEQ ID | GCTGTCAGCT (SEQ | ACCAGGAAGT (SEQ ID NO: |
| NO: 1463 | ID NO: 2775 | 4300 |
| GCCCCGCCCC (SEQ ID | AGCTGACAGG (SEQ | ACTTCCTGGT (SEQ ID NO: |
| NO: 1464 | ID NO: 2776 | 4301 |
| GGGGCGGGGC (SEQ ID | CCTGTCAGCT (SEQ | ACCCGGAAGT (SEQ ID NO: |
| NO: 1465 | ID NO: 2777 | 4302 |
| GCCCCGCCCT (SEQ ID | AGCTGACAGT (SEQ | ACTTCCGGGT (SEQ ID NO: |
| NO: 1466 | ID NO: 2778 | 4303 |
| AGGGCGGGGC (SEQ ID | ACTGTCAGCT (SEQ | AACCGGAAGT (SEQ ID NO: |
| NO: 1467 | ID NO: 2779 | 4304 |
| ATTCCCC (SEQ ID NO: | AGCTGTCAAC (SEQ | ACTTCCGGTT (SEQ ID NO: |
| 1468 | ID NO: 2780 | 4305 |
| GGGGAAT (SEQ ID NO: | GTTGACAGCT (SEQ | ACAAGGAAGT (SEQ ID NO: |
| 1469 | ID NO: 2781 | 4306 |
| TGGCAGTTGG (SEQ ID | AGCTGTCAAG (SEQ | ACTTCCTTGT (SEQ ID NO: |
| NO: 1470 | ID NO: 2782 | 4307 |
| CCAACTGCCA (SEQ ID | CTTGACAGCT (SEQ | ACCAGGAAGT (SEQ ID NO: |
| NO: 1471 | ID NO: 2783 | 4308 |
| CCAAGGTCA (SEQ ID | AGCTGTCAAT (SEQ | ACTTCCTGGT (SEQ ID NO: |
| NO: 1502 | ID NO: 2784 | 4309 |
| TGACCTTGG (SEQ ID | ATTGACAGCT (SEQ | ACGAGGAAGT (SEQ ID NO: |
| NO: 1503 | ID NO: 2785 | 4310 |
| AGAGGTCA (SEQ ID NO: | AGCTGTCACC (SEQ | ACTTCCTCGT (SEQ ID NO: |
| 1508 | ID NO: 2786 | 4311 |
| TGACCTCT (SEQ ID NO: | GGTGACAGCT (SEQ | TGACAG (SEQ ID NO: 4314 |
| 1509 | ID NO: 2787 | |
| AGGGGTCA (SEQ ID NO: | AGCTGTCACG (SEQ | CTGTCA (SEQ ID NO: 4315 |
| 1510 | ID NO: 2788 | |
| TGACCCCT (SEQ ID NO: | CGTGACAGCT (SEQ | GCCATCTGTT (SEQ ID NO: |
| 1511 | ID NO: 2789 | 4460 |
| TGATGAC (SEQ ID NO: | AGCTGTCACT (SEQ | AACAGATGGC (SEQ ID NO: |
| 1512 | ID NO: 2790 | 4461 |
| GTCATCA (SEQ ID NO: | AGTGACAGCT (SEQ | AAAGAGGTCA (SEQ ID NO: |
| 1513 | ID NO: 2791 | 4470 |
| AGATCAAAG (SEQ ID | AGCTGTCAGC (SEQ | TGACCTCTTT (SEQ ID NO: |
| NO: 1514 | ID NO: 2792 | 4471 |
| CTTTGATCT (SEQ ID NO: | GCTGACAGCT (SEQ | ATAGAGGTCA (SEQ ID NO: |
| 1515 | ID NO: 2793 | 4472 |
| ACATCAAAG (SEQ ID | AGCTGTCAGG (SEQ | TGACCTCTAT (SEQ ID NO: |
| NO: 1516 | ID NO: 2794 | 4473 |
| CTTTGATGT (SEQ ID NO: | CCTGACAGCT (SEQ | ATTTTCCATT (SEQ ID NO: |
| 1517 | ID NO: 2795 | 4474 |
| AACAGCTGAT (SEQ ID | AGCTGTCAGT (SEQ | AATGGAAAAT (SEQ ID NO: |
| NO: 1590 | ID NO: 2796 | 4475 |
| ATCAGCTGTT (SEQ ID | ACTGACAGCT (SEQ | AACAGGAAGT (SEQ ID NO: |
| NO: 1591 | ID NO: 2797 | 4476 |
| ATCAGCTGAT (SEQ ID | CGCTGACAAC (SEQ | ACTTCCTGTT (SEQ ID NO: |
| NO: 1592 | ID NO: 2798 | 4477 |
| ATCAGCTGAT (SEQ ID | GTTGTCAGCG (SEQ | CCACGCCCAC (SEQ ID NO: |
| NO: 1593 | ID NO: 2799 | 4478 |
| TTGACAAG (SEQ ID NO: | CGCTGACAAG (SEQ | GTGGGCGTGG (SEQ ID NO: |
| 1618 | ID NO: 2800 | 4479 |
| CTTGTCAA (SEQ ID NO: | CTTGTCAGCG (SEQ | CCACGCCCCC (SEQ ID NO: |
| 1619 | ID NO: 2801 | 4480 |
| TTGACATG (SEQ ID NO: | CGCTGACAAT (SEQ | GGGGGCGTGG (SEQ ID NO: |
| 1620 | ID NO: 2802 | 4821 |
| CATGTCAA (SEQ ID NO: | ATTGTCAGCG (SEQ | ACTTCCTGA (SEQ ID NO: |
| 1621 | ID NO: 2803 | 4822 |
| AAGTGG (SEQ ID NO: | CGCTGACACC (SEQ | TCAGGAAGT (SEQ ID NO: |
| 1626 | ID NO: 2804 | 4823 |
| CCACTT (SEQ ID NO: | GGTGTCAGCG (SEQ | ACTTCCTGC (SEQ ID NO: |
| 1627 | ID NO: 2805 | 4824 |
| AAGTGC (SEQ ID NO: | CGCTGACACG (SEQ | GCAGGAAGT (SEQ ID NO: |
| 1628 | ID NO: 2806 | 4825 |
| GCACTT (SEQ ID NO: | CGTGTCAGCG (SEQ | ACTTCCTGT (SEQ ID NO: |
| 1629 | ID NO: 2807 | 4826 |
| CTCCCG (SEQ ID NO: | CGCTGACACT (SEQ | ACAGGAAGT (SEQ ID NO: |
| 1642 | ID NO: 2808 | 4827 |
| CGGGAG (SEQ ID NO: | AGTGTCAGCG (SEQ | ATTTCCTGA (SEQ ID NO: |
| 1643 | ID NO: 2809 | 4828 |
| CTCCCC (SEQ ID NO: | CGCTGACAGC (SEQ | TCAGGAAAT (SEQ ID NO: |
| 1644 | ID NO: 2810 | 4829 |
| GGGGAG (SEQ ID NO: | GCTGTCAGCG (SEQ | ATTTCCTGC (SEQ ID NO: |
| 1645 | ID NO: 2811 | 4830 |
| TTTCTGC (SEQ ID NO: | CGCTGACAGG (SEQ | GCAGGAAAT (SEQ ID NO: |
| 1646 | ID NO: 2812 | 4831 |
| GCAGAAA (SEQ ID NO: | CCTGTCAGCG (SEQ | ATTTCCTGT (SEQ ID NO: |
| 1647 | ID NO: 2813 | 4832 |
| AACAGCTGTC (SEQ ID | CGCTGACAGT (SEQ | ACAGGAAAT (SEQ ID NO: |
| NO: 1730 | ID NO: 2814 | 4833 |
| GACAGCTGTT (SEQ ID | ACTGTCAGCG (SEQ | AATTTCCAC (SEQ ID NO: |
| NO: 1731 | ID NO: 2815 | 4874 |
| AGCAGCTGG (SEQ ID | CGCTGTCAAC (SEQ | GTGGAAATT (SEQ ID NO: |
| NO: 1738 | ID NO: 2816 | 4875 |
| CCAGCTGCT (SEQ ID | GTTGACAGCG (SEQ | AATTTCCAT (SEQ ID NO: |
| NO: 1739 | ID NO: 2817 | 4876 |
| AGCAGCTGC (SEQ ID | CGCTGTCAAG (SEQ | ATGGAAATT (SEQ ID NO: |
| NO: 1740 | ID NO: 2818 | 4877 |
| GCAGCTGCT (SEQ ID | CTTGACAGCG (SEQ | AATTTCCGC (SEQ ID NO: |
| NO: 1741 | ID NO: 2819 | 4878 |
| CCCACCCAAC (SEQ ID | CGCTGTCAAT (SEQ | GCGGAAATT (SEQ ID NO: |
| NO: 1750 | ID NO: 2820 | 4879 |
| GTTGGGTGGG (SEQ ID | ATTGACAGCG (SEQ | AATTTCCGT (SEQ ID NO: |
| NO: 1751 | ID NO: 2821 | 4880 |
| CCCTCCCAAC (SEQ ID | CGCTGTCACC (SEQ | ACGGAAATT (SEQ ID NO: |
| NO: 1752 | ID NO: 2822 | 4881 |
| GTTGGGAGGG (SEQ ID | GGTGACAGCG (SEQ | TATTTCCAC (SEQ ID NO: |
| NO: 1753 | ID NO: 2823 | 4882 |
| TCACCTCC (SEQ ID NO: | CGCTGTCACG (SEQ | GTGGAAATA (SEQ ID NO: |
| 1762 | ID NO: 2824 | 4883 |
| GGAGGTGA (SEQ ID NO: | CGTGACAGCG (SEQ | TATTTCCAT (SEQ ID NO: |
| 1763 | ID NO: 2825 | 4884 |
| AGATAA (SEQ ID NO: | CGCTGTCACT (SEQ | ATGGAAATA (SEQ ID NO: |
| 1814 | ID NO: 2826 | 4885 |
| TTATCT (SEQ ID NO: | AGTGACAGCG (SEQ | TATTTCCGC (SEQ ID NO: |
| 1815 | ID NO: 2827 | 4886 |
| TGATAA (SEQ ID NO: | CGCTGTCAGC (SEQ | GCGGAAATA (SEQ ID NO: |
| 1816 | ID NO: 2828 | 4887 |
| TTATCA (SEQ ID NO: | GCTGACAGCG (SEQ | TATTTCCGT (SEQ ID NO: |
| 1817 | ID NO: 2829 | 4888 |
| GTAAACAG (SEQ ID NO: | CGCTGTCAGG (SEQ | ACGGAAATA (SEQ ID NO: |
| 1890 | ID NO: 2830 | 4889 |
| CTGTTTAC (SEQ ID NO: | CCTGACAGCG (SEQ | ACTTCCGGC (SEQ ID NO: |
| 1891 | ID NO: 2831 | 4892 |
| GGTCATT (SEQ ID NO: | CGCTGTCAGT (SEQ | GCCGGAAGT (SEQ ID NO: |
| 1924 | ID NO: 2832 | 4893 |
| AATGACC (SEQ ID NO: | ACTGACAGCG (SEQ | ACTTCCGGT (SEQ ID NO: |
| 1925 | ID NO: 2833 | 4894 |
| CCCCGCCC (SEQ ID NO: | GGCTGACAAC (SEQ | ACCGGAAGT (SEQ ID NO: |
| 1926 | ID NO: 2834 | 4895 |
| GGGCGGGG (SEQ ID NO: | GTTGTCAGCC (SEQ | ATTTCCGGC (SEQ ID NO: |
| 1927 | ID NO: 2835 | 4896 |
| CCCCTCCC (SEQ ID NO: | GGCTGACAAG (SEQ | GCCGGAAAT (SEQ ID NO: |
| 1928 | ID NO: 2836 | 4897 |
| GGGAGGGG (SEQ ID NO: | CTTGTCAGCC (SEQ | ATTTCCGGT (SEQ ID NO: |
| 1929 | ID NO: 2837 | 4898 |
| ACCGTTA (SEQ ID NO: | GGCTGACAAT (SEQ | ACCGGAAAT (SEQ ID NO: |
| 1942 | ID NO: 2838 | 4899 |
| TAACGGT (SEQ ID NO: | ATTGTCAGCC (SEQ | GCTTCCGGC (SEQ ID NO: |
| 1943 | ID NO: 2839 | 4900 |
| TCCGTTA (SEQ ID NO: | GGCTGACACC (SEQ | GCCGGAAGC (SEQ ID NO: |
| 1944 | ID NO: 2840 | 4901 |
| TAACGGA (SEQ ID NO: | GGTGTCAGCC (SEQ | GCTTCCGGT (SEQ ID NO: |
| 1945 | ID NO: 2841 | 4902 |
| AACAGCTGA (SEQ ID | GGCTGACACG (SEQ | ACCGGAAGC (SEQ ID NO: |
| NO: 1948 | ID NO: 2842 | 4903 |
| TCAGCTGTT (SEQ ID | CGTGTCAGCC (SEQ | GTTTCCGGC (SEQ ID NO: |
| NO: 1949 | ID NO: 2843 | 4904 |
| AACAGCTGG (SEQ ID | GGCTGACACT (SEQ | GCCGGAAAC (SEQ ID NO: |
| NO: 1950 | ID NO: 2844 | 4905 |
| CCAGCTGTT (SEQ ID | AGTGTCAGCC (SEQ | GTTTCCGGT (SEQ ID NO: |
| NO: 1951 | ID NO: 2845 | 4906 |
| AACAGCTGT (SEQ ID | GGCTGACAGC (SEQ | ACCGGAAAC (SEQ ID NO: |
| NO: 1952 | ID NO: 2846 | 4907 |
| ACAGCTGTT (SEQ ID | GCTGTCAGCC (SEQ | ACTTCCGGGT (SEQ ID NO: |
| NO: 1953 | ID NO: 2847 | 4908 |
| ACCAGCTGA (SEQ ID | GGCTGACAGG (SEQ | ACCCGGAAGT (SEQ ID NO: |
| NO: 1954 | ID NO: 2848 | 4909 |
| TCAGCTGGT (SEQ ID | CCTGTCAGCC (SEQ | ACTTCCGGTT (SEQ ID NO: |
| NO: 1955 | ID NO: 2849 | 4910 |
| ACCAGCTGG (SEQ ID | GGCTGACAGT (SEQ | AACCGGAAGT (SEQ ID NO: |
| NO: 1956 | ID NO: 2850 | 4911 |
| CCAGCTGGT (SEQ ID | ACTGTCAGCC (SEQ | ACTTCCTGGT (SEQ ID NO: |
| NO: 1957 | ID NO: 2851 | 4920 |
| ACCAGCTGT (SEQ ID | GGCTGTCAAC (SEQ | ACCAGGAAGT (SEQ ID NO: |
| NO: 1958 | ID NO: 2852 | 4921 |
| ACAGCTGGT (SEQ ID | GTTGACAGCC (SEQ | ACTTCCTGTT (SEQ ID NO: |
| NO: 1959 | ID NO: 2853 | 4922 |
| ATCAGCTGA (SEQ ID | GGCTGTCAAG (SEQ | AACAGGAAGT (SEQ ID NO: |
| NO: 1960 | ID NO: 2854 | 4923 |
| TCAGCTGAT (SEQ ID | CTTGACAGCC (SEQ | AACTTCCGGC (SEQ ID NO: |
| NO: 1961 | ID NO: 2855 | 4924 |
| ATCAGCTGG (SEQ ID | GGCTGTCAAT (SEQ | GCCGGAAGTT (SEQ ID NO: |
| NO: 1962 | ID NO: 2856 | 4925 |
| CCAGCTGAT (SEQ ID | ATTGACAGCC(SEQ | AACTTCCGGT (SEQ ID NO: |
| NO: 1963 | ID NO: 2857 | 4926 |
| ATCAGCTGT (SEQ ID | GGCTGTCACC (SEQ | ACCGGAAGTT (SEQ ID NO: |
| NO: 1964 | ID NO: 2858 | 4927 |
| ACAGCTGAT (SEQ ID | GGTGACAGCC (SEQ | CACTTCCGGC (SEQ ID NO: |
| NO: 1965 | ID NO: 2859 | 4928 |
| CAGATAAGG (SEQ ID | GGCTGTCACG (SEQ | GCCGGAAGTG (SEQ ID NO: |
| NO: 1974 | ID NO: 2860 | 4929 |
| CCTTATCTG (SEQ ID NO: | CGTGACAGCC (SEQ | CACTTCCGGT (SEQ ID NO: |
| 1975 | ID NO: 2861 | 4930 |
| TGCCAGCC (SEQ ID NO: | GGCTGTCACT (SEQ | ACCGGAAGTG (SEQ ID NO: |
| 1984 | ID NO: 2862 | 4931 |
| GGCTGGCA (SEQ ID NO: | AGTGACAGCC (SEQ | TACTTCCGGC (SEQ ID NO: |
| 1985 | ID NO: 2863 | 4932 |
| TGCCAGCG (SEQ ID NO: | GGCTGTCAGC (SEQ | GCCGGAAGTA (SEQ ID NO: |
| 1986 | ID NO: 2864 | 4933 |
| CGCTGGCA (SEQ ID NO: | GCTGACAGCC (SEQ | TACTTCCGGT (SEQ ID NO: |
| 1987 | ID NO: 2865 | 4934 |
| TGCCAGCT (SEQ ID NO: | GGCTGTCAGG (SEQ | ACCGGAAGTA (SEQ ID NO: |
| 1988 | ID NO: 2866 | 4935 |
| AGCTGGCA (SEQ ID NO: | CCTGACAGCC (SEQ | GGAAATTCCC (SEQ ID NO: |
| 1989 | ID NO: 2867 | 4936 |
| GTCAGCA (SEQ ID NO: | GGCTGTCAGT (SEQ | GGGAATTTCC (SEQ ID NO: |
| 2010 | ID NO: 2868 | 4937 |
| TGCTGAC (SEQ ID NO: | ACTGACAGCC (SEQ | GTGACCTTGA (SEQ ID NO: |
| 2011 | ID NO: 2869 | 4938 |
| CGGTTTCAAA (SEQ ID | ATAAACA (SEQ ID | TCAAGGTCAC (SEQ ID NO: |
| NO: 2014 | NO: 2890 | 4939 |
| TTTGAAACCG (SEQ ID | TGTTTAT (SEQ ID | AACAGGAAAT (SEQ ID NO: |
| NO: 2015 | NO: 2891 | 4940 |
| AGATAAG (SEQ ID NO: | GTAAACA (SEQ ID | ATTTCCTGTT (SEQ ID NO: |
| 2018 | NO: 2892 | 4941 |
| CTTATCT (SEQ ID NO: | TGTTTAC (SEQ ID | AAACCACAAA (SEQ ID NO: |
| 2019 | NO: 2893 | 4942 |
| TGATAAG (SEQ ID NO: | GGTCACGTGA (SEQ | TTTGTGGTTT (SEQ ID NO: |
| 2020 | ID NO: 3036 | 4977 |
| CTTATCA (SEQ ID NO: | TCACGTGACC (SEQ | ATTTCCTGT (SEQ ID NO: |
| 2021 | ID NO: 3037 | 4978 |
| TTCCAGAGAA (SEQ ID | TTTCCGGTAA (SEQ | ACAGGAAAT (SEQ ID NO: |
| NO: 2022 | ID NO: 3040 | 4979 |
| TTCTCTGGAA (SEQ ID | TTACCGGAAA (SEQ | ACCGGAAGTG (SEQ ID NO: |
| NO: 2023 | ID NO: 3041 | 4980 |
| TTCCACAGAA (SEQ ID | AACATATGGC (SEQ | CACTTCCGGT (SEQ ID NO: |
| NO: 2024 | ID NO: 3170 | 4981 |
| TTCTGTGGAA (SEQ ID | GCCATATGTT (SEQ | CAAACCACAG (SEQ ID NO: |
| NO: 2025 | ID NO: 3171 | 4982 |
| TTCCTGAGAA (SEQ ID | AACATATGGT (SEQ | CTGTGGTTTG (SEQ ID NO: |
| NO: 2026 | ID NO: 3172 | 5011 |
| TTCTCAGGAA (SEQ ID | ACCATATGTT (SEQ | AACCACAA (SEQ ID NO: |
| NO: 2027 | ID NO: 3173 | 5012 |
| TTCCTCAGAA (SEQ ID | ACCATATGGC (SEQ | TTGTGGTT (SEQ ID NO: |
| NO: 2028 | ID NO: 3174 | 5013 |
| TTCTGAGGAA (SEQ ID | GCCATATGGT (SEQ | AACCGCAA (SEQ ID NO: |
| NO: 2029 | ID NO: 3175 | 5014 |
| ACCGGAAGT (SEQ ID | ACCATATGGT (SEQ | TTGCGGTT (SEQ ID NO: |
| NO: 2030 | ID NO: 3176 | 5015 |
| ACTTCCGGT (SEQ ID | ACCATATGGT (SEQ | AAACCACAA (SEQ ID NO: |
| NO: 2031 | ID NO: 3177 | 5016 |
| GCCAATGA (SEQ ID NO: | CCACACCCAC (SEQ | TTGTGGTTT (SEQ ID NO: |
| 2068 | ID NO: 3178 | 5017 |
| TCATTGGC (SEQ ID NO: | GTGGGTGTGG (SEQ | AAACCACAG (SEQ ID NO: |
| 2069 | ID NO: 3179 | 5018 |
| GCCAATGG (SEQ ID NO: | ATCACGTGAC (SEQ | CTGTGGTTT (SEQ ID NO: |
| 2070 | ID NO: 3188 | 5019 |
| CCATTGGC (SEQ ID NO: | GTCACGTGAT (SEQ | AAACCACAT (SEQ ID NO: |
| 2071 | ID NO: 3189 | 5020 |
| GCCAATCA (SEQ ID NO: | ATCACGTGAT (SEQ | ATGTGGTTT (SEQ ID NO: |
| 2072 | ID NO: 3190 | 5021 |
| TGATTGGC (SEQ ID NO: | ATCACGTGAT (SEQ | TAACCACAA (SEQ ID NO: |
| 2073 | ID NO: 3191 | 5022 |
| GCCAATCG (SEQ ID NO: | GTCACGTGAC (SEQ | TTGTGGTTA (SEQ ID NO: |
| 2074 | ID NO: 3192 | 5023 |
| CGATTGGC (SEQ ID NO: | GTCACGTGAC (SEQ | TAACCACAG (SEQ ID NO: |
| 2075 | ID NO: 3193 | 5024 |
| CCTTATCTC (SEQ ID NO: | GTCACGTGAT (SEQ | CTGTGGTTA (SEQ ID NO: |
| 2276 | ID NO: 3259 | 5025 |
| GAGATAAGG (SEQ ID | ATCACGTGAC (SEQ | TAACCACAT (SEQ ID NO: |
| NO: 2277 | ID NO: 3258 | 5026 |
| CAACTG (SEQ ID NO: | ACCGGAAGT (SEQ ID | ATGTGGTTA (SEQ ID NO: |
| 2290 | NO: 3342 | 5027 |
| CAGTTG (SEQ ID NO: | ACTTCCGGT (SEQ ID | AACCGGAAGT (SEQ ID NO: |
| 2291 | NO: 3203 | 5028 |
| AAACCGCCA (SEQ ID | AACAGCTGC (SEQ ID | ACTTCCGGTT (SEQ ID NO: |
| NO: 2294 | NO: 3204 | 5029 |
| TGGCGGTTT (SEQ ID | GCAGCTGTT (SEQ ID | AAGGTCA (SEQ ID NO: 5030 |
| NO: 2295 | NO: 3205 | |
| AAACCGCCC (SEQ ID | AACAGCTGG (SEQ ID | TGACCTT (SEQ ID NO: 5031 |
| NO: 2296 | NO: 3206 | |
| GGGCGGTTT (SEQ ID | CCAGCTGTT (SEQ ID | AAAGGTCA (SEQ ID NO: |
| NO: 2297 | NO: 3207 | 5032 |
| AAACCGCCG (SEQ ID | AACAGCTGT (SEQ ID | TGACCTTT (SEQ ID NO: |
| NO: 2298 | NO: 3208 | 5033 |
| CGGCGGTTT (SEQ ID | ACAGCTGTT (SEQ ID | AAACCACAA (SEQ ID NO: |
| NO: 2299 | NO: 3209 | 5034 |
| AAACCGCTA (SEQ ID | ACCAGCTGC (SEQ ID | TTGTGGTTT (SEQ ID NO: |
| NO: 2300 | NO: 3210 | 5035 |
| TAGCGGTTT (SEQ ID | GCAGCTGGT (SEQ ID | AAACCACAG (SEQ ID NO: |
| NO: 2301 | NO: 3211 | 5036 |
| AAACCGCTC (SEQ ID | ACCAGCTGG (SEQ ID | CTGTGGTTT (SEQ ID NO: |
| NO: 2302 | NO: 3212 | 5037 |
| GAGCGGTTT (SEQ ID | CCAGCTGGT (SEQ ID | TAACCACAA (SEQ ID NO: |
| NO: 2303 | NO: 3213 | 5038 |
| AAACCGCTG (SEQ ID | ACCAGCTGT (SEQ ID | TTGTGGTTA (SEQ ID NO: |
| NO: 2304 | NO: 3214 | 5039 |
| CAGCGGTTT (SEQ ID | ACAGCTGGT (SEQ ID | TAACCACAG (SEQ ID NO: |
| NO: 2305 | NO: 3215 | 5040 |
| AAACCGTCA (SEQ ID | AGCAGCTGC (SEQ ID | CTGTGGTTA (SEQ ID NO: |
| NO: 2306 | NO: 3216 | 5041 |
| TGACGGTTT (SEQ ID | GCAGCTGCT (SEQ ID | ACAGGAAGTG (SEQ ID NO: |
| NO: 2307 | NO: 3217 | 5042 |
| AAACCGTCC (SEQ ID | AGCAGCTGG (SEQ ID | CACTTCCTGT (SEQ ID NO: |
| NO: 2308 | NO: 3218 | 5043 |
| GGACGGTTT (SEQ ID | CCAGCTGCT (SEQ ID | ACTTCCGGA (SEQ ID NO: |
| NO: 2309 | NO: 3219 | 5044 |
| AAACCGTCG (SEQ ID | AGCAGCTGT (SEQ ID | TCCGGAAGT (SEQ ID NO: |
| NO: 2310 | NO: 3220 | 5045 |
| CGACGGTTT (SEQ ID | ACAGCTGCT (SEQ ID | ACTTCCGGC (SEQ ID NO: |
| NO: 2311 | NO: 3221 | 5046 |
| AAACCGTTA (SEQ ID | CACAGCTGC (SEQ ID | GCCGGAAGT (SEQ ID NO: |
| NO: 2312 | NO: 3222 | 5047 |
| TAACGGTTT (SEQ ID | GCAGCTGTG (SEQ ID | ACTTCCGGT (SEQ ID NO: |
| NO: 2313 | NO: 3223 | 5048 |
| AAACCGTTC (SEQ ID | CACAGCTGG (SEQ ID | ACCGGAAGT (SEQ ID NO: |
| NO: 2314 | NO: 2224 | 5049 |
| GAACGGTTT (SEQ ID | CCAGCTGTG (SEQ ID | ATTTCCGGA (SEQ ID NO: |
| NO: 2315 | NO: 3225 | 5050 |
| AAACCGTTG (SEQ ID | CACAGCTGT (SEQ ID | TCCGGAAAT (SEQ ID NO: |
| NO: 2316 | NO: 3226 | 5051 |
| CAACGGTTT (SEQ ID | ACAGCTGTG (SEQ ID | ATTTCCGGC (SEQ ID NO: |
| NO: 2317 | NO: 3227 | 5052 |
| AAACGGCCA (SEQ ID | CCCAGCTGC (SEQ ID | GCCGGAAAT (SEQ ID NO: |
| NO: 2318 | NO: 3228 | 5053 |
| TGGCCGTTT (SEQ ID | GCAGCTGGG (SEQ ID | ATTTCCGGT (SEQ ID NO: |
| NO: 2319 | NO: 3229 | 5054 |
| AAACGGCCC (SEQ ID | CCCAGCTGG (SEQ ID | ACCGGAAAT (SEQ ID NO: |
| NO: 2320 | NO: 3230 | 5055 |
| GGGCCGTTT (SEQ ID | CCAGCTGGG (SEQ ID | GCTTCCGGA (SEQ ID NO: |
| NO: 2321 | NO: 3231 | 5056 |
| AAACGGCCG (SEQ ID | CCCAGCTGT (SEQ ID | TCCGGAAGC (SEQ ID NO: |
| NO: 2322 | NO: 3232 | 5057 |
| CGGCCGTTT (SEQ ID | ACAGCTGGG (SEQ ID | GCTTCCGGC (SEQ ID NO: |
| NO: 2323 | NO: 3233 | 5058 |
| AAACGGCTA (SEQ ID | CGCAGCTGC (SEQ ID | GCCGGAAGC (SEQ ID NO: |
| NO: 2324 | NO: 3234 | 5059 |
| TAGCCGTTT (SEQ ID | GCAGCTGCG (SEQ ID | GCTTCCGGT (SEQ ID NO: |
| NO: 2325 | NO: 3235 | 5060 |
| AAACGGCTC (SEQ ID | CGCAGCTGG (SEQ ID | ACCGGAAGC (SEQ ID NO: |
| NO: 2326 | NO: 3236 | 5061 |
| GAGCCGTTT (SEQ ID | CCAGCTGCG (SEQ ID | GTTTCCGGA (SEQ ID NO: |
| NO: 2327 | NO: 3237 | 5062 |
| AAACGGCTG (SEQ ID | CGCAGCTGT (SEQ ID | TCCGGAAAC (SEQ ID NO: |
| NO: 2328 | NO: 3238 | 5063 |
| CAGCCGTTT (SEQ ID | ACAGCTGCG (SEQ ID | GTTTCCGGC (SEQ ID NO: |
| NO: 2329 | NO: 3239 | 5064 |
| AAACGGTCA (SEQ ID | GACAGCTGC (SEQ ID | GCCGGAAAC (SEQ ID NO: |
| NO: 2330 | NO: 3240 | 5065 |
| TGACCGTTT (SEQ ID | GCAGCTGTC (SEQ ID | GTTTCCGGT (SEQ ID NO: |
| NO: 2331 | NO: 3241 | 5066 |
| AAACGGTCC (SEQ ID | GACAGCTGG (SEQ ID | ACCGGAAAC (SEQ ID NO: |
| NO: 2332 | NO: 3242 | 5067 |
| GGACCGTTT (SEQ ID | CCAGCTGTC (SEQ ID | AGAGGAAGTG (SEQ ID NO: |
| NO: 2333 | NO: 3243 | 5068 |
| AAACGGTCG (SEQ ID | GACAGCTGT (SEQ ID | CACTTCCTCT (SEQ ID NO: |
| NO: 2334 | NO: 3244 | 5069 |
| CGACCGTTT (SEQ ID | ACAGCTGTC (SEQ ID | CCGCCCACGC (SEQ ID NO: |
| NO: 2335 | NO: 3245 | 5070 |
| AAACGGTTA (SEQ ID | GCCAGCTGC (SEQ ID | GCGTGGGCGG (SEQ ID NO: |
| NO: 2336 | NO: 3246 | 5071 |
| TAACCGTTT (SEQ ID | GCAGCTGGC (SEQ ID | CCGCCCCCGC (SEQ ID NO: |
| NO: 2337 | NO: 3247 | 5072 |
| AAACGGTTC (SEQ ID | GCCAGCTGG (SEQ ID | GCGGGGGCGG (SEQ ID NO: |
| NO: 2338 | NO: 3248 | 5073 |
| GAACCGTTT (SEQ ID | CCAGCTGGC (SEQ ID | GCTGTGGTTT (SEQ ID NO: |
| NO: 2339 | NO: 3249 | 5074 |
| AAACGGTTG (SEQ ID | GCCAGCTGT (SEQ ID | AAACCACAGC (SEQ ID NO: |
| NO: 2340 | NO: 3250 | 5075 |
| CAACCGTTT (SEQ ID | ACAGCTGGC (SEQ ID | AACCGGAAGT (SEQ ID NO: |
| NO: 2341 | NO: 3251 | 5076 |
| AAACTGCCA (SEQ ID | GGCAGCTGC (SEQ ID | ACTTCCGGTT (SEQ ID NO: |
| NO: 2342 | NO: 3252 | 5077 |
| TGGCAGTTT (SEQ ID | GCAGCTGCC (SEQ ID | ACAGGAAGTG (SEQ ID NO: |
| NO: 2343 | NO: 3253 | 5078 |
| AAACTGCCC (SEQ ID | GGCAGCTGG (SEQ ID | CACTTCCTGT (SEQ ID NO: |
| NO: 2344 | NO: 3254 | 5079 |
| GGGCAGTTT (SEQ ID | CCAGCTGCC (SEQ ID | CGCCCCCTC (SEQ ID NO: |
| NO: 2345 | NO: 3255 | 5128 |
| AAACTGCCG (SEQ ID | GGCAGCTGT (SEQ ID | GAGGGGGCG (SEQ ID NO: |
| NO: 2346 | NO: 3256 | 5129 |
| CGGCAGTTT (SEQ ID | ACAGCTGCC (SEQ ID | CTCCCCCTC (SEQ ID NO: |
| NO: 2347 | NO: 3257 | 5130 |
| AAACTGCTA (SEQ ID | ATCACGTGAC (SEQ | GAGGGGGAG (SEQ ID NO: |
| NO: 2348 | ID NO: 3258 | 5131 |
| TAGCAGTTT (SEQ ID | GTCACGTGAT (SEQ | CGCCCACGC (SEQ ID NO: |
| NO: 2349 | ID NO: 3259 | 5136 |
| AAACTGCTC (SEQ ID | ATCACGTGAT (SEQ | GCGTGGGCG (SEQ ID NO: |
| NO: 2350 | ID NO: 3260 | 5137 |
| GAGCAGTTT (SEQ ID | ATCACGTGAT (SEQ | CGCCCCCGC (SEQ ID NO: |
| NO: 2351 | ID NO: 3260 | 5138 |
| AAACTGCTG (SEQ ID | TAATTA (SEQ ID NO: | GCGGGGGCG (SEQ ID NO: |
| NO: 2352 | 3262 | 5139 |
| CAGCAGTTT (SEQ ID | TAATTA (SEQ ID NO: | TGCGTGGGCG (SEQ ID NO: |
| NO: 2353 | 3263 | 5140 |
| AAACTGTCA (SEQ ID | TAATTG (SEQ ID NO: | CGCCCACGCA (SEQ ID NO: |
| NO: 2354 | 3264 | 5141 |
| TGACAGTTT (SEQ ID | CAATTA (SEQ ID NO: | TGCGTGGGTG (SEQ ID NO: |
| NO: 2355 | 3265 | 5142 |
| AAACTGTCC (SEQ ID | TTATAT (SEQ ID NO: | CACCCACGCA (SEQ ID NO: |
| NO: 2356 | 3340 | 5143 |
| GGACAGTTT (SEQ ID | ATATAA (SEQ ID NO: | |
| NO: 2357 | 3341 | |
| AAACTGTCG (SEQ ID | ACCGGAAGT (SEQ ID | |
| NO: 2358 | NO: 3342 | |
| CGACAGTTT (SEQ ID | | |
| NO: 2359 | | |

In some embodiments, the transcription initiator or transcription regulator may comprise a sequence including any sequence of SEQ ID NO: 175-9800 or a sequence with at least about 70% sequence identity thereto. For example, the transcription initiator or transcription regulator may comprise a sequence with at least about 75% sequence identity, at least about 80% sequence identity, at least about 85% sequence identity, at least about 90% sequence identity, at least 95% sequence identity, at least about 96% sequence identity, at least about 97% sequence identity, at least about 98% sequence identity, at least about 99% sequence identity, or at least 100% sequence identity to any sequence of SEQ ID NO: 175-9800.

### Cell State Sensing Constructs and Expression of Engineered Receptors

In some embodiments, the cell state sensing constructs may drive expression of an engineered antigen receptor including T cell Receptors (“TCRs”), Chimeric antigen receptors (“CARs” including all generations), B cell Receptors (“BCRs”), co-receptors of a TCR or BCR and/or any combination or hybrid thereof. In some embodiments, engineered receptors may signal or otherwise (including a dominant negative antigen sink). In some embodiments, antigen receptors may include one or more of: a DHD and/or binding site for a degrader, a binding site for an effector protein of a LOCKR switch, or a binding site for a direct or indirect signaling transducer of a safety switch. In some embodiments, antigen receptors may comprise a fully-human sequence, a humanized sequence, a sequence isolated or derived from a mammalian sequence or a non-human sequence (including a mouse sequence), a syngeneic sequence, a chimeric sequence, a recombinant sequence or a sequence derived from any species.

One example of an antigen receptor may include a chimeric antigen receptor (CAR), as seen in FIG. 1A. In some embodiments, a CAR may comprise minimally, an extracellular domain that contains a binder that specifically and selectively binds a target antigen, a transmembrane domain and an intracellular domain that contains at least one signaling domain. In some embodiments, one or more sequences of the CAR may be naturally occurring or non-naturally occurring. In some embodiments, one or more sequences of the CAR may be endogenous or exogenous to the cell expressing it. In some embodiments, one or more sequences of the CAR may be a fully-human sequence, a humanized sequence, a sequence isolated or derived from a mammalian sequence or a non-human sequence (including a mouse sequence), and/or a syngeneic sequence. In some embodiments, the CARs may be specific for one or more target antigens. For example, some CARs may have one or more binders, wherein each binder is specific for a distinct epitope on the same target antigen. In some embodiments, CARs contain a spacer to attain an optimal distance of the immune synapse. The extracellular domain of the CAR may comprise the spacer, which is positioned between the T-cell surface and a binder. If there is more than one binder, the spacer may be positioned between the T-cell surface and a first binder. In some embodiments, binders may have any structure, including but not limited to: (1) an antibody or any fragment thereof; (2) an antibody mimetic or any portion thereof; and/or (3) a de novo designed protein. Exemplary binders may include, but are not limited to: scFv, VH, VHH, monoclonal antibody and any combination thereof. In some embodiments, transmembrane domains may be isolated or derived from any protein, including any transmembrane protein. Exemplary transmembrane domains may comprise a sequence isolated or derived from a CD4 or a CD8 protein. In some embodiments, CARs may be of any CAR “generation” including, but not limited to, Gen I, Gen II, Gen III, Gen IV, and Gen V. In some embodiments, intracellular domains of the CAR may contain one or more of immunoreceptor tyrosine-based activation motif (ITAM) repeats (e.g., a single CD3ζ intracellular domain) (Gen I), ITAM repeats and one co-stimulatory molecule (e.g., CD27 or CD137 sequence) (Gen II), ITAM repeats, a first co-stimulatory molecule and a second co-stimulatory molecule (e.g., CD134 or CD137) (Gen III), ITAM repeats, a co-stimulatory molecule and a constitutively or inducibly expressed chemokine (e.g. WT IL-12) (Gen IV; also referred to as a T cell redirected for universal cytokine-mediated killing (TRUCK)), and/or ITAM repeats, a co-stimulatory molecule and a intracellular domain of cytokine receptors (e.g. WT IL-2Rβ chain fragment) (Gen V).

In some embodiments, CARs can signal or otherwise (a dominant negative antigen sink). CARs may include a DHD and/or binding site for a degrader, a binding site for an effector protein of a LOCKR switch, a logic gate or switch, or a binding site for a direct or indirect signaling transducer of a safety switch. In some embodiments, CARs may be split CARs in which a final activity depends upon the binding and interaction of two domains or two receptors.

To avoid CAR T cell exhaustion, it would be beneficial to have a system including one or more exemplary constructs that may sense exhaustion and shut down stimulation of the CAR. Disclosed herein are one or more exemplary engineered (non-naturally occurring) constructs and methods of use that may be used to shut down stimulation antigen receptors including chimeric antigen receptors in response to sensing the cell state including exhaustion.

### Cell State Sensing Constructs and Expression of Fusion Proteins

In some embodiments, the cell state sensing constructs may drive expression of a fusion protein having and engineered receptor (e.g. CAR or TCR) and degradation domain. For example, in some embodiments, the polypeptide may be fused to a degradation domain for targeted degradation of a molecule of interest. In some embodiments, the degradation domain comprises a degron. As provided herein, a degron is a minimal element of a protein that is sufficient for cell-mediated degradation. In some embodiments, the cell-mediated degradation is dependent on ubiquitin. In some embodiments, the cell-mediated degradation is not dependent on ubiquitin.

In some embodiments, the one or more constructs may include a polynucleotide set that includes a polynucleotide encoding a degradation initiator fused to a binding element that is specific for a native motif on an endogenous molecule of interest. The binding domain may, for example, recognize and bind a specific region on the molecule of interest or a specific modification on the molecule of interest. Interaction of the binding element with the native motif on the molecule recruits the degradation initiator to the molecule to initiate degradation.

In some embodiments, the degradation domain comprises a ligase or ligase-binding domain. In some embodiments, the polypeptides of the one or more constructs may be used as binding elements, wherein the polypeptides are fused to a ligase domain (or functional variant thereof). Binding of the polypeptides of the one or more constructs to the protein of interest brings the modified ligase into proximity with the targeted protein, thereby facilitating ubiquitination and subsequent degradation of the protein target.

In some embodiments, the ligase of the one or more constructs may include an E3 ligase. E3 ligases control substrate specificity and the topology of ubiquitination. For example, in a cellular ubiquitin proteasome system, an E3 ligase protein recruits an E2 ubiquitin-conjugating enzyme that has been loaded with ubiquitin, recognizes a protein substrate, and assists or directly catalyzes the transfer of ubiquitin from the E2 to the protein substrate, thereby targeting the protein for degradation. An E3 ligase domain and/or a variant thereof may be selected based on the intended protein targeted for degradation. For example, an E3 ligase and/or a set of E3 ligases may be selected for modulating the stability and/or half-life of a targeted transmembrane protein. In another example, an E3 ligase and/or a set of E3 ligases may be selected for modulating the stability and/or half-life of a cytoplasmic protein (e.g., a transcription factor). In some embodiments, the one or more constructs may include one or more synthetic degraders that may be useful for regulating the activity of a range of target molecules (e.g., proteins) in various synthetic biology applications, such as therapeutic applications. In one embodiment, the one or more synthetic degraders may be used in a cell therapy application to program a population of cells for performing and/or modulating a therapeutic function.

In some embodiments, the one or more synthetic degraders may be used to regulate (degrade) T cell receptors (TCRs) and/or chimeric antigen receptors (CARs) cells used in cancer therapy. More specifically, the synthetic degrader system of the disclosure may be used to degrade CARs and/or TCRs and inhibit excessive CAR/TCR signaling that may result in undesirable exhaustion phenotypes observed in many existing immune cell therapies.

### Cell State Sensing Constructs and Expression of Cytokines

In some embodiments, the cell state sensing constructs may drive expression of one or more cytokines. For example, in some embodiments, the transcription initiator and/or transcription regulator may be operably linked to one or more cytokines. The one or more cytokines may include IL-2, IL-12, IL-15, IL-18, IL-21, IL-23, Interferon alpha (α), Interferon beta (β), Interferon gamma (γ), and Interferon omega (ω).

### Exemplary Constructs Comprising Circuit Components

The constructs of the disclosure are non-naturally occurring and comprise one or more circuits that together regulate the expression of selected proteins of interest (referred to below as the effector component). In some embodiments, the circuits may comprise a response component (e.g., any one of the transcription initiator/transcription regulator/EP pair/promoter/enhancers provided above) and a effector component. The response component is capable of modifying an activity of the effector component. The response component is capable of modifying the activity of the effector component in response to a signal from a receptor including an antigen receptor, in response to an epigenetic change, in response to an induction or suppression of transcription, or in response to a change in cell state. In some embodiments, the activity of the response component is capable of inducing a change in an activity of the effector component. For example, the response component may include a transcription initiator that comprises a concatemer that includes one or more sequences from an untranslated region of a transcription factor, the transcription initiator being operably linked to a CAR and the effector component may include the CAR. The transcription initiator, the response component, is capable of modifying the activity of the CAR, the effector component, and the activity of the transcription initiator is capable of inducing a change in the activity of the CAR through increasing transcription of the CAR. In another example, the response component may include a constitutive on transcription initiator operably linked to a CAR and the effector component may include an inducible transcription initiator operably linked to a degrader. Antigen stimulation may lead to an activated response through the CAR, response component, wherein endogenous signaling networks may induce expression of the degrader, the effector component, thereby lowering expression of the CAR. It can be appreciated that any of the constructs in the disclosure may be configured to encode circuit components and are considered. For example, the response component may comprise one or more of a sequence of an untranslated region of a transcription factor, a sequence comprising a binding site for a transcription factor, a promoter, an enhancer, and a suppresser. In some embodiments, the response element may comprise an epigenetic modifier, a promoter including a minimal promoter. In some embodiments, the minimal promoter may comprise a sequence of any one or more sequences of SEQ ID NO: 9788, 9789, or 9790. In some embodiments, the response component may comprise a transcription initiator including a concatemerized promoter comprising a sequence of any one or more sequences of SEQ ID NOS: 175-9781. In some embodiments, the response component may comprise a transcription initiator and a transcription regulator including an enhancer-promoter pair. In some embodiments, the response component may comprise a sequence of any one or more sequences of SEQ ID NO: 1-174. In some embodiments, the effector component may comprise a degrader. In some embodiments, the effector component may include a degrader operably linked to a transcription initiator comprising a sequence of any one or more of SEQ ID NOS: 1-9781. In some embodiments, the signal from a receptor comprises a signal from a CAR. In some embodiments, the cell state or the change in cell state may comprise any cell state including unstimulated, stimulation on, stimulation off, exhaustion, or rest.

### Inducible CAR Systems

In some embodiments, the circuits are inducible receptor systems, e.g., a inducible CAR system. Within the one or more constructs of the inducible CAR systems, the components of response elements are combined to regulate transcription of one or more genes (e.g., the CAR, and a degrader). For example, response elements may be operable linked to one or more genes of the construct, wherein the one or more genes may include a CAR and/or a degrader, and the response elements regulate transcription of either the CAR or the degrader, or both.

Table 4 lists four different constructs of a inducible CAR system and the expression behavior generated by the four different CAR constructs.

TABLE 4
CAR constructs of Inducible CAR System

| CAR Constructs | Behavior | Degrader |
|---|---|---|
| CAR-1 | Constitutive | No |
| CAR-1D | Constitutive | Yes |
| CAR-2 | Stimulation On | No |
| CAR-2D | Stimulation On | Yes |

For example, a first CAR (“CAR-1”) may have a transcription initiator that demonstrates “constitutive on behavior”, wherein the transcription initiator drives CAR after antigen stimulation. In the absence of any dynamic regulation of the CAR, CAR Expression may remain at a consistent level over time. However, the consistent CAR expression in the presence of consistent antigen stimulation may lead to an exhausted cell state. A degrader may be added to the CAR-1 construct (“CAR-1D”), wherein expression of the degrader reduces or pulses CAR expression, allowing the cell to transition to a rested cell state. After the rested cell state, the re-activation of the CAR with antigen stimulation may lead to an increase in CAR expression. The CAR-1D construct may cycle through multiple pulses of CAR expression over time.

The presence of the degrader may allow the CAR expression to be reduced to a variety of levels including almost entirely, to baseline, a slight reduction in comparison to peak CAR expression levels, or anywhere in between.

A second CAR construct (“CAR-2”) may have a promoter that demonstrates a stimulation on behavior wherein stimulation of the CAR drives an increase of expression of the CAR through the regulated promoter. The promoter of CAR-2 may drive transcription at a higher level than the promoter of CAR-1, leading to a marked increase in overall CAR transcription levels. In the absence of any regulation of the CAR, CAR expression may also remain at a consistent level over time without a reduction to baseline levels. The introduction of a degrader into the inducible receptor system construct (“CAR-2D”) may lead to the CAR expression pulsing over time. The addition of the degrader to CAR-2 may lead to a decrease in CAR expression to about baseline levels.

The level of pulsing (e.g., the change in CAR expression from a maximal CAR expression value to a minimal CAR expression value) and the rate of pulsing (e.g., the frequency in change of CAR expression) may be varied. In some embodiments, the level of pulsing and rate of pulsing may be varied using different transcription initiators, different transcription regulators, or different Enhancer-Promoter Pairs.

### Inducible Constructs Comprising Transcription Initiators and Transcription Regulators or EP Pairs and Expression of Fusion Proteins

In some embodiments, the circuits may drive expression of an engineered antigen receptor including T cell Receptors (“TCRs”). Chimeric antigen receptors (“CARs” including all generations), B cell Receptors (“BCRs”), co-receptors of a TCR or BCR and/or any combination or hybrid thereof. In some embodiments, engineered receptors may signal or otherwise (including a dominant negative antigen sink). In some embodiments, antigen receptors may include one or more of a DHD and/or binding site for a degrader, a binding site for an effector protein of a LOCKR switch, or a binding site for a direct or indirect signaling transducer of a safety switch. In some embodiments, antigen receptors may comprise a fully-human sequence, a humanized sequence, a sequence isolated or derived from a mammalian sequence or a non-human sequence (including a mouse sequence), a syngeneic sequence, a chimeric sequence, a recombinant sequence or a sequence derived from any species.

One example of an antigen receptor may include a chimeric antigen receptor (CAR). In some embodiments, a CAR may comprise minimally, an extracellular domain that contains a binder that specifically and selectively binds a target antigen, a transmembrane domain and an intracellular domain that contains at least one signaling domain. In some embodiments, one or more sequences of the CAR may be naturally occurring or non-naturally occurring. In some embodiments, one or more sequences of the CAR may be endogenous or exogenous to the cell expressing it. In some embodiments, one or more sequences of the CAR may be a fully-human sequence, a humanized sequence, a sequence isolated or derived from a mammalian sequence or a non-human sequence (including a mouse sequence), and/or a syngeneic sequence. In some embodiments, the CARs may be specific for one or more target antigens. For example, some CARs may have one or more binders, wherein each binder is specific for a distinct epitope on the same target antigen. In some embodiments, CARs contain a spacer to attain an optimal distance of the immune synapse. The extracellular domain of the CAR may comprise the spacer, which is positioned between the T-cell surface and a binder. If there is more than one binder, the spacer may be positioned between the T-cell surface and a first binder. In some embodiments, binders may have any structure, including but not limited to: (1) an antibody or any fragment thereof: (2) an antibody mimetic or any portion thereof; and/or (3) a de novo designed protein. Exemplary binders may include, but are not limited to: scFv, VH, VHH, monoclonal antibody and any combination thereof. In some embodiments, transmembrane domains may be isolated or derived from any protein, including any transmembrane protein. Exemplary transmembrane domains may comprise a sequence isolated or derived from a CD4 or a CD8 protein. In some embodiments, CARs may be of any CAR “generation” including, but not limited to, Gen I, Gen II, Gen III, Gen IV, and Gen V. In some embodiments, intracellular domains of the CAR may contain one or more of: immunoreceptor tyrosine-based activation motif (ITAM) repeats (e.g., a single CD3ζ intracellular domain) (Gen I), ITAM repeats and one co-stimulatory molecule (e.g., CD27 or CD137 sequence) (Gen II), ITAM repeats, a first co-stimulatory molecule and a second co-stimulatory molecule (e.g., CD134 or CD137) (Gen III), ITAM repeats, a co-stimulatory molecule and a constitutively or inducibly expressed chemokine (e.g. WT IL-12) (Gen IV; also referred to as a T cell redirected for universal cytokine-mediated killing (TRUCK)), and/or ITAM repeats, a co-stimulatory molecule and a intracellular domain of cytokine receptors (e.g. WT IL-2Rβ chain fragment) (Gen V).

In some embodiments, CARs can signal or otherwise (a dominant negative antigen sink). CARs may include a DHD and/or binding site for a degrader, a binding site for an effector protein of a LOCKR switch, a logic gate or switch, or a binding site for a direct or indirect signaling transducer of a safety switch. In some embodiments, CARs may be split CARs in which a final activity depends upon the binding and interaction of two domains or two receptors.

To avoid CAR T cell exhaustion, it would be beneficial to have a system including one or more exemplary constructs that may sense the cell state of exhaustion and shut down stimulation of the CAR. Disclosed herein are one or more exemplary engineered (non-naturally occurring) constructs and methods of use that may be used to shut down stimulation antigen receptors including chimeric antigen receptors in response to sensing the cell state including exhaustion.

### Inducible Constructs and Expression of Fusion Proteins

In some embodiments, the inducible constructs may drive expression of a fusion protein having and engineered receptor (e.g. CAR or TCR) and degradation domain. For example, in some embodiments, the polypeptide may be fused to a degradation domain for targeted degradation of a molecule of interest. In some embodiments, the degradation domain comprises a degron. As provided herein, a degron is a minimal element of a protein that is sufficient for cell-mediated degradation. In some embodiments, the cell-mediated degradation is dependent on ubiquitin. In some embodiments, the cell-mediated degradation is not dependent on ubiquitin.

In some embodiments, the one or more constructs may include a polynucleotide set that includes a polynucleotide encoding a degradation initiator fused to a binding element that is specific for a native motif on an endogenous molecule of interest. The binding domain may, for example, recognize and bind a specific region on the molecule of interest or a specific modification on the molecule of interest. Interaction of the binding element with the native motif on the molecule recruits the degradation initiator to the molecule to initiate degradation.

In some embodiments, the degradation domain comprises a ligase or ligase-binding domain. In some embodiments, the polypeptides of the one or more constructs may be used as binding elements, wherein the polypeptides are fused to a ligase domain (or functional variant thereof). Binding of the polypeptides of the one or more constructs to the protein of interest brings the modified ligase into proximity with the targeted protein, thereby facilitating ubiquitination and subsequent degradation of the protein target.

In some embodiments, the ligase of the one or more constructs may include an E3 ligase. E3 ligases control substrate specificity and the topology of ubiquitination. For example, in a cellular ubiquitin proteasome system, an E3 ligase protein recruits an E2 ubiquitin-conjugating enzyme that has been loaded with ubiquitin, recognizes a protein substrate, and assists or directly catalyzes the transfer of ubiquitin from the E2 to the protein substrate, thereby targeting the protein for degradation. An E3 ligase domain and/or a variant thereof may be selected based on the intended protein targeted for degradation. For example, an E3 ligase and/or a set of E3 ligases may be selected for modulating the stability and/or half-life of a targeted transmembrane protein. In another example, an E3 ligase and/or a set of E3 ligases may be selected for modulating the stability and/or half-life of a cytoplasmic protein (e.g., a transcription factor). In some embodiments, the one or more constructs may include one or more synthetic degraders that may be useful for regulating the activity of a range of target molecules (e.g., proteins) in various synthetic biology applications, such as therapeutic applications. In one embodiment, the one or more synthetic degraders may be used in a cell therapy application to program a population of cells for performing and/or modulating a therapeutic function.

In some embodiments, the one or more synthetic degraders may be used to regulate (degrade) T cell receptors (TCRs) and/or chimeric antigen receptors (CARs) cells used in cancer therapy. More specifically, the synthetic degrader system of the disclosure may be used to degrade CARs and/or TCRs and inhibit excessive CAR/TCR signaling that may result in undesirable exhaustion phenotypes observed in many existing immune cell therapies.

It can be appreciated that within a construct, the regulated promoter or the EP pair may drive transcription of any reporter gene or gene. In a construct comprising a inducible receptor system, the regulated promoter or EP pair may drive transcription of a CAR or degrader. For example, in some embodiments, the regulated promoter or EP pair may drive expression of a CAR or a polypeptide including a fusion protein having degradation domain. In some embodiments, the polypeptide may be fused to a degradation domain for targeted degradation of a molecule of interest. In some embodiments, the degradation domain comprises a degron. As provided herein, a degron is a minimal element of a protein that is sufficient for cell-mediated degradation. In some embodiments, the cell-mediated degradation may or may not be dependent on ubiquitin.

In some embodiments, the inducible receptor system of the disclosure may be useful for regulating the activity of a range of target molecules (e.g., proteins) in various synthetic biology applications, such as therapeutic applications. For example, in one embodiment, the inducible receptor system of the disclosure may be used in a cell therapy application to program a population of cells for performing and/or modulating a therapeutic function. In one aspect, the inducible receptor system of the disclosure may be used to regulate T cell receptors (TCRs) and/or chimeric antigen receptors (CARs) cells used in cancer therapy. More specifically, the inducible receptor system of the disclosure may be used to degrade CARs and/or TCRs and inhibit excessive CAR/TCR signaling that may result in undesirable exhaustion phenotypes observed in many existing T cell therapies.

### Vectors

The disclosure provides vectors comprising one or more of the nucleic acid sequence constructs of the disclosure. In some embodiments, the vector is an expression vector, e.g., a plasmid. In some embodiments, expression vectors of the disclosure may be expressed in host cells. Host cells may, for example, be prokaryotic cells, such as bacteria cells; or eukaryotic cells, such as yeast cells, plant cells, or mammalian cells. Examples of mammalian cells suitable for use with the disclosure include human primate, and non-human primate cells. Other mammalian cells include mouse, rat, pig, rabbit, sheep, and goat cells. In some cases, the cells are synthetic cells. A host cell may, for example, be selected from the group consisting of immune cell, cardiac cell, lung cell, muscle cell, epithelial cell, pancreatic cell, skin cell, CNS cell, neuron, myocyte, skeletal muscle cell, smooth muscle cell, liver cell, kidney cell and glial cell.

In some embodiments, the host cell is an immune cell. Immune cells may include stem, progenitor and/or differentiated cells. Immune cells may be isolated and/or derived from a cell of the innate and/or adaptive immune systems. Immune cells may be isolated and/or derived from a cell of the bone marrow or an immune cell containing organ (e.g., a thymus, a liver, tonsils, a lymph node, a spleen and/or blood). In some embodiments, cells of the innate immune system include, but are not limited to, granulocytes (including basophils, eosinophils, and neutrophils), monocytes (that become macrophages), macrophages, mast cells, antigen presenting cells (“APCs”) including dendritic cells, and natural killer (“NK”) cells, which also play a role in adaptive immunity. In some embodiments, cells of the adaptive immune system include, but are not limited to, hematopoietic stem cells (“HSCs”), T Lymphocytes (e.g., T cells), B lymphocytes (e.g., B cells), and NK cells.

In some embodiments, the host cell is a white blood cell. In some embodiments, the host cell is a lymphocyte. In some embodiments, the host cell is a T cell. In some embodiments, the host cell is a CD8+ T cell. In some embodiments, the host cell is CD4+ T cell. In some embodiments, the host cell is a regulatory T cell. In some embodiments, the host cell is a Gamma delta T cell. In some embodiments, the host cell is an alpha beta T cell. In some embodiments, a host cell is a human cell ex vivo. In some embodiments, a host cell is a human cell in vivo.

In some embodiments, the host cell is modified to express an exogenous receptor. In some embodiments, the host cell is modified to express a chimeric antigen receptor (CAR). In some embodiments, the host cell is modified to express an engineered T cell receptor (TCR). In some embodiments, a host cell is a human cell ex vivo. In some embodiments, a host cell is a human cell in vivo.

In some embodiments, the host cell is modified to express an exogenous receptor. In some embodiments, the host cell is modified to express a chimeric antigen receptor (CAR). In some embodiments, the host cell is modified to express an engineered T cell receptor (TCR).

In some embodiments, the vector is used to deliver the constructs of the disclosure and include non-viral vectors comprising one or more of constructs described herein. Non-limiting examples of non-viral vectors of the disclosure include, organic particles, inorganic particles (e.g., gold particles), nanoparticles, lipid-based vectors, micelles, endosomes, exosomes, liposomes, polymer-based vectors, polymersomes, peptide-based vectors, and cell-penetrating peptides.

### Pharmaceutical Compositions

Also provided herein are pharmaceutical compositions. In some embodiments, the pharmaceutical compositions of the disclosure comprise any one or more of the sequences, nucleotides, constructs, vectors, and cells of the disclosure in combination with a pharmaceutically-acceptable excipient thereof.

### Methods of Use

In some embodiments, provided herein is a method comprising the use of the constructs of the disclosure to respond to cell state and drive expression. For example, in some embodiments, the constructs are used to reduce exhaustion in an immune cell comprising using the constructs to drive expression of desired downstream genes during periods of stimulation of a TCR or CAR expressed by the immune cell. The constructs provided herein are responsive to cell exhaustion, cell rest, antigen receptor activation, cell stimulation, and epigenetic regulation. In some embodiments, the constructs disclosed herein may be used in the treatment of cancers including solid cancers and hematological cancers, or the treatment of autoimmune diseases.

Likewise, provided herein are the use of such constructs, vectors comprising the constructs described herein, and cells comprising the vectors described herein.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### ENUMERATED EMBODIMENTS

Embodiment I-1 A nucleic acid construct comprising a transcription initiator or the transcription initiator and a transcription regulator, wherein each of the transcription regulator and transcription initiator comprises a concatemer of transcription factor binding motifs for binding more than one unique transcription factor, or for binding the same transcription factor at more than one site, and wherein the construct is non-naturally occurring.

Embodiment I-2 The construct of embodiment I-1, comprising the transcription regulator and the transcription initiator.

Embodiment I-3 The construct of embodiment I-1, wherein the transcription regulator is located 5′ of the transcription initiator.

Embodiment I-4 The construct of embodiment I-1, wherein the transcription regulator is located 3′ of the transcription initiator.

Embodiment I-5 The construct of embodiment I-1, wherein the construct does not comprise the combination of:
a transcription regulator comprising an NFAT sequence, an IRF4 sequence, an AICE sequence, an ISRE sequence; and
a transcription initiator comprising a human beta globin sequence.

Embodiment I-6 The construct of embodiment I-1, wherein the construct does not comprise the sequence of

(SEQ ID NO: 9786)
GGAGGAAAAACTGTTTCATACAGAAGGCGTGGAGGAAAAACTGTTTCATA
CAGAAGGCGTGGAGGAAAAACTGTTTCATACAGAAGGCGTGGAGGAAAAA
CTGTTTCATACAGAAGGCGTGAATTCAGGGCTGGGCATAAAAGTCAGGGC
AGAGCCATCTATTGCTTACATTTGCTTCTGACACAACTGTGTTCACTAGC
AACCTCAAACAGACACC.

Embodiment I-7 The construct of embodiment I-1, wherein the construct does not comprise the combination of:
a transcription regulator comprising an NFAT sequence; and
a transcription initiator comprising a YB-TATA sequence.

Embodiment I-8 The construct of embodiment I-1, wherein the construct does not comprise the sequence of

(SEQ ID NO: 9787)
GGAGGAAAAACTGTTTCATACAGAAGGCGTGGAGGAAAAACTGTTTCATA
CAGAAGGCGTGGAGGAAAAACTGTTTCATACAGAAGGCGTGGAGGAAAAA
CTGTTTCATACAGAAGGCGTGAATTCCTAGAGGGTATATAATGGGGGCCA
CTAGTCTACTACCAGAAAGCTTGGTACCGAGCTCGGATCCAGCCACC.

Embodiment I-9 The construct of any one of embodiments I-1 to I-8, wherein the concatemer of the transcription regulator comprises two or more repeated sequences.

Embodiment I-10 The construct of embodiment I-9, wherein concatemer of transcription factor binding motifs binds more than one unique transcription factor.

Embodiment I-11 The construct of embodiment I-9, wherein concatemer of transcription factor binding motifs binds the same transcription factor at more than one site.

Embodiment I-12 The construct of embodiment I-9, wherein the two or more repeated sequences comprise the same sequence.

Embodiment I-13 The construct of embodiment I-9, wherein the two or more repeated sequences do not comprise the same sequence.

Embodiment I-14 The construct of any one of embodiments 1-9 to I-13, wherein the concatemer comprises a linking sequence positioned between the repeated sequences.

Embodiment I-15 The construct of embodiment I-14, wherein the linking sequence comprises one or more of TACGCT, TGATCT, TGCTTT, and TGCCCGT.

Embodiment I-16 The construct of any one of embodiments 1-1 to I-15, wherein the transcription regulator comprises a sequence isolated or derived from one or more of the untranslated genomic sequence of BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D.

Embodiment I-17 The construct of any one of embodiments I-1 to I-16, wherein the transcription regulator comprises a sequence isolated or derived from one or more of the untranslated genomic sequence of NFAT, NFkB, REL, RELA, IRF2, GATA3 and ATF3.

Embodiment I-18 The construct of any one of embodiments I-1 to I-16, wherein the transcription regulator comprises sequences derived from the untranslated genomic sequence of NFAT, optionally comprising a sequence of any one or more of sequences of SEQ ID NOS: 3430-3437, 3802-3803, 3940-3947, 4312-4313, 4808-4815, 4874-4889, 4298-4299, 4364-4379, 8576-8583, 8948-8949, 9444-9445, and 9510-9525.

Embodiment I-19 The construct of any one of embodiments I-1 to I-16, wherein the transcription regulator comprises sequences derived from the untranslated genomic sequence of NFkB, optionally comprising a sequence of any one or more of sequences of SEQ ID NO: 550-553, 1060-1063, and 5696-5699.

Embodiment I-20 The construct of any one of embodiments I-1 to I-16, wherein the transcription regulator comprises sequences derived from the untranslated genomic sequence of RELA, optionally comprising a sequence of any one or more of sequences of SEQ ID NO: 958-959, 3486-3501,4426-4427, 4472-4473, 4484-4499, 1468-1469, 3996-4011, 4936-4937, 4982-4983, 4994-5009, 5697-5698, 8632-8647, 9572-9573, 9618-9619, and 9630-9645.

Embodiment I-21 The construct of any one of embodiments I-1 to I-16, wherein the transcription regulator comprises sequences derived from the untranslated genomic sequence of IRF2, optionally comprising a sequence of any one or more of sequences of SEQ ID NO: 2384-2447, 4430-4461, 2894-2957, 4940-4971, 7530-7593, and 9576-9607.

Embodiment I-22 The construct of any one of embodiments I-1 to I-16, wherein the transcription regulator comprises sequences derived from the untranslated genomic sequence of GATA3, optionally comprising a sequence of any one or more of sequences of SEQ ID NO: 1048-1079, 3566-3709, 1558-1589, 4076-4219, 6194-6225, and 8712-8855.

Embodiment I-23 The construct of any one of embodiments 1-1 to I-16, wherein the transcription regulator comprises sequences derived from the untranslated genomic sequence of ATF3, optionally comprising a sequence of any one or more of sequences of SEQ ID NO: 124-159, 3140-3147, 634-669, 3650-3657, 5179-5214, and 8286-8293.

Embodiment I-24 The construct of any one of embodiments I-1 to I-23, wherein (a) the transcription regulator is inducible or (b) the construct is inducible.

Embodiment I-25 The construct of any one of embodiments I-1 to I-24, wherein the transcription initiator comprises a classical promoter.

Embodiment I-26 The construct of embodiment I-25, wherein the promoter comprises a minimal promoter.

Embodiment I-27 The construct of embodiment I-26, wherein the minimal promoter comprises a sequence isolated or derived from one or more of minimal promoter-1 (“minP1”), YB-TATA and human beta globin.

Embodiment I-28 The construct of embodiment I-27, wherein the minimal promoter comprises one or more of:
MinP1 having a sequence of AGAGGGTATATAAAAGCTCGACTTCCAG (SEQ ID NO: 9788):
MinP2=having a sequence of TAGAGGGTATATAATGGGGGCCACTAGTCTACTACCAGAAAGCTTGGTACCGAGCT CGGATCCAGCCACC (SEQ ID NO: 9789); and
MinP3=having a sequence of

(SEQ ID NO: 9790)
CTAGAGGGTATATAATGGGGGCCACTAGTCTACTACCAGAAAGCTTGGTA
CCGAGCTCGGATCCAGCCACC.

Embodiment I-29 The construct of any one of embodiments I-1 to I-28, wherein the transcription initiator comprises a transcription factor binding motif.

Embodiment I-30 The construct of embodiment I-29, wherein the transcription initiator comprises a motif isolated or derived from the untranslated genomic sequence of a transcription factor or comprises a mammalian promoter sequence.

Embodiment I-31 The construct of embodiment I-29, wherein the motif comprises a sequence isolated or derived from one or more of the untranslated genomic sequence of BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D.

Embodiment I-32 The construct of any one of embodiments I-1 to I-31, wherein the transcription initiator comprises a sequence isolated or derived from one or more of the untranslated genomic sequence of NFAT, NFkB, REL, RELA, IRF2, GATA3 and ATF3.

Embodiment I-33 The construct of any one of embodiments I-1 to I-31, wherein the transcription initiator comprises sequences derived from the untranslated genomic sequence of NFAT, optionally comprising a sequence of any one or more of sequences of SEQ ID NOS: 3430-3437, 3802-3803, 3940-3947, 4312-4313, 4808-4815, 4874-4889, 4298-4299, 4364-4379, 8576-8583, 8948-8949, 9444-9445, and 9510-9525.

Embodiment I-34 The construct of any one of embodiments I-1 to I-31, wherein the transcription initiator comprises sequences derived from the untranslated genomic sequence of NFkB, optionally comprising a sequence of any one or more of sequences of SEQ ID NO: 550-553, 1060-1063, and 5696-5699.

Embodiment I-35 The construct of any one of embodiments I-1 to I-31, wherein the transcription initiator comprises sequences derived from the untranslated genomic sequence of REL, optionally comprising a sequence of any one or more of sequences of SEQ ID NO: 958-959, 3486-3501, 4426-4427, 4472-4473, 4484-4499, 1468-1469, 3996-4011, 4936-4937, 4982-4983, 4994-5009, 5697-5698, 8632-8647, 9572-9573, 9618-9619, and 9630-9645.

Embodiment I-36 The construct of any one of embodiments I-1 to I-31, wherein the transcription initiator comprises sequences derived from the untranslated genomic sequence of RELA, optionally comprising a sequence of any one or more of sequences of SEQ ID NO: 4484-4499, 4994-5009, and 9630-9645.

Embodiment I-37 The construct of any one of embodiments I-1 to I-31, wherein the transcription initiator comprises sequences derived from the untranslated genomic sequence of IRF2, optionally comprising a sequence of any one or more of sequences of SEQ ID NO: 2384-2447, 4430-4461, 2894-2957, 4940-4971, 7530-7593, and 9576-9607.

Embodiment I-38 The construct of any one of embodiments I-1 to I-31, wherein the transcription initiator comprises sequences derived from the untranslated genomic sequence of GATA3, optionally comprising a sequence of any one or more of sequences of SEQ ID NO: 1048-1079, 3566-3709, 1558-1589, 4076-4219, 6194-6225, and 8712-8855.

Embodiment I-39 The construct of any one of embodiments 1-1 to I-31, wherein the transcription initiator comprises sequences derived from the untranslated genomic sequence of ATF3, optionally comprising a sequence of any one or more of sequences of SEQ ID NO: 124-159, 3140-3147, 634-669, 3650-3657, 5179-5214, and 8286-8293.

Embodiment I-40 The construct of any one of embodiments I-1 to I-39, wherein the transcription initiator comprises the sequence of any one or more of SEQ ID NOS: 175-9781, or concatemers of the same.

Embodiment I-41 The construct of any one of embodiments I-1 to I-40, wherein the construct further comprises an integration sequence capable of inducing or increasing insertion of the construct into a target site.

Embodiment I-42 The construct of embodiment I-41, wherein a vector comprises the target site.

Embodiment I-43 The construct of embodiment I-41, wherein a chromosome comprises the target site.

Embodiment I-44 The construct of any one of embodiments 1-41 to I-43, wherein the integration sequence comprises a first or 5′ integration sequence and a second or 3′ integration sequence.

Embodiment I-45 The construct of any one of embodiments 1-41 to I-44, wherein the integration sequence comprises a homologous sequence corresponding to an insertion site to facilitate homologous recombination.

Embodiment I-46 The construct of any one of embodiments I-41 to I-44, wherein the integration sequence comprises a transposable element to facilitate transposition.

Embodiment I-47 The construct of embodiment I-46, wherein the transposable element comprises an insertion sequence and/or a transposon sequence.

Embodiment I-48 The construct of any one of embodiments 1-41 to I-44, wherein the integration sequence comprises a first 5′ terminal repeat sequence or a second 3′ terminal repeat sequence.

Embodiment I-49 The construct of embodiment I-48, wherein the integration sequence comprises a first 5′ terminal repeat sequence and a second 3′ terminal repeat sequence.

Embodiment I-50 The construct of embodiment I-48 or I-49, wherein the first 5′ terminal repeat sequence or the second 3′ terminal repeat sequence comprises a long terminal repeat (LTR) sequence.

Embodiment I-51 The construct of embodiment I-48 or I-49, wherein the first 5′ terminal repeat sequence or the second 3′ terminal repeat sequence comprises an inverted terminal repeat (ITR) sequence.

Embodiment I-52 The construct of any one of embodiments I-1 to I-51, further comprising a reporter sequence.

Embodiment I-53 The construct of embodiment I-52, wherein the reporter sequence comprises a fluorescent protein.

Embodiment I-54 The construct of embodiment I-53, wherein the reporter sequence comprises a green fluorescent protein (GFP).

Embodiment I-55 A vector comprising the construct of any one of embodiments I-1 to I-54.

Embodiment I-56 The vector of embodiment I-55, wherein the vector is a mammalian expression vector capable of expressing the construct in a mammalian cell.

Embodiment I-57 The vector of embodiment I-55 or 1-56, wherein the vector is an expression vector capable of expressing the construct in a human cell.

Embodiment I-58 The vector of embodiment I-57, wherein the vector is a delivery vector capable of delivering the construct to a mammalian cell.

Embodiment I-59 The vector of embodiment I-55 or 1-58, wherein the vector is a delivery vector capable of delivering the construct to a human cell.

Embodiment I-60 A library comprising a construct of any one of embodiments I-11-I-54, or a vector of any one of embodiments I-55 to I-59.

Embodiment I-61 The library of embodiment I-60, comprising a plurality of constructs of any one of embodiments I-1-I-54, or a plurality of vectors of any one of embodiments I-55-I-59.

Embodiment I-62 The library of embodiment I-60 or 1-61, comprising a plurality of constructs, each sequence according to any one of embodiments I-1-I-54.

Embodiment I-63 The library of embodiment I-60 or 1-61, comprising a plurality of vectors, each vector according to any one of embodiments I-55-I-59.

Embodiment I-64 A cell comprising a construct of any one of embodiments I-1 to I-54; a vector of any one of embodiments 1-55 to I-59; or a library of any one of embodiments I-60 to I-63.

Embodiment I-65 The cell of embodiment I-64, wherein the cell is an immune cell.

Embodiment I-66 The cell of embodiment I-64 or 1-65, wherein the cell is a stem cell, a progenitor cell, a precursor cell or a naive cell.

Embodiment I-67 The cell of embodiment I-64 or 1-65, wherein the cell is a differentiated cell.

Embodiment I-68 The cell of any one of embodiments I-64 to I-67, wherein the cell is a T-cell, a B-cell, a macrophage or an NK cell.

Embodiment I-69 The cell of any one of embodiments 1-64 to I-67, wherein the cell is a T-cell.

Embodiment I-70 The cell of any one of embodiments I-64 to I-67, wherein the cell is an NK cell.

Embodiment I-71 The cell of any one of embodiments 1-64 to I-67, wherein the cell is in vitro or ex vivo.

Embodiment I-72 The cell of any one of embodiments I-64 to I-67, wherein the cell is a primary cell.

Embodiment I-73 The cell of any one of embodiments 1-64 to I-67, wherein the cell is a cultured cell.

Embodiment I-74 A use of a construct of any one of embodiments I-1 to I-54, a vector of any one of embodiments 1-55 to I-59; a library of any one of embodiments 1-60 to I-63; or a cell of any one of embodiments 1-64 to I-73, for selecting a sequence that responds to one or more of antigen receptor activation, cell stimulation, cell exhaustion, cell rest, and epigenetic regulation.

Embodiment I-75 The use of embodiment I-74, wherein cell stimulation is early stimulation.

Embodiment I-76 The use of embodiment I-74, wherein cell stimulation is late stimulation.

Embodiment I-77 The use of embodiment I-74, wherein the antigen receptor is a chimeric antigen receptor.

Embodiment I-78 The use of embodiment I-74, wherein the cell is a T cell and wherein the antigen receptor is an endogenous or exogenous T cell receptor (TCR).

Embodiment I-79 The use of a construct of any one of embodiments 1-1 to I-54, a vector of any one of embodiments 1-55 to I-59; a library of any one of embodiments I-60 to I-63: or a cell of any one of embodiments 1-64 to I-73, for regulating transcription of an exogenous protein, in response to one or more of antigen receptor activation, cell stimulation, cell exhaustion, cell rest, and epigenetic regulation.

Embodiment I-80 The use of embodiment I-79, wherein the exogenous protein is one or more of an engineered receptor, a cytokine, or a degrader.

Embodiment II-1 An inducible nucleic acid construct comprising a transcription regulating enhancer sequence and a transcription initiating promoter sequence (an EP-pairing), wherein the construct is non-naturally occurring.

Embodiment II-2 The construct of embodiment II-1, wherein the EP-pairing comprises an EP pairing sequence of any one or more sequences of SEQ ID NOS: 1-174, or a sequence comprising 90% sequence identity thereto.

Embodiment II-3 The construct of embodiment II-1, wherein the EP-pairing comprises an enhancer sequence comprising at least 70% sequence identity to of any one or more of the enhancer sequences of SEQ ID NOS: 1-174.

Embodiment II-4 The construct of embodiment II-1, wherein the EP-pairing comprises a promoter sequence comprising at least 70% sequence identity to of any one or more of the promoter sequences of SEQ ID NOS: 1-174.

Embodiment II-5 The construct of embodiment II-1, wherein the enhancer sequence is positioned 5′ to the promoter sequence.

Embodiment II-6 The construct of embodiment II-1, wherein the enhancer sequence is positioned 3′ to the promoter sequence.

Embodiment II-7 The construct of embodiment II-1, wherein the EP-pairing comprises between about 100 base pairs and about 1500 base pairs.

Embodiment II-8 The construct of embodiment II-1, wherein the EP-pairing comprises between about 500 base pairs and about 1250 base pairs.

Embodiment II-9 The construct of embodiment II-1, wherein the EP-pairing comprises between about 900 base pairs and about 1100 base pairs.

Embodiment II-10 The construct of any one of embodiments II-1 to II-9, wherein the construct further comprises a barcode sequence.

Embodiment II-11 The construct of embodiment II-10, wherein the barcode sequence is positioned 5′ to the EP-pairing.

Embodiment II-12 The construct of embodiment II-10, wherein the barcode sequence is positioned 3′ to the EP-pairing.

Embodiment II-13 The construct of any one of embodiments II-1 to II-12, wherein the construct further comprises a 5′ untranslated region (UTR) positioned 3′ to the EP-pairing.

Embodiment II-14 The construct of any one of embodiments II-1 to II-12, wherein the construct further comprises a 5′ untranslated region (UTR) positioned 3′ to the barcode sequence.

Embodiment II-15 The construct of any one of embodiments II-1 to II-14, wherein the construct further comprises an integration sequence capable of inducing or increasing insertion of the construct into a target site.

Embodiment II-16 The construct of embodiment II-15, wherein a target site is a target site in a vector.

Embodiment II-17 The construct of embodiment II-15, wherein a target site is a target site in a chromosome.

Embodiment II-18 The construct of any one of embodiments II-15 to II-17, wherein the integration sequence comprises a first or 5′ integration sequence and a second or 3′ integration sequence.

Embodiment II-19 The construct of any one of embodiments II-15 to II-18, wherein the integration sequence comprises a homologous sequence corresponding to an insertion site to facilitate homologous recombination.

Embodiment II-20 The construct of any one of embodiments II-15 to II-18, wherein the integration sequence comprises a transposable element to facilitate transposition.

Embodiment II-21 The construct of embodiment II-20, wherein the transposable element comprises an insertion sequence and/or a transposon sequence.

Embodiment II-22 The construct of any one of embodiments II-15 to II-18, wherein the integration sequence comprises a first 5′ terminal repeat sequence or a second 3′ terminal repeat sequence.

Embodiment II-23 The construct of embodiment II-21 or II-22, wherein the first 5′ terminal repeat sequence or the second 3′ terminal repeat sequence comprises a long terminal repeat (LTR) sequence.

Embodiment II-24 The construct of embodiment II-21 or II-22, wherein the first 5′ terminal repeat sequence or the second 3′ terminal repeat sequence comprises an inverted terminal repeat (ITR) sequence.

Embodiment II-25 The construct of any one of embodiments II-1 to II-24, further comprising a reporter sequence.

Embodiment II-26 The construct of embodiment II-25, wherein the reporter sequence comprises a fluorescent protein.

Embodiment II-27 The construct of embodiment II-26, wherein the reporter sequence comprises a green fluorescent protein (GFP).

Embodiment II-28 The construct of any one of embodiments II-1 to II-27, wherein the enhancer sequence is isolated or derived from a sequence of one or more of human chromosome 1, 2, 3, 4, 5, 6, 7, 8, 9, 10, 11, 12, 13, 14, 15, 16, 17, 18, 19, 20, 21, 22, or 23.

Embodiment II-29 The construct of any one of embodiments II-1 to II-28, wherein the enhancer sequence is a minimal sequence demonstrating an enhancer function or activity.

Embodiment II-30 The construct of embodiment II-28 or II-29, wherein the enhancer sequence comprises at least about 50 base pairs (bp), at least about 100 bp, at least about 150 bp, at least about 200 bp, at least about 250 bp, at least about 300 bp, at least about 350 bp, at least about 400 bp, at least about 450 bp, at least about 500 bp or any number of base pairs in between.

Embodiment II-31 The construct of any one of embodiments II-1 to II-30, wherein the promoter sequence is isolated or derived from a sequence of one or more of human chromosome 1, 2, 3, 4, 5, 6, 7, 8, 9, 10, 11, 12, 13, 14, 15, 16, 17, 18, 19, 20, 21, 22, or 23.

Embodiment II-32 The construct of any one of embodiments II-1 to II-31, wherein the promoter sequence is a minimal sequence demonstrating a promoter function or activity.

Embodiment II-33 The construct of embodiment II-31 or II-32, wherein the promoter sequence comprises at least about 50 base pairs (bp), at least about 100 bp, at least about 150 bp, at least about 200 bp, at least about 250 bp, at least about 300 bp, at least about 350 bp, at least about 400 bp, at least about 450 bp, at least about 500 bp or any number of base pairs in between.

Embodiment II-34 A vector comprising the construct of any one of embodiments II-1 to II-33.

Embodiment II-35 The vector of embodiment II-34, wherein the transcription regulating enhancer sequence and/or the transcription initiating promoter sequence is operably linked to a nucleic acid sequence encoding of any one or more of a chimeric antigen receptor (CAR), an exogenous T-cell receptor (TCR), a cytokine, or a degrader.

Embodiment II-36 The vector of any one of embodiments II-34 to II-35, wherein the vector is a mammalian expression vector capable of expressing the construct in a mammalian cell.

Embodiment II-37 The vector of any one of embodiments II-34 to II-36, wherein the vector is an expression vector capable of expressing the construct in a human cell.

Embodiment II-38 The vector of any one of embodiments II-34 to II-35, wherein the vector is a delivery vector capable of delivering the construct to a mammalian cell.

Embodiment II-39 The vector of any one of embodiments II-34 to II-35, wherein the vector is a delivery vector capable of delivering the construct to a human cell.

Embodiment II-40 A library comprising a construct of any one of embodiments II-1 to II-33 or a vector of any one of embodiments II-34 to II-39.

Embodiment II-41 The library of embodiment II-40, comprising a plurality of constructs of any one of embodiments II-1 to II-33 or a plurality of vectors of any one of embodiments II-34 to II-39.

Embodiment II-42 The library of embodiment II-40 or II-41, comprising a plurality of constructs, each sequence according to any one of embodiments II-1 to II-33.

Embodiment II-43 The library of embodiment II-42, wherein each construct comprises a unique barcode sequence.

Embodiment II-44 The library of embodiment II-40 or II-41, comprising a plurality of vectors, each vector according to any one of embodiments II-34 to II-39.

Embodiment II-45 A cell comprising
a construct of any one of embodiments II-1 to II-33;
a vector of any one of embodiments II-34 to II-39; or
a library of any one of embodiments II-40 to II-44.

Embodiment II-46 The cell of embodiment II-45, wherein the cell is an immune cell.

Embodiment II-47 The cell of embodiment II-45, wherein the cell is a stem cell, a progenitor cell, a precursor cell or a naive cell.

Embodiment II-48 The cell of embodiment II-45, wherein the cell is a differentiated cell.

Embodiment II-49 The cell of any one of embodiments II-45 to II-48, wherein the cell is a T-cell, a B-cell, a macrophage or an NK cell.

Embodiment II-50 The cell of any one of embodiments II-45 to II-48, wherein the cell is a T-cell.

Embodiment II-51 The cell of any one of embodiments II-45 to II-48, wherein the cell is an NK cell.

Embodiment II-52 The cell of any one of embodiments II-45 to II-48, wherein the cell is in vitro or ex vivo.

Embodiment II-53 The cell of any one of embodiments II-45 to II-48, wherein the cell is a primary cell.

Embodiment II-54 The cell of any one of embodiments II-45-II-48, wherein the cell is a cultured cell.

Embodiment II-55 A use of a construct of any one of embodiments II-1 to II-33, a vector of any one of embodiments II-34 to II-39; or a library of any one of embodiments II-40 to II-44 or a cell of any one of embodiments II-45 to II-54 for selecting an EP pairing that responds to one or more of antigen receptor activation, cell stimulation, cell exhaustion, cell rest, epigenetic regulation.

Embodiment II-56 The use of embodiment II-55, wherein cell stimulation is early stimulation.

Embodiment II-57 The use of embodiment II-55, wherein cell stimulation is late stimulation.

Embodiment II-58 The use of embodiment II-55, wherein the antigen receptor is a chimeric antigen receptor (CAR).

Embodiment II-59 The use of embodiment II-55, wherein the cell is a T cell and wherein the antigen receptor is an endogenous or exogenous T cell receptor (TCR).

Embodiment III-1 An inducible construct comprising a nucleic acid/nucleotide/polynucleotide sequence encoding a circuit component, wherein the circuit component comprises
a response component, wherein the response component is capable of modifying an activity of the effector component in response to:
a signal from a receptor,
an epigenetic change,
an induction or a suppression of transcription, or
a change in cell state, and
an effector component, wherein the activity of the response component is capable of inducing a change in an activity of the effector component; and
wherein the construct is non-naturally occurring.

Embodiment III-2 The construct of embodiment III-1, wherein the response component comprises one or more of a sequence of an untranslated region of a transcription factor (TF), a sequence comprising a binding site for a TF, a promoter, an enhancer, and a suppressor.

Embodiment III-3 The construct of embodiment III-1, wherein the response component comprises an epigenetic modifier.

Embodiment III-4 The construct of embodiment III-1, wherein the response component comprises a transcription initiator (e.g., promoter) or a transcription initiator (e.g., promoter) and a transcription regulator (e.g., enhancer).

Embodiment III-5 The construct of embodiment III-4, wherein the promoter comprises a minimal promoter.

Embodiment III-6 The construct of embodiment III-5, wherein the minimal promoter comprises one or more of
MinP1=having a sequence of AGAGGGTATATAAAAGCTCGACTTCCAG (SEQ ID NO: 9788):
MinP2=having a sequence of

(SEQ ID NO: 9789)
TAGAGGGTATATAATGGGGGCCACTAGTCTACTACCAGAAAGCTTGGTAC
CGAGCTCGGATCCAGCCACC;

MinP3=having a sequence of

(SEQ ID NO: 9790)
CTAGAGGGTATATAATGGGGGCCACTAGTCTACTACCAGAAAGCTTGGTA
CCGAGCTCGGATCCAGCCACC.

Embodiment III-7 The construct of embodiment III-4 or III-5, wherein the promoter comprises an inducible promoter.

Embodiment III-8 The construct of embodiment III-4, wherein the transcription initiator comprises a concatemerized sequence.

Embodiment III-9 The construct of embodiment III-8, wherein the concatemerized sequence comprises a sequence of any one or more sequences of SEQ ID NOS:176-9781 or a sequence with at least about 70% sequence identity thereto.

Embodiment III-10 The construct of embodiment III-8, wherein the transcription initiator comprises sequences derived from the untranslated genomic sequence of IRF2, optionally comprising a sequence of any one or more of sequences of SEQ ID NO: 2384-2447, 4430-4461, 2894-2957, 4940-4971, 7530-7593, and 9576-9607.

Embodiment III-11 The construct of embodiment III-4, wherein the response component comprises a pairing of an enhancer and a promoter (an EP pair).

Embodiment III-12 The construct of embodiment III-11, wherein the EP pair comprises a sequence of any one or more sequences of SEQ ID NOS: 1-175 or a sequence with at least about 90% sequence identity thereto.

Embodiment III-13 The construct of embodiment III-11, wherein the EP pair comprises an enhancer sequence of any one or more enhancer sequences of SEQ ID NOS: 1-174 or a sequence with at least about 70% sequence identity thereto; and/or wherein the EP pair comprises a promoter sequence of any one or more of the promoter sequences of SEQ ID NOS: 1-174 or a sequence with at least about 70% sequence identity thereto.

Embodiment III-14 The construct of any one of embodiments III-1 to III-13, wherein the effector component comprises a degrader.

Embodiment III-15 The construct of any one of embodiments III-1 to III-13, wherein the signal from a receptor comprises a signal from a chimeric antigen receptor (CAR).

Embodiment III-16 The construct of any one of embodiments III-1 to 111-15, wherein the cell state comprises one or more of the following:
Unstimulated;
Stimulation On;
Stimulation Off;
Exhaustion; and
Rest.

Embodiment III-17 A cell comprising the construct of any one of embodiments III-1 to III-16.

Embodiment III-18 The cell of embodiment III-17, wherein the cell is an immune cell.

Embodiment III-19 The cell of embodiment III-17 or III-18, wherein the cell is a stem cell, a progenitor cell, a precursor cell or a naive cell.

Embodiment III-20 The cell of embodiment III-17 or III-18, wherein the cell is a differentiated cell.

Embodiment III-21 The cell of any one of embodiments 111-17 to III-20, wherein the cell is a T-cell, a B-cell, a macrophage or an NK cell.

Embodiment III-22 The cell of any one of embodiments III-17 to III-21, wherein the cell is a T-cell.

Embodiment III-23 The cell of any one of embodiments 111-17 to III-21, wherein the cell is an NK cell.

Embodiment III-24 The cell of any one of embodiments III-17 to III-21, wherein the cell is in vitro or ex vivo.

Embodiment III-25 The cell of any one of embodiments 111-17 to III-21, wherein the cell is a primary cell.

Embodiment III-26 The cell of any one of embodiments III-17 to III-21, wherein the cell is a cultured cell.

Embodiment III-27 The use of any one of the constructs, circuits, vectors, or cells of embodiments III-1 to III-26, to regulate expression of an effector component in response to a cell state, in a inducible manner.

### EXAMPLES

### Example 1: General Methods

Arrayed Screen Method, Functional Testing of Enhancer-Promoter (EP) Pair-CAR circuits in vitro: T cells were cultured in T-cell media (TCM) (OpTmizer basal media (Thermo)+OpTmizer cell supplement (Thermo)+Immune Cell Serum Replacement (Thermo)+2 mM L-Glutamine (Gibco)+2 mM GlutMAX (Thermo)) with 200 IU/mL recombinant IL-2 (R&D Systems), 1200 IU/mL recombinant IL-7 (R&D Systems) and 200 IU/mL recombinant IL-15 (R&D Systems). T cells were thawed into TCM prewarmed at 37 °C on day 0 of T cell production, and immediately activated for 24 hours with 1:100 dilution of TransAct™ (Miltenyi). 24 hours after activation T cells were resuspended at 3e6 cells/mL in TCM+1× TransAct™, and then transduced at a multiplicity of infection (MOI) of 4 with single lentiviral vectors that expressed unique promoter-driven “tool” (i.e., ROR1) CARs and a truncated CD19 (tCD19) transduction marker. Lentiviral copy numbers (VCN) were predetermined by titering on Jurkat cells, and measuring VCN by droplet digital polymerase chain reaction (ddPCR). 24 hours post-transduction, T cells were expanded into 2 mL TCM with cytokine in a 24-well plate, which turned off activation. On day 4 post-thaw, T cells were split 1:2 by volume into fresh TCM with cytokine.

On day 7 post-thaw, T cells were harvested and assessed for transduction efficiency by flow cytometry, and for viability and counts by Acridine Orange/Propidium Iodide (AOPI) staining (Nexcelom Biosciences) on a Cellometer (Nexcelom Biosciences). Given their previously characterized activity 6 days post-transduction MND CAR and 10×NFkB CAR samples were used to assess transduction efficiency. T cells were subsequently used in the downstream passage stimulation assay.

The passage stimulation assay was set up in 1640-RPMI+10% FBS. Exemplary target cells (i.e., H1975-mKate cells of epithelial origin isolated from the lungs of a nonsmoking female with non-small cell lung cancer), maintained below passage 20, were seeded at 20,000 cells/well in a 96-well moat plate (Eppendorf), and allowed to adhere to the plate for 2 hours at 37 °C 3 mL of 1×PBS was added to each moat well of the plates to prevent evaporation from wells. T cells were seeded at a 1:2 effector:target (E:T) ratio based on number of viable cells from AOPI counts. The final volume in each well was 200 μL. Co-culture plates were imaged in the red channel on an IncuCyte© Sx5 (Sartorius) every 6 hours for 3-4 days. 24 hours after the initial co-culture setup 30 μL of supernatant was collected from each well, and stored at −80 °C. After at least 24 hours, the supernatants were diluted 1:5 on MSD plates, and concentrations of IFN-gamma and IL-2 were determined according to manufacturer protocols. 3-4 days after initial co-culture setup new plates of target cells at 20,000 cells/well were prepared as described previously. The original co-cultures were gently resuspended, and 50% of the volume was transferred onto the freshly plated target cells. These plates were then imaged on the IncuCyte© Sx5 every 6 hours for 3-4 days. This concluded round 1, and began round 2. Excess cells from the round 1 plate were harvested and stained for transduction marker and CAR expression, and analyzed by flow cytometry for surface marker expression and T cell expansion. The passage stimulation assay continued for 4-5 rounds.

Tumor spheroid generation: Tumor spheroids were generated by adding 1e4 target cells in 100 uL R10 media to wells of a 96 well round bottom ultra-low attachment plate (Sbio). PBS was added to edge wells. The plate was centrifuged at 1,000×g for 10 min before transferring to a 37 °C incubator for a 72 hour incubation.

Spheroid killing assay: Transduced T cells (described above) were resuspended in R10 media at 1e4/mL. Each sample was serially diluted twice to yield cell suspensions at 5e3/mL and 2.5e3/mL as well. 100 μL of each sample dilution was added to 3 wells of spheroids to measure E:T ratios of 1:10, 1:20, and 1:40. Plates were imaged in an IncuCyte© Sx5 every 6 hours for one week. Mean total red fluorescence integrated intensity (RCU×um2) was calculated for 3 technical replicates per sample and normalized to baseline for visualization. At the end of the assay, plates were centrifuged and each well was resuspended in Cell Staining Buffer (BioLegend). Triplicates from the 1:10 E:T wells were combined and stained for surface markers for 20 min at 4 °C. Cells were then fixed with Fluorofix™ Fixation Buffer (BioLegend) at 37 °C for 20 min at 4 °C, washed and resuspended in CSB for analysis by flow cytometry. Data were acquired on a Ze5 (Biorad) and analyzed in FlowJo software.

Cell Line Generation for IncuCyte© imaging (RK):Lentivirus was prepared using suspension adapted 293 Ts. CAR virus was concentrated 10× using LentiX, and EP-mkate2 viruses were prepared unconcentrated in an arrayed 96 well plate format. Primary T cells were co-transduced in a 96wp format with MOI 4 MND-CAR and 50 uL EP-mKate2 library. 24 hours post transduction, Cells were expanded to 24 well plate format. 5 days post transduction, cells were enriched for CAR positive cells using Stem cell “EasySep™ Human CD19 Positive Selection Kit II” (Cat no #17854). Cells were expanded in TCM+cytokine for 11-14 days.

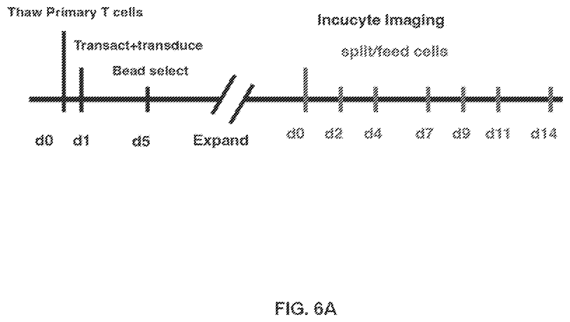

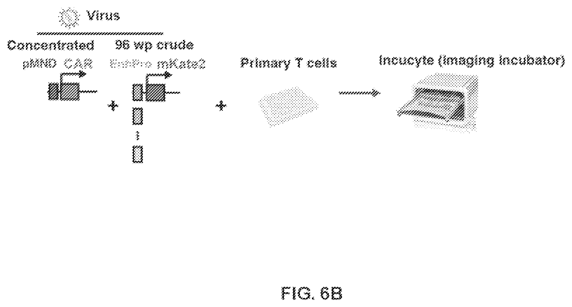

Incucyte imaging experiment (RK): II-14 days post thaw. T cells were harvested. Cells were plated at 100 k cells per well in a 96 well plate Nunc Edge on to either 1 ug/mL of ROR1 antigen coated plates, or uncoated plates. Cells were imaged using every 4 hours using an IncuCyte© continuous imaging incubator using the red and phase channel On Day 2, 4, 7, 9, and 11 of the assay, stimulated cells were split onto fresh antigen plates; Non stimulated cells were fed fresh media+cytokine. Time courses were completed after 14 days. Normalized fluorescence (FIGS. 6A-B) was computed by taking the ratio of integrated red intensity to phase confluence.

### Example 2: Enhancer-Promoter Library Generation

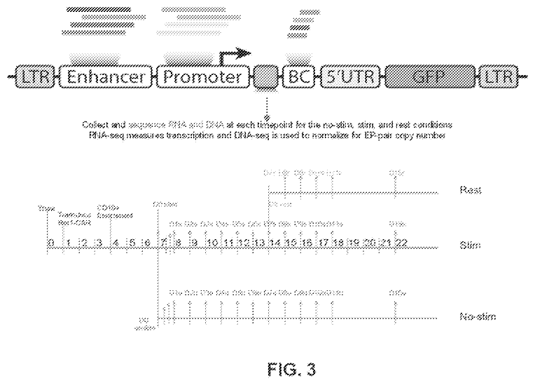

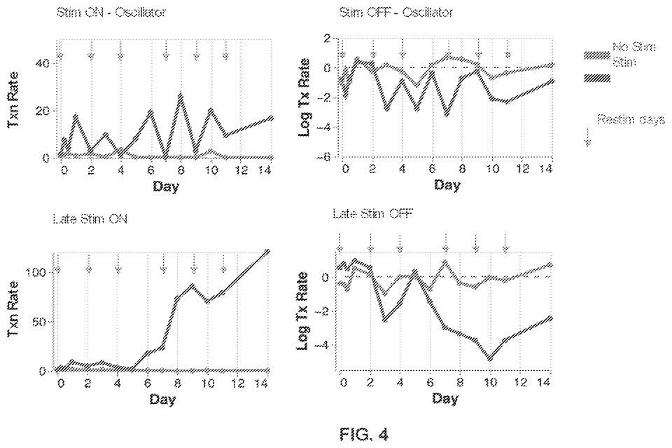

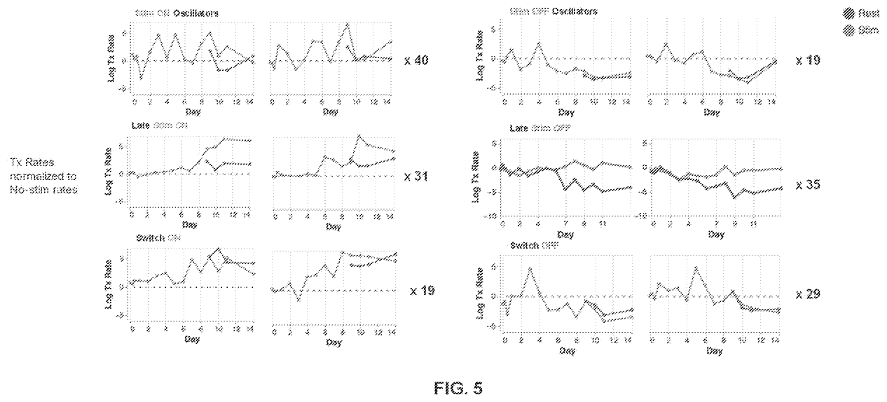

As shown in FIG. 3, over one million constructs were generated for use in a Massively Parallel Reporter Assay (MPRA). RNA-seq was used to measure transcription and DNA-seq was used to normalize for Enhancer-Promoter (EP)-pair copy number. Approximately one million combinations of fifty thousand distinct enhancer sequences were used to form unique combinations with nine thousand promoter sequences in the format of the construct depicted in this figure. The library of over one million constructs was tested functionally to identify constructs with inducible patterns of activity under conditions or induced cell states, such as stimulation, no stimulation, pulsed stimulation, sustained stimulation, rest and exhaustion. Briefly, the MPRA included a thaw of T-cells at day 0, transduction of the thawed cells with a “tool” CAR at day 1 and CD19+ enrichment at day 4 (see Example 1 for detailed protocols). Each of the Stimulation or No Stimulation conditions began at day 7, relative to Day 0 thaw. The rest condition began at day 14, relative to the Day 0 thaw.

### Example 3: Initial Screen of EP-Pairings

As shown in FIG. 6, each EP-pair was characterized functionally for activity both relevant and advantageous for the endogenous regulation (e.g., induced regulation from the activity of an endogenous circuit, a modified endogenous circuit and/or an exogenous circuit as well as the initiation or change of a cell state) of exogenous constructs (e.g., a chimeric receptor, a cytokine or an exogenous regulator). See Example 1 for the experimental protocol. Briefly, this initial screen was designed to (1) determine which EP-Pairs can drive protein expression and (2) roughly classify EP-pairings into categories of stim-ON, stim-OFF and oscillatory based on qualitative behavior patterns.

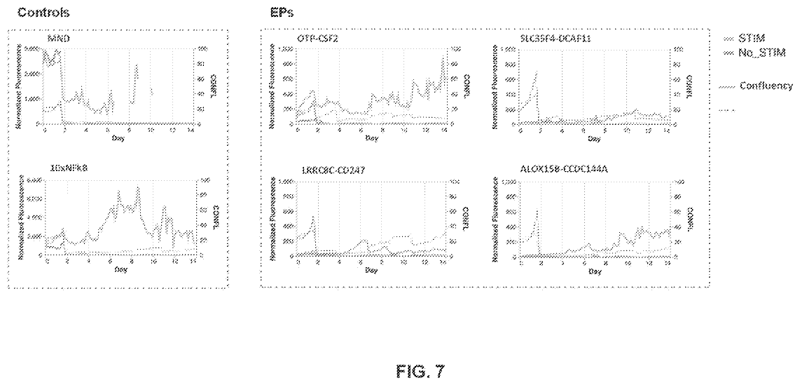

FIG. 7 provides a series of graphs depicting the activity of either control constructs or constructs comprising EP-Pairs of the disclosure under both stimulation and no stimulation conditions. For each plot, normalized fluorescence (shown in arbitrary units of fluorescence) and confluence (shown as a percent) are shown as a function of time (measured in days). With respect to the Control constructs, MND activity is known to be constitutive whereas the activity of concatemerized NFkB (10×NFkB) has been shown in this disclosure to be inducible in the presence of antigen and, therefore, to follow a Stim-ON pattern. With respect to the EP-Pair constructs, low confluence in this experiment made it difficult to identify constructs that are active under no stim conditions. However, several EP-pair constructs were observed to have inducible activity under stimulation conditions.

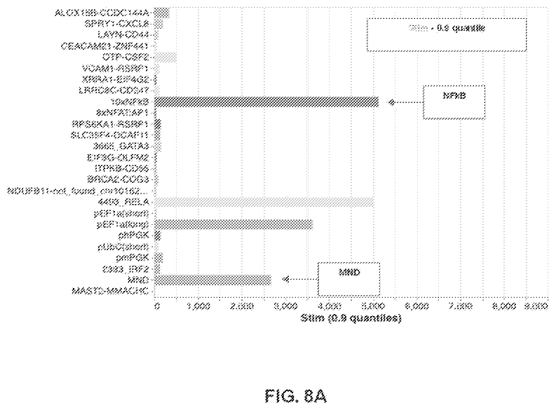

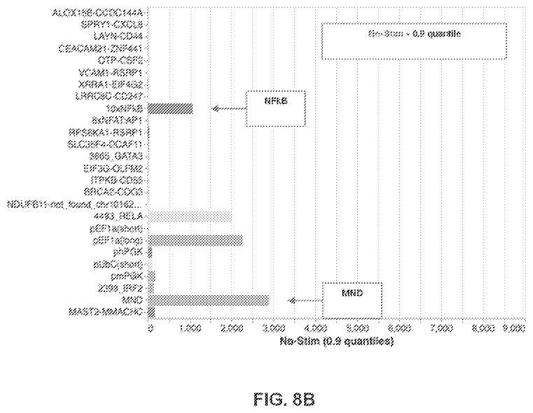

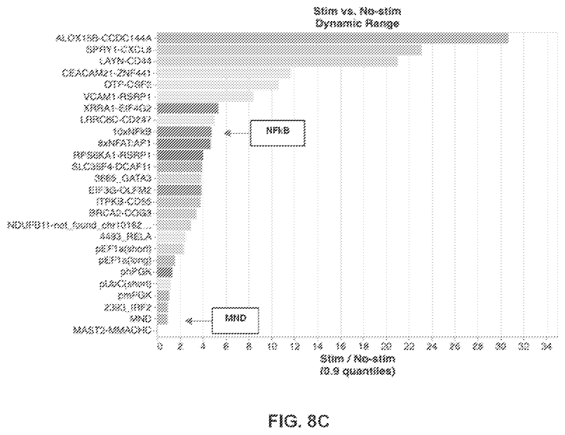

FIG. 8A-C provides a series of plots showing the dynamic range of EP-Pairings characterized according to the protocol described in FIG. 6A-B. FIG. 8A shows the maximal expression of each construct in this study, with annotations to the control constructs of MND and 10×NFkB. FIG. 8B shows the minimal expression of each construct in this study, with annotations to the control constructs of MND and 10×NFkB. FIG. 8C shows the range of expression of each construct in this study, with annotations to the control constructs of MND and 10×NFkB. In some embodiments, a construct with a greater dynamic range, including minimal expression under no stimulation conditions, is a desirable construct for stim-ON circuits.

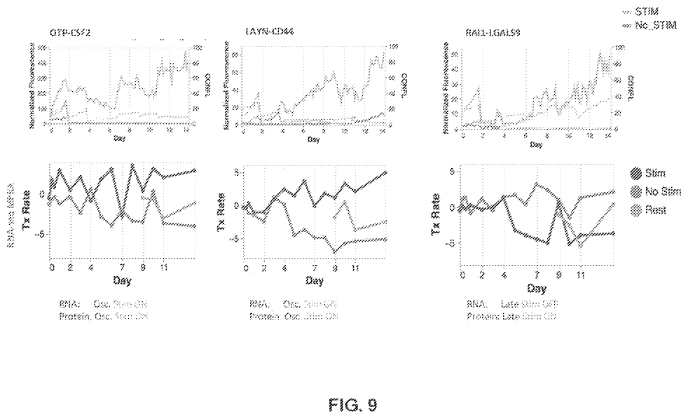

FIG. 9 is a series of diagrams depicting both RNA and protein produced from exemplary constructs comprising an EP-pairing of the disclosure. As shown in the right-hand panel, not all RNA and protein data align qualitatively. However, each construct of the disclosure can be functionally characterized with respect to transcriptional activity as a function of cell activity (e.g., contacting an antigen) or cell state (e.g., stimulation and/or exhaustion). In some embodiments, the presence or absence of a 5′ UTR sequence, or the composition of the 5′ UTR sequence, affects the translational behavior of a transcript produced under the control of a construct of the disclosure.

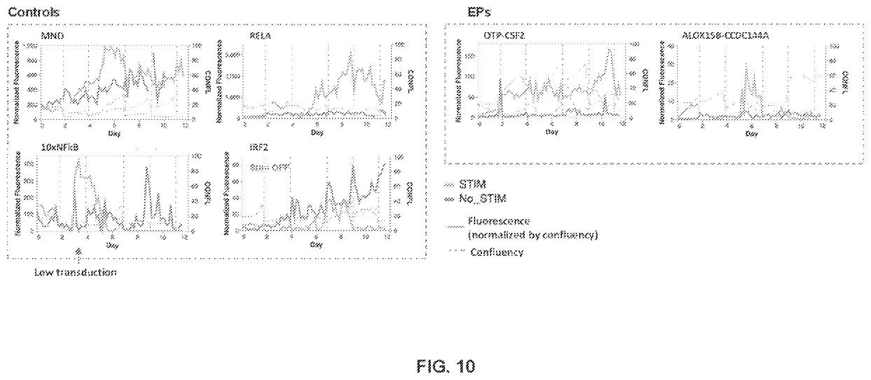

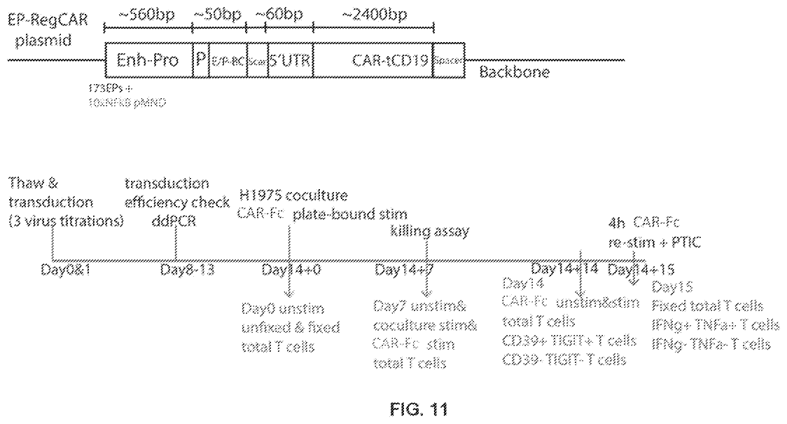

FIG. 10 is a repeat of the study performed in FIG. 9. Of note, with higher confluency compared the study of FIG. 9, this study provides data for the no-stimulation condition.

### Example 3: Functional Activity of EP-Pairs

As show in FIG. 11, a pooled screen was conducted with constructs comprising EP-pairs of the disclosure (see SEQ ID Nos 1-174), each driving expression of a “tool” CAR in a T-cell (top) and an experimental design for the screen (bottom). Control constructs include the constitutive MND promoter and the inducible 10×NFkB promoter.

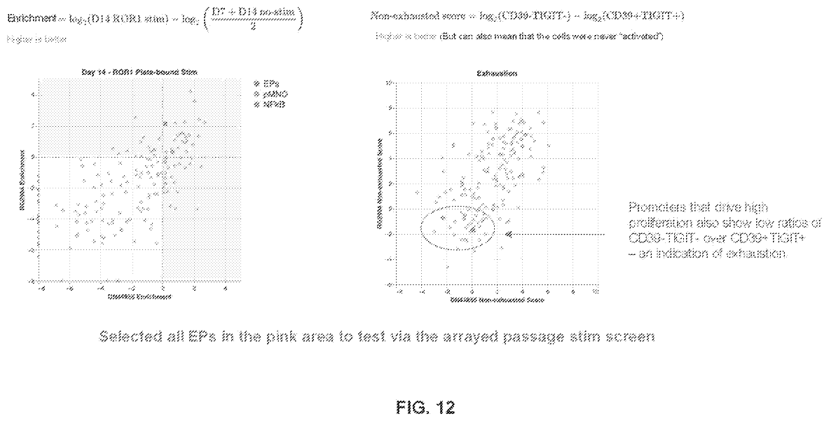

FIG. 12 is a pair of graphs showing the selection of candidate constructs for enrichment, based, in part, upon the negative selection of exhausted T-cells (identified as being CD39+TIGIT+, in this embodiment).

### Example 4: Target Cell Killing and Cytokine Production Assays

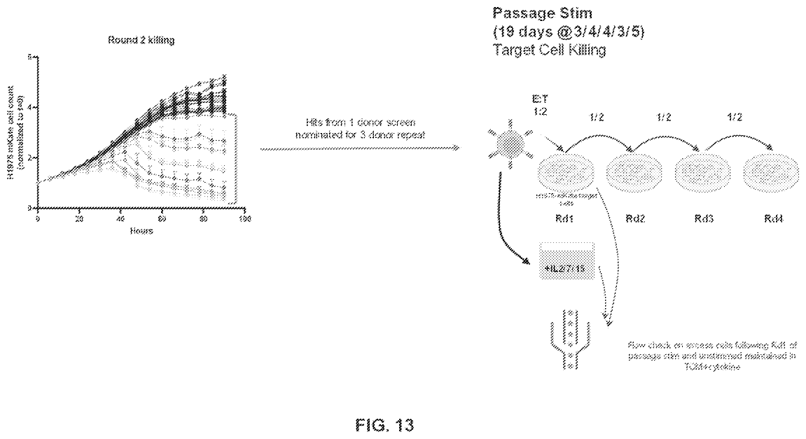

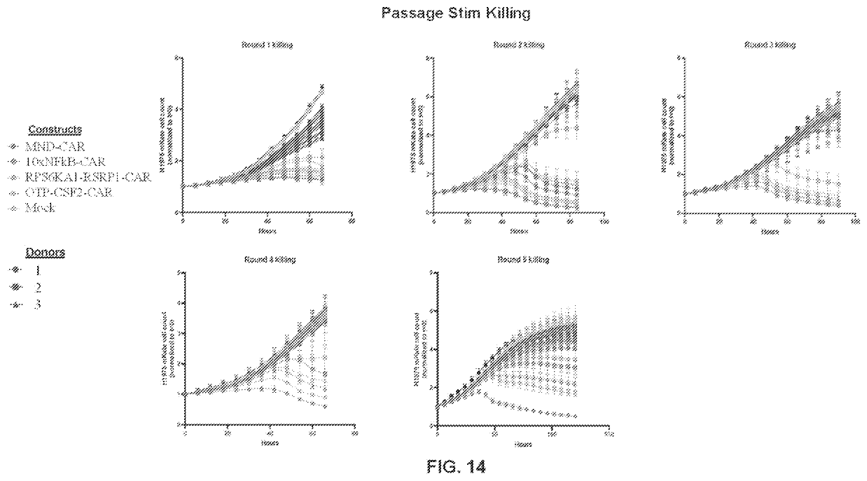

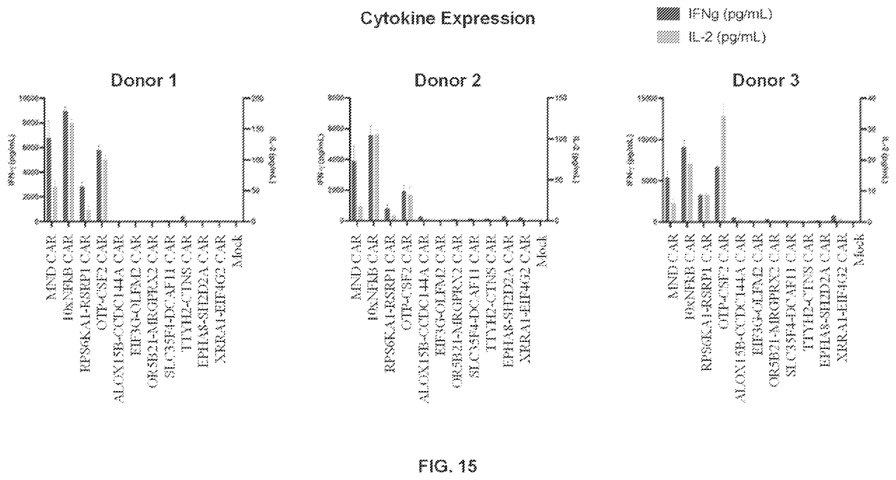

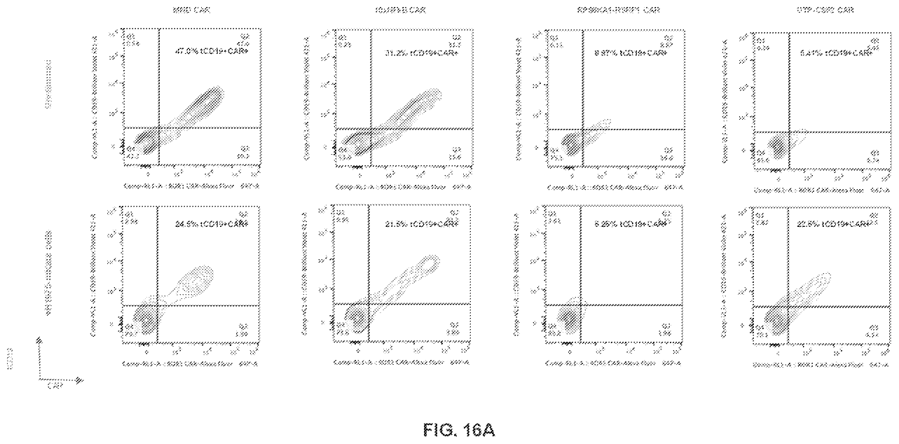

As shown in FIG. 13, a confirmational, three donor, assay was performed using the best performing EP-pairs identified in a preliminary screen using a single donor only. In the preliminary screen, the test constructs included 26 constructs comprising EP-Pairs driving a tool CAR construct, and four control constructs (MND and 10×NFkB promoters driving expressing of a tool CAR, each control promoter with and without a barcode). The preliminary screen was designed to identify those constructs that demonstrated most efficacious killing of a target cancer cell in vitro, compared to the activity of each of the control constructs. In this confirmational screen, 9 constructs comprising selected EP-Pairs driving a tool CAR construct (see FIGS. 14 and 15) and 2 control constructs were used (same as in the preliminary screen).

FIG. 14 is a series of graphs depicting target cell killing data from each round of the confirmational study described in FIG. 13. In each round, the ability of each construct to drive expression of a tool CAR and to kill a target cancer cell (H1975 mKate cells) is expressed as the target cell count normalized to time=zero as a function of time (expressed in hours). The data demonstrate that the EP Pairing of “OTP-CSF2” shows comparable killing to the construct comprising the control 10×NFkB promoter constructing driving expression of the same CAR. The data further show that the EP Pairing of “RPS6KA1-RSRP1” may be used a regulated alternative to the constitutive control promoter, MND.

FIG. 15 is a series of graphs depicting cytokine secretion (IL-2 and Interferon-gamma) from T-cells expressing each of the test constructs described in FIG. 13, in each of the three donors. The data demonstrate that the EP Pairings of “OTP-CSF2” and “RPS6KAT-RSRP1” show at least comparable functional activity to the control constructs having the MND and 10×NFkB promoters, respectively. Moreover, when the EP Pairing of “OTP-CSF2” drives expression of the CAR, the T-cell demonstrates higher IL-2 expression than when the the MND control drives expression of the same CAR. The EP Pairing of “RPS6KA1-RSRP1” demonstrates higher overall cytokine expression than the other seven constructs tested, which is consistent with the target cell killing data presented in FIG. 14.

### Example 5: Regulated CAR Circuits

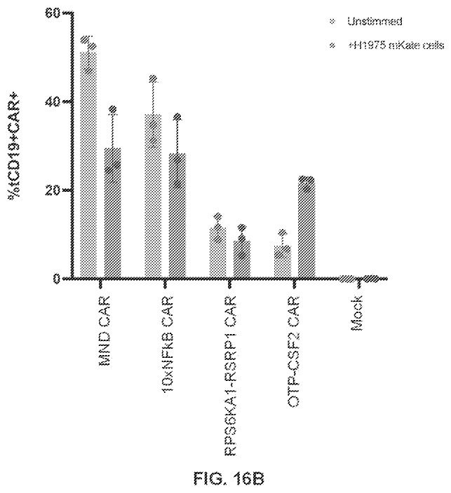

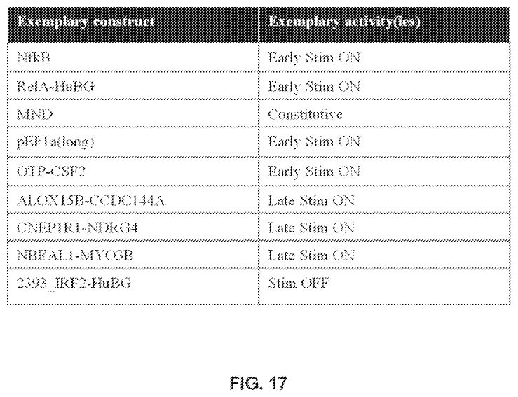

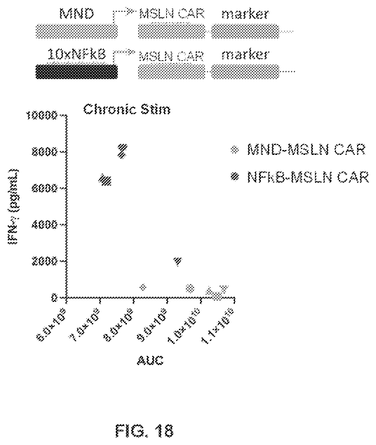

As shown in FIGS. 16A-16B and 18, EP-pairs of the disclosure inducibly regulate exogenous elements within a T-cell, including, for example, a CAR. The ability to regulate expression of various exogenous elements in response to changes of endogenous state leads to better functional outcomes.

CAR expression after one round of antigen stimulation for each of the control constructs (comprising MND and 10×NFkB, respectively) and two test constructs (comprising the EP Pairing of “OTP-CSF2” and “RPS6KA1-RSRP1”, respectively). FIG. 16A shows a series of cell sorting plots for both unstimulated (top) and stimulated (+H1975-mKate target cells, bottom), in which the levels of the CD19 marker are represented on the Y-axis and the levels of CAR expression are represented on the X-axis. See FIG. 11 for a schematic of the constructs/markers used. Shown in this figure is representative data from Donor 2. FIG. 16B is a graph showing the aggregate and quantitative data from all three donors in this study. For each condition and each construct, the data represent the Mean±SEM % tCD19+CAR+ across 3 donors. The data, taken together, demonstrate that (1) the “OTP-CSF2” construct shows Stim ON activity after 1 round of passage stimulation and that (2) “RPS6KA1-RSRP1” has different expression dynamics than the MND control construct, but a similar functional readout as the MND control construct.

FIG. 18 is a pair of schematic diagrams and a corresponding graph depicting exemplary uses for the constructs of the disclosure within cellular circuits. The data demonstrate that, under chronic stimulation conditions in vitro, the NFkB-regulated MSLN CAR shows improved killing and cytokine production when compared to a the same MSLN CAR under the control of MND constitutive expression.

1. A nucleic acid construct comprising a transcription initiator or the transcription initiator and a transcription regulator, wherein each of the transcription regulator and transcription initiator comprises a concatemer of transcription factor binding motifs that bind more than one unique transcription factor, or that bind the same transcription factor at more than one site, and wherein the construct is non-naturally occurring.

2. (canceled)

3. (canceled)

4. (canceled)

5. (canceled)

6. (canceled)

7. (canceled)

8. (canceled)

9. The construct of claim 1, wherein the concatemer comprises ten repeated sequences.

10. (canceled)

11. (canceled)

12. (canceled)

13. (canceled)

14. The construct of claim 9, wherein the concatemer comprises a linking sequence positioned between the repeated sequences.

15. (canceled)

16. The construct of claim 1, wherein the transcription regulator comprises a sequence isolated or derived from one or more of the untranslated genomic sequence of BACH2, BARX1, BATF, ELF1, ELF2, Elf4, Elk1, ERF, ETV1, Fli1, FOXP1, GABPA, GATA3, IRF1, IRF2, IRF5, IRF7, IRF9, MAF, MAFF, Maz, Mef2d, MLX, MYB, NFAT, NFATC3, NFkB, NR4A1, Nur77, PATZ1, REL, RELA, RORa, RORg, RORgt, STAT2, Tbox, TFEB, TOX, USF1, ZBTB2, ZKSCAN3, ZNF12, ZNF140, ZNF263, ZNF282, ZNF304, ZNF398, ZNF708, and ZNF75D.

17. (canceled)

18. (canceled)

19. (canceled)

20. The construct of claim 1, wherein the transcription regulator comprises sequences derived from the untranslated genomic sequence of RELA, comprising a sequence of any one or more of sequences of SEQ ID NO: 958-959, 3486-3501, 4426-4427, 4472-4473, 4484-4499, 1468-1469, 3996-4011, 4936-4937, 4982-4983, 4994-5009, 5697-5698, 8632-8647, 9572-9573, 9618-9619, and 9630-9645.

21. (canceled)

22. (canceled)

23. (canceled)

24. The construct of claim 1, wherein (a) the transcription regulator is inducible or (b) the construct is inducible.

25. The construct of claim 1, wherein the transcription initiator comprises a minimal promoter.

26. (canceled)

27. (canceled)

28. (canceled)

29. (canceled)

30. (canceled)

31. (canceled)

32. (canceled)

33. (canceled)

34. (canceled)

35. (canceled)

36. The construct of claim 1, wherein the transcription initiator comprises sequences derived from the untranslated genomic sequence of RELA, comprising a sequence of any one or more of sequences of SEQ ID NO: 4484-4499, 4994-5009, and 9630-9645.

37. (canceled)

38. (canceled)

39. (canceled)

40. (canceled)

41. The construct of claim 1, wherein the construct further comprises an integration sequence capable of inducing or increasing insertion of the construct into a target site.

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A vector comprising the construct of claim 1.

56. (canceled)

57. (canceled)

58. (canceled)

59. (canceled)

60. A library comprising the vector of claim 55.

61. (canceled)

62. (canceled)

63. (canceled)

64. A cell comprising
the construct of claim 1.

65. (canceled)

66. (canceled)

67. (canceled)

68. (canceled)

69. (canceled)

70. (canceled)

71. (canceled)

72. (canceled)

73. (canceled)

74. A method for selecting a sequence that responds to one or more of antigen receptor activation, cell stimulation, cell exhaustion, cell rest, and epigenetic regulation, the method comprising using the construct of claim 1.

75. (canceled)

76. (canceled)

77. (canceled)

78. (canceled)

79. A method for regulating transcription of an exogenous protein, in response to one or more of antigen receptor activation, cell stimulation, cell exhaustion, cell rest, and epigenetic regulation comprising using the construct of claim 1.

80. (canceled)

81. An inducible nucleic acid construct comprising a transcription regulating enhancer sequence and a transcription initiating promoter sequence (an EP-pairing), wherein the construct is non-naturally occurring.

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. A vector comprising the construct of claim 81.

115. (canceled)

116. (canceled)

117. (canceled)

118. (canceled)

119. (canceled)

120. A library comprising the construct of claim 81.

121. (canceled)

122. (canceled)

123. (canceled)

124. (canceled)

125. A cell comprising
the construct of claim 81.

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. An inducible construct comprising a nucleotide sequence encoding a circuit component, wherein the circuit component comprises
(a) a response component, wherein the response component is capable of modifying an activity of the effector component in response to:
(i) a signal from a receptor,
(ii) an epigenetic change,
(iii) an induction or a suppression of transcription, or
(iv) a change in cell state, and
(b) an effector component, wherein the response component is capable of inducing a change in an activity of the effector component; and
wherein the construct is non-naturally occurring.

141. (canceled)

142. (canceled)

143. (canceled)

144. (canceled)

145. (canceled)

146. (canceled)

147. (canceled)

148. (canceled)

149. (canceled)

150. (canceled)

151. (canceled)

152. (canceled)

153. (canceled)

154. (canceled)

155. (canceled)

156. A cell comprising the construct of claim 140.

157. (canceled)

158. (canceled)

159. (canceled)

160. (canceled)

161. (canceled)

162. (canceled)

163. (canceled)

164. (canceled)

165. (canceled)

166. (canceled)
